(19) United States
(12) Patent Application Publication
(10) Pub. No.: US 2006/0160851 A1
(43) Pub. Date: Jul. 20, 2006

(54) Substituted piperidine carbamates

(75) Inventors: Soren Ebdrup, Roskilde (DK); Per Vedso, Varlose (DK); Poul Jacobsen, Slangerup (DK); Flemming Elmelund Nielsen, Virum (DK); Johannes Cornelis de Jong, Bagsvaerd (DK)

Correspondence Address: NOVO NORDISK, INC.;PATENT DEPARTMENT, 100 COLLEGE ROAD WEST, PRINCETON, NJ, 08540, US

(73) Assignee: Novo Nordisk A/S, Bagsvard (DK)

(21) Appl. No.: 11/301,166

(22) Filed: Dec. 12, 2005

Related U.S. Application Data
(63) Continuation of application No. PCT/DK04/00398, filed on Jun. 10, 2004.

(30) Foreign Application Priority Data
Jun. 12, 2003 (DK) PA 2003 00880
Jun. 10, 2004 (DK) 2004/000398

(51) Int. Cl. A61K 31/4545; C07D 401/02

(52) U.S. Cl. 514/316; 514/318; 546/194; 546/189

(57) ABSTRACT

Novel substituted piperidine carbamates, pharmaceutical compositions comprising them and use thereof in the treatment and/or prevention of diseases and disorders related to hormone sensitive lipase. More particularly, the compounds are useful for the treatment and/or prevention of diseases and disorders in which modulation of the activity of hormone sensitive lipase is beneficial.

### CROSS-REFERENCE TO RELATED APPLICATIONS

This application is a continuation of International Application No. PCT/DK2004/000398, filed Jun. 10, 2004, which claims priority to Danish Patent Application No.: PA 2003 00880, filed Jun. 12, 2003, and U.S. Patent Application No. 60/478,561, filed Jun. 13, 2003.

### FIELD OF THE INVENTION

The present invention relates to novel substituted piperidine carbamates, to pharmaceutical compositions comprising these compounds, to the use of these compounds as pharmaceutical compositions, and to methods of treatment employing these compounds and compositions. The present compounds show inhibition of hormone sensitive lipase. As a result, the compounds have useful for the treatment and/or prevention of diseases and disorders related to hormone sensitive lipase.

### BACKGROUND OF THE INVENTION

The overall energy homeostasis of a mammalian system requires a high degree of regulation to ensure the availability of the appropriate substrate at the appropriate time. Plasma glucose levels rise during the post-prandial state, to return to pre-prandial levels within 2-3 hours. During these 2-3 hours, insulin promotes glucose uptake by skeletal muscle and adipose tissue and decreases the release of free fatty acids (FFA) from adipocytes, to ensure that the two substrates do not compete with each other. When plasma glucose levels fall, an elevation in plasma FFA is necessary to switch from glucose to fat utilization by the various tissues.

In individuals with insulin resistance, FFA levels do not fall in response to insulin, as they do in normal individuals, preventing the normal utilization of glucose by skeletal muscle, adipose and liver. Furthermore, there is a negative correlation between insulin sensitivity and plasma FFA levels.

Hormone-sensitive lipase (HSL) is an enzyme, expressed in adipose tissue, macrophages, muscle, adrenal, testis and islets (Kraemer and Shen, J. Lipid Res. 2002, 43, 1585-1594). In the adipocytes HSL catalyses the conversion of triglycerides to glycerol and fatty acids. It is through the regulation of this enzyme that the levels of circulating FFA are modulated. Insulin leads to the inactivation of HSL with a subsequent fall in plasma FFA levels during the post-prandial state, followed by the activation of the enzyme when the insulin concentration falls and catecholamines rise during the post-absorptive period. The activation of HSL leads to an increase in plasma FFA, as they become the main source of energy during fasting.

The activation-inactivation of HSL is primarily mediated through the cAMP-protein kinase A and AMP-dependent kinase pathways. There are compounds like nicotinic acid and its derivatives, that decrease the activation of HSL via these pathways and cause a decrease in lipolysis that leads to a reduction in the FFA levels. These drugs have a beneficial effect in the utilization of glucose and in the normalization of the excess triglyceride synthesis seen in patients with elevated FFA. However, since these pathways are used by other processes in the body, these drugs have severe side effects.

Piperidines have previously been prepared, and their properties have been investigated for a number of pharmaceutical applications, e.g. CNS disorders

Several publications disclose the preparation and use of HSL inhibitors (WO 01/87843, WO 01/17981, WO 01/66531, WO 01/83497, and WO 01/26664). However, the structures of these compounds are very different from that of the present compounds. Thus, none of the HSL inhibitors disclosed in these publications contain piperidine and carbamate substructures as in the compounds of the present invention.

We have found potent piperidine compounds that specifically inhibit the lipolytic activity of HSL and are expected to decrease plasma FFA levels. These compounds can be used to treat disorders where a decreased level of plasma FFA is desired, such as insulin resistance, syndrome X, dyslipidemia, abnormalities of lipoprotein metabolism.

One object of the present invention is to provide compounds and pharmaceutical compositions that inhibit the lipolytic activity of HSL. A further object is to provide compounds which have good pharmaceutical properties such as solubility, bioavailability, specificity etc.

### DEFINITIONS

The following is a detailed definition of the terms used to describe the compounds of the invention.

The term “halogen” in the present context designates an atom selected from the group consisting of F, Cl, Br and I.

The term “C₁₋₆alkyl” in the present context designates a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₂₋₆-alkyl” in the present context designates a saturated, branched or straight hydrocarbon group having from 2 to 6 carbon atoms. Representative examples include, but are not limited to, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆-alkoxy” in the present context designates a group —O—C₁₋₆-alkyl wherein C₁₋₆-alkyl is as defined above. Representative examples include, but are not limited to, methoxy, ethoxy, n-propoxy, isopropoxy, butoxy, isobutoxy, sec-butoxy, tert-butoxy, n-pentoxy, isopentoxy, neopentoxy, tert-pentoxy, n-hexoxy, isohexoxy and the like.

The term “C₂₋₆-alkenyl” as used herein, represent an olefinically unsaturated branched or straight hydrocarbon group having from 2 to 6 carbon atoms and at least one double bond. Examples of such groups include, but are not limited to, vinyl, 1-propenyl, 2-propenyl, allyl, iso-propenyl, 1,3-butadienyl, 1-butenyl, hexenyl, pentenyl and the like.

The term “C₃₋₁₀-cycloalkyl” as used herein represents a saturated mono-, bi-, tri- or spirocarbocyclic group having from 3 to 10 carbon atoms. Representative examples are cyclopropyl, cyclobutyl, cyclopentyl, cyclohexyl, cycloheptyl, cyclooctyl, cyclononyl, cyclodecyl, bicyclo[3.2.1]octyl, spiro[4.5]decyl, norpinyl, norbonyl, norcaryl, adamantyl and the like.

The term “C₃₋₈-heterocyclyl” as used herein represents a saturated 3 to 8 membered ring containing one or more heteroatoms selected from nitrogen, oxygen and sulfur. Representative examples are pyrrolidyl, piperidyl, piperazinyl, morpholinyl, thiomorpholinyl, aziridinyl, tetrahydrofuranyl and the like.

The term “aryl” as used herein represents a carbocyclic aromatic ring system being either monocyclic, bicyclic, or polycyclic, such as phenyl, biphenyl, naphthyl, anthracenyl, phenanthrenyl, fluorenyl, indenyl, pentalenyl, azulenyl, biphenylenyl and the like. Aryl is also intended to include the partially hydrogenated derivatives of the carbocyclic aromatic systems enumerated above. Non-limiting examples of such partially hydrogenated derivatives are 1,2,3,4-tetrahydronaphthyl, 1,4-dihydronaphthyl and the like.

The term “heteroaryl” as used herein represents a heterocyclic aromatic ring system containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuranyl, benzothiophenyl(thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the heterocyclic systems enumerated above. Non-limiting examples of such partially hydrogenated derivatives are 2,3-dihydrobenzofuranyl, 3,4-dihydroisoquinolinyl, pyrrolinyl, pyrazolinyl, indolinyl, oxazolidinyl, oxazolinyl, oxazepinyl and the like.

The term “perhalomethyl” as used herein designates a methyl moiety substituted with three halogen atoms. Non-limiting examples of perhalomethyl are CF₃, CCl₃, and CF₂Cl.

The term “perhalomethoxy” as used herein designates a perhalomethyl linked via an oxygen atom, e.g. —O—CF₃, —O—CCl₃, and —O—CF₂Cl

The term “ring system” as used herein includes aromatic as well as non-aromatic ring moieties, which may be monocyclic, bicyclic or polycyclic, and they encompass moieties with zero, one or more hetereatoms selected from nitrogen, oxygen and sulphur. Non-limiting examples of such ring systems are aryl, C₃₋₈-heterocyclyl and heteroaryl.

The term “heterocyclic system” as used herein includes aromatic as well as non-aromatic ring moieties, which may be monocyclic, bicyclic or polycyclic, and containing in their ring structure one or more heteroatoms selected from nitrogen, oxygen and sulfur. Non-limiting examples of such heterocyclic systems are C₃₋₈-heterocyclyl and heteroaryl.

Certain of the above defined terms may occur more than once in the structural formulae, and upon such occurrence each term shall be defined independently of the other.

The term “optionally substituted” as used herein means that the groups in question are either unsubstituted or substituted with one or more of the substituents specified. When the groups in question are substituted with more than one substituent the substituents may be the same or different.

The terms “disease”, “condition” and “disorder” as used herein are used interchangeably to specifiy a state of a patient which is not the normal physiological state of man.

The term “treatment” as used herein means the management and care of a patient having developed a disease, condition or disorder, as well as the management and care of an individual at risk of developing the disease, condition or disorder prior to the clinical onset of said disease, condition or disorder. The purpose of treatment is to combat the disease, condition or disorder, as well as to to combat the development of the disease, condition or disorder. Treatment includes the administration of the active compounds to prevent or delay the onset of the symptoms or complications and to eliminate or control the disease, condition or disorder as well as to alleviate the symptoms or complications associated with the disease, condition or disorder.

The term “effective amount” as used herein means a dosage which is sufficient in order for the treatment of the patient to be effective compared with no treatment.

The term “modulate” as used herein means to influence, i.e. to modulate a parameter means to influence that parameter in a desired way. Examples are to modulate insulin secretion from beta cells and to modulate the plasma level of free fatty acids.

The term “medicament” as used herein means a pharmaceutical composition suitable for administration of the pharmaceutically active compound to a patient.

The term “pharmaceutically acceptable” as used herein means suited for normal pharmaceutical applications, i.e. giving rise to no adverse events in patients etc.

### DESCRIPTION OF THE INVENTION

The present invention relates to compounds of the general formula I 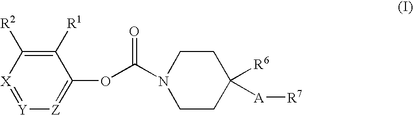
wherein

R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

X is N or C—R³; Y is N or C—R⁴; Z is N or C—R⁵;

R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

R⁶ is hydrogen or fluor;

A is selected from —O—, —S—, —S(═O)—, —S(═O)₂—, —CH₂O—, —CH₂S—, —CH₂CH₂—N(R⁸)—, —CH₂CH F—N(R⁸)—, —CH₂CF₂—N(R⁸)—, and —CHFCH₂—N(R⁸)—;

R⁸ is selected from hydrogen, C₁₋₆alkyl, C₂₋₆-alkenyl, aryl, heteroaryl, C₃₋₈-heterocyclyl and C₃₋₁₀-cycloalkyl, wherein each of C₁₋₆alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;

R⁷ is selected from aryl or heteroaryl, which may optionally be substituted with one or more substituents selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;

with the proviso that X, Y and Z are not all CH;

as well as diastereomers, enantiomers or tautomeric forms thereof including mixtures of these, pharmaceutically acceptable salts thereof, pharmaceutically acceptable solvates thereof, or polymorphs.

The present invention relates to compounds of the general formula I 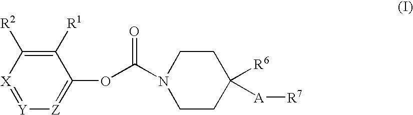
wherein

R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

X is N or C—R³; Y is N or C—R⁴; Z is N or C—R⁵;

R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

R⁶ is hydrogen or fluor;

A is selected from —O—, —S—, —S(═O)—, —S(═O)₂—, —CH₂O—, —CH₂S—, —CH₂CH₂—N(R⁸)—, —CH₂CHF—N(R⁸)—, —CH₂CF₂—N(R⁸)—, and —CHFCH₂—N(R⁸)—;

R⁸ is selected from hydrogen, C₁₋₆-alkyl, C₂₋₆-alkenyl, aryl, heteroaryl, C₃₋₈-heterocyclyl and C₃₋₁₀-cycloalkyl, wherein each of C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;

R⁷ is selected from aryl or heteroaryl, which may optionally be substituted with one or more substituents selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆alkyl, perhalomethyl and perhalomethoxy;

with the proviso that X, Y and Z are not all CH;

and with the proviso that said compound is not 4-(2-phenylethyl)-piperidine-1-carboxylic acid 3-(4-methyl-1-piperazinyl)phenyl ester or 4-(2-Pyrrolidin-1-yl-ethyl)-piperidine-1-carboxylic acid 4-(5-trifluoromethyl-pyridin-2-yloxy)-phenyl ester;

as well as diastereomers, enantiomers or tautomeric forms thereof including mixtures of these, pharmaceutically acceptable salts thereof, pharmaceutically acceptable solvates thereof, or polymorphs.

In another aspect the present invention relates to a compound of the general formula (I): 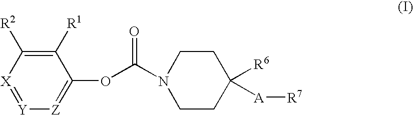
wherein

R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆alkoxy, C₂₋₆-alkenyl, aryl, heteroaryl, C₃₋₈-heterocyclyl, and C₃₋₁₀-cycloalkyl;

X is N or C—R³; Y is N or C—R⁴; Z is N or C—R⁵;

R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl; R⁶ is hydrogen or fluor;

A is —CH₂—;

R⁸ is selected from hydrogen, C₁₋₆-alkyl, C₂₋₆-alkenyl, aryl, heteroaryl, C₃₋₈-heterocyclyl and C₃₋₁₀-cycloalkyl, wherein each of C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is option substituted with one or more substituents independently selected from hydroxy, sulfanyl, oxo, halogen, amino, C₁₋₆-alkyl, perhalomethyl and perhalomethoxy;

R⁷ is selected from aryl or heteroaryl, which may optionally be substituted with one or more substituents selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;

with the proviso that X, Y and Z are not all CH;

and with the proviso that said compound is not

1-Piperidinecarboxylic acid, 4-(phenylmethyl)-, 4-nitrophenyl ester,

4-Benzylpiperidine-1-carboxylic acid 4-(5-trifluoromethylpyridin-2-yloxy)phenyl ester, 1-Piperidinecarboxylic acid, 4-[[4-[(dimethylamino)methyl]phenyl]methyl]-, 4-methylphenyl ester, or 1-Piperidinecarboxylic acid, 4-[[4-(1-piperidinylmethyl)phenyl]methyl]-, 4-chlorophenyl ester

as well as diastereomers, enantiomers or tautomeric forms thereof including mixtures of these, pharmaceutically acceptable salts thereof, pharmaceutically acceptable solvates thereof, or polymorphs.

In one embodiment the invention is concerned with compounds wherein R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

In another embodiment the invention is concerned with compounds wherein R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl.

In another embodiment the invention is concerned with compounds wherein R¹ is hydrogen.

In another embodiment the invention is concerned with compounds wherein R² is hydrogen.

In another embodiment the invention is concerned with compounds wherein R¹ is hydrogen and R² is hydrogen.

In another embodiment the invention is concerned with compounds wherein R² is selected from the group consisting of

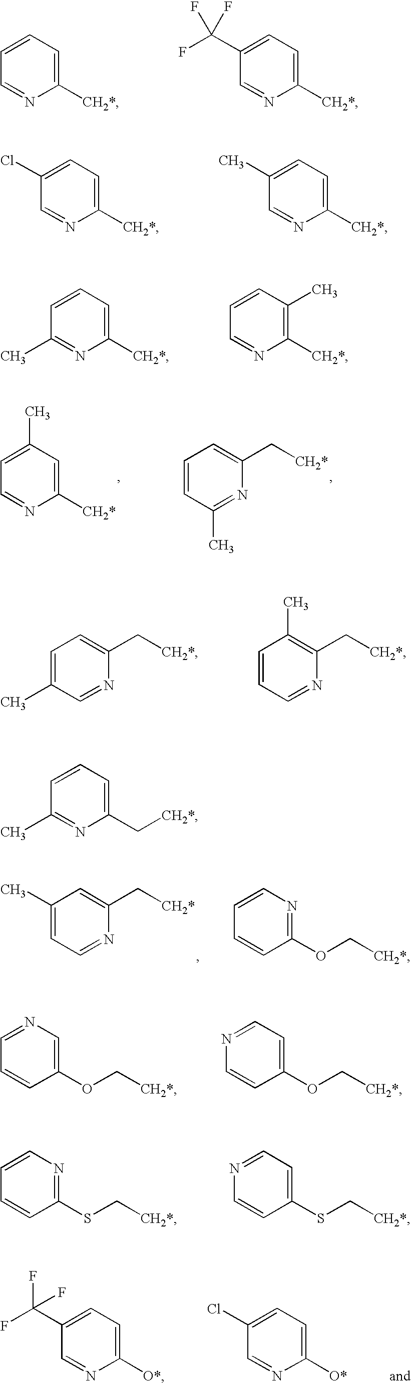

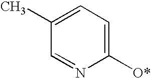

In another embodiment the invention is concerned with compounds wherein R ² is selected from the group consisting of

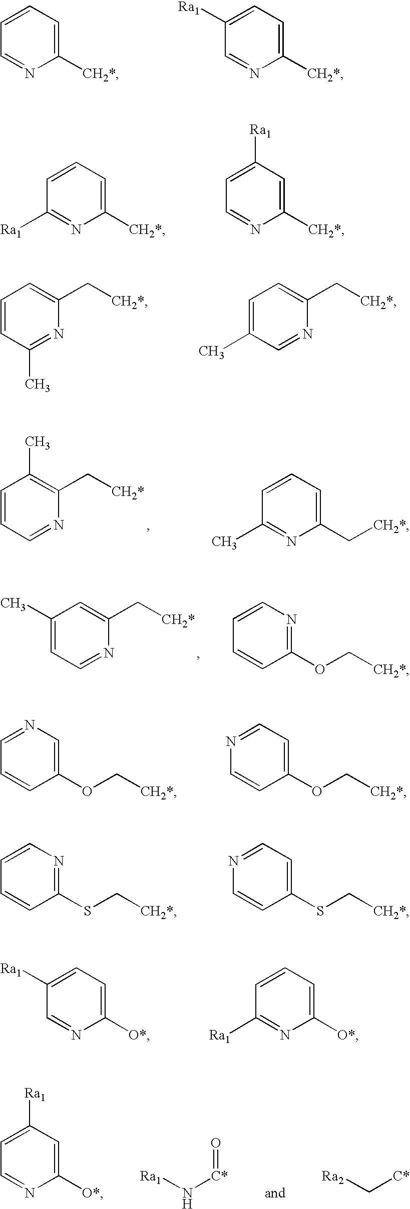

wherein Ra1 is selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

wherein Ra2 is selected from C₁₋₆-alkoxy, wherein C₁₋₆-alkoxy may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

In another embodiment the invention is concerned with compounds wherein R² is selected from the group consisting

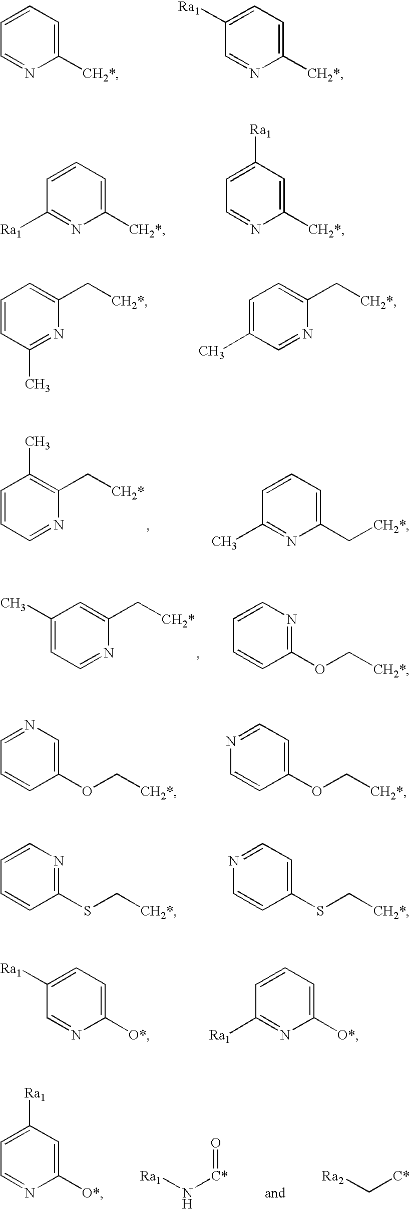

wherein Ra1 is selected from sulfanyl, halogen, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

wherein Ra2 is selected from C₁₋₆-alkoxy, wherein C₁₋₆-alkoxy may optionally be substituted with one or more substituents independently selected from hydroxy, sulfanyl, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

In another embodiment the invention is concerned with compounds wherein R² is selected from the group consisting of

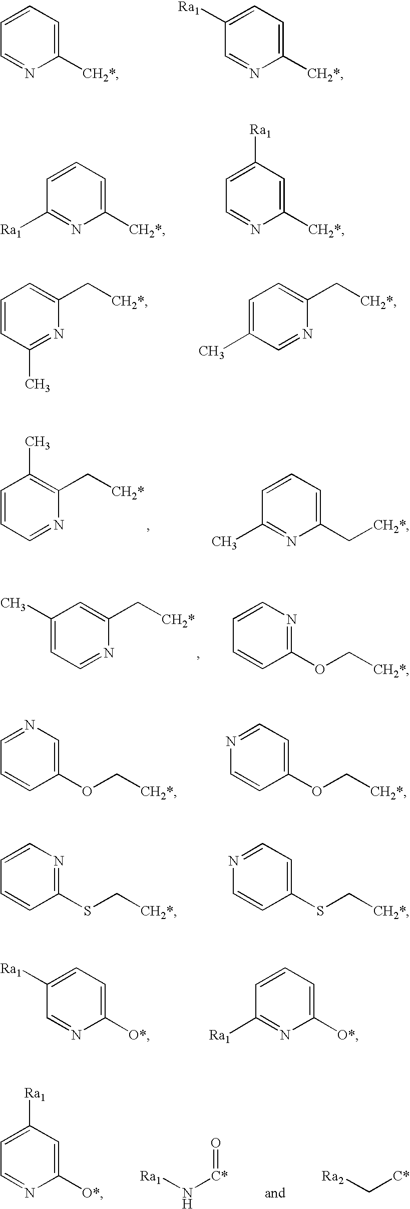

wherein Ra1 is selected from halogen, perhalomethyl, C₁₋₆alkyl, C₁₋₆alkoxy, C₂₋₆-alkenyl, heteroaryl, C₃₋₈-heterocyclyl, and C₃₋₁₀-cycloalkyl;

wherein Ra2 is selected from C₁₋₆alkoxy, wherein C₁₋₆-alkoxy may optionally be substituted with one or more substituents independently selected from hydroxy, halogen, amino, perhalomethyl, C₁₋₆alkyl, C₁₋₆alkoxy, C₂₋₆-alkenyl, heteroaryl, C₃₋₈-heterocyclyl, and C₃₋₁₀-cycloalkyl;

In another embodiment the invention is concerned with compounds wherein R² is selected from the group consisting of

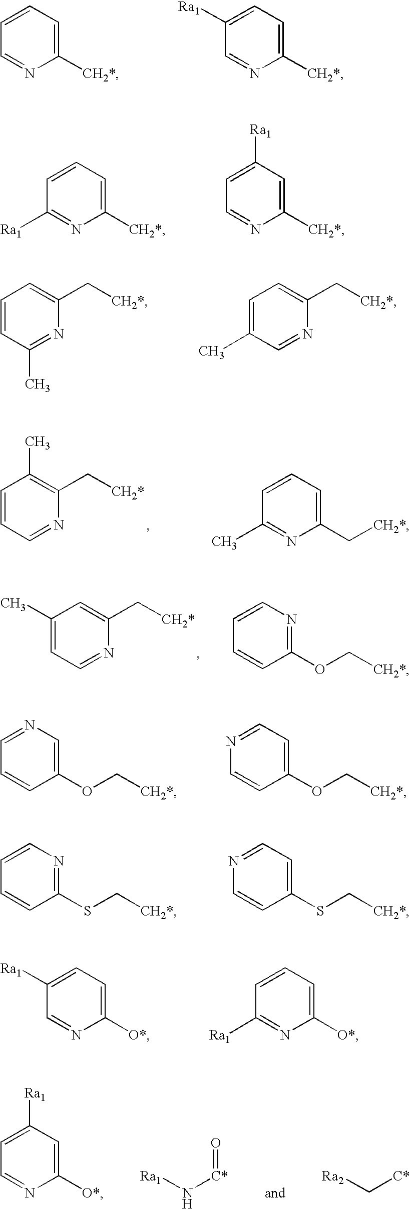

wherein Ra1 is selected from halogen, perhalomethyl, C₁₋₆-alkyl, C₁₋₆-alkoxy, heteroaryl, and C₃₋₈-heterocyclyl;

wherein Ra2 is selected from C₁₋₃₋alkoxy, wherein C₁₋₃-alkoxy may optionally be substituted with one or more substituents independently selected from hydroxy, halogen, amino, C₁₋₆-alkyl, C₁₋₆-alkoxy, heteroaryl and C₃₋₈-heterocyclyl;

In another embodiment the invention is concerned with compounds wherein R² is selected from the group consisting of

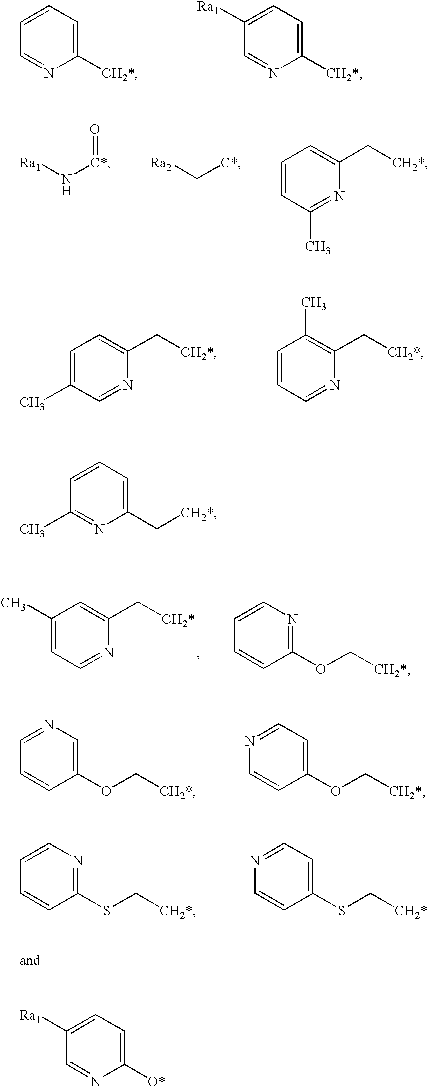

wherein Ra1 is selected from halogen, perhalomethyl, C₁₋₆alkyl, C₁₋₆-alkoxy, heteroaryl, and C₃₋₈-heterocyclyl;

wherein Ra2 is selected from C₁₋₃-alkoxy, wherein C₁₋₃-alkoxy may optionally be substituted with one or more substituents independently selected from hydroxy, halogen, amino, C₁₋₆-alkyl, C₁₋₆-alkoxy, heteroaryl and C₃₋₈-heterocyclyl;

In another embodiment the invention is concerned with compounds wherein R² is selected from the group consisting of

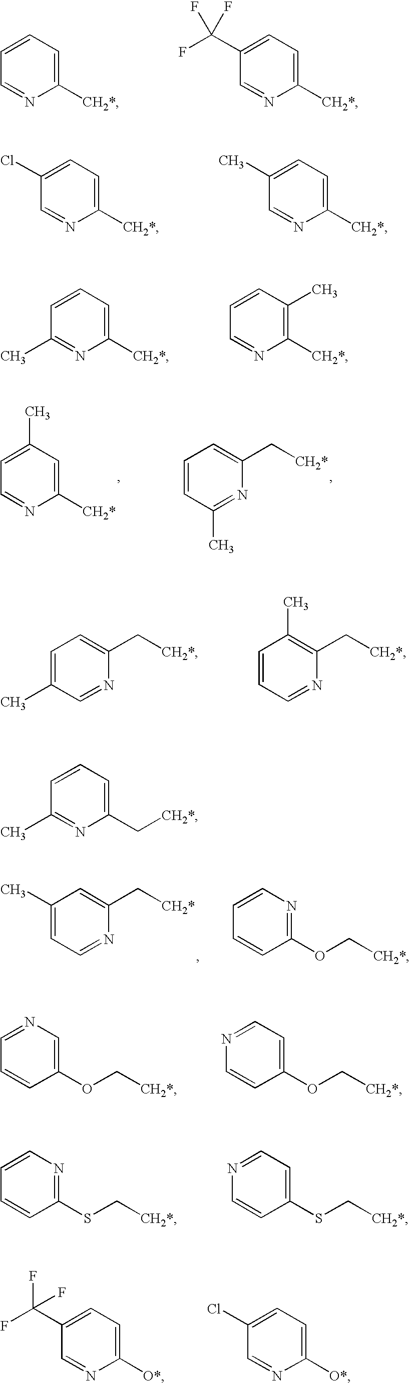

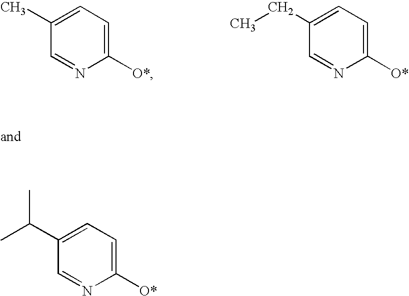

In another embodiment the invention is concerned with compounds wherein R² is selected from the group consisting of

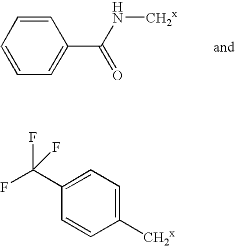

In another embodiment the invention is concerned with compounds wherein R² is selected from the group consisting of

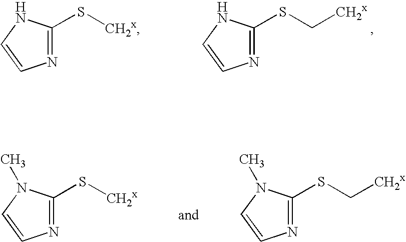

In another embodiment the invention is concerned with compounds wherein R² is selected from the group consisting of

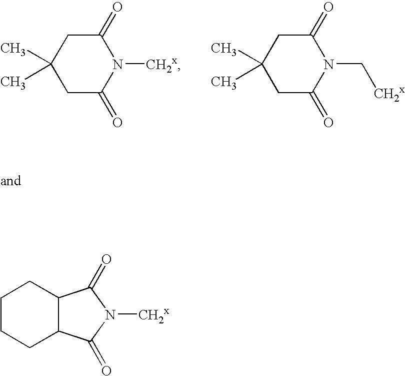

In another embodiment the invention is concerned with compounds wherein X is N.

In another embodiment the invention is concerned with compounds wherein X is CH.

In another embodiment the invention is concerned with compounds wherein X is C—R³.

In another embodiment the invention is concerned with compounds wherein R³ is selected from the group consisting of

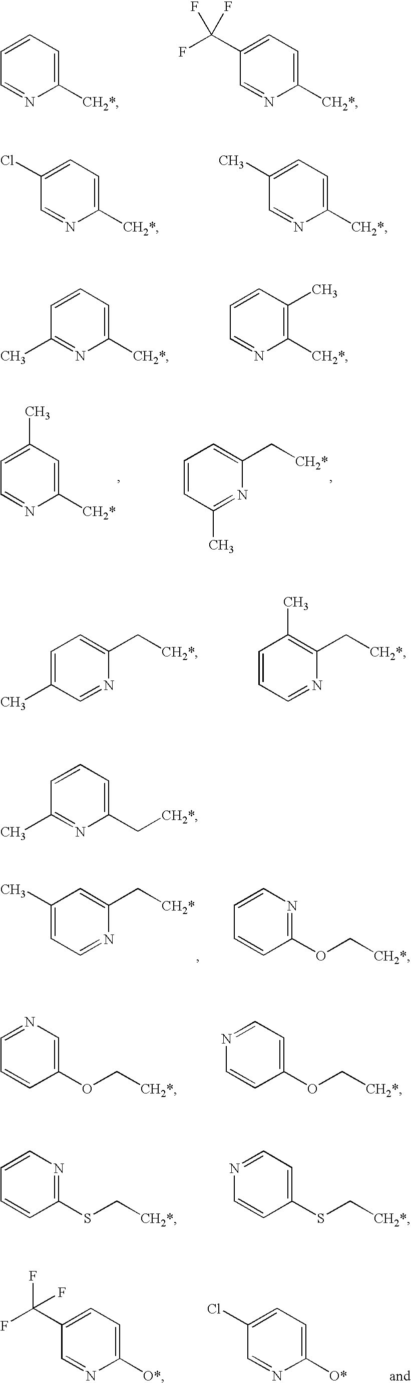

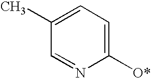

In another embodiment the invention is concerned with compounds wherein R³ is selected from the group consisting of

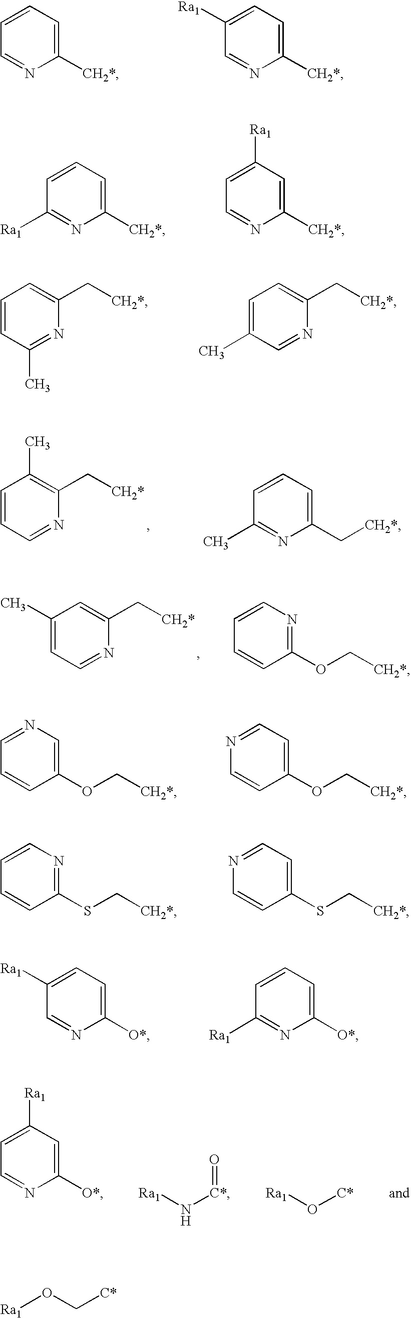

wherein Ra1 is selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;

In another embodiment the invention is concerned with compounds wherein R³ is selected from the group consisting of

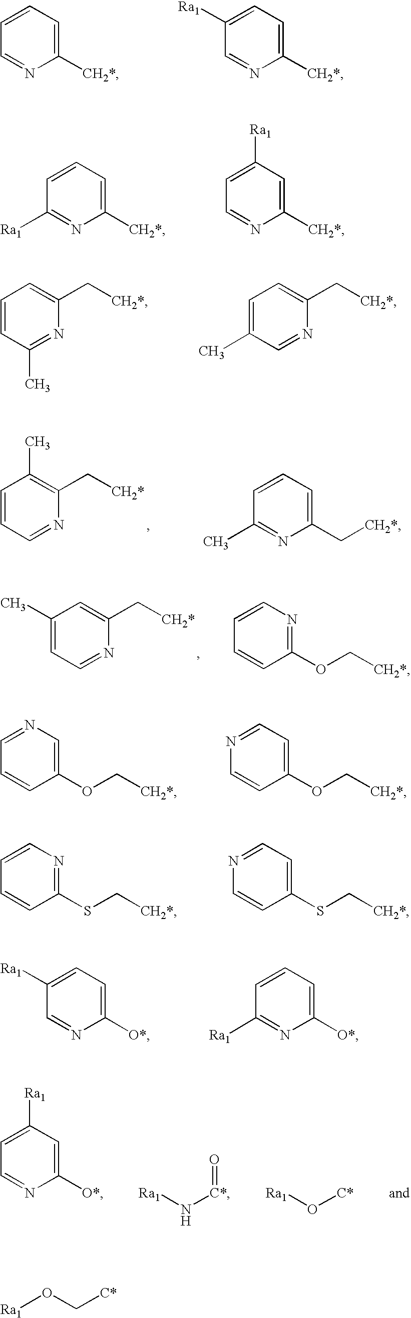

wherein Ra1 is selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, halogen, amino, perhalomethyl, perhalomethoxy, C₁₋₆-alkyl, C₁₋₆-alkoxy, aryl, heteroaryl, C₃₋₈-heterocyclyl, and C₃₋₁₀-cycloalkyl;

In another embodiment the invention is concerned with compounds wherein R³ is selected from the group consisting of

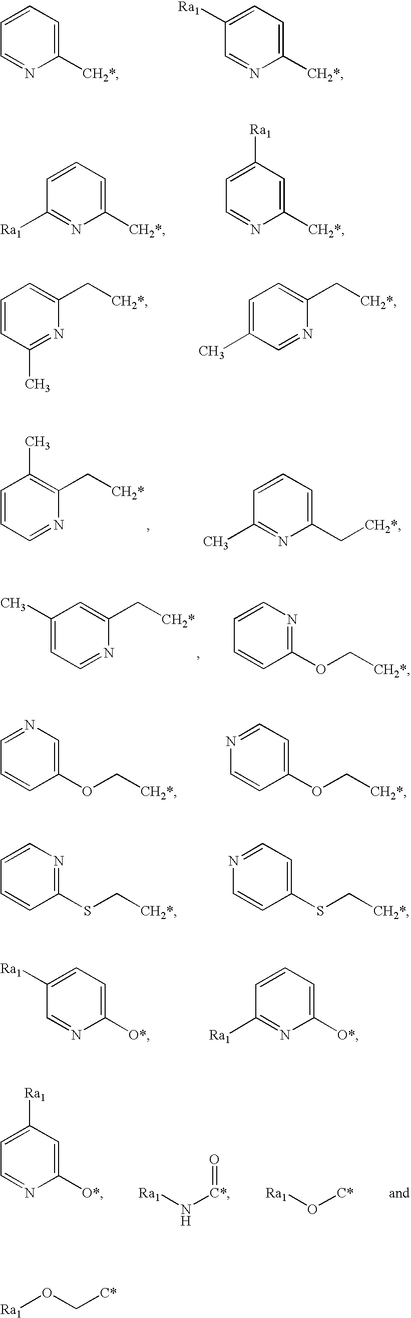

wherein Ra1 is selected from sulfanyl, halogen, amino, sulfo, C₁₋₆-alkyl, C₂₋₆-alkenyl, aryl, heteroaryl, C₃₋₈heterocyclyl and C₃₋₁₀-cycloalkyl, wherein each of sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₈-heterocyclyl, and C₃₋₇-cycloalkyl;

In another embodiment the invention is concerned with compounds wherein R³ is selected from the group consisting of

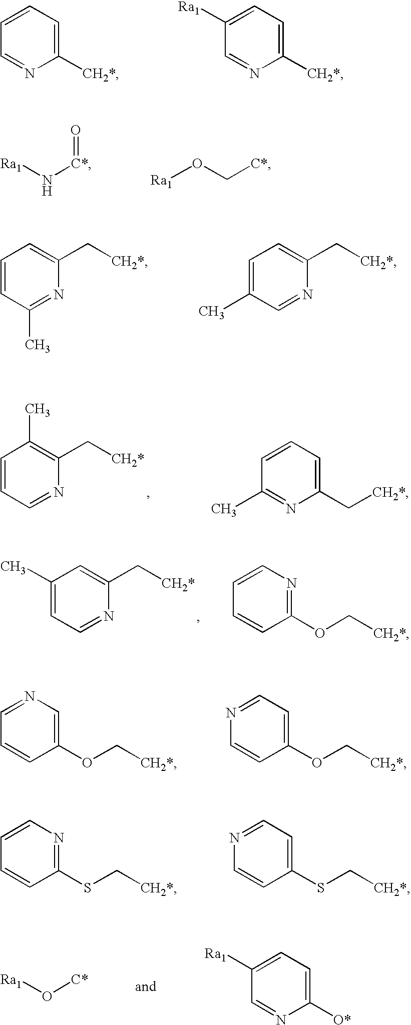

wherein Ra1 is selected from sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₈-heterocyclyl, and C₃₋₇-cycloalkyl;

In another embodiment the invention is concerned with compounds wherein R³ is selected from the group consisting of

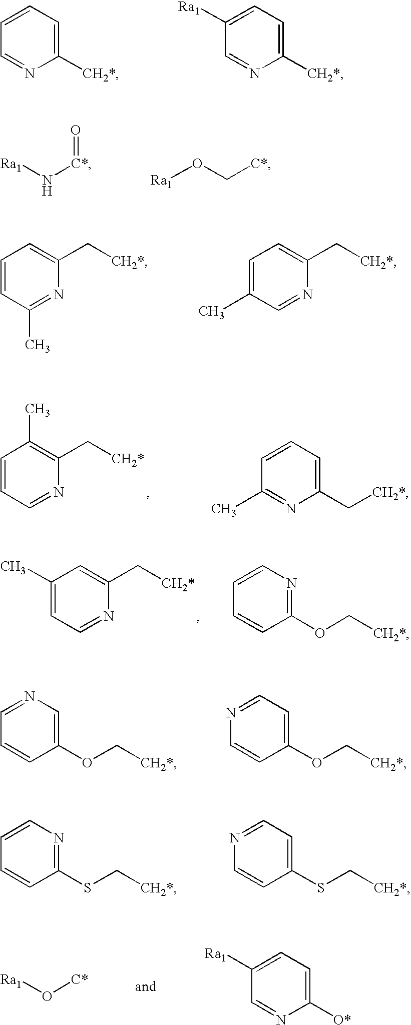

wherein Ra1 is selected from sulfanyl, halogen, C₁₋₆-alkyl, aryl, heteroaryl, C₃₋₈-heterocyclyl and C₃₋₇-cycloalkyl, wherein each of sulfanyl, C₁₋₆-alkyl, aryl, heteroaryl, C₃₋₈-heterocyclyl and C₃₋₇-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₈-heterocyclyl, and C₃₋₇-cycloalkyl;

In another embodiment the invention is concerned with compounds wherein R³ is selected from the group consisting of

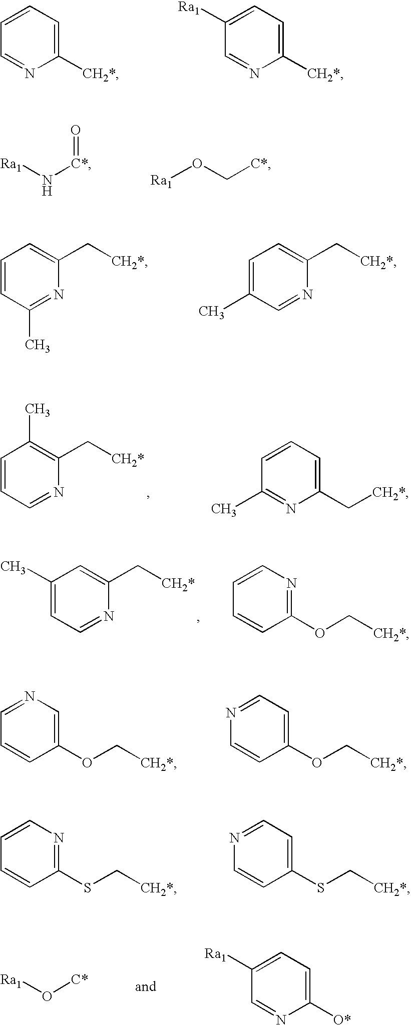

wherein Ra1 is selected from halogen, C₁₋₆alkyl, aryl, heteroaryl, C₃₋₆-heterocyclyl and C₃₋₄-cycloalkyl, wherein each of C₁₋₆alkyl, aryl, heteroaryl, C₃₋₆-heterocyclyl and C₃₋₄-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₆-heterocyclyl, and C₃₋₆-cycloalkyl;

In another embodiment the invention is concerned with compounds wherein R³ is selected from the group consisting of

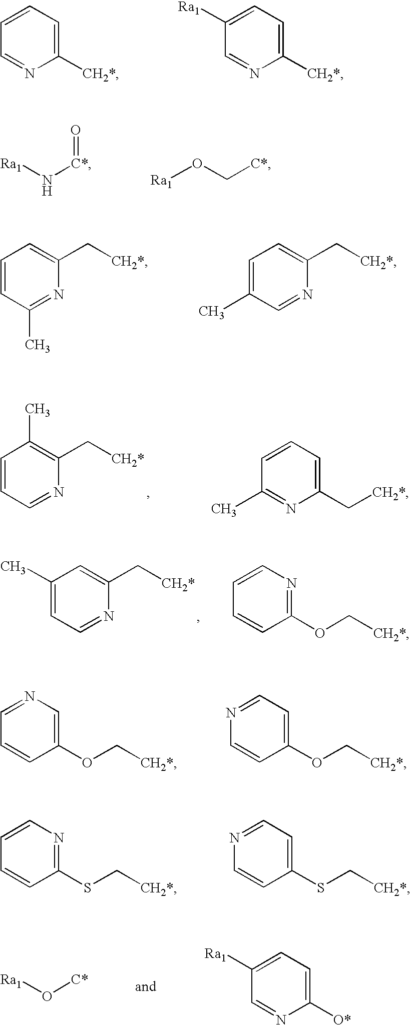

wherein Ra1 is selected from halogen, C₁₋₆-alkyl, aryl, heteroaryl, C₃₋₆-heterocyclyl and C₃₋₄-cycloalkyl, wherein each of C₁₋₆-alkyl, aryl, heteroaryl may optionally be substituted with one or more substituents independently selected from hydroxy, halogen, perhalomethyl, perhalomethoxy, C₁₋₄-alkyl, C₁₋₄-alkoxy, C₃₋₆-heterocyclyl, and C₃₋₆-cycloalkyl;

In another embodiment the invention is concerned with compounds wherein R³ is selected from the group consisting of

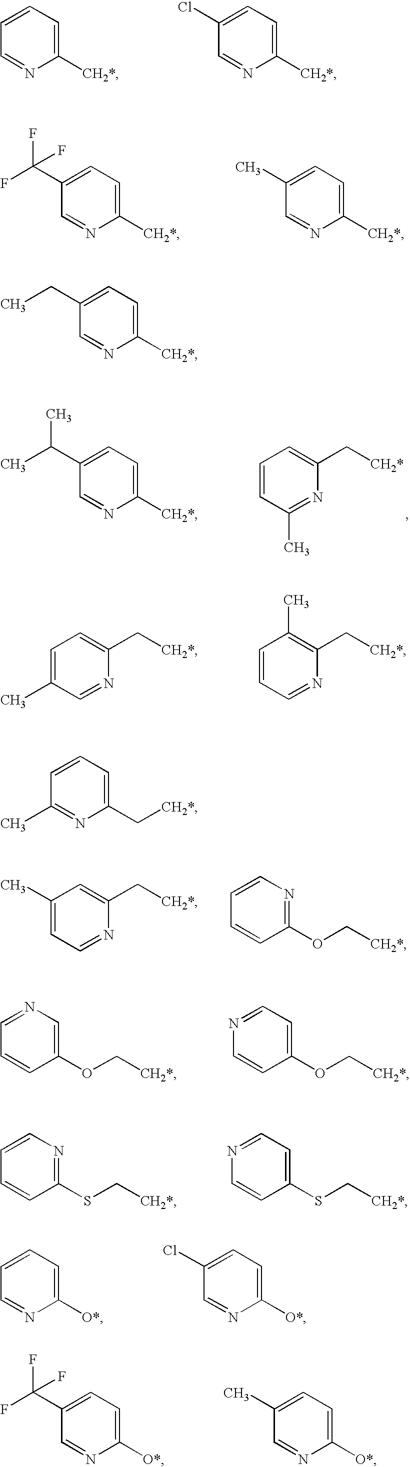

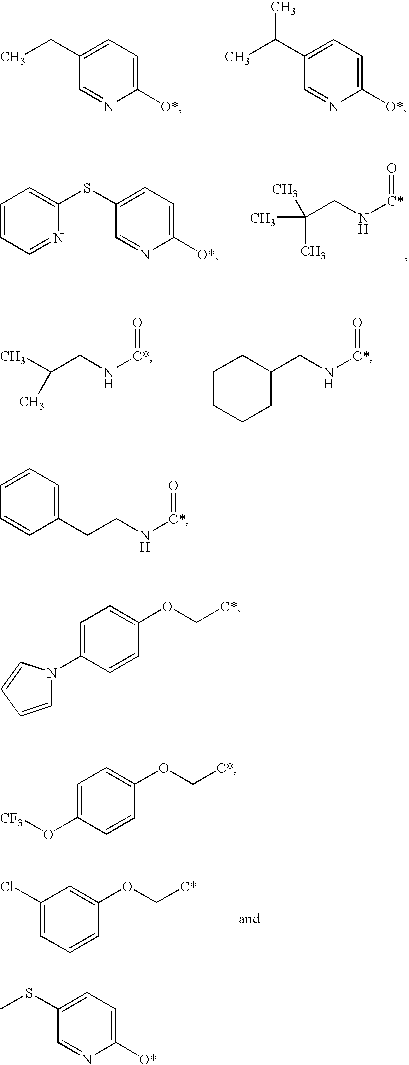

In another embodiment the invention is concerned with compounds wherein R³ is selected from the group consisting of

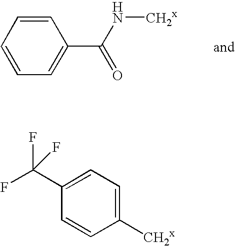

In another embodiment the invention is concerned with compounds wherein R³ is selected from the group consisting of

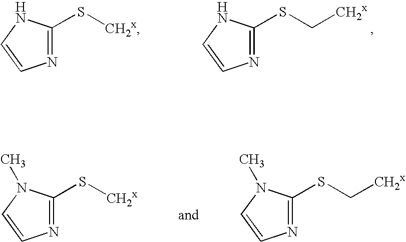

In another embodiment the invention is concerned with compounds wherein R³ is selected from the group consisting of

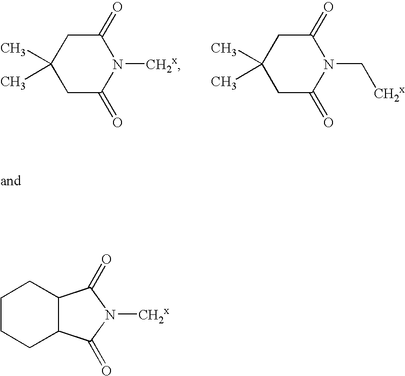

In another embodiment the invention is concerned with compounds wherein R³ is selected from the group consisting of

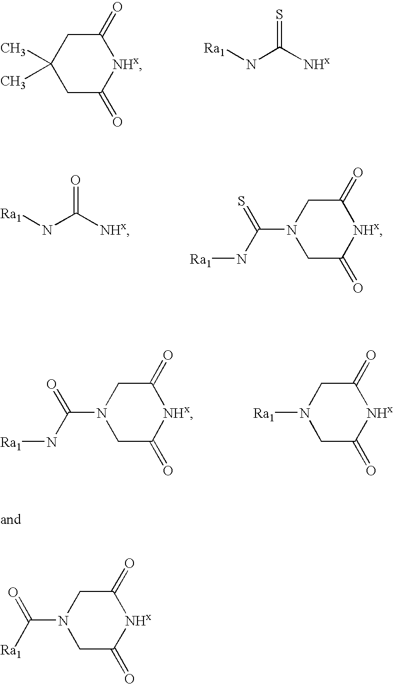

wherein Ra₁ is selected from C₁₋₆-alkyl, aryl, heteroaryl, C₃₋₆-heterocyclyl and C₃₋₇-cycloalkyl.

In another embodiment the invention is concerned with compounds wherein Z is N.

In another embodiment the invention is concerned with compounds wherein Z is C—R⁵.

In another embodiment the invention is concerned with compounds wherein Z is CH.

In another embodiment the invention is concerned with compounds wherein Y is N.

In another embodiment the invention is concerned with compounds wherein Y is CH.

In another embodiment the invention is concerned with compounds wherein Y is C—R⁴.

In another embodiment the invention is concerned with compounds wherein only one of X, Y and Z is N.

In another embodiment the invention is concerned with compounds wherein X is C—R³, Y is C—R⁴ and Z is C—R⁵.

In another embodiment the invention is concerned with compounds wherein Y and Z are both CH.

In another embodiment the invention is concerned with compounds wherein X is C—R³.

In another embodiment the invention is concerned with compounds wherein R³ is selected from the group consisting of

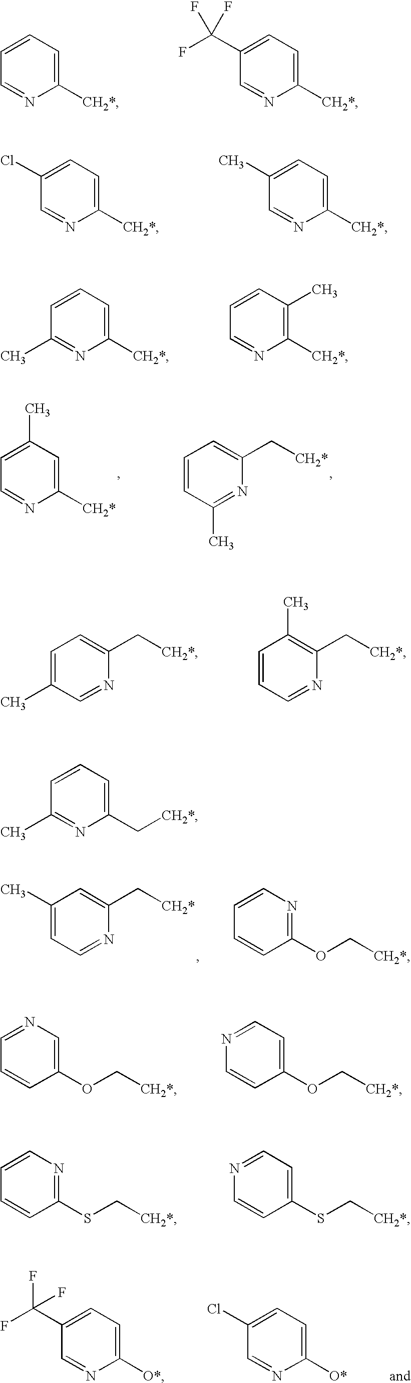

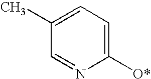

In another embodiment the invention is concerned with compounds wherein R³ is selected from the group consisting of

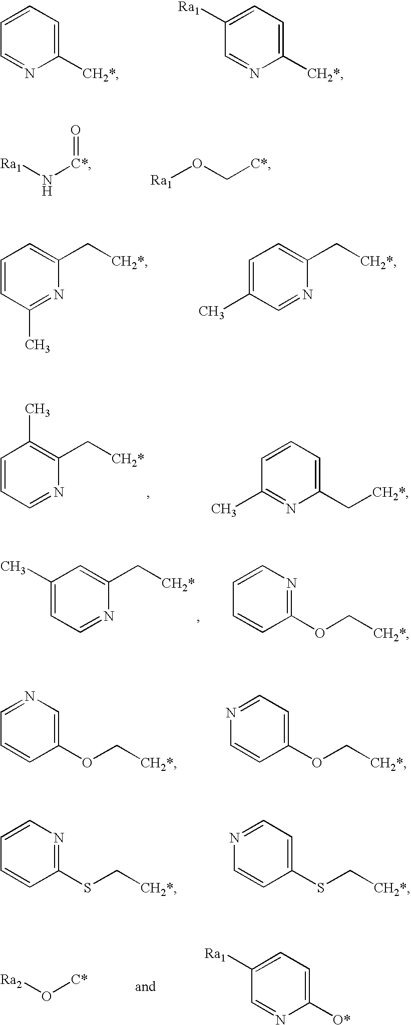

wherein Ra1 is selected from halogen, C₁₋₆-alkyl, aryl, heteroaryl, C₃₋₆-heterocyclyl and C₃₋₄-cycloalkyl, wherein each of C₁₋₆alkyl, aryl, heteroaryl, C₃₋₆-heterocyclyl and C₃₋₄-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₆-heterocyclyl, and C₃₋₆-cycloalkyl;

wherein Ra2 is selected from C₁₋₆-alkyl, aryl, heteroaryl, C₃₋₆-heterocyclyl and C₃₋₇-cycloalkyl, wherein each of C₁₋₆-alkyl, aryl, heteroaryl, C₃₋₆-heterocyclyl and C₃₋₇-cycloalkyl may optionally be substituted with one or more substituents independently selected from hydroxy, halogen, perhalomethyl, perhalomethoxy, C₁₋₄-alkyl, C₁₋₄-alkoxy, aryl, heteroaryl, C₃₋₆-heterocyclyl, and C₃₋₆-cycloalkyl;

In another embodiment the invention is concerned with compounds wherein R³ is selected from the group consisting of

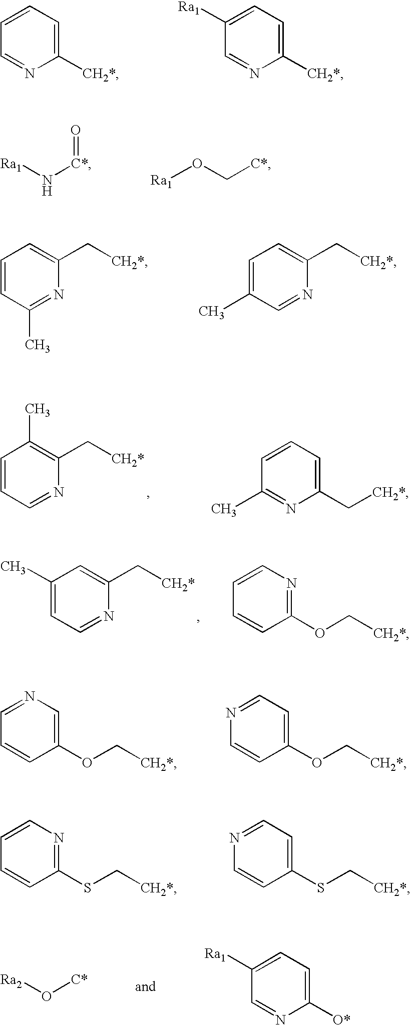

wherein Ra1 is selected from halogen, C₁₋₆-alkyl, aryl, heteroaryl, C₃₋₆-heterocyclyl and C₃₋₄-cycloalkyl, wherein each of C₁₋₆alkyl, aryl, heteroaryl may optionally be substituted with one or more substituents independently selected from hydroxy, halogen, perhalomethyl, perhalomethoxy, C₁₋₄-alkyl, C₁₋₄-alkoxy, C₃₋₆-heterocyclyl, and C₃₋₆-cycloalkyl;

wherein Ra2 is selected from C₁₋₆-alkyl, aryl, heteroaryl, C₃₋₆′-heterocyclyl and C₃₋₇-cycloalkyl, wherein each of C₁₋₆-alkyl, aryl, heteroaryl may optionally be substituted with one or more substituents independently selected from hydroxy, halogen, perhalomethyl, perhalomethoxy, C₁₋₄-alkyl, C₁₋₄-alkoxy, C₃₋₆-heterocyclyl, and C₃₋₆-cycloalkyl;

In another embodiment the invention is concerned with compounds wherein R³ is selected from the group consisting of

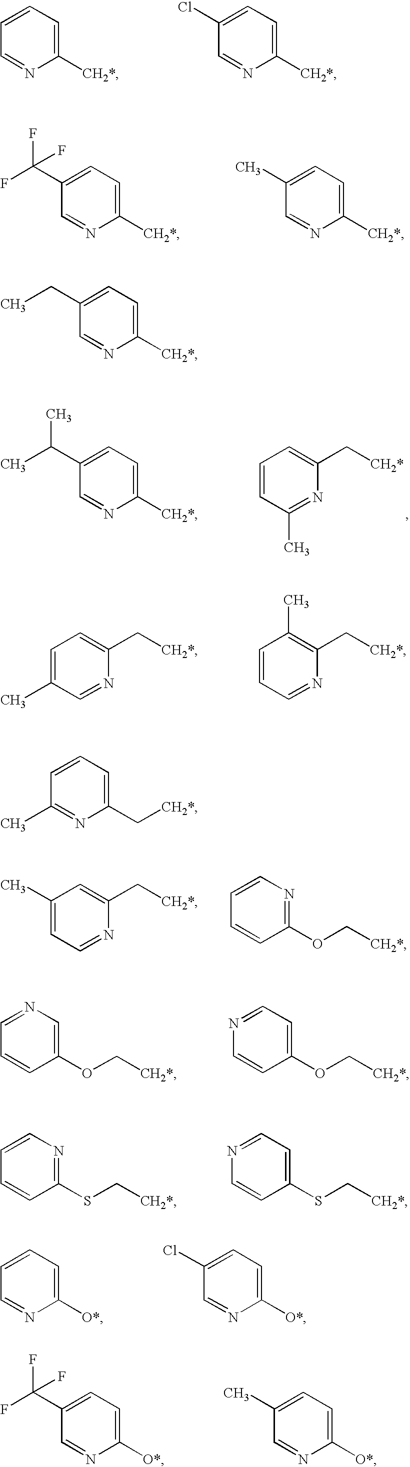

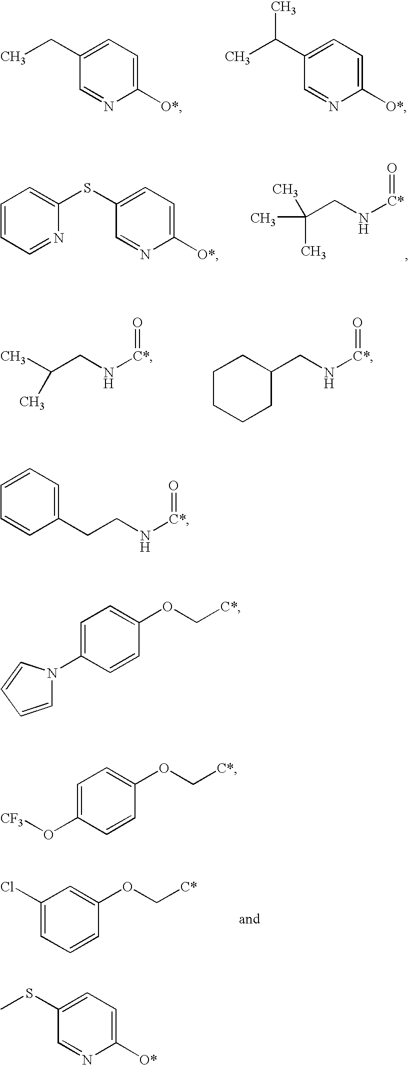

In another embodiment the invention is concerned with compounds wherein R³ is selected from the group consisting of

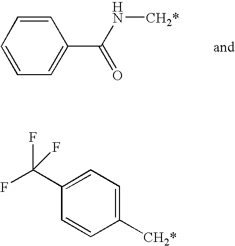

In another embodiment the invention is concerned with compounds wherein R ³ is selected from the group consisting of

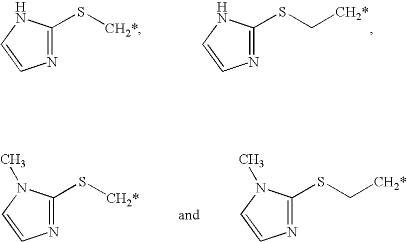

In another embodiment the invention is concerned with compounds wherein R³ is selected from the group consisting of

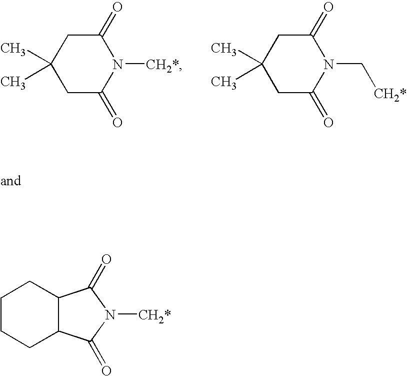

In another embodiment the invention is concerned with compounds wherein R³ is selected from the group consisting of

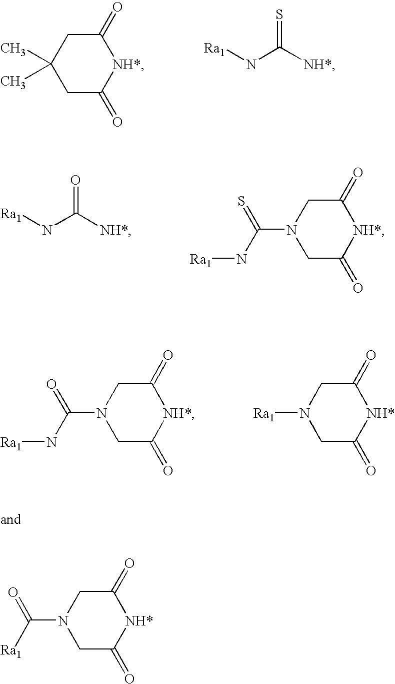

wherein Ra₁ is selected from C₁₋₆-alkyl, aryl, heteroaryl, C₃₋₆-heterocyclyl and C₃₋₇-cycloalkyl.

In another embodiment the invention is concerned with compounds, wherein R⁶ is H.

In another embodiment the invention is concerned with compounds wherein —R⁷ is selected from the group consisting of

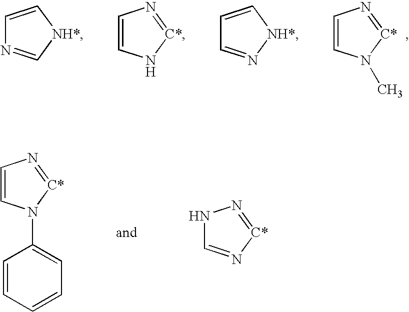

In another embodiment the invention is concerned with compounds wherein, —R⁷ is selected from the group consisting of

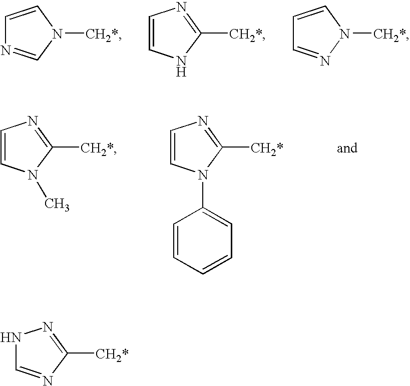

In another embodiment the invention is concerned with compounds wherein —R⁷ is selected from the group consisting of

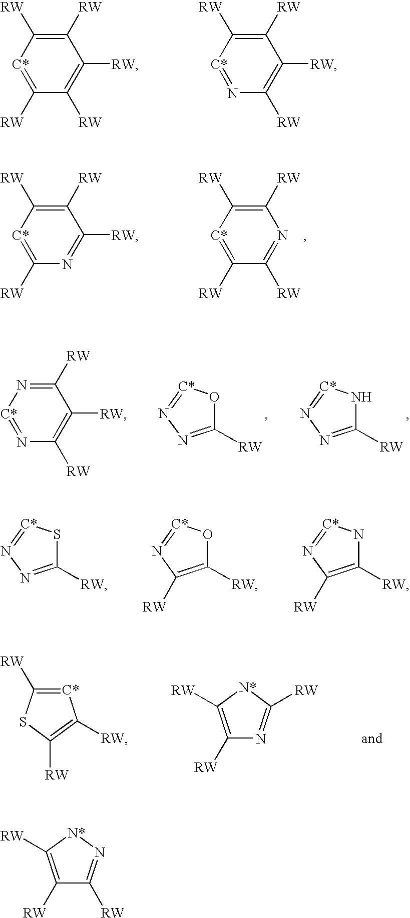

RW is selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆alkyl, perhalomethyl and perhalomethoxy;

In another embodiment the invention is concerned with compounds wherein —R⁷ is selected from the group consisting of

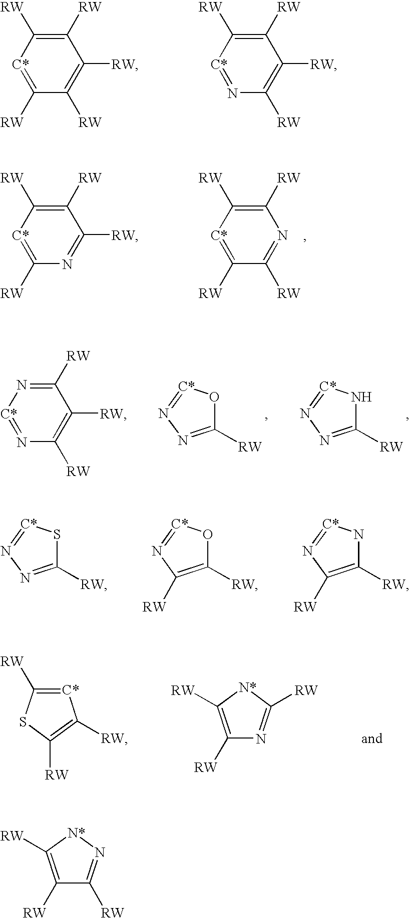

RW is selected from hydroxy, sulfanyl, halogen, amino, sulfo, C₁₋₆alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl,

In another embodiment the invention is concerned with compounds wherein —R⁷ is selected from the group consisting of

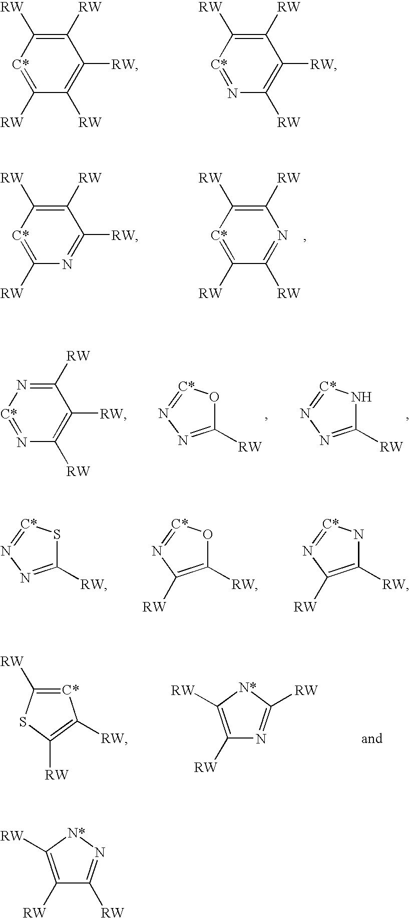

RW is selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl

In another embodiment the invention is concerned with compounds wherein —R⁷ is selected from the group consisting of

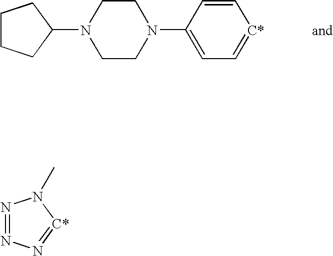

In another embodiment the invention is concerned with compounds wherein —R⁷ is selected from the group consisting of

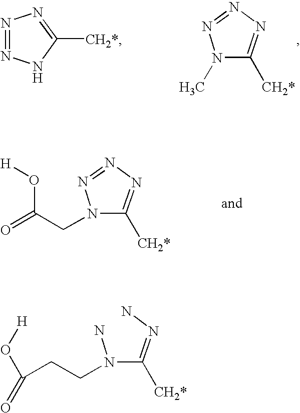

In another embodiment the present invention is concerned with compounds wherein —R⁷ is selected from the group consisting of

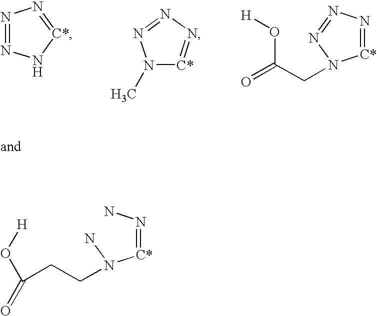

In another embodiment the invention is concerned with compounds wherein —R⁷ is selected from the group consisting of

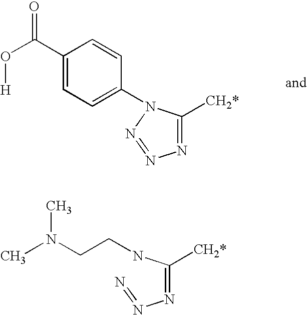

In another embodiment the invention is concerned with compounds wherein —R⁷ is selected from the group consisting of

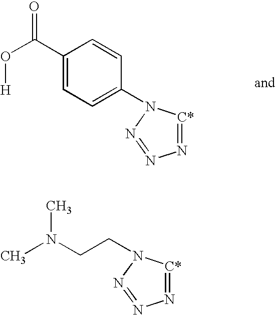

In another embodiment the invention is concerned with compounds wherein —R⁷ is selected from the group consisting of

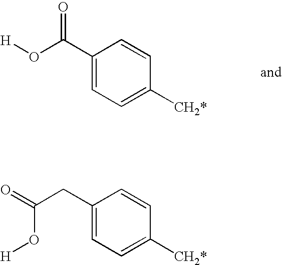

In another embodiment the invention is concerned with compounds wherein —R⁷ is selected from the group consisting of

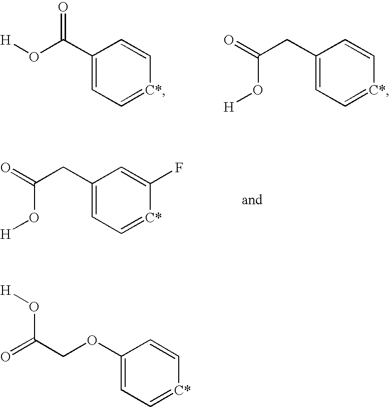

In another embodiment the invention is concerned with compounds wherein —R⁷ is selected from the group consisting of

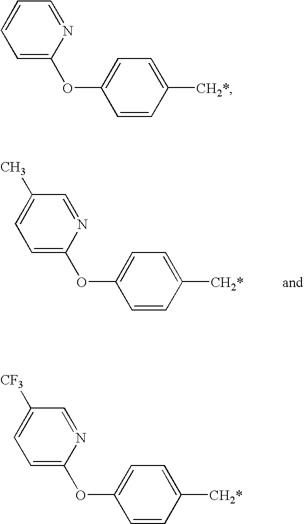

In another embodiment the invention is concerned with compounds wherein —R⁷ is selected from the group consisting of

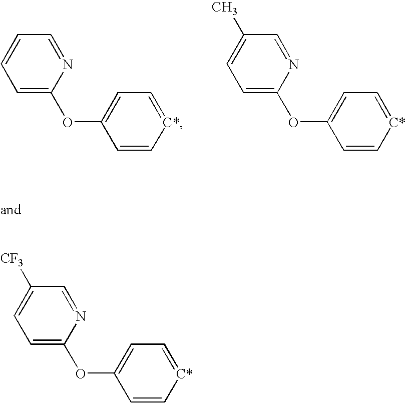

In another embodiment the invention is concerned with compounds wherein —R⁷ is selected from the group consisting of

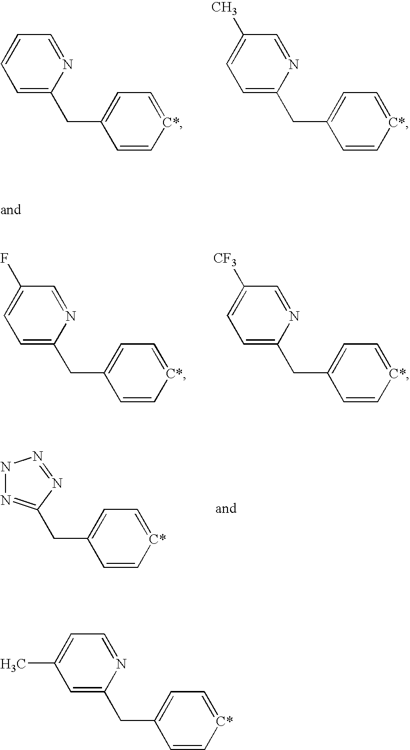

In another embodiment the invention is concerned with compounds wherein A is —O—.

In another embodiment the invention is concerned with compounds wherein A is —S—, —S(═O)— or —S(═O)₂—.

In another embodiment the invention is concerned with compounds wherein A is —S—.

In another embodiment the invention is concerned with compounds wherein A is —CH₂O— or —CH₂S—.

In another embodiment the invention is concerned with compounds wherein A is selected from —CH₂CH₂—N(R⁸)—, —CH₂CH F—N(R⁸)—, —CH₂CF₂—N(R⁸)—, or —CHFCH₂—N(R⁸)—.

In another embodiment the invention is concerned with compounds wherein R⁸ is selected from hydrogen, C₁₋₆-alkyl, C₂₋₆-alkenyl, C₁₋₆-alkyl which has been substituted with one or more halogens, or C₂₋₆-alkenyl which has been substituted with one or more halogens.

In another embodiment the invention is concerned with compounds, having one free —COOH group.

In another embodiment the invention is concerned with compounds having one free amino group, or one monosubstituted amino group or one disubstituted amino group.

In another embodiment the invention is concerned with compounds having one substituted or unsubstituted pyridine ring.

In another embodiment the invention is concerned with compounds having one substituted or unsubstituted imidazole ring.

In another embodiment the invention is concerned with compounds wherein the molar weight of said compound is less than 650 g/mole.

The property cLog P of a compound which has no ionisable group is calculated using Sybyl 6.6 from Tripos Corporation, version 4.0 (provided by Biobyte Corp., Claremont Calif., USA).

In another embodiment the invention is concerned with compounds wherein the compound contains no ionisable group and wherein cLog P is in the range from 1.0 to 5.0.

In another embodiment the invention is concerned with compounds wherein the compound contains no ionisable group and wherein cLog P is in the range from 1.0 to 6.0.

In another embodiment the invention is concerned with compounds wherein the compound contains no ionisable group and wherein cLog P is in the range from 1.0 to 4.0.

A number of other properties of the compounds are calculated using Sybyl 6.6. from Tripos Corporation, i.e. the number of H-bond donors, the number of H-bond acceptors, the number of rotatable bonds. The polar surface area (PSA) is calculated using the SAVol program Based on SAVol 3.7 using Allinger vdw radii. Polar atoms are oxygens, nitrogens, plus hydrogens attached to O and N developed by R. S. Pearlman, J. M. Skell and F. Deanda, Laboratory for Molecular Graphics and Theoretical Modeling, College of Pharmacy, University of Texas, Austin, Tex. 78712, U.S.A.

In another embodiment the invention is concerned with compounds wherein the ACD LogD is in the range from 0.8 to 3.0.

In another embodiment the invention is concerned with compounds wherein the ACD LogD is in the range from 0.8 to 4.0.

In another embodiment the invention is concerned with compounds wherein the number of H-bond donors is 0, 1, 2 or 3.

In another embodiment the invention is concerned with compounds wherein the number of H-bond donors is 1, 2 or 3.

In another embodiment the invention is concerned with compounds wherein the number of H-bond acceptors is in the range from 4 to 9.

In another embodiment the invention is concerned with compounds wherein the number of H-bond acceptors is in the range from 6 to 8.

In another embodiment the invention is concerned with compounds wherein the number of rotatable bonds of said compound is in the range from 4 to 14.

In another embodiment the invention is concerned with compounds wherein the number of rotatable bonds of said compound is in the range from 8 to 12.

In another embodiment the invention is concerned with compounds wherein the polar surface area (PSA) is in the range from 50 Å² to 120 Å².

In another embodiment the invention is concerned with compounds wherein the polar surface area (PSA) is in the range from 70 Å² to 120 Å².

In another embodiment the invention is concerned with a compound selected from the group consisting of

4-[2-(Benzyl-methyl-amino)-ethyl]-piperidine-1-carboxylic acid 4-(5-trifluoromethyl-pyridin-2-yloxy)-phenyl ester,4-(Pyridin-2-ylsulfanyl)-piperidine-1-carboxylic acid 4-(5-trifluoromethyl-pyridin-2-yloxy)-phenyl ester, 4-(1-Pyridin-3-yl-1H-imidazol-2-ylsulfanyl)-piperidine-1-carboxylic acid 4-(5-trifluoromethyl-pyridin-2-yloxy)-phenyl ester, 4-(1-Methyl-1H-imidazol-2-ylsulfanyl)-piperidine-1-carboxylic acid 4-(5-trifluoromethyl-pyridin-2-yloxy)-phenyl ester, 4-[4-(4-Methyl-piperazine-1-carbonyl)-phenoxy]-piperidine-1-carboxylic acid 4-(5-trifluoromethyl-pyridin-2-yloxy)-phenyl ester,

4-(1H-Imidazol-2-ylsulfanyl)-piperidine-1-carboxylic acid 4-(5-trifluoromethyl-pyridin-2-yloxy)-phenyl ester,

4-(4-Piperazin-1-yl-phenoxy)-piperidine-1-carboxylic acid 4-(5-trifluoromethyl-pyridin-2-yloxy)-phenyl ester,

4-(Pyrazol-1-yloxy)-piperidine-1-carboxylic acid 4-(5-trifluoromethyl-pyridin-2-yloxy)-phenyl ester,

4-(Imidazol-1-yloxy)-piperidine-1-carboxylic acid 4-(5-trifluoromethyl-pyridin-2-yloxy)-phenyl ester,

4-(Pyridin-3-yloxy)-piperidine-1-carboxylic acid 4-(5-trifluoromethyl-pyridin-2-yloxy)-phenyl ester,

4-[4-(2-Dimethylamino-ethyl)-phenoxy]-piperidine-1-carboxylic acid 4-(5-trifluoromethyl-pyridin-2-yloxy)-phenyl ester,

4-(5-Phenylsulfanyl-pyrazol-1-yloxy)-piperidine-1-carboxylic acid 4-(5-trifluoromethyl-pyridin-2-yloxy)-phenyl ester,

4-(4-imidazol-1-yl-phenoxy)-piperidine-1-carboxylic acid 4-(5-trifluoromethyl-pyridin-2-yloxy)-phenyl ester,

4-(Pyridin-2-yloxy)-piperidine-1-carboxylic acid 4-(5-trifluoromethyl-pyridin-2-yloxy)-phenyl ester,

4-(3-Pyridin-2-yl-pyrazol-1-yloxy)-piperidine-1-carboxylic acid 4-(5-trifluoomethyl-pyridin-2-yloxy)-phenyl ester,

4-(2-Phenylsulfanyl-imidazol-1-yloxy)-piperidine-1-carboxylic acid 4-(5-trifluoromethyl-pyridin-2-yloxy)-phenyl ester,

4-(2-Chloro-imidazol-1-yloxy)-piperidine-1-carboxylic acid 4-(5-trifluoromethyl-pyridin-2-yloxy)-phenyl ester,

4-[2-(4-Methoxy-phenyl)-imidazol-1-yloxy]-piperidine-1-carboxylic acid 4-(5-trifluoromethyl-pyridin-2-yloxy)-phenyl ester,

4-(2-Methyl-imidazol-1-yloxy)-piperidine-1-carboxylic acid 4-(5-trifluorometyhl-pyridin-2-yloxy)-phenyl ester,

4-(Pyridin-4-ylsulfanyl)-piperidine-1-carboxylic acid 4-(5-trifluoromethyl-pyridin-2-yloxy)-phenyl ester,

4-(4-Pyridin-3-ylmethyl-phenoxy)-piperidine-1-carboxylic acid 4-(5-trifluoromethyl-pyridin-2-yloxy)-phenyl ester,

4-[4-(4-Cyclopentyl-piperazin-1-yl)-phenoxy]-piperidine-1-carboxylic acid 4-(5-trifluoromethyl-pyridin-2-yloxy)-phenyl ester,

4-(4-Pyridin-2-ylmethyl-phenoxy)-piperidine-1-carboxylic acid 4-(5-trifluoromethyl-pyridin-2-yloxy)-phenyl ester,

4-[4-(5-Methyl-pyridin-2-ylmethyl)-phenoxy]-piperidine-1-carboxylic acid 4-(5-trifluoromethyl-pyridin-2-yloxy)-phenyl ester,

4-(5-Pyridin-4-yl-[1,3,4]oxadiazol-2-ylsulfanyl)-piperidine-1-carboxylic acid 4-(5-trifluorpyridin-2-yloxy)-phenyl ester,

4-(5-Pyridin-4-yl-1H-[1,2,4]triazol-3-ylsulfanyl)-piperidine-1-carboxylic acid 4-(5-pyridin-2-yloxy)-phenyl ester, 4-(1-Methyl-1H-tetrazol-5-ylsulfanyl)-piperidine-1-carboxylic acid 4-(5-trifluoromethyl-pyridin-2-yloxy)-phenyl ester, 4-[4-(4-Ethoxycarbonyl-oxazol-2-yl)-phenoxy]-piperidine-1--carboxylic acid 4-(5-trifluoromethyl-pyridin-2-yloxy)-phenyl ester, 4-(4′-Methoxycarbonyl-biphenyl-4-yloxy)-piperidine-1-carboxylic acid 4-(5-trifluoromethyl-pyridin-2-yloxy)-phenyl ester,

5-{1-[4-(5-Trifluoromethyl-pyridin-2-yloxy)-phenoxycarbonyl]-piperidin-4-yloxy}-1H-indole-carboxylic acid ethyl ester,

4-[4-(2-Ethoxycarbonyl-ethyl)-phenoxy]-piperidine-1-carboxylic acid 4-(5-trifluoromethyl-pyridin-2-yloxy)-phenyl ester,

4-(3-Ethoxycarbonyl-phenoxy)-piperidine-1-carboxylic acid 4-(5-trifluoromethyl-pyridin-2-yloxy)-phenyl ester,

4-(4-Ethoxycarbonyl-1H-imidazol-2-ylsulfanyl)-piperidine-1-carboxylic acid 4-(5-trifluoromethyl-pyridin-2-yloxy)-phenyl ester,

4-(4-Allyloxycarbonylmethyl-phenoxy)-piperidine-1-carboxylic acid 4-(5-trifluoromethyl-pyridin-2-yloxy)-phenyl ester,

4-(4′-Carboxy-biphenyl-4-yloxy)-piperidine-1-carboxylic acid 4-(5-trifluoromethyl-pyridin-2-yloxy)-phenyl ester,

4-[4-(4-Carboxy-oxazol-2-yl)-phenoxy]-piperidine-1-carboxylic acid 4-(5-trifluoromethyl-pyridin-2-yloxy)-phenyl ester,

4-[4-(2-Carboxy-ethyl)-phenoxy]-piperidine-1-carboxylic acid 4-(5-trifluoromethyl-pyridin-2-yloxy)-phenyl ester,

4-(3-Carboxy-phenoxy)-piperidine-1-carboxylic acid 4-(5-trifluoromethyl-pyridin-2-yloxy)-phenyl ester,

4-(4-Carboxy-phenoxy)-piperidine-1-carboxylic acid 4-(5-trifluoromethyl-pyridin-2-yloxy)-phenyl ester,

4-(4-Carboxymethyl-phenoxy)-piperidine-1-carboxylic acid 4-(5-trifluoromethyl-pyridin-2-yloxy)-phenyl ester,

4-(1H-imidazol-2-ylsulfanylmethyl)-piperidine-1-carboxylic acid 4-(5-trifluoromethyl-pyridin-2-yloxy)-phenyl ester,

4-(4-Allyloxycarbonylmethyl-phenoxymethyl)-piperidine-1-carboxylic acid 4-(5-trifluoromethyl-pyridin-2-yloxy)-phenyl ester,

4-(4-Allyloxycarbonyl-phenoxymethyl)-piperidine-1-carboxylic acid 4-(5-trifluoromethyl-pyridin-2-yloxy)-phenyl ester,

4-(4-Carboxymethyl-phenoxymethyl)-piperidine-1-carboxylic acid 4-(5-trifluoromethyl-pyridin-2-yloxy)-phenyl ester,

4-(4-Carboxy-phenoxymethyl)-piperidine-1-carboxylic acid 4-(5-trifluoromethyl-pyridin-2-yloxy)-phenyl ester,

4-(4-Allyloxycarbonyl-phenoxy)-piperidine-1-carboxylic acid 4-(4-trifluoromethyl-benzyl)-phenyl ester,

4-(4-Carboxy-phenoxy)-piperidine-1-carboxylic acid 4-(4-trifluoromethyl-benzyl)-phenyl ester

4-(Pyrazol-1-yloxy)-piperidine-1-carboxylic acid 4-(5-methyl-pyridin-2-ylmethyl)-phenyl ester

4-(Imidazol-1-yloxy)-piperidine-1-carboxylic acid 4-(6-methyl-pyridin-3-ylmethyl)-phenyl ester,

4-Phenoxy-piperidine-1-carboxylic acid 4-(6-methyl-pyridin-3-ylmethyl)-phenyl ester,

4-(1H-Imidazol-2-ylsulfanyl)-piperidine-1-carboxylic acid 4-(5-methyl-pyridin-2-ylmethyl)-phenyl ester,

4-(4-Allyloxycarbonylmethyl-phenoxy)-piperidine-1-carboxylic acid 4-(5-methyl-pyridin-2-ylmethyl)-phenyl ester,

4-(4-Carboxymethyl-phenoxy)-piperidine-1-carboxylic acid 4-(5-methyl-pyridin-2-ylmethyl)-phenyl ester,

4-(1-Methyl-i H-imidazol-2-ylsulfanyl)-piperidine-1-carboxylic acid 4-[2-(4,4-dimethyl-2,6-piperidin-1-yl)-ethyl]-phenyl ester,

4-(4-Allyloxycarbonylmethyl-phenoxy)-piperidine-1-carboxylic acid 4-[2-(4,4-dimethyl-2,6-dioxo-piperidin-1-yl)-ethyl]-phenyl ester,

4-(1H-Imidazol-2-ylsulfanyl)-piperidine-1-carboxylic acid 4-[2-(4,4-dimethyl-2,6-dioxo-piperidin-1-yl)-ethyl]-phenyl ester,

4-(4-Carboxymethyl-phenoxy)-piperidine-1-carboxylic acid 4-[2-(4,4-dimethyl-2,6-dioxo-piperidin-1-yl)-ethyl]-phenyl ester,

4-(4-Allyloxycarbonyl-phenoxy)-piperidine-1-carboxylic acid 4-[2-(4,4-dimethyl-2,6-dioxo-piperidin-1-yl)-ethyl]-phenyl ester,

4-(4-Carboxy-phenoxy)-piperidine-1-carboxylic acid 4-[2-(4,4-dimethyl-2,6-dioxo-piperidin-1-yl)-ethyl]-phenyl ester,

4-(1H-[1,2,4]Triazol-3-ylsulfanyl)-piperidine-1-carboxylic acid 4-(5-methyl-pyridin-2-ylmethyl phenyl ester,4-(Pyrazol-1-yloxy)-piperidine-1-carboxylic acid 5-benzoylamino-pyridin-2-yl ester,

4-(Pyrazol-1-yloxy)-piperidine-1-carboxylic acid 4,4-dimethyl-2,6-dioxo-3,4,5,6-tetrahydro-2H-[1,3′]bipyridinyl-6′-yl ester,

4-[2-(3,4-Dihydro-1H-isoquinolin-2-yl)-ethyl]-piperidine-1-carboxylic acid 4-(5-trifluoromethyl-pyridin-2-yloxy)-phenyl ester,

4-(2-Piperidin-1-yl-ethyl)-piperidine-1-carboxylic acid 4-(5-trifluoromethyl-pyridin-2-yloxy)-phenyl ester,

4-(1H-Benzoimidazol-2-ylsulfanyl)-piperidine-1-carboxylic acid 4-(5-trifluoromethyl-pyridin-2-yloxy)-phenyl ester

4-(Thiazolo[4,5-b]pyridin-2-ylsulfanyl)-piperidine-1-carboxylic acid 4-(5-trifluoromethyl-pyridin-2-yloxy)-phenyl ester

and

4-(7H-Purin-6-ylsulfanyl)-piperidine-1-carboxylic acid 4(5-trifluoromethyl-pyridin-2-yloxy)-phenyl ester

In another embodiment the invention is concerned with a compound selected from the group consisting of

4-(1-Methyl-1H-tetrazol-5-ylsulfanyl)-piperidine-1-carboxylic acid 4-(5-methyl-pyridin-2-ylmethyl)-phenyl ester

4-[1-(2-Dimethylamino-ethyl)-1H-tetrazol-5-ylsulfanyl]-piperidine-1-carboxylic acid 4-(5-methyl-pyridin-2-ylmethyl)-phenyl ester

4-(Imidazol-1-yloxy)-piperidine-1-carboxylic acid 4-[2-(4,4-dimethyl-2,6-dioxo-piperidin-1-yl)-ethyl]-phenyl ester

4-(Pyrazol-1-yloxy)-piperidine-1-carboxylic acid 4-[2-(4,4-dimethyl-2,6-dioxo-piperidin-1-yl)-ethyl]-phenyl ester

4-(1H-[1,2,4]Triazol-3-ylsulfanyl)-piperidine-1-carboxylic acid 4-(5-trifluoromethyl-pyridin-2-yloxy)-phenyl ester

4-[1-(4-Carboxy-phenyl)-1H-tetrazol-5-ylsulfanyl]-piperidine-1-carboxylic acid 4-(5-trifluoromethyl-pyridin-2-yloxy)-phenyl ester

4-[4-(Pyridin-2-yloxy)-phenoxy]-piperidine-1-carboxylic acid 4-(5-trifluoromethyl-pyridin-2-yloxy)-phenyl ester

4-[4-(5-Methyl-pyridin-2-yloxy)-phenoxy]-piperidine-1-carboxylic acid 4-(5-trifluoromethyl-pyridin-2-yloxy)-phenyl ester

4-(1-Methyl-1H-imidazol-2-ylsulfanylmethyl)-piperidine-1-carboxylic acid 4-(5-trifluoromethyl-pyridin-2-yloxy)-phenyl ester

4-(Imidazol-1-yloxymethyl)-piperidine-1-carboxylic acid 4-(5-trifluoromethyl-pyridin-2-yloxy)-phenyl ester

4-[4-(Pyridin-2-yloxy)-phenoxymethyl]-piperidine-1-carboxylic acid 4-(5-trifluoromethyl-pyridin-2-yloxy)-phenyl ester

4-[4-(5-Methyl-pyridin-2-yloxy)-phenoxymethyl]-piperidine-1-carboxylic acid 4-(5-trifluoromethyl-pyridin-2-yloxy)-phenyl ester

4-(1-Methyl-1H-tetrazol-5-ylsulfanylmethyl)-piperidine-1-carboxylic acid 4-(5 -trifluoromethyl-pyridin-2-yloxy)-phenyl ester

4-[1-(2-Dimethylamino-ethyl)-1H-tetrazol-5-ylsulfanyl]-piperidine-1-carboxylic acid 4-(5-trifluoromethyl-pyridin-2-yloxy)-phenyl ester

4-[l-(2-Carboxy-ethyl)-1H-tetrazol-5-ylsulfanyl]-piperidine-1-carboxylic acid 4-(5-trifluoromethyl-pyridin-2-yloxy)-phenyl ester

4-(1-Carboxymethyl-1H-tetrazol-5-ylsulfanyl)-piperidine-1-carboxylic acid 4-(5-trifluoromethyl-pyridin-2-yloxy)-phenyl ester

4-(Pyrazol-1-yloxymethyl)-piperidine-1-carboxylic acid 4-(5-trifluoromethyl-pyridin-2-yloxy)-phenyl ester

4-(1H-[1,2,4]Triazol-3-ylsulfanylmethyl)-piperidine-1-carboxylic acid 4-(5-trifluoromethyl-pyridin-2-yloxy)-phenyl ester

4-(1H-[1,2,4]Triazol-3-ylsulfanyl)-piperidine-1-carboxylic acid 4-(5-chloro-pyridin-2-yloxy)-phenyl ester

4-(1H-Imidazol-2-ylsulfanyl)-piperidine-1-carboxylic acid 4-(5-chloro-pyridin-2-yloxy)-phenyl ester

4-(1-Methyl-1H-imidazol-2-ylsulfanyl)-piperidine-1-carboxylic acid 4-(5-chloro-pyridin-2-yloxy)-phenyl ester

4-(1-Methyl-1H-tetrazol-5-ylsulfanyl)-piperidine-1-carboxylic acid 4-(5-chloro-pyridin-2-yloxy)-phenyl ester

4-(imidazol-1-yloxy)-piperidine-1-carboxylic acid 4-(5-chloro-pyridin-2-yloxy)-phenyl ester

4-[1-(4-Carboxy-phenyl)-1H-tetrazol-5-ylsulfanyl]-piperidine-1-carboxylic acid 4-(5-chloro-pyridin-2-yloxy)-phenyl ester

4-(4-Carboxymethyl-phenoxy)-piperidine-1-carboxylic acid 4-(5-chloro-pyridin-2-yloxy)-phenyl ester

4-(Pyrazol-1-yloxy)-piperidine-1-carboxylic acid 4-(5-chloro-pyridin-2-yloxy)-phenyl ester

4-(4-Carboxy-phenoxy)-piperidine-1-carboxylic acid 4-(5-chloro-pyridin-2-yloxy)-phenyl ester

4-[4-(Pyridin-2-yloxy)-phenoxy]-piperidine-1-carboxylic acid 4-(5-chloro-pyridin-2-yloxy)-phenyl ester

4-[4-(5-Methyl-pyridin-2-yloxy)-phenoxy]-piperidine-1-carboxylic acid 4-(5-chloro-pyridin-2-yloxy)-phenyl ester

4-(4-Carboxy-phenoxymethyl)-piperidine-1-carboxylic acid 4-(5-chloro-pyridin-2-yloxy)-phenyl ester

4-(4-Carboxymethyl-phenoxymethyl)-piperidine-1-carboxylic acid 4-(5-chloro-pyridin-2-yloxy)-phenyl ester

4-(1H-Imidazol-2-ylsulfanylmethyl)-piperidine-1-carboxylic acid 4-(5-chloro-pyridin-2-yloxy)-phenyl ester

4-(1-Methyl-1H-imidazol-2-ylsulfanylmethyl)-piperidine-1-carboxylic acid 4-(5-chloro-pyridin-2-yloxy)-phenyl ester

4-(Imidazol-1-yloxymethyl)-piperidine-1-carboxylic acid 4-(5-chloro-pyridin-2-yloxy)-phenyl ester

4-[4-(Pyridin-2-yloxy)-phenoxymethyl]-piperidine-1-carboxylic acid 4-(5-chloro-pyridin-2-yloxy)-phenyl ester

4-[4-(5-Methyl-pyridin-2-yloxy)-phenoxymethyl]-piperidine-1-carboxylic acid 4-(5-chloro-pyridin-2-yloxy)-phenyl ester

4-(1-Methyl-1H-tetrazol-5-ylsulfanylmethyl)-piperidine-1-carboxylic acid 4-(5-chloro-pyridin-2-yloxy)-phenyl ester

4-[1-(2-Dimethylamino-ethyl)-1H-tetrazol-5-ylsulfanyl]-piperidine-1-carboxylic acid 4-(5-chloro-pyridin-2-yloxy)-phenyl ester

4-[1-(2-Carboxy-ethyl)-1H-tetrazol-5-ylsulfanyl]-piperidine-1-carboxylic acid 4-(5-chloro-pyridin-2-yloxy)-phenyl ester

4-(1-Carboxymethyl-1H-tetrazol-5-ylsulfanyl)-piperidine-1-carboxylic acid 4-(5-chloro-pyridin-2-yloxy)-phenyl ester

4-(Pyrazol-1-yloxymethyl)-piperidine-1-carboxylic acid 4-(5-chloro-pyridin-2-yloxy)-phenyl ester

4-(1H-[1,2,4]Triazol-3-ylsulfanylmethyl)-piperidine-1-carboxylic acid 4-(5-chloro-pyridin-2-yloxy)-phenyl ester

4-[2-(Benzyl-methyl-amino)-ethyl]-piperidine-1-carboxylic acid 4-(5-chloro-pyridin-2-yloxy)-phenyl ester

4-(1H-[1,2,4]Triazol-3-ylsulfanyl)-piperidine-1-carboxylic acid 4-(3,3-dimethyl-butylcarbamoyl)-phenyl ester

4-(1H-Imidazol-2-ylsulfanyl)-piperidine-1-carboxylic acid 4-(3,3-dimethyl-butylcarbamoyl)-phenyl ester

4-(1-Methyl-1H-imidazol-2-ylsulfanyl)-piperidine-1-carboxylic acid 4-(3,3-dimethyl-butylcarbamoyl)-phenyl ester

4-(1-Methyl-1H-tetrazol-5-ylsulfanyl)-piperidine-1-carboxylic acid 4-(3,3-dimethyl-butylcarbamoyl)-phenyl ester

4-(Imidazol-1-yloxy)-piperidine-1-carboxylic acid 4-(3,3-dimethyl-butylcarbamoyl)-phenyl ester

4-[1-(4-Carboxy-phenyl)-1H-tetrazol-5-ylsulfanyl]-piperidine-1-carboxylic acid 4-(3,3-dimethyl-butylcarbamoyl)-phenyl ester

4-(4-Carboxymethyl-phenoxy)-piperidine-1-carboxylic acid 4-(3,3-dimethyl-butylcarbamoyl)-phenyl ester

4-(Pyrazol-1-yloxy)-piperidine-1-carboxylic acid 4-(3,3-dimethyl-butylcarbamoyl)-phenyl ester

4-(4-Carboxy-phenoxy)-piperidine-1-carboxylic acid 4-(3,3-dimethyl-butylcarbamoyl)-phenyl ester

4-[4-(Pyridin-2-yloxy)-phenoxy]-piperidine-1-carboxylic acid 4-(3,3-dimethyl-butylcarbamoyl)-phenyl ester

4-[4-(5-Methyl-pyridin-2-yloxy)-phenoxy]-piperidine-1-carboxylic acid 4-(3,3-dimethyl-butylcarbamoyl)-phenyl ester

4-(4-Carboxy-phenoxymethyl)-piperidine-1-carboxylic acid 4-(3,3-dimethyl-butylcarbamoyl)-phenyl ester

4-(4-Carboxymethyl-phenoxymethyl)-piperidine-1-carboxylic acid 4-(3,3-dimethyl-butylcarbamoyl)-phenyl ester

4-(1H-Imidazol-2-ylsulfanylmethyl)-piperidine-1-carboxylic acid 4-(3,3-dimethyl-butylcarbamoyl)-phenyl ester

4-(1-Methyl-1H-imidazol-2-ylsulfanylmethyl)-piperidine-1-carboxylic acid 4-(3,3-dimethyl-butylcarbamoyl)-phenyl ester

4-(Imidazol-1-yloxymethyl)-piperidine-1-carboxylic acid 4-(3,3-dimethyl-butylcarbamoyl)-phenyl ester

4-[4-(Pyridin-2-yloxy)-phenoxymethyl]-piperidine-1-carboxylic acid 4-(3,3-dimethyl-butylcarbamoyl)-phenyl ester

4-[4-(5-Methyl-pyridin-2-yloxy)-phenoxymethyl]-piperidine-1-carboxylic acid 4-(3,3-dimethyl-butylcarbamoyl)-phenyl ester

4-(1-Methyl-1H-tetrazol-5-ylsulfanylmethyl)-piperidine-1-carboxylic acid 4-(3,3-dimethyl-butylcarbamoyl)-phenyl ester

4-[1-(2-Dimethylamino-ethyl)-1H-tetrazol-5-ylsulfanyl]-piperidine-1-carboxylic acid 4-(3,3-dimethyl-butylcarbamoyl)-phenyl ester

4-[1-(2-Carboxy-ethyl)-1H-tetrazol-5-ylsulfanyl]-piperidine-1-carboxylic acid 4-(3,3-dimethyl-butylcarbamoyl)-phenyl ester

4-(1-Carboxymethyl-1H-tetrazol-5-ylsulfanyl)-piperidine-1-carboxylic acid 4-(3,3-dimethyl-butylcarbamoyl)-phenyl ester

4-(Pyrazol-1-yloxymethyl)-piperidine-1-carboxylic acid 4-(3,3-dimethyl-butylcarbamoyl)-phenyl ester

4-(1H-[1,2,4]Triazol-3-ylsulfanylmethyl)-piperidine-1-carboxylic acid 4-(3,3-dimethyl-butylcarbamoyl)-phenyl ester

4-[2-(Benzyl-methyl-amino)-ethyl]-piperidine-1-carboxylic acid 4-(3,3-dimethyl-butylcarbamoyl)-phenyl ester

4-(1-Methyl-1H-imidazol-2-ylsulfanyl)-piperidine-1-carboxylic acid 4-(5-methyl-pyridin-2-ylmethyl)-phenyl ester

4-[1-(4-Carboxy-phenyl)-1H-tetrazol-5-ylsulfanyl]-piperidine-1-carboxylic acid 4-(5-methyl-pyridin-2-ylmethyl)-phenyl ester

4-(4-Carboxy-phenoxy)-piperidine-1-carboxylic acid 4-(5-methyl-pyridin-2-ylmethyl)-phenyl ester

4-[4-(Pyridin-2-yloxy)-phenoxy]-piperidine-1-carboxylic acid 4-(5-methyl-pyridin-2-ylmethyl)-phenyl ester

4-[4-(5-Methyl-pyridin-2-yloxy)-phenoxy]-piperidine-1-carboxylic acid 4-(5-methyl-pyridin-2-ylmethyl)-phenyl ester

4-(4-Carboxy-phenoxymethyl)-piperidine-1-carboxylic acid 4-(5-methyl-pyridin-2-ylmethyl)-phenyl ester

4-(4-Carboxymethyl-phenoxymethyl)-piperidine-1-carboxylic acid 4-(5-methyl-pyridin-2-ylmethyl)-phenyl ester

4-(1H-Imidazol-2-ylsulfanylmethyl)-piperidine-1-carboxylic acid 4-(5-methyl-pyridin-2-ylmethyl)-phenyl ester

4-(1-Methyl-1H-imidazol-2-ylsulfanylmethyl)-piperidine-1-carboxylic acid 4-(5-methyl-pyridin-2-ylmethyl)-phenyl ester

4-(Imidazol-1-yloxymethyl)-piperidine-1-carboxylic acid 4-(5-methyl-pyridin-2-ylmethyl)-phenyl ester

4-[4-(Pyridin-2-yloxy)-phenoxymethyl]-piperidine-1l-carboxylic acid 4-(5-methyl-pyridin-2-ylmethyl)-phenyl ester

4-[4-(5-Methyl-pyridin-2-yloxy)-phenoxymethyl]-piperidine-1-carboxylic acid 4-(5-methyl-pyridin-2-ylmethyl)-phenyl ester

4-(1-Methyl-1H-tetrazol-5-ylsulfanylmethyl)-piperidine-1-carboxylic acid 4-(5-methyl-pyridin-2-ylmethyl)-phenyl ester

4-[1-(2-Carboxy-ethyl)-1H-tetrazol-5-ylsulfanyl]-piperidine-1-carboxylic acid 4-(5-methyl-pyridin-2-ylmethyl)-phenyl ester

4-(1-Carboxymethyl-1H-tetrazol-5-ylsulfanyl)-piperidine-1-carboxylic acid 4-(5-methyl-pyridin-2-ylmethyl)-phenyl ester

4-(Pyrazol-1-yloxymethyl)-piperidine-1-carboxylic acid 4-(5-methyl-pyridin-2-ylmethyl)-phenyl ester

4-(1H-[1,2,4]Triazol-3-ylsulfanylmethyl)-piperidine-1-carboxylic acid 4-(5-methyl-pyridin-2-ylmethyl)-phenyl ester

4-[2-(Benzyl-methyl-amino)-ethyl]-piperidine-1-carboxylic acid 4-(5-methyl-pyridin-2-ylmethyl)-phenyl ester

4-(1H-[1,2,4]Triazol-3-ylsulfanyl)-piperidine-1-carboxylic acid 4-(4-trifluoromethyl-benzyl)-phenyl ester

4-(1H-Imidazol-2-ylsulfanyl)-piperidine-1-carboxylic acid 4-(4-trifluoromethyl-benzyl)-phenyl ester

4-(1-Methyl-1H-imidazol-2-ylsulfanyl)-piperidine-1-carboxylic acid 4-(4-trifluoromethyl-benzyl)-phenyl ester

4-(1-Methyl-1H-tetrazol-5-ylsulfanyl)-piperidine-1-carboxylic acid 4-(4-trifluoromethyl-benzyl)-phenyl ester

4-(Imidazol-1-yloxy)-piperidine-1-carboxylic acid 4-(4-trifluoromethyl-benzyl)-phenyl ester

4-[1-(4-Carboxy-phenyl)-1H-tetrazol-5-ylsulfanyl]-piperidine-1-carboxylic acid 4-(4-trifluoromethyl-benzyl)-phenyl ester

4-(4-Carboxymethyl-phenoxy)-piperidine-1-carboxylic acid 4-(4-trifluoromethyl-benzyl)-phenyl ester

4-(Pyrazol-1-yloxy)-piperidine-1-carboxylic acid 4-(4-trifluoromethyl-benzyl)-phenyl ester

4-[4-(Pyridin-2-yloxy)-phenoxy]-piperidine-1-carboxylic acid 4-(4-trifluoromethyl-benzyl)-phenyl ester

4-[4-(5-Methyl-pyridin-2-yloxy)-phenoxy]-piperidine-1-carboxylic acid 4-(4-trifluoromethyl-benzyl)-phenyl ester

4-(4-Carboxy-phenoxymethyl)-piperidine-1-carboxylic acid 4-(4-trifluoromethyl-benzyl)-phenyl ester

4-(4-Carboxymethyl-phenoxymethyl)-piperidine-1-carboxylic acid 4-(4-trifluoromethyl-benzyl)-phenyl ester

4-(1H-Imidazol-2-ylsulfanylmethyl)-piperidine-1-carboxylic acid 4-(4-trifluoromethyl-benzyl)-phenyl ester

4-(1-Methyl-1H-imidazol-2-ylsulfanylmethyl)-piperidine-1-carboxylic acid 4-(4-trifluoromethyl-benzyl)-phenyl ester

4-(Imidazol-1-yloxymethyl)-piperidine-1-carboxylic acid 4-(4-trifluoromethyl-benzyl)-phenyl ester

4-[4-(Pyridin-2-yloxy)-phenoxymethyl]-piperidine-1-carboxylic acid 4-(4-trifluoromethyl-benzyl)-phenyl ester

4-[4-(5-Methyl-pyridin-2-yloxy)-phenoxymethyl]-piperidine-1-carboxylic acid 4-(4-trifluoromethyl-benzyl)-phenyl ester

4-(1-Methyl-1H-tetrazol-5-ylsulfanylmethyl)-piperidine-1-carboxylic acid 4-(4-trifluoromethyl-benzyl)-phenyl ester

4-[1-(2-Dimethylamino-ethyl)-1H-tetrazol-5-ylsulfanyl]-piperidine-1-carboxylic acid 4-(4-trifluoromethyl-benzyl)-phenyl ester

4-[1-(2-Carboxy-ethyl)-1H-tetrazol-5-ylsulfanyl]-piperidine-1-carboxylic acid 4-(4-trifluoromethyl-benzyl)-phenyl ester

4-(1-Carboxymethyl-1H-tetrazol-5-ylsulfanyl)-piperidine-1-carboxylic acid 4-(4-trifluoromethyl-benzyl)-phenyl ester

4-(Pyrazol-1-yloxymethyl)-piperidine-1-carboxylic acid 4-(4-trifluoromethyl-benzyl)-phenyl ester

4-(1H-[1,2,4]Triazol-3-ylsulfanylmethyl)-piperidine-1-carboxylic acid 4-(4-trifluoromethyl-benzyl)-phenyl ester

4-[2-(Benzyl-methyl-amino)-ethyl]-piperidine-1-carboxylic acid 4-(4-trifluoromethyl-benzyl)-phenyl ester

4-(1H-[1,2,4]Triazol-3-ylsulfanyl)-piperidine-1-carboxylic acid 4,4-dimethyl-2,6-dioxo-3,4,5,6-tetrahydro-2H-[1,3′]bipyridinyl-6′-yl ester

4-(1H-Imidazol-2-ylsulfanyl)-piperidine-1-carboxylic acid 4,4-dimethyl-2,6-dioxo-3,4,5,6-tetrahydro-2H-[1,3′]bipyridinyl-6′-yl ester

4-(1-Methyl-1H-imidazol-2-ylsulfanyl)-piperidine-1-carboxylic acid 4,4-dimethyl-2,6-dioxo-3,4,5,6-tetrahydro-2H-[1,3′]bipyridinyl-6′-yl ester

4-(1-Methyl-1H-tetrazol-5-ylsulfanyl)-piperidine-1-carboxylic acid 4,4-dimethyl-2,6-dioxo-3,4,5,6-tetrahydro-2H-[1,3′]bipyridinyl-6′-yl ester

4-(Imidazol-1-yloxy)-piperidine-1-carboxylic acid 4,4-dimethyl-2,6-dioxo-3,4,5,6-tetrahydro-2H-[1,3′]bipyridinyl-6′-yl ester

4-[1-(4-Carboxy-phenyl)-1H-tetrazol-5-ylsulfanyl]-piperidine-1-carboxylic acid 4,4-dimethyl-2,6-dioxo-3,4,5,6-tetrahydro-2H-[1,3′]bipyridinyl-6′-yl ester

4-(4-Carboxymethyl-phenoxy)-piperidine-1-carboxylic acid 4,4-dimethyl-2,6-dioxo-3,4,5,6-tetrahydro-2H-[1,3′]bipyridinyl-6′-yl ester

4-(4-Carboxy-phenoxy)-piperidine-1-carboxylic acid 4,4-dimethyl-2,6-dioxo-3,4,5,6-tetrahydro-2H-[1,3′]bipyridinyl-6′-yl ester

4-[4-(Pyridin-2-yloxy)-phenoxy]-piperidine-1-carboxylic acid 4,4-dimethyl-2,6-dioxo-3,4,5,6-tetrahydro-2H-[1,3′]bipyridinyl-6′-yl ester

4-[4-(5-Methyl-pyridin-2-yloxy)-phenoxy]-piperidine-1-carboxylic acid 4,4-dimethyl-2,6-dioxo-3,4,5,6-tetrahydro-2H-[1,3′]bipyridinyl-6′-yl ester

4-(4-Carboxy-phenoxymethyl)-piperidine-1-carboxylic acid 4,4-dimethyl-2,6-dioxo-3,4,5,6-tetrahydro-2H-[1,3′]bipyridinyl-6′-yl ester

4-(4-Carboxymethyl-phenoxymethyl)-piperidine-1-carboxylic acid 4,4-dimethyl-2,6-dioxo-3,4,5,6-tetrahydro-2H-[1,3′]bipyridinyl-6′-yl ester

4-(1H-Imidazol-2-ylsulfanylmethyl)-piperidine-1-carboxylic acid 4,4-dimethyl-2,6-dioxo-3,4,5,6-tetrahydro-2H-[1,3′]bipyridinyl-6′-yl ester

4-(1-Methyl-1H-imidazol-2-ylsulfanylmethyl)-piperidine-1-carboxylic acid 4,4-dimethyl-2,6-dioxo-3,4,5,6-tetrahydro-2H-[1,3′]bipyridinyl-6′-yl ester

4-(Imidazol-1-yloxymethyl)-piperidine-1-carboxylic acid 4,4-dimethyl-2,6-dioxo-3,4,5,6-tetrahydro-2H-[1,3′]bipyridinyl-6′-yl ester

4-[4-(Pyridin-2-yloxy)-phenoxymethyl]-piperidine-1-carboxylic acid 4,4-dimethyl-2,6-dioxo-3,4,5,6-tetrahydro-2H-[1,3′]bipyridinyl-6′-yl ester

4-[4-(5-Methyl-pyridin-2-yloxy)-phenoxymethyl]-piperidine-1-carboxylic acid 4,4-dimethyl-2,6-dioxo-3,4,5,6-tetrahydro-2H-[1,3′]bipyridinyl-6′-yl ester

4-(1-Methyl-1H-tetrazol-5-ylsulfanylmethyl)-piperidine-1-carboxylic acid 4,4-dimethyl-2,6-dioxo-3,4,5,6-tetrahydro-2H-[1,3′]bipyridinyl-6′-yl ester

4-[1-(2-Dimethylamino-ethyl)-1H-tetrazol-5-ylsulfanyl]-piperidine-1-carboxylic acid 4,4-dimethyl-2,6-dioxo-3,4,5,6-tetrahydro-2H-[1,3]bipyridinyl-6′-yl ester

4-[1-(2-Carboxy-ethyl)-1H-tetrazol-5-ylsulfanyl]-piperidine-1-carboxylic acid 4,4-dimethyl-2,6-dioxo-3,4,5,6-tetrahydro-2H-[1,3′]bipyridinyl-6′-yl ester

4-(1-Carboxymethyl-1H-tetrazol-5-ylsulfanyl)-piperidine-1-carboxylic acid 4,4-dimethyl-2,6-dioxo-3,4,5,6-tetrahydro-2H-[1,3′]bipyridinyl-6′-yl ester

4-(Pyrazol-1-yloxymethyl)-piperidine-1-carboxylic acid 4,4-dimethyl-2,6-dioxo-3,4,5,6-tetrahydro-2H-[1,3′]bipyridinyl-6′-yl ester

4-(1H-[1,2,4]Triazol-3-ylsulfanylmethyl)-piperidine-1-carboxylic acid 4,4-dimethyl-2,6-dioxo-3,4,5,6-tetrahydro-2H-[1,3′]bipyridinyl-6′-yl ester

4-[2-(Benzyl-methyl-amino)-ethyl]-piperidine-1-carboxylic acid 4,4-dimethyl-2,6-dioxo-3,4,5,6-tetrahydro-2H-[1,3′]bipyridinyl-6′-yl ester

4-(1H-[1,2,4]Triazol-3-ylsulfanyl)-piperidine-1-carboxylic acid 5-[2-(4,4-dimethyl-2,6-dioxo-piperidin-1-yl)-ethyl]-pyridin-2-yl ester

4-(1H-Imidazol-2-ylsulfanyl)-piperidine-1-carboxylic acid 5-[2-(4,4-dimethyl-2,6-dioxo-piperidin-1-yl)-ethyl]-pyridin-2-yl ester

4-(1-Methyl-1H-imidazol-2-ylsulfanyl)-piperidine-1-carboxylic acid 5-[2-(4,4-dimethyl-2,6-dioxo-piperidin-1-yl)-ethyl]-pyridin-2-yl ester

4-(1-Methyl-1H-tetrazol-5-ylsulfanyl)-piperidine-1-carboxylic acid 5-[2-(4,4-dimethyl-2,6-dioxo-piperidin-1-yl)-ethyl]-pyridin-2-yl ester

4-(Imidazol-1-yloxy)-piperidine-1-carboxylic acid 5-[2-(4,4-dimethyl-2,6-dioxo-piperidin-1-yl)-ethyl]-pyridin-2-yl ester

4-[1-(4-Carboxy-phenyl)-1H-tetrazol-5-ylsulfanyl]-piperidine-1-carboxylic acid 5-[2-(4,4-dimethyl-2,6-dioxo-piperidin-1-yl)-ethyl]-pyridin-2-yl ester

4-(4-Carboxymethyl-phenoxy)-piperidine-1-carboxylic acid 5-[2-(4,4-dimethyl-2,6-dioxo-piperidin-1-yl)-ethyl]-pyridin-2-yl ester

4-(Pyrazol-1-yloxy)-piperidine-1-carboxylic acid 5-[2-(4,4-dimethyl-2,6-dioxo-piperidin-1-yl)-ethyl]-pyridin-2-yl ester

4-(4-Carboxy-phenoxy)-piperidine-1-carboxylic acid 5-[2-(4,4-dimethyl-2,6-dioxo-piperidin-1-yl)-ethyl]-pyridin-2-yl ester

4-[4-(Pyridin-2-yloxy)-phenoxy]-piperidine-1-carboxylic acid 5-[2-(4,4-dimethyl-2,6-dioxo-piperidin-1-yl)-ethyl]-pyridin-2-yl ester 4-[4-(5-Methyl-pyridin-2-yloxy)-phenoxy]-piperidine-1-carboxylic acid 5-[2-(4,4-dimethyl-2,6-dioxo-piperidin-1-yl)-ethyl]-pyridin-2-yl ester

4-(4-Carboxy-phenoxymethyl)-piperidine-1-carboxylic acid 5-[2-(4,4-dimethyl-2,6-dioxo-piperidin-1-yl)-ethyl]-pyridin-2-yl ester

4-(4-Carboxymethyl-phenoxymethyl)-piperidine-1-carboxylic acid 5-[2-(4,4-dimethyl-2,6-dioxo-piperidin-1-yl)-ethyl]-pyridin-2-yl ester

4-(1H-Imidazol-2-ylsulfanylmethyl)-piperidine-1-carboxylic acid 5-[2-(4,4-dimethyl-2,6-dioxo-piperidin-1-yl)-ethyl]-pyridin-2-yl ester

4-(1-Methyl-1H-imidazol-2-ylsulfanylmethyl)-piperidine-1-carboxylic acid 5-[2-(4,4-dimethyl-2,6-dioxo-piperidin-1-yl)-ethyl]-pyridin-2-yl ester

4-(Imidazol-1-yloxymethyl)-piperidine-1-carboxylic acid 5-[2-(4,4-dimethyl-2,6-dioxo-piperidin-1-yl)-ethyl]-pyridin-2-yl ester

4-[4-(Pyridin-2-yloxy)-phenoxymethyl]-piperidine-1-carboxylic acid 5-[2-(4,4-dimethyl-2,6-dioxo-piperidin-1-yl)-ethyl]-pyridin-2-yl ester

4-[4-(5-Methyl-pyridin-2-yloxy)-phenoxymethyl]-piperidine-1-carboxylic acid 5-[2-(4,4-dimethyl-2,6-dioxo-piperidin-1-yl)-ethyl]-pyridin-2-yl ester

4-(1-Methyl-1H-tetrazol-5-ylsulfanylmethyl)-piperidine-1-carboxylic acid 5-[2-(4,4-dimethyl-2,6-dioxo-piperidin-1-yl)-ethyl]-pyridin-2-yl ester

4-[1-(2-Dimethylamino-ethyl)-1H-tetrazol-5-ylsulfanyl]-piperidine-1-carboxylic acid 5-[2-(4,4-dimethyl-2,6-dioxo-piperidin-1-yl)-ethyl]-pyridin-2-yl ester

4-[1-(2-Carboxy-ethyl)-1H-tetrazol-5-ylsulfanyl]-piperidine-1-carboxylic acid 5-[2-(4,4-dimethyl-2,6-dioxo-piperidin-1-yl)-ethyl]-pyridin-2-yl ester

4-(1-Carboxymethyl-1H-tetrazol-5-ylsulfanyl)-piperidine-1-carboxylic acid 5-[2-(4,4-dimethyl-2,6-dioxo-piperidin-1-yl)-ethyl]-pyridin-2-yl ester

4-(Pyrazol-1-yloxymethyl)-piperidine-1-carboxylic acid 5-[2-(4,4-dimethyl-2,6-dioxo-piperidin-1-yl)-ethyl]-pyridin-2-yl ester

4-(1H-[1,2,4]Triazol-3-ylsulfanylmethyl)-piperidine-1-carboxylic acid 5-[2-(4,4-dimethyl-2,6-dioxo-piperidin-1-yl)-ethyl]-pyridin-2-yl ester

4-[2-(Benzyl-methyl-amino)-ethyl]-piperidine-1-carboxylic acid 5-[2-(4,4-dimethyl-2,6-dioxo-piperidin-1-yl)-ethyl]-pyridin-2-yl ester

4-(1H-[1,2,4]Triazol-3-ylsulfanyl)-piperidine-1-carboxylic acid 5-benzoylamino-pyridin-2-yl ester

4-(1H-Imidazol-2-ylsulfanyl)-piperidine-1-carboxylic acid 5-benzoylamino-pyridin-2-yl ester

4-(1-Methyl-1H-imidazol-2-ylsulfanyl)-piperidine-1-carboxylic acid 5-benzoylamino-pyridin-2-yl ester

4-(1-Methyl-1H-tetrazol-5-ylsulfanyl)-piperidine-1-carboxylic acid 5-benzoylamino-pyridin-2-yl ester

4-(Imidazol-1-yloxy)-piperidine-1-carboxylic acid 5-benzoylamino-pyridin-2-yl ester

4-[1-(4-Carboxy-phenyl)-l H-tetrazol-5-ylsulfanyl]-piperidine-1-carboxylic acid 5-benzoylamino-pyridin-2-yl ester

4-(4-Carboxymethyl-phenoxy)-piperidine-1-carboxylic acid 5-benzoylamino-pyridin-2-yl ester

4-(4-Carboxy-phenoxy)-piperidine-1-carboxylic acid 5-benzoylamino-pyridin-2-yl ester

4-[4-(Pyridin-2-yloxy)-phenoxy]-piperidine-1-carboxylic acid 5-benzoylamino-pyridin-2-yl ester

4-[4-(5-Methyl-pyridin-2-yloxy)-phenoxy]-piperidine-1-carboxylic acid 5-benzoylamino-pyridin-2-yl ester

4-(4-Carboxy-phenoxymethyl)-piperidine-1-carboxylic acid 5-benzoylamino-pyridin-2-yl ester

4-(4-Carboxymethyl-phenoxymethyl)-piperidine-1-carboxylic acid 5-benzoylamino-pyridin-2-yl ester

4-(1H-I midazol-2-ylsulfanylmethyl)-piperidine-1-carboxylic acid 5-benzoylamino-pyridin-2-yl ester

4-(1-Methyl-1H-imidazol-2-ylsulfanylmethyl)-piperidine-1-carboxylic acid 5-benzoylamino-pyridin-2-yl ester

4-(Imidazol-1-yloxymethyl)-piperidine-1-carboxylic acid 5-benzoylamino-pyridin-2-yl ester

4-[4-(Pyridin-2-yloxy)-phenoxymethyl]-piperidine-1-carboxylic acid 5-benzoylamino-pyridin-2-yl ester

4-[4-(5-Methyl-pyridin-2-yloxy)-phenoxymethyl]-piperidine-1-carboxylic acid 5-benzoylamino-pyridin-2-yl ester

4-(1-Methyl-1H-tetrazol-5-ylsulfanylmethyl)-piperidine-1-carboxylic acid 5-benzoylamino-pyridin-2-yl ester

4-[1-(2-Dimethylamino-ethyl)-1H-tetrazol-5-ylsulfanyl]-piperidine-1-carboxylic acid 5-benzoylamino-pyridin-2-yl ester

4-[1-(2-Carboxy-ethyl)-1H-tetrazol-5-ylsulfanyl]-piperidine-1-carboxylic acid 5-benzoylamino-pyridin-2-yl ester

4-(1-Carboxymethyl-1H-tetrazol-5-ylsulfanyl)-piperidine-1-carboxylic acid 5-benzoylamino-pyridin-2-yl ester

4-(Pyrazol-1-yloxymethyl)-piperidine-1-carboxylic acid 5-benzoylamino-pyridin-2-yl ester

4-(1H-[1,2,4]Triazol-3-ylsulfanylmethyl)-piperidine-1-carboxylic acid 5-benzoylamino-pyridin-2-yl ester

4-[2-(Benzyl-methyl-amino)-ethyl]-piperidine-1-carboxylic acid 5-benzoylamino-pyridin-2-yl ester

4-(1H-[1,2,4]Triazol-3-ylsulfanyl)-piperidine-1-carboxylic acid 5-(3-tert-butyl-thioureido)-pyridin-2-yl ester

4-(1H-Imidazol-2-ylsulfanyl)-piperidine-1-carboxylic acid 5-(3-tert-butyl-thioureido)-pyridin-2-yl ester

4-(1-Methyl-1H-imidazol-2-ylsulfanyl)-piperidine-1-carboxylic acid 5-(3-tert-butyl-thioureido)-pyridin-2-yl ester

4-(1-Methyl-1H-tetrazol-5-ylsulfanyl)-piperidine-1-carboxylic acid 5-(3-tert-butyl-thioureido)-pyridin-2-yl ester

4-(Imidazol-1-yloxy)-piperidine-1-carboxylic acid 5-(3-tert-butyl-thioureido)-pyridin-2-yl ester

4-[1-(4-Carboxy-phenyl)-1H-tetrazol-5-ylsulfanyl]-piperidine-1-carboxylic acid 5-(3-tert-butyl-thioureido)-pyridin-2-yl ester

4-(4-Carboxymethyl-phenoxy)-piperidine-1-carboxylic acid 5-(3-tert-butyl-thioureido)-pyridin-2-yl ester

4-(Pyrazol-1-yloxy)-piperidine-1-carboxylic acid 5-(3-tert-butyl-thioureido)-pyridin-2-yl ester

4-(4-Carboxy-phenoxy)-piperidine-1-carboxylic acid 5-(3-tert-butyl-thioureido)-pyridin-2-yl ester

4-[4-(Pyridin-2-yloxy)-phenoxy]-piperidine-1-carboxylic acid 5-(3-tert-butyl-thioureido)-pyridin-2-yl ester

4-[4-(5-Methyl-pyridin-2-yloxy)-phenoxy]-piperidine-1-carboxylic acid 5-(3-tert-butyl-thioureido)-pyridin-2-yl ester

4-(4-Carboxy-phenoxymethyl)-piperidine-1-carboxylic acid 5-(3-tert-butyl-thioureido)-pyridin-2-yl ester

4-(4-Carboxymethyl-phenoxymethyl)-piperidine-1-carboxylic acid 5-(3-tert-butyl-thioureido)-pyridin-2-yl ester

4-(1H-Imidazol-2-ylsulfanylmethyl)-piperidine-1-carboxylic acid 5-(3-tert-butyl-thioureido)-pyridin-2-yl ester

4-(1-Methyl-1H-imidazol-2-ylsulfanylmethyl)-piperidine-1-carboxylic acid 5-(3-tert-butyl-thioureido)-pyridin-2-yl ester

4-(Imidazol-1-yloxymethyl)-piperidine-1-carboxylic acid 5-(3-tert-butyl-thioureido)-pyridin-2-yl ester 4-[4-(Pyridin-2-yloxy)-phenoxymethyl]-piperidine-1-carboxylic acid 5-(3-tert-butyl-thioureido)-pyridin-2-yl ester

4-[4-(5-Methyl-pyridin-2-yloxy)-phenoxymethyl]-piperidine-1-carboxylic acid 5-(3-tert-butyl-thioureido)-pyridin-2-yl ester

4-(1-Methyl-1H-tetrazol-5-ylsulfanylmethyl)-piperidine-1-carboxylic acid 5-(3-tert-butyl-thioureido)-pyridin-2-yl ester

4-[1-(2-Dimethylamino-ethyl)-1H-tetrazol-5-ylsulfanyl]-piperidine-1-carboxylic acid 5-(3-tert-butyl-thioureido)-pyridin-2-yl ester

4-[1-(2-Carboxy-ethyl)-1H-tetrazol-5-ylsulfanyl]-piperidine-1-carboxylic acid 5-(3-tert-butyl-thioureido)-pyridin-2-yl ester

4-(1-Carboxymethyl-1H-tetrazol-5-ylsulfanyl)-piperidine-1-carboxylic acid 5-(3-tert-butyl-thioureido)-pyridin-2-yl ester

4-(Pyrazol-1-yloxymethyl)-piperidine-1-carboxylic acid 5-(3-tert-butyl-thioureido)-pyridin-2-yl ester

4-(1H-[1,2,4]Triazol-3-ylsulfanylmethyl)-piperidine-1-carboxylic acid 5-(3-tert-butyl-thioureido)-pyridin-2-yl ester

4-[2-(Benzyl-methyl-amino)-ethyl]-piperidine-1-carboxylic acid 5-(3-tert-butyl-thioureido)-pyridin-2-yl ester

4-(1H-[1,2,4]Triazol-3-ylsulfanyl)-piperidine-1-carboxylic acid 4-[2-(pyridin-4-ylsulfanyl)-ethyl]-phenyl ester

4-(1H-Imidazol-2-ylsulfanyl)-piperidine-1-carboxylic acid 4-[2-(pyridin-4-ylsulfanyl)-ethyl]-phenyl ester

4-(1-Methyl-1H-imidazol-2-ylsulfanyl)-piperidine-1-carboxylic acid 4-[2-(pyridin-4-ylsulfanyl)-ethyl]-phenyl ester

4-(1-Methyl-1H-tetrazol-5-ylsulfanyl)-piperidine-1-carboxylic acid 4-[2-(pyridin-4-ylsulfanyl)-ethyl]-phenyl ester

4-(Imidazol-1-yloxy)-piperidine-1-carboxylic acid 4-[2-(pyridin-4-ylsulfanyl)-ethyl]-phenyl ester

4-[1-(4-Carboxy-phenyl)-1H-tetrazol-5-ylsulfanyl]-piperidine-1-carboxylic acid 4-[2-(pyridin-4-ylsulfanyl)-ethyl]-phenyl ester

4-(4-Carboxymethyl-phenoxy)-piperidine-1-carboxylic acid 4-[2-(pyridin-4-ylsulfanyl)-ethyl]-phenyl ester

4-(Pyrazol-1-yloxy)-piperidine-1-carboxylic acid 4-[2-(pyridin-4-ylsulfanyl)-ethyl]-phenyl ester

4-(4-Carboxy-phenoxy)-piperidine-1-carboxylic acid 4-[2-(pyridin-4-ylsulfanyl)-ethyl]-phenyl ester

4-[4-(Pyridin-2-yloxy)-phenoxy]-piperidine-1-carboxylic acid 4-[2-(pyridin-4-ylsulfanyl)-ethyl]-phenyl ester

4-[4-(5-Methyl-pyridin-2-yloxy)-phenoxy]-piperidine-1-carboxylic acid 4-[2-(pyridin-4-ylsulfanyl)-ethyl]-phenyl ester

4-(4-Carboxy-phenoxymethyl)-piperidine-1-carboxylic acid 4-[2-(pyridin-4-ylsulfanyl)-ethyl]-phenyl ester

4-(4-Carboxymethyl-phenoxymethyl)-piperidine-1-carboxylic acid 4-[2-(pyridin-4-ylsulfanyl)-ethyl]-phenyl ester

4-(1H-Imidazol-2-ylsulfanylmethyl)-piperidine-1-carboxylic acid 4-[2-(pyridin-4-ylsulfanyl)-ethyl]-phenyl ester

4-(1-Methyl-1H-imidazol-2-ylsulfanylmethyl)-piperidine-1-carboxylic acid 4-[2-(pyridin-4-ylsulfanyl)-ethyl]-phenyl ester

4-(Imidazol-1-yloxymethyl)-piperidine-1-carboxylic acid 4-[2-(pyridin-4-ylsulfanyl)-ethyl]-phenyl ester

4-[4-(Pyridin-2-yloxy)-phenoxymethyl]-piperidine-1-carboxylic acid 4-[2-(pyridin-4-ylsulfanyl)-ethyl]-phenyl ester

4-[4-(5-Methyl-pyridin-2-yloxy)-phenoxymethyl]-piperidine-1-carboxylic acid 4-[2-(pyridin-4-ylsulfanyl)-ethyl]-phenyl ester

4-(1-Methyl-1H-tetrazol-5-ylsulfanylmethyl)-piperidine-1-carboxylic acid 4-[2-(pyridin-4-ylsulfanyl)-ethyl]-phenyl ester

4-[1-(2-Dimethylamino-ethyl)-1H-tetrazol-5-ylsulfanyl]-piperidine-1-carboxylic acid 4-[2-(pyridin-4-ylsulfanyl)-ethyl]-phenyl ester

4-[1-(2-Carboxy-ethyl)-1H-tetrazol-5-ylsulfanyl]-piperidine-1-carboxylic acid 4-[2-(pyridin-4-ylsulfanyl)-ethyl]-phenyl ester

4-(1-Carboxymethyl-1H-tetrazol-5-ylsulfanyl)-piperidine-1-carboxylic acid 4-[2-(pyridin-4-ylsulfanyl)-ethyl]-phenyl ester

4-(Pyrazol-1-yloxymethyl)-piperidine-1-carboxylic acid 4-[2-(pyridin-4-ylsulfanyl)-ethyl]-phenyl ester

4-(1H-[1,2,4]Triazol-3-ylsulfanylmethyl)-piperidine-1-carboxylic acid 4-[2-(pyridin-4-ylsulfanyl)-ethyl]-phenyl ester

4-[2-(Benzyl-methyl-amino)-ethyl]-piperidine-1-carboxylic acid 4-[2-(pyridin-4-ylsulfanyl)-ethyl]-phenyl ester

4-(1H-[1,2,4]Triazol-3-ylsulfanyl)-piperidine-1-carboxylic acid 4-(1-methyl-1H-imidazol-2-ylsulfanylmethyl)-phenyl ester

4-(1H-Imidazol-2-ylsulfanyl)-piperidine-1-carboxylic acid 4-(1-methyl-1H-imidazol-2-ylsulfanylmethyl)-phenyl ester

4-(1-Methyl-1H-imidazol-2-ylsulfanyl)-piperidine-1-carboxylic acid 4-(1-methyl-1H-imidazol-2-ylsulfanylmethyl)-phenyl ester

4-(1-Methyl-1H-tetrazol-5-ylsulfanyl)-piperidine-1-carboxylic acid 4-(1-methyl-1H-imidazol-2-ylsulfanylmethyl)-phenyl ester

4-(Imidazol-1-yloxy)-piperidine-1-carboxylic acid 4-(1-methyl-1H-imidazol-2-ylsulfanylmethyl)-phenyl ester 4-[1-(4-Carboxy-phenyl)-1H-tetrazol-5-ylsulfanyl]-piperidine-1-carboxylic acid 4-(1-methyl-1H-imidazol-2-ylsulfanylmethyl)-phenyl ester

4-(4-Carboxymethyl-phenoxy)-piperidine-1-carboxylic acid 4-(1-methyl-1H-imidazol-2-ylsulfanylmethyl)-phenyl ester

4-(Pyrazol-1-yloxy)-piperidine-1-carboxylic acid 4-(1-methyl-1H-imidazol-2-ylsulfanylmethyl)-phenyl ester

4-(4-Carboxy-phenoxy)-piperidine-1-carboxylic acid 4-(1-methyl-1H-imidazol-2-ylsulfanylmethyl)-phenyl ester

4-[4-(Pyridin-2-yloxy)-phenoxy]-piperidine-1-carboxylic acid 4-(1-methyl-1H-imidazol-2-ylsulfanylmethyl)-phenyl ester

4-[4-(5-Methyl-pyridin-2-yloxy)-phenoxy]-piperidine-1-carboxylic acid 4-(1-methyl-1H-imidazol-2-ylsulfanylmethyl)-phenyl ester

4-(4-Carboxy-phenoxymethyl)-piperidine-1-carboxylic acid 4-(1-methyl-1H-imidazol-2-ylsulfanylmethyl)-phenyl ester

4-(4-Carboxymethyl-phenoxymethyl)-piperidine-1-carboxylic acid 4-(1-methyl-1H-imidazol-2-ylsulfanylmethyl)-phenyl ester

4-(1H-Imidazol-2-ylsulfanylmethyl)-piperidine-1-carboxylic acid 4-(1-methyl-1H-imidazol-2-ylsulfanylmethyl)-phenyl ester

4-(1-Methyl-1H-imidazol-2-ylsulfanylmethyl)-piperidine-1-carboxylic acid 4-(1-methyl-1H-imidazol-2-ylsulfanylmethyl)-phenyl ester

4-(Imidazol-1-yloxymethyl)-piperidine-1-carboxylic acid 4-(1-methyl-1H-imidazol-2-ylsulfanylmethyl)-phenyl ester

4-[4-(Pyridin-2-yloxy)-phenoxymethyl]-piperidine-1-carboxylic acid 4-(1-methyl-1H-imidazol-2-ylsulfanylmethyl)-phenyl ester

4-[4-(5-Methyl-pyridin-2-yloxy)-phenoxymethyl]-piperidine-1-carboxylic acid 4-(1-methyl-1H-imidazol-2-ylsulfanylmethyl)-phenyl ester

4-(1-Methyl-1H-tetrazol-5-ylsulfanylmethyl)-piperidine-1-carboxylic acid 4-(1-methyl-1H-imidazol-2-ylsulfanylmethyl)-phenyl ester

4-[1-(2-Dimethylamino-ethyl)-1H-tetrazol-5-ylsulfanyl]-piperidine-1-carboxylic acid 4-(1-methyl-1H-imidazol-2-ylsulfanylmethyl)-phenyl ester

4-[1-(2-Carboxy-ethyl)-1H-tetrazol-5-ylsulfanyl]-piperidine-1-carboxylic acid 4-(1-methyl-1H-imidazol-2-ylsulfanylmethyl)-phenyl ester

4-(1-Carboxymethyl-1H-tetrazol-5-ylsulfanyl)-piperidine-1-carboxylic acid 4-(1-methyl-1H-imidazol-2-ylsulfanylmethyl)-phenyl ester

4-(Pyrazol-1-yloxymethyl)-piperidine-1-carboxylic acid 4-(1-methyl-1H-imidazol-2-ylsulfanylmethyl)-phenyl ester

4-(1H-[1,2,4]Triazol-3-ylsulfanylmethyl)-piperidine-1-carboxylic acid 4-(1-methyl-1H-imidazol-2-ylsulfanylmethyl)-phenyl ester

4-[2-(Benzyl-methyl-amino)-ethyl]-piperidine-1-carboxylic acid 4-(1-methyl-1H-imidazol-2-ylsulfanylmethyl)-phenyl ester

4-(1H-[1,2,4]Triazol-3-ylsulfanyl)-piperidine-1-carboxylic acid 4-benzoylamino-phenyl ester

4-(1H-Imidazol-2-ylsulfanyl)-piperidine-1-carboxylic acid 4-benzoylamino-phenyl ester

4-(1-Methyl-1H-imidazol-2-ylsulfanyl)-piperidine-1-carboxylic acid 4-benzoylamino-phenyl ester

4-(1-Methyl-1H-tetrazol-5-ylsulfanyl)-piperidine-1-carboxylic acid 4-benzoylamino-phenyl ester

4-(Imidazol-1-yloxy)-piperidine-1-carboxylic acid 4-benzoylamino-phenyl ester

4-[1-(4-Carboxy-phenyl)-1H-tetrazol-5-ylsulfanyl]-piperidine-1-carboxylic acid 4-benzoylamino-phenyl ester

4-(4-Carboxymethyl-phenoxy)-piperidine-1-carboxylic acid 4-benzoylamino-phenyl ester

4-(Pyrazol-1-yloxy)-piperidine-1-carboxylic acid 4-benzoylamino-phenyl ester

4-(4-Carboxy-phenoxy)-piperidine-1-carboxylic acid 4-benzoylamino-phenyl ester

4-[4-(Pyridin-2-yloxy)-phenoxy]-piperidine-1-carboxylic acid 4-benzoylamino-phenyl ester

4-[4-(5-Methyl-pyridin-2-yloxy)-phenoxy]-piperidine-1-carboxylic acid 4-benzoylamino-phenyl ester

4-(4-Carboxy-phenoxymethyl)-piperidine-1-carboxylic acid 4-benzoylamino-phenyl ester

4-(4-Carboxymethyl-phenoxymethyl)-piperidine-1-carboxylic acid 4-benzoylamino-phenyl ester

4-(1H-Imidazol-2-ylsulfanylmethyl)-piperidine-1-carboxylic acid 4-benzoylamino-phenyl ester

4-(1-Methyl-1H-imidazol-2-ylsulfanylmethyl)-piperidine-1-carboxylic acid 4-benzoylamino-phenyl ester

4-(Imidazol-1-yloxymethyl)-piperidine-1-carboxylic acid 4-benzoylamino-phenyl ester

4-[4-(Pyridin-2-yloxy)-phenoxymethyl]-piperidine-1-carboxylic acid 4-benzoylamino-phenyl ester

4-[4-(5-Methyl-pyridin-2-yloxy)-phenoxymethyl]-piperidine-1-carboxylic acid 4-benzoylamino-phenyl ester

4-(1-Methyl-1H-tetrazol-5-ylsulfanylmethyl)-piperidine-1-carboxylic acid 4-benzoylamino-phenyl ester

4-[1-(2-Dimethylamino-ethyl)-1H-tetrazol-5-ylsulfanyl]-piperidine-1-carboxylic acid 4-benzoylamino-phenyl ester

4-[1-(2-Carboxy-ethyl)-1H-tetrazol-5-ylsulfanyl]-piperidine-1-carboxylic acid 4-benzoylamino-phenyl ester

4-(1-Carboxymethyl-1H-tetrazol-5-ylsulfanyl)-piperidine-1-carboxylic acid 4-benzoylamino-phenyl ester

4-(Pyrazol-1-yloxymethyl)-piperidine-1-carboxylic acid 4-benzoylamino-phenyl ester

4-(1H-[1,2,4]Triazol-3-ylsulfanylmethyl)-piperidine-1-carboxylic acid 4-benzoylamino-phenyl ester

4-[2-(Benzyl-methyl-amino)-ethyl]-piperidine-1-carboxylic acid 4-benzoylamino-phenyl ester

4-(1H-[1,2,4]Triazol-3-ylsulfanyl)-piperidine-1-carboxylic acid 5-(1,3-dioxo-octahydro-isoindol-2-yl)-pyridin-2-yl ester

4-(1H-Imidazol-2-ylsulfanyl)-piperidine-1-carboxylic acid 5-(1,3-dioxo-octahydro-isoindol-2-yl)-pyridin-2-yl ester

4-(1-Methyl-1H-imidazol-2-ylsulfanyl)-piperidine-1-carboxylic acid 5-(1,3-dioxo-octahydro-isoindol-2-yl)-pyridin-2-yl ester

4-(1-Methyl-1H-tetrazol-5-ylsulfanyl)-piperidine-1-carboxylic acid 5-(1,3-dioxo-octahydro-isoindol-2-yl)-pyridin-2-yl ester

4-(Imidazol-1-yloxy)-piperidine-1-carboxylic acid 5-(1,3-dioxo-octahydro-isoindol-2-yl)-pyridin-2-yl ester

4-[1-(4-Carboxy-phenyl)-1H-tetrazol-5-ylsulfanyl]-piperidine-1-carboxylic acid 5-(1,3-dioxo-octahydro-isoindol-2-yl)-pyridin-2-yl ester

4-(4-Carboxymethyl-phenoxy)-piperidine-1-carboxylic acid 5-(1,3-dioxo-octahydro-isoindol-2-yl)-pyridin-2-yl ester

4-(Pyrazol-1-yloxy)-piperidine-1-carboxylic acid 5-(1,3-dioxo-octahydro-isoindol-2-yl)-pyridin-2-yl ester

4-(4-Carboxy-phenoxy)-piperidine-1-carboxylic acid 5-(1,3-dioxo-octahydro-isoindol-2-yl)-pyridin-2-yl ester

4-[4-(Pyridin-2-yloxy)-phenoxy]-piperidine-1-carboxylic acid 5-(1,3-dioxo-octahydro-isoindol-2-yl)-pyridin-2-yl ester

4-[4-(5-Methyl-pyridin-2-yloxy)-phenoxy]-piperidine-1-carboxylic acid 5-(1,3-dioxo-octahydro-isoindol-2-yl)-pyridin-2-yl ester

4-(4-Carboxy-phenoxymethyl)-piperidine-1--carboxylic acid 5-(1,3-dioxo-octahydro-isoindol-2-yl)-pyridin-2-yl ester

4-(4-Carboxymethyl-phenoxymethyl)-piperidine-i1-carboxylic acid 5-(1,3-dioxo-octahydro-isoindol-2-yl)-pyridin-2-yl ester

4-(1H-Imidazol-2-ylsulfanylmethyl)-piperidine-1-carboxylic acid 5-(1,3-dioxo-octahydro-isoindol-2-yl)-pyridin-2-yl ester

4-(1-Methyl-1H-imidazol-2-ylsulfanylmethyl)-piperidine-1-carboxylic acid 5-(1,3-dioxo-octahydro-isoindol-2-yl)-pyridin-2-yl ester

4-(Imidazol-1-yloxymethyl)-piperidine-1-carboxylic acid 5-(1,3-dioxo-octahydro-isoindol-2-yl)-pyridin-2-yl ester

4-[4-(Pyridin-2-yloxy)-phenoxymethyl]-piperidine-1-carboxylic acid 5-(1,3-dioxo-octahydro-isoindol-2-yl)-pyridin-2-yl ester

4-[4-(5-Methyl-pyridin-2-yloxy)-phenoxymethyl]-piperidine-1-carboxylic acid 5-(1,3-dioxo-octahydro-isoindol-2-yl)-pyridin-2-yl ester

4-(1-Methyl-1H-tetrazol-5-ylsulfanylmethyl)-piperidine-1-carboxylic acid 5-(1,3-dioxo-octahydro-isoindol-2-yl)-pyridin-2-yl ester

4-[1-(2-Dimethylamino-ethyl)-1H-tetrazol-5-ylsulfanyl]-piperidine-1-carboxylic acid 5-(1,3-dioxo-octahydro-isoindol-2-yl)-pyridin-2-yl ester

4-[1-(2-Carboxy-ethyl)-1H-tetrazol-5-ylsulfanyl]-piperidine-1-carboxylic acid 5-(1,3-dioxo-octahydro-isoindol-2-yl)-pyridin-2-yl ester

4-(1-Carboxymethyl-1H-tetrazol-5-ylsulfanyl)-piperidine-1-carboxylic acid 5-(1,3-dioxo-octahydro-isoindol-2-yl)-pyridin-2-yl ester

4-(Pyrazol-1-yloxymethyl)-piperidine-1-carboxylic acid 5-(1,3-dioxo-octahydro-isoindol-2-yl)-pyridin-2-yl ester

4-(1H-[1,2,4]Triazol-3-ylsulfanylmethyl)-piperidine-1-carboxylic acid 5-(1,3-dioxo-octahydro-isoindol-2-yl)-pyridin-2-yl ester

4-[2-(Benzyl-methyl-amino)-ethyl]-piperidine-1-carboxylic acid 5-(1,3-dioxo-octahydro-isoindol-2-yl)-pyridin-2-yl ester

4-(1H-[1,2,4]Triazol-3-ylsulfanyl)-piperidine-1-carboxylic acid quinolin-7-yl ester

4-(1H-I midazol-2-ylsulfanyl)-piperidine-1-carboxylic acid quinolin-7-yl ester

4-(1-Methyl-1H-imidazol-2-ylsulfanyl)-piperidine-1-carboxylic acid quinolin-7-yl ester

4-(1-Methyl-1H-tetrazol-5-ylsulfanyl)-piperidine-1-carboxylic acid quinolin-7-yl ester

4-(Imidazol-1-yloxy)-piperidine-1-carboxylic acid quinolin-7-yl ester

4-[1-(4-Carboxy-phenyl)-1H-tetrazol-5-ylsulfanyl]-piperidine-1-carboxylic acid quinolin-7-yl ester

4-(4-Carboxymethyl-phenoxy)-piperidine-1-carboxylic acid quinolin-7-yl ester

4-(Pyrazol-1-yloxy)-piperidine-1-carboxylic acid quinolin-7-yl ester

4-(4-Carboxy-phenoxy)-piperidine-1-carboxylic acid quinolin-7-yl ester

4-[4-(Pyridin-2-yloxy)-phenoxy]-piperidine-1-carboxylic acid quinolin-7-yl ester

4-[4-(5-Methyl-pyridin-2-yloxy)-phenoxy]-piperidine-1-carboxylic acid quinolin-7-yl ester

4-(4-Carboxy-phenoxymethyl)-piperidine-1-carboxylic acid quinolin-7-yl ester

4-(4-Carboxymethyl-phenoxymethyl)-piperidine-1-carboxylic acid quinolin-7-yl ester

4-(1H-Imidazol-2-ylsulfanylmethyl)-piperidine-1-carboxylic acid quinolin-7-yl ester

4-(1-Methyl-1H-imidazol-2-ylsulfanylmethyl)-piperidine-1-carboxylic acid quinolin-7-yl ester

4-(Imidazol-1-yloxymethyl)-piperidine-1-carboxylic acid quinolin-7-yl ester

4-[4-(Pyridin-2-yloxy)-phenoxymethyl]-piperidine-1-carboxylic acid quinolin-7-yl ester

4-[4-(5-Methyl-pyridin-2-yloxy)-phenoxymethyl]-piperidine-1-carboxylic acid quinolin-7-yl ester

4-(1-Methyl-1H-tetrazol-5-ylsulfanylmethyl)-piperidine-1-carboxylic acid quinolin-7-yl ester

4-[1-(2-Dimethylamino-ethyl)-1H-tetrazol-5-ylsulfanyl]-piperidine-1-carboxylic acid quinolin-7-yl ester

4-[1-(2-Carboxy-ethyl)-1H-tetrazol-5-ylsulfanyl]-piperidine-1-carboxylic acid quinolin-7-yl ester

4-(1-Carboxymethyl-1H-tetrazol-5-ylsulfanyl)-piperidine-1-carboxylic acid quinolin-7-yl ester

4-(Pyrazol-1-yloxymethyl)-piperidine-1-carboxylic acid quinolin-7-yl ester

4-(1H-[1,2,4]Triazol-3-ylsulfanylmethyl)-piperidine-1-carboxylic acid quinolin-7-yl ester

4-[2-(Benzyl-methyl-amino)-ethyl]-piperidine-1-carboxylic acid quinolin-7-yl ester

4-(1H-[1,2,4]Triazol-3-ylsulfanyl)-piperidine-1-carboxylic acid 4-(1,1,3,3-tetramethyl-butylcarbamoyl)-phenyl ester

4-(1H-Imidazol-2-ylsulfanyl)-piperidine-1-carboxylic acid 4-(1,1,3,3-tetramethyl-butylcarbamoyl)-phenyl ester

4-(1-Methyl-1H-imidazol-2-ylsulfanyl)-piperidine-1-carboxylic acid 4-(1,1,3,3-tetramethyl-butylcarbamoyl)-phenyl ester

4-(1-Methyl-1H-tetrazol-5-ylsulfanyl)-piperidine-1-carboxylic acid 4-(1,1,3,3-tetramethyl-butylcarbamoyl)-phenyl ester

4-(Imidazol-1-yloxy)-piperidine-1-carboxylic acid 4-(1,1,3,3-tetramethyl-butylcarbamoyl)-phenyl ester

4-[1-(4-Carboxy-phenyl)-1H-tetrazol-5-ylsulfanyl]-piperidine-1-carboxylic acid 4-(1,1,3,3-tetramethyl-butylcarbamoyl)-phenyl ester

4-(4-Carboxymethyl-phenoxy)-piperidine-1-carboxylic acid 4-(1,1,3,3-tetramethyl-butylcarbamoyl)-phenyl ester

4-(Pyrazol-1-yloxy)-piperidine-1-carboxylic acid 4-(1,1,3,3-tetramethyl-butylcarbamoyl)-phenyl ester

4-(4-Carboxy-phenoxy)-piperidine-1-carboxylic acid 4-(1,1,3,3-tetramethyl-butylcarbamoyl)-phenyl ester

4-[4-(Pyridin-2-yloxy)-phenoxy]-piperidine-1-carboxylic acid 4-(1,1,3,3-tetramethyl-butylcarbamoyl)-phenyl ester

4-[4-(5-Methyl-pyridin-2-yloxy)-phenoxy]-piperidine-1-carboxylic acid 4-(1,1,3,3-tetramethyl-butylcarbamoyl)-phenyl ester

4-(4-Carboxy-phenoxymethyl)-piperidine-1-carboxylic acid 4-(1,1,3,3-tetramethyl-butylcarbamoyl)-phenyl ester

4-(4-Carboxymethyl-phenoxymethyl)-piperidine-1-carboxylic acid 4-(1,1,3,3-tetramethyl-butylcarbamoyl)-phenyl ester

4-(1H-Imidazol-2-ylsulfanylmethyl)-piperidine-1-carboxylic acid 4-(1,1,3,3-tetramethyl-butylcarbamoyl)-phenyl ester

4-(1-Methyl-1H-imidazol-2-ylsulfanylmethyl)-piperidine-1-carboxylic acid 4-(1,1,3,3-tetramethyl-butylcarbamoyl)-phenyl ester

4-(Imidazol-1-yloxymethyl)-piperidine-1-carboxylic acid 4-(1,1,3,3-tetramethyl-butylcarbamoyl)-phenyl ester

4-[4-(Pyridin-2-yloxy)-phenoxymethyl]-piperidine-1-carboxylic acid 4-(1,1,3,3-tetramethyl-butylcarbamoyl)-phenyl ester

4-[4-(5-Methyl-pyridin-2-yloxy)-phenoxymethyl]-piperidine-1-carboxylic acid 4-(1,1,3,3-tetramethyl-butylcarbamoyl)-phenyl ester

4-(1-Methyl-1H-tetrazol-5-ylsulfanylmethyl)-piperidine-1-carboxylic acid 4-(1,1,3,3-tetramethyl-butylcarbamoyl)-phenyl ester

4-[1-(2-Dimethylamino-ethyl)-1H-tetrazol-5-ylsulfanyl]-piperidine-1-carboxylic acid 4-(1,1,3,3-tetramethyl-butylcarbamoyl)-phenyl ester

4-[1-(2-Carboxy-ethyl)-1H-tetrazol-5-ylsulfanyl]-piperidine-1-carboxylic acid 4-(1,1,3,3-tetramethyl-butylcarbamoyl)-phenyl ester

4-(1-Carboxymethyl-1H-tetrazol-5-ylsulfanyl)-piperidine-1-carboxylic acid 4-(1,1,3,3-tetramethyl-butylcarbamoyl)-phenyl ester

4-(Pyrazol-1-yloxymethyl)-piperidine-1-carboxylic acid 4-(1,1,3,3-tetramethyl-butylcarbamoyl)-phenyl ester

4-(1H-[1,2,4]Triazol-3-ylsulfanylmethyl)-piperidine-1-carboxylic acid 4-(1,1,3,3-tetramethyl-butylcarbamoyl)-phenyl ester

4-[2-(Benzyl-methyl-amino)-ethyl]-piperidine-1-carboxylic acid 4-(1,1,3,3-tetramethyl-butylcarbamoyl)-phenyl ester

4-(1H-[1,2,4]Triazol-3-ylsulfanyl)-piperidine-1-carboxylic acid 4-(cyclohexanecarbonyl-amino)-phenyl ester

4-(1H-Imidazol-2-ylsulfanyl)-piperidine-1-carboxylic acid 4-(cyclohexanecarbonyl-amino)-phenyl ester

4-(1-Methyl-1H-imidazol-2-ylsulfanyl)-piperidine-1-carboxylic acid 4-(cyclohexanecarbonyl-amino)-phenyl ester

4-(1-Methyl-1H-tetrazol-5-ylsulfanyl)-piperidine-1-carboxylic acid 4-(cyclohexanecarbonyl-amino)-phenyl ester

4-(Imidazol-1-yloxy)-piperidine-1-carboxylic acid 4-(cyclohexanecarbonyl-amino)-phenyl ester

4-[1-(4-Carboxy-phenyl)-1H-tetrazol-5-ylsulfanyl]-piperidine-1-carboxylic acid 4-(cyclohexanecarbonyl-amino)-phenyl ester

4-(4-Carboxymethyl-phenoxy)-piperidine-1-carboxylic acid 4-(cyclohexanecarbonyl-amino)-phenyl ester

4-(Pyrazol-1-yloxy)-piperidine-1-carboxylic acid 4-(cyclohexanecarbonyl-amino)-phenyl ester

4-(4-Carboxy-phenoxy)-piperidine-1-carboxylic acid 4-(cyclohexanecarbonyl-amino)-phenyl ester

4-[4-(Pyridin-2-yloxy)-phenoxy]-piperidine-1-carboxylic acid 4-(cyclohexanecarbonyl-amino)-phenyl ester

4-[4-(5-Methyl-pyridin-2-yloxy)-phenoxy]-piperidine-1-carboxylic acid 4-(cyclohexanecarbonyl-amino)-phenyl ester

4-(4-Carboxy-phenoxymethyl)-piperidine-1-carboxylic acid 4-(cyclohexanecarbonyl-amino)-phenyl ester

4-(4-Carboxymethyl-phenoxymethyl)-piperidine-1-carboxylic acid 4-(cyclohexanecarbonyl-amino)-phenyl ester

4-(1H-Imidazol-2-ylsulfanylmethyl)-piperidine-1-carboxylic acid 4-(cyclohexanecarbonyl-amino)-phenyl ester

4-(1-Methyl-1H-imidazol-2-ylsulfanylmethyl)-piperidine-1-carboxylic acid 4-(cyclohexanecarbonyl-amino)-phenyl ester

4-(Imidazol-1-yloxymethyl)-piperidine-1-carboxylic acid 4-(cyclohexanecarbonyl-amino)-phenyl ester

4-[4-(Pyridin-2-yloxy)-phenoxymethyl]-piperidine-1-carboxylic acid 4-(cyclohexanecarbonyl-amino)-phenyl ester

4-[4-(5-Methyl-pyridin-2-yloxy)-phenoxymethyl]-piperidine-1-carboxylic acid 4-(cyclohexanecarbonyl-amino)-phenyl ester

4-(1-Methyl-1H-tetrazol-5-ylsulfanylmethyl)-piperidine-1-carboxylic acid 4-(cyclohexanecarbonyl-amino)-phenyl ester

4-[1-(2-Dimethylamino-ethyl)-1H-tetrazol-5-ylsulfanyl]-piperidine-1-carboxylic acid 4-(cyclohexanecarbonyl-amino)-phenyl ester

4-[1-(2-Carboxy-ethyl)-1H-tetrazol-5-ylsulfanyl]-piperidine-1-carboxylic acid 4-(cyclohexanecarbonyl-amino)-phenyl ester

4-(1-Carboxymethyl-1H-tetrazol-5-ylsulfanyl)-piperidine-1-carboxylic acid 4-(cyclohexanecarbonyl-amino)-phenyl ester

4-(Pyrazol-1-yloxymethyl)-piperidine-1-carboxylic acid 4-(cyclohexanecarbonyl-amino)-phenyl ester

4-(1H-[1,2,4]Triazol-3-ylsulfanylmethyl)-piperidine-1-carboxylic acid 4-(cyclohexanecarbonyl-amino)-phenyl ester

4-[2-(Benzyl-methyl-amino)-ethyl]-piperidine-1-carboxylic acid 4-(cyclohexanecarbonyl-amino)-phenyl ester

4-(1H-[1,2,4]Triazol-3-ylsulfanyl)-piperidine-1-carboxylic acid 4-[2-(4,4-dimethyl-2,6-dioxo-piperidin-1-yl)-ethyl]-phenyl ester

4-(1-Methyl-1H-tetrazol-5-ylsulfanyl)-piperidine-1-carboxylic acid 4-[2-(4,4-dimethyl-2,6-dioxo-piperidin-1-yl)-ethyl]-phenyl ester

4-[1-(4-Carboxy-phenyl)-1H-tetrazol-5-ylsulfanyl]-piperidine-1-carboxylic acid 4-[2-(4,4-dimethyl-2,6-dioxo-piperidin-1-yl)-ethyl]-phenyl ester

4-[4-(Pyridin-2-yloxy)-phenoxy]-piperidine-1-carboxylic acid 4-[2-(4,4-dimethyl-2,6-dioxo-piperidin-1-yl)-ethyl]-phenyl ester 4-[4-(5-Methyl-pyridin-2-yloxy)-phenoxy]-piperidine-1-carboxylic acid 4-[2-(4,4-dimethyl-2,6-dioxo-piperidin-1-yl)-ethyl]-phenyl ester

4-(4-Carboxy-phenoxymethyl)-piperidine-1-carboxylic acid 4-[2-(4,4-dimethyl-2,6-dioxo-piperidin-1-yl)-ethyl]-phenyl ester

4-(4-Carboxymethyl-phenoxymethyl)-piperidine-1-carboxylic acid 4-[2-(4,4-dimethyl-2,6-dioxo-piperidin-1-yl)-ethyl]-phenyl ester

4-(1H-Imidazol-2-ylsulfanylmethyl)-piperidine-1-carboxylic acid 4-[2-(4,4-dimethyl-2,6-dioxo-piperidin-1-yl)-ethyl]-phenyl ester

4-(1-Methyl-1H-imidazol-2-ylsulfanylmethyl)-piperidine-1-carboxylic acid 4-[2-(4,4-dimethyl-2,6-dioxo-piperidin-1-yl)-ethyl]-phenyl ester

4-(Imidazol-1-yloxymethyl)-piperidine-1--carboxylic acid 4-[2-(4,4-dimethyl-2,6-dioxo-piperidin-1-yl)-ethyl]-phenyl ester

4-[4-(Pyridin-2-yloxy)-phenoxymethyl]-piperidine-1-carboxylic acid 4-[2-(4,4-dimethyl-2,6-dioxo-piperidin-1-yl)-ethyl]-phenyl ester

4-[4-(5-Methyl-pyridin-2-yloxy)-phenoxymethyl]-piperidine-1-carboxylic acid 4-[2-(4,4-dimethyl-2,6-dioxo-piperidin-1-yl)-ethyl]-phenyl ester

4-(1-Methyl-1H-tetrazol-5-ylsulfanylmethyl)-piperidine-1-carboxylic acid 4-[2-(4,4-dimethyl-2,6-dioxo-piperidin-1-yl)-ethyl]-phenyl ester

4-[1-(2-Dimethylamino-ethyl)-1H-tetrazol-5-ylsulfanyl]-piperidine-1-carboxylic acid 4-[2-(4,4-dimethyl-2,6-dioxo-piperidin-1-yl)-ethyl]-phenyl ester

4-[1-(2-Carboxy-ethyl)-1H-tetrazol-5-ylsulfanyl]-piperidine-1-carboxylic acid 4-[2-(4,4-dimethyl-2,6-dioxo-piperidin-1-yl)-ethyl]-phenyl ester

4-(1-Carboxymethyl-1H-tetrazol-5-ylsulfanyl)-piperidine-1-carboxylic acid 4-[2-(4,4-dimethyl-2,6-dioxo-piperidin-1-yl)-ethyl]-phenyl ester

4-(Pyrazol-1-yloxymethyl )-piperidine-1-carboxylic acid 4-[2-(4,4-dimethyl-2,6-dioxo-piperidin-1-yl)-ethyl]-phenyl ester

4-(1H-[1,2,4]Triazol-3-ylsulfanylmethyl)-piperidine-1-carboxylic acid 4-[2-(4,4-dimethyl-2,6-dioxo-piperidin-1-yl)-ethyl]-phenyl ester 4-[2-(Benzyl-methyl-amino)-ethyl]-piperidine-1-carboxylic acid 4-[2-(4,4-dimethyl-2,6-dioxo-piperidin-1-yl)-ethyl]-phenyl ester

4-(1H-[1,2,4]Triazol-3-ylsulfanyl)-piperidine-1-carboxylic acid 4-(6-methyl-pyridin-2-ylmethyl)-phenyl ester

4-(1H-Imidazol-2-ylsulfanyl)-piperidine-1-carboxylic acid 4-(6-methyl-pyridin-2-ylmethyl)-phenyl ester

4-(1-Methyl-1H-imidazol-2-ylsulfanyl)-piperidine-1-carboxylic acid 4-(6-methyl-pyridin-2-ylmethyl)-phenyl ester

4-(1-Methyl-1H-tetrazol-5-ylsulfanyl)-piperidine-1-carboxylic acid 4-(6-methyl-pyridin-2-ylmethyl)-phenyl ester

4-(Imidazol-1-yloxy)-piperidine-1-carboxylic acid 4-(6-methyl-pyridin-2-ylmethyl)-phenyl ester

4-[1-(4-Carboxy-phenyl)-1H-tetrazol-5-ylsulfanyl]-piperidine-1-carboxylic acid 4-(6-methyl-pyridin-2-ylmethyl)-phenyl ester

4-(4-Carboxymethyl-phenoxy)-piperidine-1-carboxylic acid 4-(6-methyl-pyridin-2-ylmethyl)-phenyl ester

4-(Pyrazol-1-yloxy)-piperidine-1-carboxylic acid 4-(6-methyl-pyridin-2-ylmethyl)-phenyl ester

4-(4-Carboxy-phenoxy)-piperidine-1-carboxylic acid 4-(6-methyl-pyridin-2-ylmethyl)-phenyl ester

4-[4-(Pyridin-2-yloxy)-phenoxy]-piperidine-1-carboxylic acid 4-(6-methyl-pyridin-2-ylmethyl)-phenyl ester

4-[4-(5-Methyl-pyridin-2-yloxy)-phenoxy]-piperidine-1-carboxylic acid 4-(6-methyl-pyridin-2-ylmethyl)-phenyl ester

4-(4-Carboxy-phenoxymethyl)-piperidine-1-carboxylic acid 4-(6-methyl-pyridin-2-ylmethyl)-phenyl ester

4-(4-Carboxymethyl-phenoxymethyl)-piperidine-1-carboxylic acid 4-(6-methyl-pyridin-2-ylmethyl)-phenyl ester

4-(1H-Imidazol-2-ylsulfanylmethyl)-piperidine-1-carboxylic acid 4-(6-methyl-pyridin-2-ylmethyl)-phenyl ester

4-(1-Methyl-1H-imidazol-2-ylsulfanylmethyl)-piperidine-1-carboxylic acid 4-(6-methyl-pyridin-2-ylmethyl)-phenyl ester

4-(Imidazol-1-yloxymethyl)-piperidine-1-carboxylic acid 4-(6-methyl-pyridin-2-ylmethyl)-phenyl ester

4-[4-(Pyridin-2-yloxy)-phenoxymethyl]-piperidine-1-carboxylic acid 4-(6-methyl-pyridin-2-ylmethyl)-phenyl ester

4-[4-(5-Methyl-pyridin-2-yloxy)-phenoxymethyl]-piperidine-1-carboxylic acid 4-(6-methyl-pyridin-2-ylmethyl)-phenyl ester

4-(1-Methyl-1H-tetrazol-5-ylsulfanylmethyl)-piperidine-1-carboxylic acid 4-(6-methyl-pyridin-2-ylmethyl)-phenyl ester

4-[1-(2-Dimethylamino-ethyl)-1H-tetrazol-5-ylsulfanyl]-piperidine-1-carboxylic acid 4-(6-methyl-pyridin-2-ylmethyl)-phenyl ester

4-[1-(2-Carboxy-ethyl)-1H-tetrazol-5-ylsulfanyl]-piperidine-1-carboxylic acid 4-(6-methyl-pyridin-2-ylmethyl)-phenyl ester

4-(1-Carboxymethyl-1H-tetrazol-5-ylsulfanyl)-piperidine-1-carboxylic acid 4-(6-methyl-pyridin-2-ylmethyl)-phenyl ester

4-(Pyrazol-1-yloxymethyl)-piperidine-1-carboxylic acid 4-(6-methyl-pyridin-2-ylmethyl)-phenyl ester

4-(1H-[1,2,4]Triazol-3-ylsulfanylmethyl)-piperidine-1-carboxylic acid 4-(6-methyl-pyridin-2-ylmethyl)-phenyl ester

4-[2-(Benzyl-methyl-amino)-ethyl]-piperidine-1-carboxylic acid 4-(6-methyl-pyridin-2-ylmethyl)-phenyl ester

4-(1H-[1,2,4]Triazol-3-ylsulfanyl)-piperidine-1-carboxylic acid 4-(1H-imidazol-2-ylsulfanylmethyl)-phenyl ester

4-(1H-Imidazol-2-ylsulfanyl)-piperidine-1-carboxylic acid 4-(1H-imidazol-2-ylsulfanylmethyl)-phenyl ester

4-(1-Methyl-1H-imidazol-2-ylsulfanyl)-piperidine-1-carboxylic acid 4-(1H-imidazol-2-ylsulfanylmethyl)-phenyl ester

4-(1-Methyl-1H-tetrazol-5-ylsulfanyl)-piperidine-1-carboxylic acid 4-(1H-imidazol-2-ylsulfanylmethyl)-phenyl ester

4-(Imidazol-1-yloxy)-piperidine-1-carboxylic acid 4-(1H-imidazol-2-ylsulfanylmethyl)-phenyl ester

4-[1-(4-Carboxy-phenyl)-1H-tetrazol-5-ylsulfanyl]-piperidine-1-carboxylic acid 4-(1H-imidazol-2-ylsulfanylmethyl)-phenyl ester

4-(4-Carboxymethyl-phenoxy)-piperidine-1-carboxylic acid 4-(1H-imidazol-2-ylsulfanylmethyl)-phenyl ester

4-(Pyrazol-1-yloxy)-piperidine-1-carboxylic acid 4-(1H-imidazol-2-ylsulfanylmethyl)-phenyl ester

4-(4-Carboxy-phenoxy)-piperidine-1-carboxylic acid 4-(1H-imidazol-2-ylsulfanylmethyl)-phenyl ester

4-[4-(Pyridin-2-yloxy)-phenoxy]-piperidine-1-carboxylic acid 4-(1H-imidazol-2-ylsulfanylmethyl)-phenyl ester

4-[4-(5-Methyl-pyridin-2-yloxy)-phenoxy]-piperidine-1-carboxylic acid 4-(1H-imidazol-2-ylsulfanylmethyl)-phenyl ester

4-(4-Carboxy-phenoxymethyl)-piperidine-1-carboxylic acid 4-(1H-imidazol-2-ylsulfanylmethyl)-phenyl ester

4-(4-Carboxymethyl-phenoxymethyl)-piperidine-1-carboxylic acid 4-(1H-imidazol-2-ylsulfanylmethyl)-phenyl ester

4-(1H-Imidazol-2-ylsulfanylmethyl)-piperidine-1-carboxylic acid 4-(1H-imidazol-2-ylsulfanylmethyl)-phenyl ester

4-(1-Methyl-1H-imidazol-2-ylsulfanylmethyl)-piperidine-1-carboxylic acid 4-(1H-imidazol-2-ylsulfanylmethyl)-phenyl ester

4-(Imidazol-1-yloxymethyl)-piperidine-1-carboxylic acid 4-(1H-imidazol-2-ylsulfanylmethyl)-phenyl ester

4-[4-(Pyridin-2-yloxy)-phenoxymethyl]-piperidine-1-carboxylic acid 4-(1H-imidazol-2-ylsulfanylmethyl)-phenyl ester

4-[4-(5-Methyl-pyridin-2-yloxy)-phenoxymethyl]-piperidine-1-carboxylic acid 4-(1H-imidazol-2-ylsulfanylmethyl)-phenyl ester

4-(1-Methyl-1H-tetrazol-5-ylsulfanylmethyl)-piperidine-1-carboxylic acid 4-(1H-imidazol-2-ylsulfanylmethyl)-phenyl ester

4-[1-(2-Dimethylamino-ethyl)-1H-tetrazol-5-ylsulfanyl]-piperidine-1-carboxylic acid 4-(1H-imidazol-2-ylsulfanylmethyl)-phenyl ester

4-[1-(2-Carboxy-ethyl)-1H-tetrazol-5-ylsulfanyl]-piperidine-1-carboxylic acid 4-(1H-imidazol-2-ylsulfanylmethyl)-phenyl ester

4-(1-Carboxymethyl-1H-tetrazol-5-ylsulfanyl)-piperidine-1-carboxylic acid 4-(1H-imidazol-2-ylsulfanylmethyl)-phenyl ester

4-(Pyrazol-1-yloxymethyl)-piperidine-1-carboxylic acid 4-(1H-imidazol-2-ylsulfanylmethyl)-phenyl ester

4-(1H-[1,2,4]Triazol-3-ylsulfanylmethyl)-piperidine-1-carboxylic acid 4-(1H-imidazol-2-ylsulfanylmethyl)-phenyl ester

4-[2-(Benzyl-methyl-amino)-ethyl]-piperidine-1-carboxylic acid 4-(1H-imidazol-2-ylsulfanylmethyl)-phenyl ester

4-(1H-[1,2,4]Triazol-3-ylsulfanyl)-piperidine-1-carboxylic acid 4-[2-(1H-imidazol-2-ylsulfanyl)-ethyl]-phenyl ester

4-(1H-Imidazol-2-ylsulfanyl)-piperidine-1-carboxylic acid 4-[2-(1H-imidazol-2-ylsulfanyl)-ethyl]-phenyl ester

4-(1-Methyl-1H-imidazol-2-ylsulfanyl)-piperidine-1-carboxylic acid 4-[2-(1H-imidazol-2-ylsulfanyl)-ethyl]-phenyl ester

4-(1-Methyl-1H-tetrazol-5-ylsulfanyl)-piperidine-1-carboxylic acid 4-[2-(1H-imidazol-2-ylsulfanyl)-ethyl]-phenyl ester

4-(Imidazol-1-yloxy)-piperidine-1-carboxylic acid 4-[2-(1H-imidazol-2-ylsulfanyl)-ethyl]-phenyl ester

4-[1-(4-Carboxy-phenyl)-1H-tetrazol-5-ylsulfanyl]-piperidine-1-carboxylic acid 4-[2-(1H-imidazol-2-ylsulfanyl)-ethyl]-phenyl ester

4-(4-Carboxymethyl-phenoxy)-piperidine-1-carboxylic acid 4-[2-(1H-imidazol-2-ylsulfanyl)-ethyl]-phenyl ester

4-(Pyrazol-1-yloxy)-piperidine-1-carboxylic acid 4-[2-(1H-imidazol-2-ylsulfanyl)-ethyl]-phenyl ester

4-(4-Carboxy-phenoxy)-piperidine-1-carboxylic acid 4-[2-(1H-imidazol-2-ylsulfanyl)-ethyl]-phenyl ester

4-[4-(Pyridin-2-yloxy)-phenoxy]-piperidine-1-carboxylic acid 4-[2-(1H-imidazol-2-ylsulfanyl)-ethyl]-phenyl ester

4-[4-(5-Methyl-pyridin-2-yloxy)-phenoxy]-piperidine-1-carboxylic acid 4-[2-(1H-imidazol-2-ylsulfanyl)-ethyl]-phenyl ester

4-(4-Carboxy-phenoxymethyl)-piperidine-1-carboxylic acid 4-[2-(1H-imidazol-2-ylsulfanyl)-ethyl]-phenyl ester

4-(4-Carboxymethyl-phenoxymethyl)-piperidine-1-carboxylic acid 4-[2-(1H-imidazol-2-ylsulfanyl)-ethyl]-phenyl ester

4-(1H-Imidazol-2-ylsulfanylmethyl)-piperidine-1-carboxylic acid 4-[2-(1H-imidazol-2-ylsulfanyl)-ethyl]-phenyl ester

4-(1-Methyl-1H-imidazol-2-ylsulfanylmethyl)-piperidine-1-carboxylic acid 4-[2-(1H-imidazol-2-ylsulfanyl)-ethyl]-phenyl ester

4-(Imidazol-1-yloxymethyl)-piperidine-1-carboxylic acid 4-[2-(1H-imidazol-2-ylsulfanyl)-ethyl]-phenyl ester

4-[4-(Pyridin-2-yloxy)-phenoxymethyl]-piperidine-1-carboxylic acid 4-[2-(1H-imidazol-2-ylsulfanyl)-ethyl]-phenyl ester

4-[4-(5-Methyl-pyridin-2-yloxy)-phenoxymethyl]-piperidine-1-carboxylic acid 4-[2-(1H-imidazol-2-ylsulfanyl)-ethyl]-phenyl ester

4-(1-Methyl-1H-tetrazol-5-ylsulfanylmethyl)-piperidine-1-carboxylic acid 4-[2-(1H-imidazol-2-ylsulfanyl)-ethyl]-phenyl ester

4-[1-(2-Dimethylamino-ethyl)-1H-tetrazol-5-ylsulfanyl]-piperidine-1-carboxylic acid 4-[2-(1H-imidazol-2-ylsulfanyl)-ethyl]-phenyl ester

4-[1-(2-Carboxy-ethyl)-1H-tetrazol-5-ylsulfanyl]-piperidine-1-carboxylic acid 4-[2-(1H-imidazol-2-ylsulfanyl)-ethyl]-phenyl ester

4-(1-Carboxymethyl-1H-tetrazol-5-ylsulfanyl)-piperidine-1-carboxylic acid 4-[2-(1H-imidazol-2-ylsulfanyl)-ethyl]-phenyl ester

4-(Pyrazol-1-yloxymethyl)-piperidine-1-carboxylic acid 4-[2-(1H-imidazol-2-ylsulfanyl)-ethyl]-phenyl ester

4-(1H-[1,2,4]Triazol-3-ylsulfanylmethyl)-piperidine-1-carboxylic acid 4-[2-(1H-imidazol-2-ylsulfanyl)-ethyl]-phenyl ester

4-[2-(Benzyl-methyl-amino)-ethyl]-piperidine-1-carboxylic acid 4-[2-(1H-imidazol-2-ylsulfanyl)-ethyl]-phenyl ester

4-(1H-[1,2,4]Triazol-3-ylsulfanyl)-piperidine-1-carboxylic acid 6-(4-isopropyl-5-oxo-2-thioxo-imidazolidin-1-yl)-pyridin-3-yl ester

4-(1H-Imidazol-2-ylsulfanyl)-piperidine-1-carboxylic acid 6-(4-isopropyl-5-oxo-2-thioxo-imidazolidin-1-yl)-pyridin-3-yl ester

4-(1-Methyl-1H-imidazol-2-ylsulfanyl)-piperidine-1-carboxylic acid 6-(4-isopropyl-5-oxo-2-thioxo-imidazolidin-1-yl)-pyridin-3-yl ester

4-(1-Methyl-1H-tetrazol-5-ylsulfanyl)-piperidine-1-carboxylic acid 6-(4-isopropyl-5-oxo-2-thioxo-imidazolidin-1-yl)-pyridin-3-yl ester

4-(Imidazol-1-yloxy)-piperidine-1-carboxylic acid 6-(4-isopropyl-5-oxo-2-thioxo-imidazolidin-1-yl)-pyridin-3-yl ester

4-[1-(4-Carboxy-phenyl)-1H-tetrazol-5-ylsulfanyl]-piperidine-1-carboxylic acid 6-(4-isopropyl-5-oxo-2-thioxo-imidazolidin-1-yl)-pyridin-3-yl ester

4-(4-Carboxymethyl-phenoxy)-piperidine-1-carboxylic acid 6-(4-isopropyl-5-oxo-2-thioxo-imidazolidin-1-yl)-pyridin-3-yl ester

4-(Pyrazol-1-yloxy)-piperidine-1-carboxylic acid 6-(4-isopropyl-5-oxo-2-thioxo-imidazolidin-1-yl)-pyridin-3-yl ester

4-(4-Carboxy-phenoxy)-piperidine-1-carboxylic acid 6-(4-isopropyl-5-oxo-2-thioxo-imidazolidin-1-yl)-pyridin-3-yl ester

4-[4-(Pyridin-2-yloxy)-phenoxy]-piperidine-1-carboxylic acid 6-(4-isopropyl-5-oxo-2-thioxo-imidazolidin-1-yl)-pyridin-3-yl ester

4-[4-(5-Methyl-pyridin-2-yloxy)-phenoxy]-piperidine-1-carboxylic acid 6-(4-isopropyl-5-oxo-2-thioxo-imidazolidin-1-yl)-pyridin-3-yl ester

4-(4-Carboxy-phenoxymethyl)-piperidine-1-carboxylic acid 6-(4-isopropyl-5-oxo-2-thioxo-imidazolidin-1-yl)-pyridin-3-yl ester

4-(4-Carboxymethyl-phenoxymethyl)-piperidine-1-carboxylic acid 6-(4-isopropyl-5-oxo-2-thioxo-imidazolidin-1-yl)-pyridin-3-yl ester

4-(1H-Imidazol-2-ylsulfanylmethyl)-piperidine-1-carboxylic acid 6-(4-isopropyl-5-oxo-2-thioxo-imidazolidin-1-yl)-pyridin-3-yl ester

4-(1-Methyl-1H-imidazol-2-ylsulfanylmethyl)-piperidine-1-carboxylic acid 6-(4-isopropyl-5-oxo-2-thioxo-imidazolidin-1-yl)-pyridin-3-yl ester

4-(Imidazol-1-yloxymethyl)-piperidine-1-carboxylic acid 6-(4-isopropyl-5-oxo-2-thioxo-imidazolidin-1-yl)-pyridin-3-yl ester

4-[4-(Pyridin-2-yloxy)-phenoxymethyl]-piperidine-1-carboxylic acid 6-(4-isopropyl-5-oxo-2-thioxo-imidazolidin-1-yl)-pyridin-3-yl ester

4-[4-(5-Methyl-pyridin-2-yloxy)-phenoxymethyl]-piperidine-1-carboxylic acid 6-(4-isopropyl-5-oxo-2-thioxo-imidazolidin-1-yl)-pyridin-3-yl ester

4-(1-Methyl-1H-tetrazol-5-ylsulfanylmethyl)-piperidine-1-carboxylic acid 6-(4-isopropyl-5-oxo-2-thioxo-imidazolidin-1-yl)-pyridin-3-yl ester

4-[1-(2-Dimethylamino-ethyl)-1H-tetrazol-5-ylsulfanyl]-piperidine-1-carboxylic acid 6-(4-isopropyl-5-oxo-2-thioxo-imidazolidin-1-yl)-pyridin-3-yl ester

4-[1-(2-Carboxy-ethyl)-1H-tetrazol-5-ylsulfanyl]-piperidine-1-carboxylic acid 6-(4-isopropyl-5-oxo-2-thioxo-imidazolidin-1-yl)-pyridin-3-yl ester

4-(1-Carboxymethyl-1H-tetrazol-5-ylsulfanyl)-piperidine-1-carboxylic acid 6-(4-isopropyl-5-oxo-2-thioxo-imidazolidin-1-yl)-pyridin-3-yl ester

4-(Pyrazol-1-yloxymethyl)-piperidine-1-carboxylic acid 6-(4-isopropyl-5-oxo-2-thioxo-imidazolidin-1-yl)-pyridin-3-yl ester

4-(1H-[1,2,4]Triazol-3-ylsulfanylmethyl)-piperidine-1-carboxylic acid 6-(4-isopropyl-5-oxo-2-thioxo-imidazolidin-1-yl)-pyridin-3-yl ester

4-[2-(Benzyl-methyl-amino)-ethyl]-piperidine-1-carboxylic acid 6-(4-isopropyl-5-oxo-2-thioxo-imidazolidin-1-yl)-pyridin-3-yl ester 4-(1H-[1,2,4]Triazol-3-ylsulfanyl)-piperidine-1-carboxylic acid 6-(4-morpholin-4-ylmethyl-benzoylamino)-pyridin-3-yl ester

4-(1H-Imidazol-2-ylsulfanyl)-piperidine-1-carboxylic acid 6-(4-morpholin-4-ylmethyl-benzoylamino)-pyridin-3-yl ester

4-(1-Methyl-1H-imidazol-2-ylsulfanyl)-piperidine-1-carboxylic acid 6-(4-morpholin-4-ylmethyl-benzoylamino)-pyridin-3-yl ester

4-(1-Methyl-1H-tetrazol-5-ylsulfanyl)-piperidine-1-carboxylic acid 6-(4-morpholin-4-ylmethyl-benzoylamino)-pyridin-3-yl ester

4-(Imidazol-1-yloxy)-piperidine-1-carboxylic acid 6-(4-morpholin-4-ylmethyl-benzoylamino)-pyridin-3-yl ester

4-[1-(4-Carboxy-phenyl)-1H-tetrazol-5-ylsulfanyl]-piperidine-1-carboxylic acid 6-(4-morpholin-4-ylmethyl-benzoylamino)-pyridin-3-yl ester

4-(4-Carboxymethyl-phenoxy)-piperidine-1-carboxylic acid 6-(4-morpholin-4-ylmethyl-benzoylamino)-pyridin-3-yl ester

4-(Pyrazol-1-yloxy)-piperidine-1-carboxylic acid 6-(4-morpholin-4-ylmethyl-benzoylamino)-pyridin-3-yl ester

4-(4-Carboxy-phenoxy)-piperidine-1-carboxylic acid 6-(4-morpholin-4-ylmethyl-benzoylamino)-pyridin-3-yl ester

4-[4-(Pyridin-2-yloxy)-phenoxy]-piperidine-1-carboxylic acid 6-(4-morpholin-4-ylmethyl-benzoylamino)-pyridin-3-yl ester

4-[4-(5-Methyl-pyridin-2-yloxy)-phenoxy]-piperidine-1-carboxylic acid 6-(4-morpholin-4-ylmethyl-benzoylamino)-pyridin-3-yl ester

4-(4-Carboxy-phenoxymethyl)-piperidine-1-carboxylic acid 6-(4-morpholin-4-ylmethyl-benzoylamino)-pyridin-3-yl ester

4-(4-Carboxymethyl-phenoxymethyl)-piperidine-1-carboxylic acid 6-(4-morpholin-4-ylmethyl-benzoylamino)-pyridin-3-yl ester

4-(1H-Imidazol-2-ylsulfanylmethyl)-piperidine-1-carboxylic acid 6-(4-morpholin-4-ylmethyl-benzoylamino)-pyridin-3-yl ester

4-(1-Methyl-1H-imidazol-2-ylsulfanylmethyl)-piperidine-1-carboxylic acid 6-(4-morpholin-4-ylmethyl-benzoylamino)-pyridin-3-yl ester

4-(Imidazol-1-yloxymethyl)-piperidine-1-carboxylic acid 6-(4-morpholin-4-ylmethyl-benzoylamino)-pyridin-3-yl ester

4-[4-(Pyridin-2-yloxy)-phenoxymethyl]-piperidine-1-carboxylic acid 6-(4-morpholin-4-ylmethyl-benzoylamino)-pyridin-3-yl ester

4-[4-(5-Methyl-pyridin-2-yloxy)-phenoxymethyl]-piperidine-1-carboxylic acid 6-(4-morpholin-4-ylmethyl-benzoylamino)-pyridin-3-yl ester

4-(1-Methyl-1H-tetrazol-5-ylsulfanylmethyl)-piperidine-1-carboxylic acid 6-(4-morpholin-4-ylmethyl-benzoylamino)-pyridin-3-yl ester

4-[1-(2-Dimethylamino-ethyl)-1H-tetrazol-5-ylsulfanyl]-piperidine-1-carboxylic acid 6-(4-morpholin-4-ylmethyl-benzoylamino)-pyridin-3-yl ester

4-[1-(2-Carboxy-ethyl)-1H-tetrazol-5-ylsulfanyl]-piperidine-1-carboxylic acid 6-(4-morpholin-4-ylmethyl-benzoylamino)-pyridin-3-yl ester

4-(1-Carboxymethyl-1H-tetrazol-5-ylsulfanyl)-piperidine-1-carboxylic acid 6-(4-morpholin-4-ylmethyl-benzoylamino)-pyridin-3-yl ester

4-(Pyrazol-1-yloxymethyl)-piperidine-1-carboxylic acid 6-(4-morpholin-4-ylmethyl-benzoylamino)-pyridin-3-yl ester

4-(1H-[1,2,4]Triazol-3-ylsulfanylmethyl)-piperidine-1-carboxylic acid 6-(4-morpholin-4-ylmethyl-benzoylamino)-pyridin-3-yl ester

4-[2-(Benzyl-methyl-amino)-ethyl]-piperidine-1-carboxylic acid 6-(4-morpholin-4-ylmethyl-benzoylamino)-pyridin-3-yl ester

4-(1H-[1,2,4]Triazol-3-ylsulfanyl)-piperidine-1-carboxylic acid 4-(4-morpholin-4-ylmethyl-benzoylamino)-phenyl ester

4-(1H-Imidazol-2-ylsulfanyl)-piperidine-1-carboxylic acid 4-(4-morpholin-4-ylmethyl-benzoylamino)-phenyl ester

4-(1-Methyl-1H-imidazol-2-ylsulfanyl)-piperidine-1-carboxylic acid 4-(4-morpholin-4-ylmethyl-benzoylamino)-phenyl ester

4-(1-Methyl-1H-tetrazol-5-ylsulfanyl)-piperidine-1-carboxylic acid 4-(4-morpholin-4-ylmethyl-benzoylamino)-phenyl ester

4-(Imidazol-1-yloxy)-piperidine-1-carboxylic acid 4-(4-morpholin-4-ylmethyl-benzoylamino)-phenyl ester

4-[1-(4-Carboxy-phenyl)-1H-tetrazol-5-ylsulfanyl]-piperidine-1-carboxylic acid 4-(4-morpholin-4-ylmethyl-benzoylamino)-phenyl ester

4-(4-Carboxymethyl-phenoxy)-piperidine-1-carboxylic acid 4-(4-morpholin-4-ylmethyl-benzoylamino)-phenyl ester

4-(Pyrazol-1-yloxy)-piperidine-1-carboxylic acid 4-(4-morpholin-4-ylmethyl-benzoylamino)-phenyl ester

4-(4-Carboxy-phenoxy)-piperidine-1-carboxylic acid 4-(4-morpholin-4-ylmethyl-benzoylamino)-phenyl ester

4-[4-(Pyridin-2-yloxy)-phenoxy]-piperidine-1-carboxylic acid 4-(4-morpholin-4-ylmethyl-benzoylamino)-phenyl ester

4-[4-(5-Methyl-pyridin-2-yloxy)-phenoxy]-piperidine-1-carboxylic acid 4-(4-morpholin-4-ylmethyl-benzoylamino)-phenyl ester

4-(4-Carboxy-phenoxymethyl)-piperidine-i1-carboxylic acid 4-(4-morpholin-4-ylmethyl-benzoylamino)-phenyl ester

4-(4-Carboxymethyl-phenoxymethyl)-piperidine-1-carboxylic acid 4-(4-morpholin-4-ylmethyl-benzoylamino)-phenyl ester

4-(1H-Imidazol-2-ylsulfanylmethyl)-piperidine-1-carboxylic acid 4-(4-morpholin-4-ylmethyl-benzoylamino)-phenyl ester

4-(1-Methyl-1H-imidazol-2-ylsulfanylmethyl)-piperidine-1-carboxylic acid 4-(4-morpholin-4-ylmethyl-benzoylamino)-phenyl ester

4-(Imidazol-1-yloxymethyl)-piperidine-1-carboxylic acid 4-(4-morpholin-4-ylmethyl-benzoylamino)-phenyl ester

4-[4-(Pyridin-2-yloxy)-phenoxymethyl]-piperidine-1-carboxylic acid 4-(4-morpholin-4-ylmethyl-benzoylamino)-phenyl ester

4-[4-(5-Methyl-pyridin-2-yloxy)-phenoxymethyl]-piperidine-1-carboxylic acid 4-(4-morpholin-4-ylmethyl-benzoylamino)-phenyl ester

4-(1-Methyl-1H-tetrazol-5-ylsulfanylmethyl)-piperidine-1-carboxylic acid 4-(4-morpholin-4-ylmethyl-benzoylamino)-phenyl ester

4-[1-(2-Dimethylamino-ethyl)-1H-tetrazol-5-ylsulfanyl]-piperidine-1-carboxylic acid 4-(4-morpholin-4-ylmethyl-benzoylamino)-phenyl ester

4-[1-(2-Carboxy-ethyl)-1H-tetrazol-5-ylsulfanyl]-piperidine-1-carboxylic acid 4-(4-morpholin-4-ylmethyl-benzoylamino)-phenyl ester

4-(1-Carboxymethyl-1H-tetrazol-5-ylsulfanyl)-piperidine-1-carboxylic acid 4-(4-morpholin-4-ylmethyl-benzoylamino)-phenyl ester

4-(Pyrazol-1-yloxymethyl)-piperidine-1-carboxylic acid 4-(4-morpholin-4-ylmethyl-benzoylamino)-phenyl ester

4-(1H-[1,2,4]Triazol-3-ylsulfanylmethyl)-piperidine-1-carboxylic acid 4-(4-morpholin-4-ylmethyl-benzoylamino)-phenyl ester

4-[2-(Benzyl-methyl-amino)-ethyl]-piperidine-1-carboxylic acid 4-(4-morpholin-4-ylmethyl-benzoylamino)-phenyl ester

4-(1H-[1,2,4]Triazol-3-ylsulfanyl)-piperidine-1-carboxylic acid 6-(4-piperidin-1-ylmethyl-benzoylamino)-pyridin-3-yl ester

4-(1H-Imidazol-2-ylsulfanyl)-piperidine-1-carboxylic acid 6-(4-piperidin-1-ylmethyl-benzoylamino)-pyridin-3-yl ester

4-(1-Methyl-1H-imidazol-2-ylsulfanyl)-piperidine-1-carboxylic acid 6-(4-piperidin-1-ylmethyl-benzoylamino)-pyridin-3-yl ester

4-(1-Methyl-1H-tetrazol-5-ylsulfanyl)-piperidine-1-carboxylic acid 6-(4-piperidin-1-ylmethyl-benzoylamino)-pyridin-3-yl ester

4-(Imidazol-1-yloxy)-piperidine-1-carboxylic acid 6-(4-piperidin-1-ylmethyl-benzoylamino)-pyridin-3-yl ester

4-[1-(4-Carboxy-phenyl)-1H-tetrazol-5-ylsulfanyl]-piperidine-1-carboxylic acid 6-(4-piperidin-1-ylmethyl-benzoylamino)-pyridin-3-yl ester

4-(4-Carboxymethyl-phenoxy)-piperidine-1-carboxylic acid 6-(4-piperidin-1-ylmethyl-benzoylamino)-pyridin-3-yl ester

4-(Pyrazol-1-yloxy)-piperidine-1-carboxylic acid 6-(4-piperidin-1-ylmethyl-benzoylamino)-pyridin-3-yl ester

4-(4-Carboxy-phenoxy)-piperidine-1-carboxylic acid 6-(4-piperidin-1-ylmethyl-benzoylamino)-pyridin-3-yl ester

4-[4-(Pyridin-2-yloxy)-phenoxy]-piperidine-1-carboxylic acid 6-(4-piperidin-1-ylmethyl-benzoylamino)-pyridin-3-yl ester

4-[4-(5-Methyl-pyridin-2-yloxy)-phenoxy]-piperidine-1-carboxylic acid 6-(4-piperidin-1-ylmethyl-benzoylamino)-pyridin-3-yl ester

4-(4-Carboxy-phenoxymethyl)-piperidine-1-carboxylic acid 6-(4-piperidin-1-ylmethyl-benzoylamino)-pyridin-3-yl ester

4-(4-Carboxymethyl-phenoxymethyl)-piperidine-1-carboxylic acid 6-(4-piperidin-1-ylmethyl-benzoylamino)-pyridin-3-yl ester

4-(1H-Imidazol-2-ylsulfanylmethyl)-piperidine-1-carboxylic acid 6-(4-piperidin-1-ylmethyl-benzoylamino)-pyridin-3-yl ester

4-(1-Methyl-1H-imidazol-2-ylsulfanylmethyl)-piperidine-1-carboxylic acid 6-(4-piperidin-1-ylmethyl-benzoylamino)-pyridin-3-yl ester

4-(Imidazol-1-yloxymethyl)-piperidine-1-carboxylic acid 6-(4-piperidin-1-ylmethyl-benzoylamino)-pyridin-3-yl ester

4-[4-(Pyridin-2-yloxy)-phenoxymethyl]-piperidine-1-carboxylic acid 6-(4-piperidin-1-ylmethyl-benzoylamino)-pyridin-3-yl ester

4-[4-(5-Methyl-pyridin-2-yloxy)-phenoxymethyl]-piperidine-1-carboxylic acid 6-(4-piperidin-1-ylmethyl-benzoylamino)-pyridin-3-yl ester

4-(1-Methyl-1H-tetrazol-5-ylsulfanylmethyl)-piperidine-1-carboxylic acid 6-(4-piperidin-1-ylmethyl-benzoylamino)-pyridin-3-yl ester

4-[1-(2-Dimethylamino-ethyl)-1H-tetrazol-5-ylsulfanyl]-piperidine-1-carboxylic acid 6-(4-piperidin-1-ylmethyl-benzoylamino)-pyridin-3-yl ester

4-[1-(2-Carboxy-ethyl)-1H-tetrazol-5-ylsulfanyl]-piperidine-1-carboxylic acid 6-(4-piperidin-1-ylmethyl-benzoylamino)-pyridin-3-yl ester

4-(1-Carboxymethyl-1H-tetrazol-5-ylsulfanyl)-piperidine-1-carboxylic acid 6-(4-piperidin-1-ylmethyl-benzoylamino)-pyridin-3-yl ester

4-(Pyrazol-1-yloxymethyl)-piperidine-1-carboxylic acid 6-(4-piperidin-1-ylmethyl-benzoylamino)-pyridin-3-yl ester

4-(1H-[1,2,4]Triazol-3-ylsulfanylmethyl)-piperidine-1-carboxylic acid 6-(4-piperidin-1-ylmethyl-benzoylamino)-pyridin-3-yl ester

4-[2-(Benzyl-methyl-amino)-ethyl]-piperidine-1-carboxylic acid 6-(4-piperidin-1-ylmethyl-benzoylamino)-pyridin-3-yl ester

4-(1H-[1,2,4]Triazol-3-ylsulfanyl)-piperidine-1-carboxylic acid 4-(4-piperidin-1-ylmethyl-benzoylamino)-phenyl ester

4-(1H-Imidazol-2-ylsulfanyl)-piperidine-1-carboxylic acid 4-(4-piperidin-1-ylmethyl-benzoylamino)-phenyl ester

4-(1-Methyl-1H-imidazol-2-ylsulfanyl)-piperidine-1-carboxylic acid 4-(4-piperidin-1-ylmethyl-benzoylamino)-phenyl ester

4-(1-Methyl-1H-tetrazol-5-ylsulfanyl)-piperidine-1-carboxylic acid 4-(4-piperidin-1-ylmethyl-benzoylamino)-phenyl ester

4-(Imidazol-1-yloxy)-piperidine-1-carboxylic acid 4-(4-piperidin-1-ylmethyl-benzoylamino)-phenyl ester

4-[1-(4-Carboxy-phenyl)-1H-tetrazol-5-ylsulfanyl]-piperidine-1-carboxylic acid 4-(4-piperidin-1-ylmethyl-benzoylamino)-phenyl ester

4-(4-Carboxymethyl-phenoxy)-piperidine-1-carboxylic acid 4-(4-piperidin-1-ylmethyl-benzoylamino)-phenyl ester

4-(Pyrazol-1-yloxy)-piperidine-1-carboxylic acid 4-(4-piperidin-1-ylmethyl-benzoylamino)-phenyl ester

4-(4-Carboxy-phenoxy)-piperidine-1-carboxylic acid 4-(4-piperidin-1-ylmethyl-benzoylamino)-phenyl ester

4-[4-(Pyridin-2-yloxy)-phenoxy]-piperidine-1-carboxylic acid 4-(4-piperidin-1-ylmethyl-benzoylamino)-phenyl ester

4-[4-(5-Methyl-pyridin-2-yloxy)-phenoxy]-piperidine-1-carboxylic acid 4-(4-piperidin-1-ylmethyl-benzoylamino)-phenyl ester

4-(4-Carboxy-phenoxymethyl)-piperidine-1-carboxylic acid 4-(4-piperidin-1-ylmethyl-benzoylamino)-phenyl ester

4-(4-Carboxymethyl-phenoxymethyl)-piperidine-1-carboxylic acid 4-(4-piperidin-1-ylmethyl-benzoylamino)-phenyl ester

4-(1H-Imidazol-2-ylsulfanylmethyl)-piperidine-1-carboxylic acid 4-(4-piperidin-1-ylmethyl-benzoylamino)-phenyl ester

4-(1-Methyl-1H-imidazol-2-ylsulfanylmethyl)-piperidine-1-carboxylic acid 4-(4-piperidin-1-ylmethyl-benzoylamino)-phenyl ester

4-(Imidazol-1-yloxymethyl)-piperidine-1-carboxylic acid 4-(4-piperidin-1-ylmethyl-benzoylamino)-phenyl ester

4-[4-(Pyridin-2-yloxy)-phenoxymethyl]-piperidine-1-carboxylic acid 4-(4-piperidin-1-ylmethyl-benzoylamino)-phenyl ester

4-[4-(5-Methyl-pyridin-2-yloxy)-phenoxymethyl]-piperidine-1-carboxylic acid 4-(4-piperidin-1-ylmethyl-benzoylamino)-phenyl ester

4-(1-Methyl-1H-tetrazol-5-ylsulfanylmethyl)-piperidine-1-carboxylic acid 4-(4-piperidin-1-ylmethyl-benzoylamino)-phenyl ester

4-[i -(2-Dimethylamino-ethyl)-1H-tetrazol-5-ylsulfanyl]-piperidine-1-carboxylic acid 4-(4-piperidin-1-ylmethyl-benzoylamino)-phenyl ester

4-[1-(2-Carboxy-ethyl)-1H-tetrazol-5-ylsulfanyl]-piperidine-1-carboxylic acid 4-(4-piperidin-1-ylmethyl-benzoylamino)-phenyl ester

4-(1-Carboxymethyl-1H-tetrazol-5-ylsulfanyl)-piperidine-1-carboxylic acid 4-(4-piperidin-1-ylmethyl-benzoylamino)-phenyl ester

4-(Pyrazol-1-yloxymethyl)-piperidine-1-carboxylic acid 4-(4-piperidin-1-ylmethyl-benzoylamino)-phenyl ester

4-(1H-[1,2,4]Triazol-3-ylsulfanylmethyl)-piperidine-1-carboxylic acid 4-(4-piperidin-1-ylmethyl-benzoylamino)-phenyl ester

4-[2-(Benzyl-methyl-amino)-ethyl]-piperidine-1-carboxylic acid 4-(4-piperidin-1-ylmethyl-benzoylamino)-phenyl ester

4-(1H-[1,2,4]Triazol-3-ylsulfanyl)-piperidine-1-carboxylic acid 5-(4-piperidin-1-ylmethyl-benzoylamino)-pyridin-2-yl ester

4-(1H-Imidazol-2-ylsulfanyl)-piperidine-1-carboxylic acid 5-(4-piperidin-1-ylmethyl-benzoylamino)-pyridin-2-yl ester

4-(1-Methyl-1H-imidazol-2-ylsulfanyl)-piperidine-1-carboxylic acid 5-(4-piperidin-1-ylmethyl-benzoylamino)-pyridin-2-yl ester

4-(1-Methyl-1H-tetrazol-5-ylsulfanyl)-piperidine-1-carboxylic acid 5-(4-piperidin-1-ylmethyl-benzoylamino)-pyridin-2-yl ester

4-(Imidazol-1-yloxy)-piperidine-1-carboxylic acid 5-(4-piperidin-1-ylmethyl-benzoylamino)-pyridin-2-yl ester

4-[1-(4-Carboxy-phenyl)-1H-tetrazol-5-ylsulfanyl]-piperidine-1-carboxylic acid 5-(4-piperidin-1-ylmethyl-benzoylamino)-pyridin-2-yl ester

4-(4-Carboxymethyl-phenoxy)-piperidine-1-carboxylic acid 5-(4-piperidin-1-ylmethyl-benzoylamino)-pyridin-2-yl ester

4-(Pyrazol-1-yloxy)-piperidine-1-carboxylic acid 5-(4-piperidin-1-ylmethyl-benzoylamino)-pyridin-2-yl ester

4-(4-Carboxy-phenoxy)-piperidine-1-carboxylic acid 5-(4-piperidin-1-ylmethyl-benzoylamino)-pyridin-2-yl ester

4-[4-(Pyridin-2-yloxy)-phenoxy]-piperidine-1-carboxylic acid 5-(4-piperidin-1-ylmethyl-benzoylamino)-pyridin-2-yl ester

4-[4-(5-Methyl-pyridin-2-yloxy)-phenoxy]-piperidine-1-carboxylic acid 5-(4-piperidin-1-ylmethyl-benzoylamino)-pyridin-2-yl ester

4-(4-Carboxy-phenoxymethyl)-piperidine-1-carboxylic acid 5-(4-piperidin-1-ylmethyl-benzoylamino)-pyridin-2-yl ester

4-(4-Carboxymethyl-phenoxymethyl)-piperidine-1-carboxylic acid 5-(4-piperidin-1-ylmethyl-benzoylamino)-pyridin-2-yl ester

4-(1H-Imidazol-2-ylsulfanylmethyl)-piperidine-1-carboxylic acid 5-(4-piperidin-1-ylmethyl-benzoylamino)-pyridin-2-yl ester

4-(1-Methyl-1H-imidazol-2-ylsulfanylmethyl)-piperidine-1-carboxylic acid 5-(4-piperidin-1-ylmethyl-benzoylamino)-pyridin-2-yl ester

4-(Imidazol-1-yloxymethyl)-piperidine-1-carboxylic acid 5-(4-piperidin-1-ylmethyl-benzoylamino)-pyridin-2-yl ester

4-[4-(Pyridin-2-yloxy)-phenoxymethyl]-piperidine-1-carboxylic acid 5-(4-piperidin-1-ylmethyl-benzoylamino)-pyridin-2-yl ester

4-[4-(5-Methyl-pyridin-2-yloxy)-phenoxymethyl]-piperidine-1-carboxylic acid 5-(4-piperidin-1-ylmethyl-benzoylamino)-pyridin-2-yl ester

4-(1-Methyl-1H-tetrazol-5-ylsulfanylmethyl)-piperidine-1-carboxylic acid 5-(4-piperidin-1-ylmethyl-benzoylamino)-pyridin-2-yl ester

4-[1-(2-Dimethylamino-ethyl)-1H-tetrazol-5-ylsulfanyl]-piperidine-1-carboxylic acid 5-(4-piperidin-1-ylmethyl-benzoylamino)-pyridin-2-yl ester

4-[1-(2-Carboxy-ethyl)-1H-tetrazol-5-ylsulfanyl]-piperidine-1-carboxylic acid 5-(4-piperidin-1-ylmethyl-benzoylamino)-pyridin-2-yl ester

4-(1-Carboxymethyl-1H-tetrazol-5-ylsulfanyl)-piperidine-1-carboxylic acid 5-(4-piperidin-1-ylmethyl-benzoylamino)-pyridin-2-yl ester

4-(Pyrazol-1-yloxymethyl)-piperidine-1-carboxylic acid 5-(4-piperidin-1-ylmethyl-benzoylamino)-pyridin-2-yl ester

4-(1H-[1,2,4]Triazol-3-ylsulfanylmethyl)-piperidine-1-carboxylic acid 5-(4-piperidin-1-ylmethyl-benzoylamino)-pyridin-2-yl ester

4-[2-(Benzyl-methyl-amino)-ethyl]-piperidine-1-carboxylic acid 5-(4-piperidin-1-ylmethyl-benzoylamino)-pyridin-2-yl ester

4-(1H-[1,2,4]Triazol-3-ylsulfanyl)-piperidine-1-carboxylic acid 5-[4-(2-ethyl-piperidin-1-ylmethyl)-benzoylamino]-pyridin-2-yl ester

4-(1H-Imidazol-2-ylsulfanyl)-piperidine-1-carboxylic acid 5-[4-(2-ethyl-piperidin-1-ylmethyl)-benzoylamino]-pyridin-2-yl ester

4-(1-Methyl-1H-imidazol-2-ylsulfanyl)-piperidine-1-carboxylic acid 5-[4-(2-ethyl-piperidin-1-ylmethyl)-benzoylamino]-pyridin-2-yl ester

4-(1-Methyl-1H-tetrazol-5-ylsulfanyl)-piperidine-1-carboxylic acid 5-[4-(2-ethyl-piperidin-ylmethyl)-benzoylamino]-pyridin-2-yl ester

4-(Imidazol-1-yloxy)-piperidine-1-carboxylic acid 5-[4-(2-ethyl-piperidin-1-ylmethyl)-benzoylamino]-pyridin-2-yl ester

4-[1-(4-Carboxy-phenyl)-1H-tetrazol-5-ylsulfanyl]-piperidine-1-carboxylic acid 5-[4-(2-ethyl-piperidin-1-ylmethyl)-benzoylamino]-pyridin-2-yl ester

4-(4-Carboxymethyl-phenoxy)-piperidine-1-carboxylic acid 5-[4-(2-ethyl-piperidin-1-ylmethyl)-benzoylamino]-pyridin-2-yl ester

4-(Pyrazol-1-yloxy)-piperidine-1-carboxylic acid 5-[4-(2-ethyl-piperidin-1-ylmethyl)-benzoylamino]-pyridin-2-yl ester

4-(4-Carboxy-phenoxy)-piperidine-1-carboxylic acid 5-[4-(2-ethyl-piperidin-1-ylmethyl)-benzoylamino]-pyridin-2-yl ester

4-[4-(Pyridin-2-yloxy)-phenoxy]-piperidine-1-carboxylic acid 5-[4-(2-ethyl-piperidin-1-ylmethyl)-benzoylamino]-pyridin-2-yl ester

4-[4-(5-Methyl-pyridin-2-yloxy)-phenoxy]-piperidine-1-carboxylic acid 5-[4-(2-ethyl-piperidin-1-ylmethyl)-benzoylamino]-pyridin-2-yl ester

4-(4-Carboxy-phenoxymethyl)-piperidine-1-carboxylic acid 5-[4-(2-ethyl-piperidin-1-ylmethyl)-benzoylamino]-pyridin-2-yl ester

4-(4-Carboxymethyl-phenoxymethyl)-piperidine-1-carboxylic acid 5-[4-(2-ethyl-piperidin-1-ylmethyl)-benzoylamino]-pyridin-2-yl ester

4-(1H-Imidazol-2-ylsulfanylmethyl)-piperidine-1-carboxylic acid 5-[4-(2-ethyl-piperidin-1-ylmethyl)-benzoylamino]-pyridin-2-yl ester

4-(1-Methyl-1H-imidazol-2-ylsulfanylmethyl)-piperidine-1-carboxylic acid 5-[4-(2-ethyl-piperidin-1-ylmethyl)-benzoylamino]-pyridin-2-yl ester

4-(Imidazol-1-yloxymethyl)-piperidine-1-carboxylic acid 5-[4-(2-ethyl-piperidin-1-ylmethyl)-benzoylamino]-pyridin-2-yl ester

4-[4-(Pyridin-2-yloxy)-phenoxymethyl]-piperidine-1-carboxylic acid 5-[4-(2-ethyl-piperidin-1-ylmethyl)-benzoylamino]-pyridin-2-yl ester

4-[4-(5-Methyl-pyridin-2-yloxy)-phenoxymethyl]-piperidine-1-carboxylic acid 5-[4-(2-ethyl-piperidin-1-ylmethyl)-benzoylamino]-pyridin-2-yl ester

4-(1-Methyl-1H-tetrazol-5-ylsulfanylmethyl)-piperidine-1-carboxylic acid 5-[4-(2-ethyl-piperidin-1-ylmethyl)-benzoylamino]-pyridin-2-yl ester

4-[1-(2-Dimethylamino-ethyl)-1H-tetrazol-5-ylsulfanyl]-piperidine-1-carboxylic acid 5-[4-(2-ethyl-piperidin-1-ylmethyl)-benzoylamino]-pyridin-2-yl ester

4-[1-(2-Carboxy-ethyl)-1H-tetrazol-5-ylsulfanyl]-piperidine-1-carboxylic acid 5-[4-(2-ethyl-piperidin-1-ylmethyl)-benzoylamino]-pyridin-2-yl ester 4-(1-Carboxymethyl-1H-tetrazol-5-ylsulfanyl)-piperidine-1-carboxylic acid 5-[4-(2-ethyl-piperidin-1-ylmethyl)-benzoylamino]-pyridin-2-yl ester

4-(Pyrazol-1-yloxymethyl)-piperidine-1-carboxylic acid 5-[4-(2-ethyl-piperidin-1-ylmethyl)-benzoylamino]-pyridin-2-yl ester

4-(1H-[1,2,4]Triazol-3-ylsulfanylmethyl)-piperidine-1-carboxylic acid 5-[4-(2-ethyl-piperidin-1-ylmethyl)-benzoylamino]-pyridin-2-yl ester

4-[2-(Benzyl-methyl-amino)-ethyl]-piperidine-1-carboxylic acid 5-[4-(2-ethyl-piperidin-1-ylmethyl)-benzoylamino]-pyridin-2-yl ester

4-(1H-[1,2,4]Triazol-3-ylsulfanyl)-piperidine-1-carboxylic acid 5-[4-(4-methyl-piperidin-ylmethyl)-benzoylamino]-pyridin-2-yl ester

4-(1H-Imidazol-2-ylsulfanyl)-piperidine-1-carboxylic acid 5-[4-(4-methyl-piperidin-1-ylmethyl)-benzoylamino]-pyridin-2-yl ester

4-(1-Methyl-1H-imidazol-2-ylsulfanyl)-piperidine-1-carboxylic acid 5-[4-(4-methyl-piperidin-1-ylmethyl)-benzoylamino]-pyridin-2-yl ester

4-(1-Methyl-1H-tetrazol-5-ylsulfanyl)-piperidine-1-carboxylic acid 5-[4-(4-methyl-piperidin-1-ylmethyl)-benzoylamino]-pyridin-2-yl ester

4-(Imidazol-1-yloxy)-piperidine-1-carboxylic acid 5-[4-(4-methyl-piperidin-1-ylmethyl)-benzoylamino]-pyridin-2-yl ester

4-[l -(4-Carboxy-phenyl)-l H-tetrazol-5-ylsulfanyl]-piperidine-1-carboxylic acid 5-[4-(4-methyl-piperidin-1-ylmethyl)-benzoylamino]-pyridin-2-yl ester

4-(4-Carboxymethyl-phenoxy)-piperidine-1-carboxylic acid 5-[4-(4-methyl-piperidin-1-ylmethyl)-benzoylamino]-pyridin-2-yl ester

4-(Pyrazol-1-yloxy)-piperidine-1-carboxylic acid 5-[4-(4-methyl-piperidin-1-ylmethyl)-benzoylamino]-pyridin-2-yl ester

4-(4-Carboxy-phenoxy)-piperidine-1-carboxylic acid 5-[4-(4-methyl-piperidin-1-ylmethyl)-benzoylamino]-pyridin-2-yl ester

4-[4-(Pyridin-2-yloxy)-phenoxy]-piperidine-1-carboxylic acid 5-[4-(4-methyl-piperidin-1-ylmethyl)-benzoylamino]-pyridin-2-yl ester

4-[4-(5-Methyl-pyridin-2-yloxy)-phenoxy]-piperidine-1-carboxylic acid 5-[4-(4-methyl-piperidin-1-ylmethyl)-benzoylamino]-pyridin-2-yl ester

4-(4-Carboxy-phenoxymethyl)-piperidine-1-carboxylic acid 5-[4-(4-methyl-piperidin-1-ylmethyl)-benzoylamino]-pyridin-2-yl ester

4-(4-Carboxymethyl-phenoxymethyl)-piperidine-1-carboxylic acid 5-[4-(4-methyl-piperidin-1-ylmethyl)-benzoylamino]-pyridin-2-yl ester

4-(1H-Imidazol-2-ylsulfanylmethyl)-piperidine-1-carboxylic acid 5-[4-(4-methyl-piperidin-1-ylmethyl)-benzoylamino]-pyridin-2-yl ester

4-(1-Methyl-1H-imidazol-2-ylsulfanylmethyl)-piperidine-1-carboxylic acid 5-[4-(4-methyl-piperidin-1-ylmethyl)-benzoylamino]-pyridin-2-yl ester

4-(Imidazol-1-yloxymethyl)-piperidine-1-carboxylic acid 5-[4-(4-methyl-piperidin-1-ylmethyl)-benzoylamino]-pyridin-2-yl ester

4-[4-(Pyridin-2-yloxy)-phenoxymethyl]-piperidine-1-carboxylic acid 5-[4-(4-methyl-piperidin-1-ylmethyl)-benzoylamino]-pyridin-2-yl ester

4-[4-(5-Methyl-pyridin-2-yloxy)-phenoxymethyl]-piperidine-1-carboxylic acid 5-[4-(4-methyl-piperidin-1-ylmethyl)-benzoylamino]-pyridin-2-yl ester

4-(1-Methyl-1H-tetrazol-5-ylsulfanylmethyl)-piperidine-1-carboxylic acid 5-[4-(4-methyl-piperidin-1-ylmethyl)-benzoylamino]-pyridin-2-yl ester

4-[1-(2-Dimethylamino-ethyl)-1H-tetrazol-5-ylsulfanyl]-piperidine-1-carboxylic acid 5-[4-(4-methyl-piperidin-1-ylmethyl)-benzoylamino]-pyridin-2-yl ester

4-[1-(2-Carboxy-ethyl)-1H-tetrazol-5-ylsulfanyl]-piperidine-1-carboxylic acid 5-[4-(4-methyl-piperidin-1-ylmethyl)-benzoylamino]-pyridin-2-yl ester

4-(1-Carboxymethyl-1H-tetrazol-5-ylsulfanyl)-piperidine-1-carboxylic acid 5-[4-(4-methyl-piperidin-1-ylmethyl)-benzoylamino]-pyridin-2-yl ester

4-(Pyrazol-1-yloxymethyl )-piperidine-1-carboxylic acid 5-[4-(4-methyl-piperidin-1-ylmethyl)-benzoylamino]-pyridin-2-yl ester

4-(1H-[1,2,4]Triazol-3-ylsulfanylmethyl)-piperidine-1-carboxylic acid 5-[4-(4-methyl-piperidin-1-ylmethyl)-benzoylamino]-pyridin-2-yl ester

4-[2-(Benzyl-methyl-amino)-ethyl]-piperidine-1-carboxylic acid 5-[4-(4-methyl-piperidin-1-ylmethyl)-benzoylamino]-pyridin-2-yl ester

4-(1H-[1,2,4]Triazol-3-ylsulfanyl)-piperidine-1-carboxylic acid 5-[4-(3-methyl-piperidin-ylmethyl)-benzoylamino]-pyridin-2-yl ester

4-(1H-Imidazol-2-ylsulfanyl)-piperidine-1-carboxylic acid 5-[4-(3-methyl-piperidin-1-ylmethyl)-benzoylamino]-pyridin-2-yl ester

4-(1-Methyl-1H-imidazol-2-ylsulfanyl)-piperidine-1-carboxylic acid 5-[4-(3-methyl-piperidin-1-ylmethyl)-benzoylamino]-pyridin-2-yl ester

4-(1-Methyl-1H-tetrazol-5-ylsulfanyl)-piperidine-1-carboxylic acid 5-[4-(3-methyl-piperidin-1-ylmethyl)-benzoylamino]-pyridin-2-yl ester

4-(Imidazol-1-yloxy)-piperidine-1-carboxylic acid 5-[4-(3-methyl-piperidin-1-ylmethyl)-benzoylamino]-pyridin-2-yl ester

4-[1-(4-Carboxy-phenyl)-1H-tetrazol-5-ylsulfanyl]-piperidine-1-carboxylic acid 5-[4-(3-methyl-piperidin-1-ylmethyl)-benzoylamino]-pyridin-2-yl ester

4-(4-Carboxymethyl-phenoxy)-piperidine-1-carboxylic acid 5-[4-(3-methyl-piperidin-1-ylmethyl)-benzoylamino]-pyridin-2-yl ester

4-(Pyrazol-1-yloxy)-piperidine-1-carboxylic acid 5-[4-(3-methyl-piperidin-1-ylmethyl)-benzoylamino]-pyridin-2-yl ester

4-(4-Carboxy-phenoxy)-piperidine-1-carboxylic acid 5-[4-(3-methyl-piperidin-1-ylmethyl)-benzoylamino]-pyridin-2-yl ester

4-[4-(Pyridin-2-yloxy)-phenoxy]-piperidine-1-carboxylic acid 5-[4-(3-methyl-piperidin-1-ylmethyl)-benzoylamino]-pyridin-2-yl ester

4-[4-(5-Methyl-pyridin-2-yloxy)-phenoxy]-piperidine-1-carboxylic acid 5-[4-(3-methyl-piperidin-1-ylmethyl)-benzoylamino]-pyridin-2-yl ester

4-(4-Carboxy-phenoxymethyl)-piperidine-1-carboxylic acid 5-[4-(3-methyl-piperidin-1-ylmethyl)-benzoylamino]-pyridin-2-yl ester

4-(4-Carboxymethyl-phenoxymethyl)-piperidine-1-carboxylic acid 5-[4-(3-methyl-piperidin-1-ylmethyl)-benzoylamino]-pyridin-2-yl ester

4-(1H-Imidazol-2-ylsulfanylmethyl)-piperidine-1-carboxylic acid 5-[4-(3-methyl-piperidin-1-ylmethyl)-benzoylamino]-pyridin-2-yl ester

4-(1-Methyl-1H-imidazol-2-ylsulfanylmethyl)-piperidine-1-carboxylic acid 5-[4-(3-methyl-piperidin-1-ylmethyl)-benzoylamino]-pyridin-2-yl ester

4-(Imidazol-1-yloxymethyl)-piperidine-1-carboxylic acid 5-[4-(3-methyl-piperidin-1-ylmethyl)-benzoylamino]-pyridin-2-yl ester

4-[4-(Pyridin-2-yloxy)-phenoxymethyl]-piperidine-1-carboxylic acid 5-[4-(3-methyl-piperidin-1-ylmethyl)-benzoylamino]-pyridin-2-yl ester

4-[4-(5-Methyl-pyridin-2-yloxy)-phenoxymethyl]-piperidine-1-carboxylic acid 5-[4-(3-methyl-piperidin-1-ylmethyl)-benzoylamino]-pyridin-2-yl ester

4-(1-Methyl-1H-tetrazol-5-ylsulfanylmethyl)-piperidine-1-carboxylic acid 5-[4-(3-methyl-piperidin-1-ylmethyl)-benzoylamino]-pyridin-2-yl ester

4-[1-(2-Dimethylamino-ethyl)-1H-tetrazol-5-ylsulfanyl]-piperidine-1-carboxylic acid 5-[4-(3-methyl-piperidin-1-ylmethyl)-benzoylamino]-pyridin-2-yl ester

4-[1-(2-Carboxy-ethyl)-1H-tetrazol-5-ylsulfanyl]-piperidine-1-carboxylic acid 5-[4-(3-methyl-piperidin-1-ylmethyl)-benzoylamino]-pyridin-2-yl ester

4-(1-Carboxymethyl-1H-tetrazol-5-ylsulfanyl)-piperidine-1-carboxylic acid 5-[4-(3-methyl-piperidin-1-ylmethyl)-benzoylamino]-pyridin-2-yl ester

4-(Pyrazol-1-yloxymethyl )-piperidine-1-carboxylic acid 5-[4-(3-methyl-piperidin-1-ylmethyl)-benzoylamino]-pyridin-2-yl ester

4-(1H-[1,2,4]Triazol-3-ylsulfanylmethyl)-piperidine-1-carboxylic acid 5-[4-(3-methyl-piperidin-1-ylmethyl)-benzoylamino]-pyridin-2-yl ester

4-[2-(Benzyl-methyl-amino)-ethyl]-piperidine-1-carboxylic acid 5-[4-(3-methyl-piperidin-1-ylmethyl)-benzoylamino]-pyridin-2-yl ester

4-(1H-[1,2,4]Triazol-3-ylsulfanyl)-piperidine-1-carboxylic acid 5-[4-(2-methyl-piperidin-ylmethyl)-benzoylamino]-pyridin-2-yl ester

4-(1H-Imidazol-2-ylsulfanyl)-piperidine-1-carboxylic acid 5-[4-(2-methyl-piperidin-1-ylmethyl)-benzoylamino]-pyridin-2-yl ester

4-(1-Methyl-1H-imidazol-2-ylsulfanyl)-piperidine-1-carboxylic acid 5-[4-(2-methyl-piperidin-1-ylmethyl)-benzoylamino]-pyridin-2-yl ester

4-(1-Methyl-1H-tetrazol-5-ylsulfanyl)-piperidine-1-carboxylic acid 5-[4-(2-methyl-piperidin-1-ylmethyl)-benzoylamino]-pyridin-2-yl ester

4-(Imidazol-1-yloxy)-piperidine-1-carboxylic acid 5-[4-(2-methyl-piperidin-1-ylmethyl)-benzoylamino]-pyridin-2-yl ester

4-[1-(4-Carboxy-phenyl)-1H-tetrazol-5-ylsulfanyl]-piperidine-1-carboxylic acid 5-[4-(2-methyl-piperidin-1-ylmethyl)-benzoylamino]-pyridin-2-yl ester

4-(4-Carboxymethyl-phenoxy)-piperidine-1-carboxylic acid 5-[4-(2-methyl-piperidin-1-ylmethyl)-benzoylamino]-pyridin-2-yl ester

4-(Pyrazol-1-yloxy)-piperidine-1-carboxylic acid 5-[4-(2-methyl-piperidin-1-ylmethyl)-benzoylamino]-pyridin-2-yl ester

4-(4-Carboxy-phenoxy)-piperidine-1-carboxylic acid 5-[4-(2-methyl-piperidin-1-ylmethyl)-benzoylamino]-pyridin-2-yl ester

4-[4-(Pyridin-2-yloxy)-phenoxy]-piperidine-1-carboxylic acid 5-[4-(2-methyl-piperidin-1-ylmethyl)-benzoylamino]-pyridin-2-yl ester

4-[4-(5-Methyl-pyridin-2-yloxy)-phenoxy]-piperidine-1-carboxylic acid 5-[4-(2-methyl-piperidin-1-ylmethyl)-benzoylamino]-pyridin-2-yl ester

4-(4-Carboxy-phenoxymethyl)-piperidine-1-carboxylic acid 5-[4-(2-methyl-piperidin-1-ylmethyl)-benzoylamino]-pyridin-2-yl ester

4-(4-Carboxymethyl-phenoxymethyl)-piperidine-1-carboxylic acid 5-[4-(2-methyl-piperidin-1-ylmethyl)-benzoylamino]-pyridin-2-yl ester

4-(1H-Imidazol-2-ylsulfanylmethyl)-piperidine-1-carboxylic acid 5-[4-(2-methyl-piperidin-1-ylmethyl)-benzoylamino]-pyridin-2-yl ester

4-(1-Methyl-1H-imidazol-2-ylsulfanylmethyl)-piperidine-1-carboxylic acid 5-[4-(2-methyl-piperidin-1-ylmethyl)-benzoylamino]-pyridin-2-yl ester

4-(Imidazol-1-yloxymethyl)-piperidine-1-carboxylic acid 5-[4-(2-methyl-piperidin-1-ylmethyl)-benzoylamino]-pyridin-2-yl ester

4-[4-(Pyridin-2-yloxy)-phenoxymethyl]-piperidine-1-carboxylic acid 5-[4-(2-methyl-piperidin-1-ylmethyl)-benzoylamino]-pyridin-2-yl ester

4-[4-(5-Methyl-pyridin-2-yloxy)-phenoxymethyl]-piperidine-1-carboxylic acid 5-[4-(2-methyl-piperidin-1-ylmethyl)-benzoylamino]-pyridin-2-yl ester

4-(1-Methyl-1H-tetrazol-5-ylsulfanylmethyl)-piperidine-1-carboxylic acid 5-[4-(2-methyl-piperidin-1-ylmethyl)-benzoylamino]-pyridin-2-yl ester

4-[1-(2-Dimethylamino-ethyl)-1H-tetrazol-5-ylsulfanyl]-piperidine-1-carboxylic acid 5-[4-(2-methyl-piperidin-1-ylmethyl)-benzoylamino]-pyridin-2-yl ester

4-[1-(2-Carboxy-ethyl)-1H-tetrazol-5-ylsulfanyl]-piperidine-1-carboxylic acid 5-[4-(2-methyl-piperidin-1-ylmethyl)-benzoylamino]-pyridin-2-yl ester

4-(1-Carboxymethyl-1H-tetrazol-5-ylsulfanyl)-piperidine-1-carboxylic acid 5-[4-(2-methyl-piperidin-1-ylmethyl)-benzoylamino]-pyridin-2-yl ester

4-(Pyrazol-1-yloxymethyl)-piperidine-1-carboxylic acid 5-[4-(2-methyl-piperidin-1-ylmethyl)-benzoylamino]-pyridin-2-yl ester

4-(1H-[1,2,4]Triazol-3-ylsulfanylmethyl)-piperidine-1-carboxylic acid 5-[4-(2-methyl-piperidin-1-ylmethyl)-benzoylamino]-pyridin-2-yl ester

4-[2-(Benzyl-methyl-amino)-ethyl]-piperidine-1-carboxylic acid 5-[4-(2-methyl-piperidin-1-ylmethyl)-benzoylamino]-pyridin-2-yl ester

4-(1H-[1,2,4]Triazol-3-ylsulfanyl)-piperidine-1-carboxylic acid 4-(5-trifluoromethyl-pyridin-2-ylmethyl)-phenyl ester

4-(1H-Imidazol-2-ylsulfanyl)-piperidine-1-carboxylic acid 4-(5-trifluoromethyl-pyridin-2-ylmethyl)-phenyl ester

4-(1-Methyl-1H-imidazol-2-ylsulfanyl)-piperidine-1-carboxylic acid 4-(5-trifluoromethyl-pyridin-2-ylmethyl)-phenyl ester

4-(1-Methyl-1H-tetrazol-5-ylsulfanyl)-piperidine-1-carboxylic acid 4-(5-trifluoromethyl-pyridin-2-ylmethyl)-phenyl ester

4-(Imidazol-1-yloxy)-piperidine-1-carboxylic acid 4-(5-trifluoromethyl-pyridin-2-ylmethyl)-phenyl ester

4-[1-(4-Carboxy-phenyl)-1H-tetrazol-5-ylsulfanyl]-piperidine-1-carboxylic acid 4-(5-trifluoromethyl-pyridin-2-ylmethyl)-phenyl ester

4-(4-Carboxymethyl-phenoxy)-piperidine-1-carboxylic acid 4-(5-trifluoromethyl-pyridin-2-ylmethyl)-phenyl ester

4-(Pyrazol-1-yloxy)-piperidine-1-carboxylic acid 4-(5-trifluoromethyl-pyridin-2-ylmethyl)-phenyl ester

4-(4-Carboxy-phenoxy)-piperidine-1-carboxylic acid 4-(5-trifluoromethyl-pyridin-2-ylmethyl)-phenyl ester

4-[4-(Pyridin-2-yloxy)-phenoxy]-piperidine-1-carboxylic acid 4-(5-trifluoromethyl-pyridin-2-ylmethyl)-phenyl ester

4-[4-(5-Methyl-pyridin-2-yloxy)-phenoxy]-piperidine-1-carboxylic acid 4-(5-trifluoromethyl-pyridin-2-ylmethyl)-phenyl ester

4-(4-Carboxy-phenoxymethyl)-piperidine-1-carboxylic acid 4-(5-trifluoromethyl-pyridin-2-ylmethyl)-phenyl ester

4-(4-Carboxymethyl-phenoxymethyl)-piperidine-1-carboxylic acid 4-(5-trifluoromethyl-pyridin-2-ylmethyl)-phenyl ester

4-(1H-Imidazol-2-ylsulfanylmethyl)-piperidine-1-carboxylic acid 4-(5-trifluoromethyl-pyridin-2-ylmethyl)-phenyl ester

4-(1-Methyl-1H-imidazol-2-ylsulfanylmethyl)-piperidine-1-carboxylic acid 4-(5-trifluoromethyl-pyridin-2-ylmethyl)-phenyl ester

4-(Imidazol-1-yloxymethyl)-piperidine-1-carboxylic acid 4-(5-trifluoromethyl-pyridin-2-ylmethyl)-phenyl ester

4-[4-(Pyridin-2-yloxy)-phenoxymethyl]-piperidine-1-carboxylic acid 4-(5-trifluoromethyl-pyridin-2-ylmethyl)-phenyl ester

4-[4-(5-Methyl-pyridin-2-yloxy)-phenoxymethyl]-piperidine-1-carboxylic acid 4-(5-trifluoromethyl-pyridin-2-ylmethyl)-phenyl ester

4-(1-Methyl-1H-tetrazol-5-ylsulfanylmethyl)-piperidine-1-carboxylic acid 4-(5-trifluoromethyl-pyridin-2-ylmethyl)-phenyl ester

4-[1-(2-Dimethylamino-ethyl)-1H-tetrazol-5-ylsulfanyl]-piperidine-1-carboxylic acid 4-(5-trifluoromethyl-pyridin-2-ylmethyl)-phenyl ester

4-[1-(2-Carboxy-ethyl)-1H-tetrazol-5-ylsulfanyl]-piperidine-1-carboxylic acid 4-(5-trifluoromethyl-pyridin-2-ylmethyl)-phenyl ester

4-(1-Carboxymethyl-1H-tetrazol-5-ylsulfanyl)-piperidine-1-carboxylic acid 4-(5-trifluoromethyl-pyridin-2-ylmethyl)-phenyl ester

4-(Pyrazol-1-yloxymethyl)-piperidine-1-carboxylic acid 4-(5-trifluoromethyl-pyridin-2-ylmethyl)-phenyl ester

4-(1H-[1,2,4]Triazol-3-ylsulfanylmethyl)-piperidine-1-carboxylic acid 4-(5-trifluoromethyl-pyridin-2-ylmethyl)-phenyl ester

4-[2-(Benzyl-methyl-amino)-ethyl]-piperidine-1-carboxylic acid 4-(5-trifluoromethyl-pyridin-2-ylmethyl)-phenyl ester

4-(1H-[1,2,4]Triazol-3-ylsulfanyl)-piperidine-1-carboxylic acid 4-(4-methyl-pyridin-2-ylmethyl)-phenyl ester

4-(1H-Imidazol-2-ylsulfanyl)-piperidine-1-carboxylic acid 4-(4-methyl-pyridin-2-ylmethyl)-phenyl ester

4-(1-Methyl-1H-imidazol-2-ylsulfanyl)-piperidine-1-carboxylic acid 4-(4-methyl-pyridin-2-ylmethyl)-phenyl ester

4-(1-Methyl-1H-tetrazol-5-ylsulfanyl)-piperidine-1-carboxylic acid 4-(4-methyl-pyridin-2-ylmethyl)-phenyl ester

4-(Imidazol-1-yloxy)-piperidine-1-carboxylic acid 4-(4-methyl-pyridin-2-ylmethyl)-phenyl ester

4-[1-(4-Carboxy-phenyl)-1H-tetrazol-5-ylsulfanyl]-piperidine-1-carboxylic acid 4-(4-methyl-pyridin-2-ylmethyl)-phenyl ester

4-(4-Carboxymethyl-phenoxy)-piperidine-1-carboxylic acid 4-(4-methyl-pyridin-2-ylmethyl)-phenyl ester

4-(Pyrazol-1-yloxy)-piperidine-1-carboxylic acid 4-(4-methyl-pyridin-2-ylmethyl)-phenyl ester

4-(4-Carboxy-phenoxy)-piperidine-1-carboxylic acid 4-(4-methyl-pyridin-2-ylmethyl)-phenyl ester

4-[4-(Pyridin-2-yloxy)-phenoxy]-piperidine-1-carboxylic acid 4-(4-methyl-pyridin-2-ylmethyl)-phenyl ester

4-[4-(5-Methyl-pyridin-2-yloxy)-phenoxy]-piperidine-1-carboxylic acid 4-(4-methyl-pyridin-2-ylmethyl)-phenyl ester

4-(4-Carboxy-phenoxymethyl)-piperidine-1-carboxylic acid 4-(4-methyl-pyridin-2-ylmethyl)-phenyl ester

4-(4-Carboxymethyl-phenoxymethyl)-piperidine-1-carboxylic acid 4-(4-methyl-pyridin-2-ylmethyl)-phenyl ester

4-(1H-Imidazol-2-ylsulfanylmethyl)-piperidine-1-carboxylic acid 4-(4-methyl-pyridin-2-ylmethyl)-phenyl ester

4-(1-Methyl-1H-imidazol-2-ylsulfanylmethyl)-piperidine-1-carboxylic acid 4-(4-methyl-pyridin-2-ylmethyl)-phenyl ester

4-(Imidazol-1-yloxymethyl)-piperidine-1-carboxylic acid 4-(4-methyl-pyridin-2-ylmethyl)-phenyl ester

4-[4-(Pyridin-2-yloxy)-phenoxymethyl]-piperidine-1-carboxylic acid 4-(4-methyl-pyridin-2-ylmethyl)-phenyl ester

4-[4-(5-Methyl-pyridin-2-yloxy)-phenoxymethyl]-piperidine-1-carboxylic acid 4-(4-methyl-pyridin-2-ylmethyl)-phenyl ester

4-(1-Methyl-1H-tetrazol-5-ylsulfanylmethyl)-piperidine-1-carboxylic acid 4-(4-methyl-pyridin-2-ylmethyl)-phenyl ester

4-[1-(2-Dimethylamino-ethyl)-1H-tetrazol-5-ylsulfanyl]-piperidine-1-carboxylic acid 4-(4-methyl-pyridin-2-ylmethyl)-phenyl ester

4-[1-(2-Carboxy-ethyl)-1H-tetrazol-5-ylsulfanyl]-piperidine-1-carboxylic acid 4-(4-methyl-pyridin-2-ylmethyl)-phenyl ester

4-(1-Carboxymethyl-1H-tetrazol-5-ylsulfanyl)-piperidine-1-carboxylic acid 4-(4-methyl-pyridin-2-ylmethyl)-phenyl ester

4-(Pyrazol-1-yloxymethyl)-piperidine-1-carboxylic acid 4-(4-methyl-pyridin-2-ylmethyl)-phenyl ester

4-(1H-[1,2,4]Triazol-3-ylsulfanylmethyl)-piperidine-1-carboxylic acid 4-(4-methyl-pyridin-2-ylmethyl)-phenyl ester

4-[2-(Benzyl-methyl-amino)-ethyl]-piperidine-1-carboxylic acid 4-(4-methyl-pyridin-2-ylmethyl)-phenyl ester

4-(1H-[1,2,4]Triazol-3-ylsulfanyl)-piperidine-1-carboxylic acid 4-pyridin-2-ylmethyl-phenyl ester

4-(1H-Imidazol-2-ylsulfanyl)-piperidine-1-carboxylic acid 4-pyridin-2-ylmethyl-phenyl ester

4-(1-Methyl-1H-imidazol-2-ylsulfanyl)-piperidine-1-carboxylic acid 4-pyridin-2-ylmethyl-phenyl ester

4-(1-Methyl-1H-tetrazol-5-ylsulfanyl)-piperidine-1-carboxylic acid 4-pyridin-2-ylmethyl-phenyl ester

4-(Imidazol-1-yloxy)-piperidine-1-carboxylic acid 4-pyridin-2-ylmethyl-phenyl ester

4-[1-(4-Carboxy-phenyl)-1H-tetrazol-5-ylsulfanyl]-piperidine-1-carboxylic acid 4-pyridin-2-ylmethyl-phenyl ester

4-(4-Carboxymethyl-phenoxy)-piperidine-1-carboxylic acid 4-pyridin-2-ylmethyl-phenyl ester

4-(Pyrazol-1-yloxy)-piperidine-1-carboxylic acid 4-pyridin-2-ylmethyl-phenyl ester

4-(4-Carboxy-phenoxy)-piperidine-1-carboxylic acid 4-pyridin-2-ylmethyl-phenyl ester

4-[4-(Pyridin-2-yloxy)-phenoxy]-piperidine-1-carboxylic acid 4-pyridin-2-ylmethyl-phenyl ester

4-[4-(5-Methyl-pyridin-2-yloxy)-phenoxy]-piperidine-1-carboxylic acid 4-pyridin-2-ylmethyl-phenyl ester

4-(4-Carboxy-phenoxymethyl)-piperidine-1-carboxylic acid 4-pyridin-2-ylmethyl-phenyl ester

4-(4-Carboxymethyl-phenoxymethyl)-piperidine-1-carboxylic acid 4-pyridin-2-ylmethyl-phenyl ester

4-(1H-Imidazol-2-ylsulfanylmethyl)-piperidine-1-carboxylic acid 4-pyridin-2-ylmethyl-phenyl ester

4-(1-Methyl-1H-imidazol-2-ylsulfanylmethyl)-piperidine-1-carboxylic acid 4-pyridin-2-ylmethyl-phenyl ester

4-(Imidazol-1-yloxymethyl)-piperidine-1-carboxylic acid 4-pyridin-2-ylmethyl-phenyl ester

4-[4-(Pyridin-2-yloxy)-phenoxymethyl]-piperidine-1-carboxylic acid 4-pyridin-2-ylmethyl-phenyl ester

4-[4-(5-Methyl-pyridin-2-yloxy)-phenoxymethyl]-piperidine-1-carboxylic acid 4-pyridin-2-ylmethyl-phenyl ester

4-(1-Methyl-1H-tetrazol-5-ylsulfanylmethyl)-piperidine-1-carboxylic acid 4-pyridin-2-ylmethyl-phenyl ester

4-[1-(2-Dimethylamino-ethyl)-1H-tetrazol-5-ylsulfanyl]-piperidine-1-carboxylic acid 4-pyridin-2-ylmethyl-phenyl ester

4-[1-(2-Carboxy-ethyl)-1H-tetrazol-5-ylsulfanyl]-piperidine-1-carboxylic acid 4-pyridin-2-ylmethyl-phenyl ester

4-(1-Carboxymethyl-1H-tetrazol-5-ylsulfanyl)-piperidine-1-carboxylic acid 4-pyridin-2-ylmethyl-phenyl ester

4-(Pyrazol-1-yloxymethyl)-piperidine-1-carboxylic acid 4-pyridin-2-ylmethyl-phenyl ester

4-(1H-[1,2,4]Triazol-3-ylsulfanylmethyl)-piperidine-1-carboxylic acid 4-pyridin-2-ylmethyl-phenyl ester

4-[2-(Benzyl-methyl-amino)-ethyl]-piperidine-1-carboxylic acid 4-pyridin-2-ylmethyl-phenyl ester

4-(1H-[1,2,4]Triazol-3-ylsulfanyl)-piperidine-1-carboxylic acid 4-(3-methyl-pyridin-2-ylmethyl)-phenyl ester

4-(1H-Imidazol-2-ylsulfanyl)-piperidine-1-carboxylic acid 4-(3-methyl-pyridin-2-ylmethyl)-phenyl ester

4-(1-Methyl-1H-imidazol-2-ylsulfanyl)-piperidine-1-carboxylic acid 4-(3-methyl-pyridin-2-ylmethyl)-phenyl ester

4-(1-Methyl-1H-tetrazol-5-ylsulfanyl)-piperidine-1-carboxylic acid 4-(3-methyl-pyridin-2-ylmethyl)-phenyl ester

4-(Imidazol-1-yloxy)-piperidine-1-carboxylic acid 4-(3-methyl-pyridin-2-ylmethyl)-phenyl ester

4-[1-(4-Carboxy-phenyl)-1H-tetrazol-5-ylsulfanyl]-piperidine-1-carboxylic acid 4-(3-methyl-pyridin-2-ylmethyl)-phenyl ester

4-(4-Carboxymethyl-phenoxy)-piperidine-1-carboxylic acid 4-(3-methyl-pyridin-2-ylmethyl)-phenyl ester

4-(Pyrazol-1-yloxy)-piperidine-1-carboxylic acid 4-(3-methyl-pyridin-2-ylmethyl)-phenyl ester

4-(4-Carboxy-phenoxy)-piperidine-1-carboxylic acid 4-(3-methyl-pyridin-2-ylmethyl)-phenyl ester

4-[4-(Pyridin-2-yloxy)-phenoxy]-piperidine-1-carboxylic acid 4-(3-methyl-pyridin-2-ylmethyl)-phenyl ester

4-[4-(5-Methyl-pyridin-2-yloxy)-phenoxy]-piperidine-1-carboxylic acid 4-(3-methyl-pyridin-2-ylmethyl)-phenyl ester

4-(4-Carboxy-phenoxymethyl)-piperidine-1-carboxylic acid 4-(3-methyl-pyridin-2-ylmethyl)-phenyl ester

4-(4-Carboxymethyl-phenoxymethyl)-piperidine-1-carboxylic acid 4-(3-methyl-pyridin-2-ylmethyl)-phenyl ester

4-(1H-Imidazol-2-ylsulfanylmethyl)-piperidine-1-carboxylic acid 4-(3-methyl-pyridin-2-ylmethyl)-phenyl ester

4-(1-Methyl-1H-imidazol-2-ylsulfanylmethyl)-piperidine-1-carboxylic acid 4-(3-methyl-pyridin-2-ylmethyl)-phenyl ester

4-(Imidazol-1-yloxymethyl)-piperidine-1-carboxylic acid 4-(3-methyl-pyridin-2-ylmethyl)-phenyl ester

4-[4-(Pyridin-2-yloxy)-phenoxymethyl]-piperidine-1-carboxylic acid 4-(3-methyl-pyridin-2-ylmethyl)-phenyl ester

4-[4-(5-Methyl-pyridin-2-yloxy)-phenoxymethyl]-piperidine-1-carboxylic acid 4-(3-methyl-pyridin-2-ylmethyl)-phenyl ester

4-(1-Methyl-1H-tetrazol-5-ylsulfanylmethyl)-piperidine-1-carboxylic acid 4-(3-methyl-pyridin-2-ylmethyl)-phenyl ester

4-[1-(2-Dimethylamino-ethyl)-1H-tetrazol-5-ylsulfanyl]-piperidine-1-carboxylic acid 4-(3-methyl-pyridin-2-ylmethyl)-phenyl ester

4-[1-(2-Carboxy-ethyl)-1H-tetrazol-5-ylsulfanyl]-piperidine-1-carboxylic acid 4-(3-methyl-pyridin-2-ylmethyl)-phenyl ester

4-(1-Carboxymethyl-1H-tetrazol-5-ylsulfanyl)-piperidine-1-carboxylic acid 4-(3-methyl-pyridin-2-ylmethyl)-phenyl ester

4-(Pyrazol-1-yloxymethyl)-piperidine-1-carboxylic acid 4-(3-methyl-pyridin-2-ylmethyl)-phenyl ester

4-(1H-[1,2,4]Triazol-3-ylsulfanylmethyl)-piperidine-1-carboxylic acid 4-(3-methyl-pyridin-2-ylmethyl)-phenyl ester

4-[2-(Benzyl-methyl-amino)-ethyl]-piperidine-1-carboxylic acid 4-(3-methyl-pyridin-2-ylmethyl)-phenyl ester

4-(1H-[1,2,4]Triazol-3-ylsulfanyl)-piperidine-1-carboxylic acid 4-[2-(1-methyl-1H-2-ylsulfanyl)-ethyl]-phenyl ester

4-(1H-Imidazol-2-ylsulfanyl)-piperidine-1-carboxylic acid 4-[2-(1-methyl-1H-imidazol-2-ylsulfanyl)-ethyl]-phenyl ester

4-(1-Methyl-1H-imidazol-2-ylsulfanyl)-piperidine-1-carboxylic acid 4-[2-(1-methyl-1H-imidazol-2-ylsulfanyl)-ethyl]-phenyl ester

4-(1-Methyl-1H-tetrazol-5-ylsulfanyl)-piperidine-1-carboxylic acid 4-[2-(1-methyl-1H-imidazol-2-ylsulfanyl)-ethyl]-phenyl ester

4-(Imidazol-1-yloxy)-piperidine-1-carboxylic acid 4-[2-(1-methyl-1H-imidazol-2-ylsulfanyl)-ethyl]-phenyl ester

4-[1-(4-Carboxy-phenyl)-1H-tetrazol-5-ylsulfanyl]-piperidine-1-carboxylic acid 4-[2-(1-methyl-1H-imidazol-2-ylsulfanyl)-ethyl]-phenyl ester

4-(4-Carboxymethyl-phenoxy)-piperidine-1-carboxylic acid 4-[2-(1-methyl-1H-imidazol-2-ylsulfanyl)-ethyl]-phenyl ester

4-(Pyrazol-1-yloxy)-piperidine-1-carboxylic acid 4-[2-(1-methyl-1H-imidazol-2-ylsulfanyl)-ethyl]-phenyl ester

4-(4-Carboxy-phenoxy)-piperidine-1-carboxylic acid 4-[2-(1-methyl-1H-imidazol-2-ylsulfanyl)-ethyl]-phenyl ester

4-[4-(Pyridin-2-yloxy)-phenoxy]-piperidine-1-carboxylic acid 4-[2-(1-methyl-1H-imidazol-2-ylsulfanyl)-ethyl]-phenyl ester

4-[4-(5-Methyl-pyridin-2-yloxy)-phenoxy]-piperidine-1-carboxylic acid 4-[2-(1-methyl-1H-imidazol-2-ylsulfanyl)-ethyl]-phenyl ester

4-(4-Carboxy-phenoxymethyl)-piperidine-1-carboxylic acid 4-[2-(1-methyl-1H-imidazol-2-ylsulfanyl)-ethyl]-phenyl ester

4-(4-Carboxymethyl-phenoxymethyl)-piperidine-1-carboxylic acid 4-[2-(1-methyl-1H-imidazol-2-ylsulfanyl)-ethyl]-phenyl ester

4-(1H-Imidazol-2-ylsulfanylmethyl)-piperidine-1-carboxylic acid 4-[2-(1-methyl-1H-imidazol-2-ylsulfanyl)-ethyl]-phenyl ester

4-(1-Methyl-1H-imidazol-2-ylsulfanylmethyl)-piperidine-1-carboxylic acid 4-[2-(1-methyl-1H-imidazol-2-ylsulfanyl)-ethyl]-phenyl ester

4-(Imidazol-1-yloxymethyl)-piperidine-1-carboxylic acid 4-[2-(1-methyl-1H-imidazol-2-ylsulfanyl)-ethyl]-phenyl ester

4-[4-(Pyridin-2-yloxy)-phenoxymethyl]-piperidine-1-carboxylic acid 4-[2-(1-methyl-1H-imidazol-2-ylsulfanyl)-ethyl]-phenyl ester

4-[4-(5-Methyl-pyridin-2-yloxy)-phenoxymethyl]-piperidine-1-carboxylic acid 4-[2-(1-methyl-1H-imidazol-2-ylsulfanyl)-ethyl]-phenyl ester

4-(1-Methyl-1H-tetrazol-5-ylsulfanylmethyl)-piperidine-1-carboxylic acid 4-[2-(1-methyl-1H-imidazol-2-ylsulfanyl)-ethyl]-phenyl ester

4-[1-(2-Dimethylamino-ethyl)-1H-tetrazol-5-ylsulfanyl]-piperidine-1-carboxylic acid 4-[2-(1-methyl-1H-imidazol-2-ylsulfanyl)-ethyl]-phenyl ester

4-[1-(2-Carboxy-ethyl)-1H-tetrazol-5-ylsulfanyl]-piperidine-1-carboxylic acid 4-[2-(1-methyl-1H-imidazol-2-ylsulfanyl)-ethyl]-phenyl ester

4-(1-Carboxymethyl-1H-tetrazol-5-ylsulfanyl)-piperidine-1-carboxylic acid 4-[2-(1-methyl-1H-imidazol-2-ylsulfanyl)-ethyl]-phenyl ester

4-(Pyrazol-1-yloxymethyl)-piperidine-1-carboxylic acid 4-[2-(1-methyl-1H-imidazol-2-ylsulfanyl)-ethyl]-phenyl ester

4-(1H-[1,2,4]Triazol-3-ylsulfanylmethyl)-piperidine-1-carboxylic acid 4-[2-(1-methyl-imidazol-2-ylsulfanyl)-ethyl]-phenyl ester

4-[2-(Benzyl-methyl-amino)-ethyl]-piperidine-1-carboxylic acid 4-[2-(1-methyl-1H-imidazol-2-ylsulfanyl)-ethyl]-phenyl ester

4-(1H-[1,2,4]Triazol-3-ylsulfanyl)-piperidine-1-carboxylic acid 4-[2-(pyridin-2-ylsulfanyl)-ethyl]-phenyl ester

4-(1H-Imidazol-2-ylsulfanyl)-piperidine-1-carboxylic acid 4-[2-(pyridin-2-ylsulfanyl)-ethyl]-phenyl ester

4-(1-Methyl-1H-imidazol-2-ylsulfanyl)-piperidine-1-carboxylic acid 4-[2-(pyridin-2-ylsulfanyl)-ethyl]-phenyl ester

4-(1-Methyl-1H-tetrazol-5-ylsulfanyl)-piperidine-1-carboxylic acid 4-[2-(pyridin-2-ylsulfanyl)-ethyl]-phenyl ester

4-(Imidazol-1-yloxy)-piperidine-1-carboxylic acid 4-[2-(pyridin-2-ylsulfanyl)-ethyl]-phenyl ester

4-[1-(4-Carboxy-phenyl)-1H-tetrazol-5-ylsulfanyl]-piperidine-1-carboxylic acid 4-[2-(pyridin-2-ylsulfanyl)-ethyl]-phenyl ester

4-(4-Carboxymethyl-phenoxy)-piperidine-1-carboxylic acid 4-[2-(pyridin-2-ylsulfanyl)-ethyl]-phenyl ester

4-(Pyrazol-1-yloxy)-piperidine-1-carboxylic acid 4-[2-(pyridin-2-ylsulfanyl)-ethyl]-phenyl ester

4-(4-Carboxy-phenoxy)-piperidine-1-carboxylic acid 4-[2-(pyridin-2-ylsulfanyl)-ethyl]-phenyl ester

4-[4-(Pyridin-2-yloxy)-phenoxy]-piperidine-1-carboxylic acid 4-[2-(pyridin-2-ylsulfanyl)-ethyl]-phenyl ester

4-[4-(5-Methyl-pyridin-2-yloxy)-phenoxy]-piperidine-1-carboxylic acid 4-[2-(pyridin-2-ylsulfanyl)-ethyl]-phenyl ester

4-(4-Carboxy-phenoxymethyl)-piperidine-1-carboxylic acid 4-[2-(pyridin-2-ylsulfanyl)-ethyl]-phenyl ester

4-(4-Carboxymethyl-phenoxymethyl)-piperidine-1-carboxylic acid 4-[2-(pyridin-2-ylsulfanyl)-ethyl]-phenyl ester

4-(1H-Imidazol-2-ylsulfanylmethyl)-piperidine-1-carboxylic acid 4-[2-(pyridin-2-ylsulfanyl)-ethyl]-phenyl ester

4-(1-Methyl-1H-imidazol-2-ylsulfanylmethyl)-piperidine-1-carboxylic acid 4-[2-(pyridin-2-ylsulfanyl)-ethyl]-phenyl ester

4-(Imidazol-1-yloxymethyl)-piperidine-1-carboxylic acid 4-[2-(pyridin-2-ylsulfanyl)-ethyl]-phenyl ester

4-[4-(Pyridin-2-yloxy)-phenoxymethyl]-piperidine-1-carboxylic acid 4-[2-(pyridin-2-ylsulfanyl)-ethyl]-phenyl ester

4-[4-(5-Methyl-pyridin-2-yloxy)-phenoxymethyl]-piperidine-1-carboxylic acid 4-[2-(pyridin-2-ylsulfanyl)-ethyl]-phenyl ester

4-(1-Methyl-1H-tetrazol-5-ylsulfanylmethyl)-piperidine-1-carboxylic acid 4-[2-(pyridin-2-ylsulfanyl)-ethyl]-phenyl ester

4-[1-(2-Dimethylamino-ethyl)-1H-tetrazol-5-ylsulfanyl]-piperidine-1-carboxylic acid 4-[2-(pyridin-2-ylsulfanyl)-ethyl]-phenyl ester

4-[1-(2-Carboxy-ethyl)-1H-tetrazol-5-ylsulfanyl]-piperidine-1-carboxylic acid 4-[2-(pyridin-2-ylsulfanyl)-ethyl]-phenyl ester

4-(1-Carboxymethyl-1H-tetrazol-5-ylsulfanyl)-piperidine-1-carboxylic acid 4-[2-(pyridin-2-ylsulfanyl)-ethyl]-phenyl ester

4-(Pyrazol-1-yloxymethyl)-piperidine-1-carboxylic acid 4-[2-(pyridin-2-ylsulfanyl)-ethyl]-phenyl ester

4-(1H-[1,2,4]Triazol-3-ylsulfanylmethyl)-piperidine-1-carboxylic acid 4-[2-(pyridin-2-ylsulfanyl)-ethyl]-phenyl ester

4-[2-(Benzyl-methyl-amino)-ethyl]-piperidine-1-carboxylic acid 4-[2-(pyridin-2-ylsulfanyl)-ethyl]-phenyl ester

4-(1H-[1,2,4]Triazol-3-ylsulfanyl)-piperidine-1-carboxylic acid 4-[2-(pyridin-2-yloxy)-phenyl ester

4-(1H-Imidazol-2-ylsulfanyl)-piperidine-1-carboxylic acid 4-[2-(pyridin-2-yloxy)-ethyl]-phenyl ester

4-(1-Methyl-1H-imidazol-2-ylsulfanyl)-piperidine-1-carboxylic acid 4-[2-(pyridin-2-yloxy)-ethyl]-phenyl ester

4-(1-Methyl-1H-tetrazol-5-ylsulfanyl)-piperidine-1-carboxylic acid 4-[2-(pyridin-2-yloxy)-ethyl]-phenyl ester

4-(Imidazol-1-yloxy)-piperidine-1-carboxylic acid 4-[2-(pyridin-2-yloxy)-ethyl]-phenyl ester

4-[1-(4-Carboxy-phenyl)-1H-tetrazol-5-ylsulfanyl]-piperidine-1-carboxylic acid 4-[2-(pyridin-2-yloxy)-ethyl]-phenyl ester

4-(4-Carboxymethyl-phenoxy)-piperidine-1-carboxylic acid 4-[2-(pyridin-2-yloxy)-ethyl]-phenyl ester

4-(Pyrazol-1-yloxy)-piperidine-1-carboxylic acid 4-[2-(pyridin-2-yloxy)-ethyl]-phenyl ester

4-(4-Carboxy-phenoxy)-piperidine-1-carboxylic acid 4-[2-(pyridin-2-yloxy)-ethyl]-phenyl ester

4-[4-(Pyridin-2-yloxy)-phenoxy]-piperidine-1-carboxylic acid 4-[2-(pyridin-2-yloxy)-ethyl]-phenyl ester

4-[4-(5-Methyl-pyridin-2-yloxy)-phenoxy]-piperidine-1-carboxylic acid 4-[2-(pyridin-2-yloxy)-ethyl]-phenyl ester

4-(4-Carboxy-phenoxymethyl)-piperidine-1-carboxylic acid 4-[2-(pyridin-2-yloxy)-ethyl]-phenyl ester

4-(4-Carboxymethyl-phenoxymethyl)-piperidine-1-carboxylic acid 4-[2-(pyridin-2-yloxy)-ethyl]-phenyl ester

4-(1H-Imidazol-2-ylsulfanylmethyl)-piperidine-1-carboxylic acid 4-[2-(pyridin-2-yloxy)-ethyl]-phenyl ester

4-(1-Methyl-1H-imidazol-2-ylsulfanylmethyl)-piperidine-1-carboxylic acid 4-[2-(pyridin-2-yloxy)-ethyl]-phenyl ester

4-(Imidazol-1-yloxymethyl)-piperidine-1-carboxylic acid 4-[2-(pyridin-2-yloxy)-ethyl]-phenyl ester

4-[4-(Pyridin-2-yloxy)-phenoxymethyl]-piperidine-1-carboxylic acid 4-[2-(pyridin-2-yloxy)-ethyl]-phenyl ester

4-[4-(5-Methyl-pyridin-2-yloxy)-phenoxymethyl]-piperidine-1-carboxylic acid 4-[2-(pyridin-2-yloxy)-ethyl]-phenyl ester

4-(1-Methyl-1H-tetrazol-5-ylsulfanylmethyl)-piperidine-1-carboxylic acid 4-[2-(pyridin-2-yloxy)-ethyl]-phenyl ester

4-[1-(2-Dimethylamino-ethyl)-1H-tetrazol-5-ylsulfanyl]-piperidine-1-carboxylic acid 4-[2-(pyridin-2-yloxy)-ethyl]-phenyl ester

4-[1-(2-Carboxy-ethyl)-1H-tetrazol-5-ylsulfanyl]-piperidine-1-carboxylic acid 4-[2-(pyridin-2-yloxy)-ethyl]-phenyl ester

4-(1-Carboxymethyl-1H-tetrazol-5-ylsulfanyl)-piperidine-1-carboxylic acid 4-[2-(pyridin-2-yloxy)-ethyl]-phenyl ester

4-(Pyrazol-1-yloxymethyl)-piperidine-1--carboxylic acid 4-[2-(pyridin-2-yloxy)-ethyl]-phenyl ester

4-(1H-[1,2,4]Triazol-3-ylsulfanylmethyl)-piperidine-1-carboxylic acid 4-[2-(pyridin-2-ethyl]-phenyl ester

4-[2-(Benzyl-methyl-amino)-ethyl]-piperidine-1-carboxylic acid 4-[2-(pyridin-2-yloxy)-ethyl]-phenyl ester

4-(1H-[1,2,4]Triazol-3-ylsulfanyl)-piperidine-1-carboxylic acid 4-[2-(pyridin-3-yloxy)-phenyl ester

4-(1H-Imidazol-2-ylsulfanyl)-piperidine-1-carboxylic acid 4-[2-(pyridin-3-yloxy)-ethyl]-phenyl ester

4-(1-Methyl-1H-imidazol-2-ylsulfanyl)-piperidine-1-carboxylic acid 4-[2-(pyridin-3-yloxy)-ethyl]-phenyl ester

4-(1-Methyl-1H-tetrazol-5-ylsulfanyl)-piperidine-1-carboxylic acid 4-[2-(pyridin-3-yloxy)-ethyl]-phenyl ester

4-(Imidazol-1-yloxy)-piperidine-1-carboxylic acid 4-[2-(pyridin-3-yloxy)-ethyl]-phenyl ester

4-[1-(4-Carboxy-phenyl)-1H-tetrazol-5-ylsulfanyl]-piperidine-1-carboxylic acid 4-[2-(pyridin-3-yloxy)-ethyl]-phenyl ester

4-(4-Carboxymethyl-phenoxy)-piperidine-1-carboxylic acid 4-[2-(pyridin-3-yloxy)-ethyl]-phenyl ester

4-(Pyrazol-1-yloxy)-piperidine-1-carboxylic acid 4-[2-(pyridin-3-yloxy)-ethyl]-phenyl ester

4-(4-Carboxy-phenoxy)-piperidine-1-carboxylic acid 4-[2-(pyridin-3-yloxy)-ethyl]-phenyl ester

4-[4-(Pyridin-2-yloxy)-phenoxy]-piperidine-1-carboxylic acid 4-[2-(pyridin-3-yloxy)-ethyl]-phenyl ester

4-[4-(5-Methyl-pyridin-2-yloxy)-phenoxy]-piperidine-1-carboxylic acid 4-[2-(pyridin-3-yloxy)-ethyl]-phenyl ester

4-(4-Carboxy-phenoxymethyl)-piperidine-1-carboxylic acid 4-[2-(pyridin-3-yloxy)-ethyl]-phenyl ester

4-(4-Carboxymethyl-phenoxymethyl)-piperidine-1-carboxylic acid 4-[2-(pyridin-3-yloxy)-ethyl]-phenyl ester

4-(1H-Imidazol-2-ylsulfanylmethyl)-piperidine-1-carboxylic acid 4-[2-(pyridin-3-yloxy)-ethyl]-phenyl ester

4-(1-Methyl-1H-imidazol-2-ylsulfanylmethyl)-piperidine-1-carboxylic acid 4-[2-(pyridin-3-yloxy)-ethyl]-phenyl ester

4-(Imidazol-1-yloxymethyl)-piperidine-1-carboxylic acid 4-[2-(pyridin-3-yloxy)-ethyl]-phenyl ester

4-[4-(Pyridin-2-yloxy)-phenoxymethyl]-piperidine-1-carboxylic acid 4-[2-(pyridin-3-yloxy)-ethyl]-phenyl ester

4-[4-(5-Methyl-pyridin-2-yloxy)-phenoxymethyl]-piperidine-1-carboxylic acid 4-[2-(pyridin-3-yloxy)-ethyl]-phenyl ester

4-(1-Methyl-1H-tetrazol-5-ylsulfanylmethyl)-piperidine-1-carboxylic acid 4-[2-(pyridin-3-yloxy)-ethyl]-phenyl ester

4-[1-(2-Dimethylamino-ethyl)-1H-tetrazol-5-ylsulfanyl]-piperidine-1-carboxylic acid 4-[2-(pyridin-3-yloxy)-ethyl]-phenyl ester

4-[1-(2-Carboxy-ethyl)-1H-tetrazol-5-ylsulfanyl]-piperidine-1-carboxylic acid 4-[2-(pyridin-3-yloxy)-ethyl]-phenyl ester

4-(1-Carboxymethyl-1H-tetrazol-5-ylsulfanyl)-piperidine-1-carboxylic acid 4-[2-(pyridin-3-yloxy)-ethyl]-phenyl ester

4-(Pyrazol-1-yloxymethyl)-piperidine-1-carboxylic acid 4-[2-(pyridin-3-yloxy)-ethyl]-phenyl ester

4-(1H-[1,2,4]Triazol-3-ylsulfanylmethyl)-piperidine-1-carboxylic acid 4-[2-(pyridin-3-ethyl]-phenyl ester

4-[2-(Benzyl-methyl-amino)-ethyl]-piperidine-1-carboxylic acid 4-[2-(pyridin-3-yloxy)-ethyl]-phenyl ester

4-(1H-[1,2,4]Triazol-3-ylsulfanyl)-piperidine-1-carboxylic acid 4-[2-(pyridin-4-yloxy)-ethyl]-phenyl ester

4-(1H-Imidazol-2-ylsulfanyl)-piperidine-1-carboxylic acid 4-[2-(pyridin-4-yloxy)-ethyl]-phenyl ester

4-(1-Methyl-1H-imidazol-2-ylsulfanyl)-piperidine-1-carboxylic acid 4-[2-(pyridin-4-yloxy)-ethyl]-phenyl ester

4-(1-Methyl-1H-tetrazol-5-ylsulfanyl)-piperidine-1-carboxylic acid 4-[2-(pyridin-4-yloxy)-ethyl]-phenyl ester

4-(Imidazol-1-yloxy)-piperidine-1-carboxylic acid 4-[2-(pyridin-4-yloxy)-ethyl]-phenyl ester

4-[1-(4-Carboxy-phenyl)-1H-tetrazol-5-ylsulfanyl]-piperidine-1-carboxylic acid 4-[2-(pyridin-4-yloxy)-ethyl]-phenyl ester

4-(4-Carboxymethyl-phenoxy)-piperidine-1-carboxylic acid 4-[2-(pyridin-4-yloxy)-ethyl]-phenyl ester

4-(Pyrazol-1-yloxy)-piperidine-1-carboxylic acid 4-[2-(pyridin-4-yloxy)-ethyl]-phenyl ester

4-(4-Carboxy-phenoxy)-piperidine-1-carboxylic acid 4-[2-(pyridin-4-yloxy)-ethyl]-phenyl ester

4-[4-(Pyridin-2-yloxy)-phenoxy]-piperidine-1-carboxylic acid 4-[2-(pyridin-4-yloxy)-ethyl]-phenyl ester

4-[4-(5-Methyl-pyridin-2-yloxy)-phenoxy]-piperidine-1-carboxylic acid 4-[2-(pyridin-4-yloxy)-ethyl]-phenyl ester

4-(4-Carboxy-phenoxymethyl)-piperidine-1-carboxylic acid 4-[2-(pyridin-4-yloxy)-ethyl]-phenyl ester

4-(4-Carboxymethyl-phenoxymethyl)-piperidine-1-carboxylic acid 4-[2-(pyridin-4-yloxy)-ethyl]-phenyl ester

4-(1H-Imidazol-2-ylsulfanylmethyl)-piperidine-1-carboxylic acid 4-[2-(pyridin-4-yloxy)-ethyl]-phenyl ester

4-(1-Methyl-1H-imidazol-2-ylsulfanylmethyl)-piperidine-1-carboxylic acid 4-[2-(pyridin-4-yloxy)-ethyl]-phenyl ester

4-(Imidazol-1-yloxymethyl)-piperidine-1-carboxylic acid 4-[2-(pyridin-4-yloxy)-ethyl]-phenyl ester

4-[4-(Pyridin-2-yloxy)-phenoxymethyl]-piperidine-1-carboxylic acid 4-[2-(pyridin-4-yloxy)-ethyl]-phenyl ester

4-[4-(5-Methyl-pyridin-2-yloxy)-phenoxymethyl]-piperidine-1-carboxylic acid 4-[2-(pyridin-4-yloxy)-ethyl]-phenyl ester

4-(1-Methyl-1H-tetrazol-5-ylsulfanylmethyl)-piperidine-1-carboxylic acid 4-[2-(pyridin-4-yloxy)-ethyl]-phenyl ester

4-[1-(2-Dimethylamino-ethyl)-1H-tetrazol-5-ylsulfanyl]-piperidine-1-carboxylic acid 4-[2-(pyridin-4-yloxy)-ethyl]-phenyl ester

4-[1-(2-Carboxy-ethyl)-1H-tetrazol-5-ylsulfanyl]-piperidine-1-carboxylic acid 4-[2-(pyridin-4-yloxy)-ethyl]-phenyl ester

4-(1-Carboxymethyl-1H-tetrazol-5-ylsulfanyl)-piperidine-1-carboxylic acid 4-[2-(pyridin-4-yloxy)-ethyl]-phenyl ester

4-(Pyrazol-1-yloxymethyl)-piperidine-1-carboxylic acid 4-[2-(pyridin-4-yloxy)-ethyl]-phenyl ester

4-(1H-[1,2,4]Triazol-3-ylsulfanylmethyl)-piperidine-1-carboxylic acid 4-[2-(pyridin-4-ethyl]-phenyl ester

4-[2-(Benzyl-methyl-amino)-ethyl]-piperidine-1-carboxylic acid 4-[2-(pyridin-4-yloxy)-ethyl]-phenyl ester

4-(1H-[1,2,4]Triazol-3-ylsulfanyl)-piperidine-1-carboxylic acid 4-[2-(6-methyl-pyridin-2-yl-ethyl]-phenyl ester

4-(1H-Imidazol-2-ylsulfanyl)-piperidine-1-carboxylic acid 4-[2-(6-methyl-pyridin-2-yl)-ethyl]-phenyl ester

4-(1-Methyl-1H-imidazol-2-ylsulfanyl)-piperidine-1-carboxylic acid 4-[2-(6-methyl-pyridin-2-yl)-ethyl]-phenyl ester

4-(1-Methyl-1H-tetrazol-5-ylsulfanyl)-piperidine-1-carboxylic acid 4-[2-(6-methyl-pyridin-yl)-ethyl]-phenyl ester

4-(Imidazol-1-yloxy)-piperidine-1-carboxylic acid 4-[2-(6-methyl-pyridin-2-yl)-ethyl]-phenyl ester

4-[1-(4-Carboxy-phenyl)-1H-tetrazol-5-ylsulfanyl]-piperidine-1-carboxylic acid 4-[2-(6-methyl-pyridin-2-yl)-ethyl]-phenyl ester

4-(4-Carboxymethyl-phenoxy)-piperidine-1-carboxylic acid 4-[2-(6-methyl-pyridin-2-yl)-ethyl]-phenyl ester

4-(Pyrazol-1-yloxy)-piperidine-1-carboxylic acid 4-[2-(6-methyl-pyridin-2-yl)-ethyl]-phenyl ester

4-(4-Carboxy-phenoxy)-piperidine-1-carboxylic acid 4-[2-(6-methyl-pyridin-2-yl)-ethyl]-phenyl ester

4-[4-(Pyridin-2-yloxy)-phenoxy]-piperidine-1-carboxylic acid 4-[2-(6-methyl-pyridin-2-yl)-ethyl]-phenyl ester

4-[4-(5-Methyl-pyridin-2-yloxy)-phenoxy]-piperidine-1-carboxylic acid 4-[2-(6-methyl-pyridin-2-yl)-ethyl]-phenyl ester

4-(4-Carboxy-phenoxymethyl)-piperidine-1-carboxylic acid 4-[2-(6-methyl-pyridin-2-yl)-ethyl]-phenyl ester

4-(4-Carboxymethyl-phenoxymethyl)-piperidine-1-carboxylic acid 4-[2-(6-methyl-pyridin-2-yl)-ethyl]-phenyl ester

4-(1H-Imidazol-2-ylsulfanylmethyl)-piperidine-1-carboxylic acid 4-[2-(6-methyl-pyridin-2-yl)-ethyl]-phenyl ester

4-(1-Methyl-1H-imidazol-2-ylsulfanylmethyl)-piperidine-1-carboxylic acid 4-[2-(6-methyl-pyridin-2-yl)-ethyl]-phenyl ester

4-(Imidazol-1-yloxymethyl)-piperidine-1-carboxylic acid 4-[2-(6-methyl-pyridin-2-yl)-ethyl]-phenyl ester

4-[4-(Pyridin-2-yloxy)-phenoxymethyl]-piperidine-1-carboxylic acid 4-[2-(6-methyl-pyridin-2-yl)-ethyl]-phenyl ester

4-[4-(5-Methyl-pyridin-2-yloxy)-phenoxymethyl]-piperidine-1-carboxylic acid 4-[2-(6-methyl-pyridin-2-yl)-ethyl]-phenyl ester

4-(1-Methyl-1H-tetrazol-5-ylsulfanylmethyl)-piperidine-1-carboxylic acid 4-[2-(6-methyl-pyridin-2-yl)-ethyl]-phenyl ester

4-[1-(2-Dimethylamino-ethyl)-1H-tetrazol-5-ylsulfanyl]-piperidine-1-carboxylic acid 4-[2-(6-methyl-pyridin-2-yl)-ethyl]-phenyl ester

4-[1-(2-Carboxy-ethyl)-1H-tetrazol-5-ylsulfanyl]-piperidine-1-carboxylic acid 4-[2-(6-methyl-pyridin-2-yl)-ethyl]-phenyl ester

4-(1-Carboxymethyl-1H-tetrazol-5-ylsulfanyl)-piperidine-1-carboxylic acid 4-[2-(6-methyl-pyridin-2-yl)-ethyl]-phenyl ester

4-(Pyrazol-1-yloxymethyl)-piperidine-1-carboxylic acid 4-[2-(6-methyl-pyridin-2-yl)-ethyl]-phenyl ester

4-(1H-[1,2,4]Triazol-3-ylsulfanylmethyl)-piperidine-1-carboxylic acid 4-[2-(6-methyl-pyridin-2-yl)-ethyl]-phenyl ester

4-[2-(Benzyl-methyl-amino)-ethyl]-piperidine-1-carboxylic acid 4-[2-(6-methyl-pyridin-2-yl)-ethyl]-phenyl ester

4-(1H-[1,2,4]Triazol-3-ylsulfanyl)-piperidine-1-carboxylic acid 4-[2-(4-methyl-pyridin-2-yl)-ethyl]-phenyl ester

4-(1H-Imidazol-2-ylsulfanyl)-piperidine-1-carboxylic acid 4-[2-(4-methyl-pyridin-2-yl)-ethyl]-phenyl ester

4-(1-Methyl-1H-imidazol-2-ylsulfanyl)-piperidine-1-carboxylic acid 4-[2-(4-methyl-pyridin-2-yl)-ethyl]-phenyl ester

4-(1-Methyl-1H-tetrazol-5-ylsulfanyl)-piperidine-1-carboxylic acid 4-[2-(4-methyl-pyridin-yl)-ethyl]-phenyl ester

4-(Imidazol-1-yloxy)-piperidine-1-carboxylic acid 4-[2-(4-methyl-pyridin-2-yl)-ethyl]-phenyl ester

4-[1-(4-Carboxy-phenyl)-1H-tetrazol-5-ylsulfanyl]-piperidine-1-carboxylic acid 4-[2-(4-methyl-pyridin-2-yl)-ethyl]-phenyl ester

4-(4-Carboxymethyl-phenoxy)-piperidine-1-carboxylic acid 4-[2-(4-methyl-pyridin-2-yl)-ethyl]-phenyl ester

4-(Pyrazol-1-yloxy)-piperidine-1-carboxylic acid 4-[2-(4-methyl-pyridin-2-yl)-ethyl]-phenyl ester

4-(4-Carboxy-phenoxy)-piperidine-1-carboxylic acid 4-[2-(4-methyl-pyridin-2-yl)-ethyl]-phenyl ester

4-[4-(Pyridin-2-yloxy)-phenoxy]-piperidine-1-carboxylic acid 4-[2-(4-methyl-pyridin-2-yl)-ethyl]-phenyl ester

4-[4-(5-Methyl-pyridin-2-yloxy)-phenoxy]-piperidine-1-carboxylic acid 4-[2-(4-methyl-pyridin-2-yl)-ethyl]-phenyl ester

4-(4-Carboxy-phenoxymethyl)-piperidine-1-carboxylic acid 4-[2-(4-methyl-pyridin-2-yl)-ethyl]-phenyl ester

4-(4-Carboxymethyl-phenoxymethyl)-piperidine-1-carboxylic acid 4-[2-(4-methyl-pyridin-2-yl)-ethyl]-phenyl ester

4-(1H-Imidazol-2-ylsulfanylmethyl)-piperidine-1-carboxylic acid 4-[2-(4-methyl-pyridin-2-yl)-ethyl]-phenyl ester

4-(1-Methyl-1H-imidazol-2-ylsulfanylmethyl)-piperidine-1-carboxylic acid 4-[2-(4-methyl-pyridin-2-yl)-ethyl]-phenyl ester

4-(Imidazol-1-yloxymethyl)-piperidine-1-carboxylic acid 4-[2-(4-methyl-pyridin-2-yl)-ethyl]-phenyl ester

4-[4-(Pyridin-2-yloxy)-phenoxymethyl]-piperidine-1-carboxylic acid 4-[2-(4-methyl-pyridin-2-yl)-ethyl]-phenyl ester

4-[4-(5-Methyl-pyridin-2-yloxy)-phenoxymethyl]-piperidine-1-carboxylic acid 4-[2-(4-methyl-pyridin-2-yl)-ethyl]-phenyl ester

4-(1-Methyl-1H-tetrazol-5-ylsulfanylmethyl)-piperidine-1-carboxylic acid 4-[2-(4-methyl-pyridin-2-yl)-ethyl]-phenyl ester

4-[1-(2-Dimethylamino-ethyl)-1H-tetrazol-5-ylsulfanyl]-piperidine-1-carboxylic acid 4-[2-(4-methyl-pyridin-2-yl)-ethyl]-phenyl ester

4-[1-(2-Carboxy-ethyl)-1H-tetrazol-5-ylsulfanyl]-piperidine-1-carboxylic acid 4-[2-(4-methyl-pyridin-2-yl)-ethyl]-phenyl ester

4-(1-Carboxymethyl-1H-tetrazol-5-ylsulfanyl)-piperidine-1-carboxylic acid 4-[2-(4-methyl-pyridin-2-yl)-ethyl]-phenyl ester

4-(Pyrazol-1-yloxymethyl)-piperidine-1-carboxylic acid 4-[2-(4-methyl-pyridin-2-yl)-ethyl]-phenyl ester

4-(1H-[1,2,4]Triazol-3-ylsulfanylmethyl)-piperidine-1-carboxylic acid 4-[2-(4-methyl-pyridin-2-yl)-ethyl]-phenyl ester

4-[2-(Benzyl-methyl-amino)-ethyl]-piperidine-1-carboxylic acid 4-[2-(4-methyl-pyridin-2-yl)-ethyl]-phenyl ester

4-(1H-[1,2,4]Triazol-3-ylsulfanyl)-piperidine-1-carboxylic acid 4-[2-(3-methyl-pyridin-2-yl)-ethyl]-phenyl ester

4-(1H-Imidazol-2-ylsulfanyl)-piperidine-1-carboxylic acid 4-[2-(3-methyl-pyridin-2-yl)-ethyl]-phenyl ester

4-(1-Methyl-1H-imidazol-2-ylsulfanyl)-piperidine-1-carboxylic acid 4-[2-(3-methyl-pyridin-2-yl)-ethyl]-phenyl ester

4-(1-Methyl-1H-tetrazol-5-ylsulfanyl)-piperidine-1-carboxylic acid 4-[2-(3-methyl-pyridin-2-yl)-ethyl]-phenyl ester

4-(Imidazol-1-yloxy)-piperidine-1-carboxylic acid 4-[2-(3-methyl-pyridin-2-yl)-ethyl]-phenyl ester

4-[1-(4-Carboxy-phenyl)-1H-tetrazol-5-ylsulfanyl]-piperidine-1-carboxylic acid 4-[2-(3-methyl-pyridin-2-yl)-ethyl]-phenyl ester

4-(4-Carboxymethyl-phenoxy)-piperidine-1-carboxylic acid 4-[2-(3-methyl-pyridin-2-yl)-ethyl]-phenyl ester

4-(Pyrazol-1-yloxy)-piperidine-1-carboxylic acid 4-[2-(3-methyl-pyridin-2-yl)-ethyl]-phenyl ester

4-(4-Carboxy-phenoxy)-piperidine-1-carboxylic acid 4-[2-(3-methyl-pyridin-2-yl)-ethyl]-phenyl ester

4-[4-(Pyridin-2-yloxy)-phenoxy]-piperidine-1-carboxylic acid 4-[2-(3-methyl-pyridin-2-yl)-ethyl]-phenyl ester

4-[4-(5-Methyl-pyridin-2-yloxy)-phenoxy]-piperidine-1-carboxylic acid 4-[2-(3-methyl-pyridin-2-yl)-ethyl]-phenyl ester

4-(4-Carboxy-phenoxymethyl)-piperidine-1-carboxylic acid 4-[2-(3-methyl-pyridin-2-yl)-ethyl]-phenyl ester

4-(4-Carboxymethyl-phenoxymethyl)-piperidine-1-carboxylic acid 4-[2-(3-methyl-pyridin-2-yl)-ethyl]-phenyl ester

4-(1H-Imidazol-2-ylsulfanylmethyl)-piperidine-1-carboxylic acid 4-[2-(3-methyl-pyridin-2-yl)-ethyl]-phenyl ester

4-(1-Methyl-1H-imidazol-2-ylsulfanylmethyl)-piperidine-1-carboxylic acid 4-[2-(3-methyl-pyridin-2-yl)-ethyl]-phenyl ester

4-(Imidazol-1-yloxymethyl)-piperidine-1-carboxylic acid 4-[2-(3-methyl-pyridin-2-yl)-ethyl]-phenyl ester

4-[4-(Pyridin-2-yloxy)-phenoxymethyl]-piperidine-1-carboxylic acid 4-[2-(3-methyl-pyridin-2-yl)-ethyl]-phenyl ester

4-[4-(5-Methyl-pyridin-2-yloxy)-phenoxymethyl]-piperidine-1-carboxylic acid 4-[2-(3-methyl-pyridin-2-yl)-ethyl]-phenyl ester

4-(1-Methyl-1H-tetrazol-5-ylsulfanylmethyl)-piperidine-1-carboxylic acid 4-[2-(3-methyl-pyridin-2-yl)-ethyl]-phenyl ester

4-[1-(2-Dimethylamino-ethyl)-1H-tetrazol-5-ylsulfanyl]-piperidine-1-carboxylic acid 4-[2-(3-methyl-pyridin-2-yl)-ethyl]-phenyl ester

4-[1-(2-Carboxy-ethyl)-1H-tetrazol-5-ylsulfanyl]-piperidine-1-carboxylic acid 4-[2-(3-methyl-pyridin-2-yl)-ethyl]-phenyl ester

4-(1-Carboxymethyl-1H-tetrazol-5-ylsulfanyl)-piperidine-1-carboxylic acid 4-[2-(3-methyl-pyridin-2-yl)-ethyl]-phenyl ester

4-(Pyrazol-1-yloxymethyl)-piperidine-1-carboxylic acid 4-[2-(3-methyl-pyridin-2-yl)-ethyl]-phenyl ester

4-(1H-[1,2,4]Triazol-3-ylsulfanylmethyl)-piperidine-1-carboxylic acid 4-[2-(3-methyl-pyridin-2-yl)-ethyl]-phenyl ester

4-[2-(Benzyl-methyl-amino)-ethyl]-piperidine-1-carboxylic acid 4-[2-(3-methyl-pyridin-2-yl)-ethyl]-phenyl ester

4-(1H-[1,2,4]Triazol-3-ylsulfanyl)-piperidine-1-carboxylic acid 4-[2-(5-methyl-pyridin-2-ethyl]-phenyl ester

4-(1H-Imidazol-2-ylsulfanyl)-piperidine-1-carboxylic acid 4-[2-(5-methyl-pyridin-2-yl)-ethyl]-phenyl ester

4-(1-Methyl-1H-imidazol-2-ylsulfanyl)-piperidine-1-carboxylic acid 4-[2-(5-methyl-pyridin-2-yl)-ethyl]-phenyl ester

4-(1-Methyl-1H-tetrazol-5-ylsulfanyl)-piperidine-1-carboxylic acid 4-[2-(5-methyl-pyridin-yl)-ethyl]-phenyl ester

4-(Imidazol-1-yloxy)-piperidine-1-carboxylic acid 4-[2-(5-methyl-pyridin-2-yl)-ethyl]-phenyl ester

4-[1-(4-Carboxy-phenyl)-1H-tetrazol-5-ylsulfanyl]-piperidine-1-carboxylic acid 4-[2-(5-methyl-pyridin-2-yl)-ethyl]-phenyl ester

4-(4-Carboxymethyl-phenoxy)-piperidine-1-carboxylic acid 4-[2-(5-methyl-pyridin-2-yl)-ethyl]-phenyl ester

4-(Pyrazol-1-yloxy)-piperidine-1-carboxylic acid 4-[2-(5-methyl-pyridin-2-yl)-ethyl]-phenyl ester

4-(4-Carboxy-phenoxy)-piperidine-1-carboxylic acid 4-[2-(5-methyl-pyridin-2-yl)-ethyl]-phenyl ester

4-[4-(Pyridin-2-yloxy)-phenoxy]-piperidine-1-carboxylic acid 4-[2-(5-methyl-pyridin-2-yl)-ethyl]-phenyl ester

4-[4-(5-Methyl-pyridin-2-yloxy)-phenoxy]-piperidine-1-carboxylic acid 4-[2-(5-methyl-pyridin-2-yl)-ethyl]-phenyl ester

4-(4-Carboxy-phenoxymethyl)-piperidine-1-carboxylic acid 4-[2-(5-methyl-pyridin-2-yl)-ethyl]-phenyl ester

4-(4-Carboxymethyl-phenoxymethyl)-piperidine-1-carboxylic acid 4-[2-(5-methyl-pyridin-2-yl)-ethyl]-phenyl ester

4-(1H-Imidazol-2-ylsulfanylmethyl)-piperidine-1-carboxylic acid 4-[2-(5-methyl-pyridin-2-yl)-ethyl]-phenyl ester

4-(1-Methyl-1H-imidazol-2-ylsulfanylmethyl)-piperidine-1-carboxylic acid 4-[2-(5-methyl-pyridin-2-yl)-ethyl]-phenyl ester

4-(Imidazol-1-yloxymethyl)-piperidine-1-carboxylic acid 4-[2-(5-methyl-pyridin-2-yl)-ethyl]-phenyl ester

4-[4-(Pyridin-2-yloxy)-phenoxymethyl]-piperidine-1-carboxylic acid 4-[2-(5-methyl-pyridin-2-yl)-ethyl]-phenyl ester

4-[4-(5-Methyl-pyridin-2-yloxy)-phenoxymethyl]-piperidine-1-carboxylic acid 4-[2-(5-methyl-pyridin-2-yl)-ethyl]-phenyl ester

4-(1-Methyl-1H-tetrazol-5-ylsulfanylmethyl)-piperidine-1-carboxylic acid 4-[2-(5-methyl-pyridin-2-yl)-ethyl]-phenyl ester

4-[1-(2-Dimethylamino-ethyl)-1H-tetrazol-5-ylsulfanyl]-piperidine-1-carboxylic acid 4-[2-(5-methyl-pyridin-2-yl)-ethyl]-phenyl ester

4-[1-(2-Carboxy-ethyl)-1H-tetrazol-5-ylsulfanyl]-piperidine-1-carboxylic acid 4-[2-(5-methyl-pyridin-2-yl)-ethyl]-phenyl ester

4-(1-Carboxymethyl-1H-tetrazol-5-ylsulfanyl)-piperidine-1-carboxylic acid 4-[2-(5-methyl-pyridin-2-yl)-ethyl]-phenyl ester

4-(Pyrazol-1-yloxymethyl)-piperidine-1-carboxylic acid 4-[2-(5-methyl-pyridin-2-yl)-ethyl]-phenyl ester

4-(1H-[1,2,4]Triazol-3-ylsulfanylmethyl)-piperidine-1-carboxylic acid 4-[2-(5-methyl-pyridin-2-yl)-ethyl]-phenyl ester and

4-[2-(Benzyl-methyl-amino)-ethyl]-piperidine-1-carboxylic acid 4-[2-(5-methyl-pyridin-2-yl)-ethyl]-phenyl ester

In another embodiment the present invention relates to a compound selected from the group consisting of

4-(1-Methyl-1H-tetrazol-5-ylsulfanyl)-piperidine-1-carboxylic acid 4-(5-methyl-pyridin-2-ylmethyl)-phenyl ester,

4-Pyridin-2-ylmethyl-piperidine-1-carboxylic acid 4-(5-trifluoromethyl-pyridin-2-yloxy)-phenyl ester,

4-Pyridin-2-ylmethyl-piperidine-1-carboxylic acid 4-(4-trifluoromethyl-benzyl)-phenyl ester,

4-Pyrimidin-2-ylmethyl-piperidine-1-carboxylic acid 4-(5-trifluoromethyl-pyridin-2-yloxy)-phenyl ester,

4-Pyrimidin-2-ylmethyl-piperidine-1-carboxylic acid 4-(4-trifluoromethyl-benzyl)-phenyl ester,

4-Thiophen-3-ylmethyl-piperidine-1-carboxylic acid 4-(5-trifluoromethyl-pyridin-2-yloxy)-phenyl ester,

4-Thiophen-3-ylmethyl-piperidine-1-carboxylic acid 4-(4-trifluoromethyl-benzyl)-phenyl ester,

4-Thiophen-3-ylmethyl-piperidine-1-carboxylic acid 4-(5-methyl-pyridin-2-ylmethyl)-phenyl ester,

4-(5-Methyl-pyridin-2-ylmethyl)-piperidine-1-carboxylic acid 4-(4-trifluoromethyl-benzyl)-phenyl ester,

4-(5-Methyl-pyridin-2-ylmethyl)-piperidine-1-carboxylic acid 4-(5-trifluoromethyl-pyridin-2-yloxy)-phenyl ester,

4-(4-Carboxymethyl-benzyl)-piperidine-1-carboxylic acid 4-(5-trifluoromethyl-pyridin-2-yloxy)-phenyl ester,

4-(4-Carboxymethyl-benzyl)-piperidine-1-carboxylic acid 4-(4-trifluoromethyl-benzyl)-phenyl ester,

4-[4-(2H-Tetrazol-5-ylmethyl)-benzyl]-piperidine-1-carboxylic acid 4-(5-trifluoromethyl-pyridin-2-yloxy)-phenyl ester,

4-[4-(2H-Tetrazol-5-ylmethyl)-benzyl]-piperidine-1-carboxylic acid 4-(4-trifluoromethyl-benzyl)-phenyl ester,

4-(4-Methyl-pyridin-2-ylmethyl)-piperidine-1-carboxylic acid 4-(5-trifluoromethyl-pyridin-2-yloxy)-phenyl ester,

4-[4-(1H-Tetrazol-5-ylmethyl)-phenoxy]-piperidine-1-carboxylic acid 4-(5-trifluoromethyl-pyridin-2-yloxy)-phenyl ester,

4-(6-Hydroxymethyl-pyridin-2-ylmethyl)-piperidine-1-carboxylic acid 4-(5-trifluoromethyl-pyridin-2-yloxy)-phenyl ester,

4-[4-(1H-Tetrazol-5-ylmethyl)-phenoxy]-piperidine-1-carboxylic acid 4-(4-trifluoromethyl-benzyl)-phenyl ester,

4-(6-Hydroxymethyl-pyridin-2-ylmethyl)-piperidine-1-carboxylic acid 4-(4-trifluoromethyl-benzyl)-phenyl ester,

4-(4-Methyl-pyridin-2-ylmethyl)-piperidine-1-carboxylic acid 4-(4-trifluoromethyl-benzyl)-phenyl ester,

4-(4-Methyl-pyridin-2-ylmethyl)-piperidine-1-carboxylic acid 4-(5-methyl-pyridin-2-ylmethyl)-phenyl ester,

(Carboxymethyl-phenoxy)-piperidine-1-carboxylic acid 4-(3-methyl-butylcarbamoyl)-phenyl ester,

4-(4-Carboxymethyl-phenoxy)-piperidine-1-carboxylic acid 4-phenethylcarbamoyl-phenyl ester,

4-(4-Carboxymethyl-phenoxy)-piperidine-1-carboxylic acid 4-benzylcarbamoyl-phenyl ester,

4-(4-Carboxymethyl-phenoxy)-piperidine-1-carboxylic acid 4-[(bicyclo[2.2.1]hept-2-ylmethyl)-carbamoyl]-phenyl ester,

4-(4-Carboxymethyl-phenoxy)-piperidine-1-carboxylic acid 4-(cyclohexylmethyl-carbamoyl)-phenyl ester,

4-(4-Carboxymethyl-phenoxy)-piperidine-1-carboxylic acid 4-(3,3-dimethyl-butylcarbamoyl)-phenyl ester,

4-(4-Carboxymethyl-phenoxy)-piperidine-1-carboxylic acid 4-[(thiophen-2-ylmethyl)-carbamoyl]-phenyl ester,

4-(4-Carboxymethyl-phenoxy)-piperidine-1-carboxylic acid 4-(2-thiophen-2-yl-ethylcarbamoyl)-phenyl ester,

4-(4-Carboxymethyl-phenoxy)-piperidine-1-carboxylic acid 4-(2-phenoxy-ethyl)-phenyl ester,

4-(4-Carboxymethyl-phenoxy)-piperidine-1-carboxylic acid 4-[2-(4-chloro-phenoxy)-ethyl]-phenyl ester,

4-(4-Carboxymethyl-phenoxy)-piperidine-1-carboxylic acid 4-[2-(1-methyl-1H-tetrazol-5-yloxy)-ethyl]-phenyl ester,

4-(4-Carboxymethyl-phenoxy)-piperidine-1-carboxylic acid 4-[2-(3-chloro-phenoxy)-ethyl]-phenyl ester,

4-(4-Carboxymethyl-phenoxy)-piperidine-1-carboxylic acid 4-[2-(4-trifluoromethoxy-phenoxy)-ethyl]-phenyl ester,

4-(4-Carboxymethyl-phenoxy)-piperidine-1-carboxylic acid 4-[2-(4-[1,2,4]triazol-4-yl-phenoxy)-ethyl]-phenyl ester,

4-(4-Carboxymethyl-phenoxy)-piperidine-1-carboxylic acid 4-(2-phenylsulfanyl-ethyl)-phenyl ester,

4-(4-Carboxymethyl-phenoxy)-piperidine-1-carboxylic acid 4-[2-(2-chloro-phenoxy)-ethyl]-phenyl ester,

4-(4-Carboxymethyl-phenoxy)-piperidine-1-carboxylic acid 4-[2-(4-methyl-4H-[1,2,4]triazol-3-ylsulfanyl)-ethyl]-phenyl ester,

4-(4-Carboxymethyl-phenoxy)-piperidine-1-carboxylic acid 4-[2-(4-pyrrol-1-yl-phenoxy)-ethyl]-phenyl ester,

4-(4-Carboxymethyl-phenoxy)-piperidine-1-carboxylic acid 4-(3-chloro-5-trifluoromethyl-pyridin-2-ylmethyl)-phenyl ester,

4-(4-Carboxymethyl-phenoxy)-piperidine-1-carboxylic acid 4-(4-trifluoromethyl-phenoxy)-phenyl ester,

4-(1H-Imidazol-2-ylsulfanyl)-piperidine-1-carboxylic acid 4-(3,3-dimethyl-butylcarbamoyl)-phenyl ester,

4-(4-Carboxy-phenoxy)-piperidine-1-carboxylic acid 4-(3,3-dimethyl-butylcarbamoyl)-phenyl ester,

4-(4-Carboxy-benzyl)-piperidine-1-carboxylic acid 4-(5-trifluoromethyl-pyridin-2-yloxy)-phenyl ester,

4-(1H-Benzoimidazol-2-ylsulfanyl)-piperidine-1-carboxylic acid 4-(5-trifluoromethyl-pyridin-2-yloxy)-phenyl ester,

4-(4-Carboxy-1H-imidazol-2-ylsulfanyl)-piperidine-1-carboxylic acid 4-(5-trifluoromethyl-pyridin-2-yloxy)-phenyl ester,

6-{1-[4-(5-Trifluoromethyl-pyridin-2-yloxy)-phenoxycarbonyl]-piperidin-4-yloxy}-nicotinic acid,

4-(3-Carboxymethyl-phenoxy)-piperidine-1-carboxylic acid 4-(5-trifluoromethyl-pyridin-2-yloxy)-phenyl ester,

4-(4-Carboxymethyl-2-fluoro-phenoxy)-piperidine-1-carboxylic acid 4-(5-trifluoromethyl-pyridin-2-yloxy)-phenyl ester,

4-[3-(2-Carboxy-ethyl)-phenoxy]-piperidine-1-carboxylic acid 4-(5-trifluoromethyl-pyridin-2-yloxy)-phenyl ester,

4-(5-Carboxymethylsulfanyl-[1,3,4]thiadiazol-2-ylsulfanyl)-piperidine-1-carboxylic acid 4-(5-trifluoromethyl-pyridin-2-yloxy)-phenyl ester,

4-(5-carboxymethyl-4-methyl-oxazol-2-ylsulfanyl)-piperidine-1-carboxylic acid 4-(5-trifluoromethyl-pyridin-2-yloxy)-phenyl ester,

4-(4-Carboxymethoxy-phenoxy)-piperidine-1-carboxylic acid 4-(5-trifluoromethyl-pyridin-2-yloxy)-phenyl ester,

4-(4-Carboxymethyl-phenoxy)-piperidine-1-carboxylic acid 4-(3-trifluoromethyl-phenoxy)-phenyl ester,

4-(4-Carboxymethyl-phenoxy)-piperidine-1-carboxylic acid 4-(5-chloro-pyridin-2-yloxy)-phenyl ester,

4-(4-Carboxymethyl-phenoxy)-piperidine-1-carboxylic acid 4-(3-chloro-5-trifluoromethyl-pyridin-2-yloxy)-phenyl ester,

4-(4-Carboxymethyl-phenoxy)-piperidine-1-carboxylic acid 4-p-tolyloxy-phenyl ester, and

4-(4-Carboxymethyl-phenoxy)-piperidine-1-carboxylic acid 5-(3-cyclohexyl-thioureido)-pyridin-2-yl-ester,

In another aspect the present invention relates to a pharmaceutical composition comprising a compound of general formula I or a pharmaceutically acceptable salt thereof together with a pharmaceutically acceptable carrier or diluent.

In one embodiment the invention is concerned with a pharmaceutical composition in unit dosage form, comprising from about 0.05 to about 2000 mg, preferably from about 0.1 to about 500 mg and even more preferable from about 1.0 to about 100 mg of said compound according to the invention or pharmaceutically acceptable salt thereof.

In another embodiment the invention is concerned with a pharmaceutical composition for use as a medicament for inhibiting the lipolytic activity of hormone-sensitive lipase against triacylglycerols, diacylglycerols, cholesterol acyl esters or steroid acyl esters, said composition comprising a compound according to the invention or a pharmaceutically acceptable salt thereof together with a pharmaceutically acceptable carrier or diluent.

In another embodiment the invention is concerned with a pharmaceutical composition which is for oral administration.

In another embodiment the invention is concerned with a pharmaceutical composition which is for nasal, transdermal, pulmonal, or parenteral administration.

In another aspect the present invention relates to use of a compound according to the inventtion for the preparation of a pharmaceutical composition.

In one embodiment the invention is concerned with use of a compound according to the invention for inhibition of hormone sensitive lipase.

In another embodiment the invention is concerned with use of a compound according to the invention for preparation of a pharmaceutical composition for inhibition of the lipolytic activity of hormone-sensitive lipase against triacylglycerols, diacylglycerols, cholesterol acyl esters or steroid acyl esters.

In another embodiment the invention is concerned with use of a compound according to the invention for the preparation of a pharmaceutical composition for the treatment or prevention of any disorder where it is desirable to modulate the plasma level of free fatty acids, glycerol, LDL-cholesterol, HDL-cholesterol, insulin and/or glucose; and/or modulate intracellular triacylglycerol and cholesterol ester stores, intracellular level of fatty acids, fatty acid esters such as diacylglycerols, phosphatidic acids, long chain acyl-CoA's as well as citrate or malonyl-CoA; and/or increase insulin sensitivity in adipose tissue, skeletal muscle, liver or pancreatic β cells; and/or modulate insulin secretion from pancreatic β cells.

In another embodiment the invention is concerned with the above use wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

In another embodiment the invention is concerned with the use of a compound according to the invention for the preparation of a pharmaceutical composition for the treatment and/or prevention of dyslipidemia.

In another embodiment the invention is concerned with the use of a compound according to the invention for the preparation of a pharmaceutical composition for the treatment and/or prevention of hyperlipidemia.

In another embodiment the invention is concerned with the use of a compound according to the invention for the preparation of a pharmaceutical composition for the treatment and/or prevention of hyperglycemia.

In another embodiment the invention is concerned with the use of a compound according to the invention for lowering HbA_{1c}.

In another embodiment the invention is concerned with the preparation of a pharmaceutical composition for the treatment and/or prevention of diabetes type 2.

In another embodiment the invention is concerned with the use of a compound according to the invention for the preparation of a pharmaceutical composition for the treatment and/or prevention impaired glucose tolerance.

In another embodiment the invention is concerned with the use of a compound according to the invention for the preparation of a pharmaceutical composition for the treatment and/or prevention of metabolic syndrome X.

In another embodiment the invention is concerned with the use of a compound according to the invention for the preparation of a pharmaceutical composition for the treatment and/or prevention of atheroschlerosis.

In another embodiment the invention is concerned with the use of a compound according to the invention for the preparation of a pharmaceutical composition for delaying or prevention of the progression from impaired glucose tolerance to diabetes type 2.

In another embodiment the invention is concerned with the use of a compound according to the invention for the preparation of a pharmaceutical composition for delaying or prevention of the progression from non-insulin requiring diabetes type 2 to insulin requiring diabetes type 2.

In another embodiment the invention is concerned with the use according to above indicationns wherein a further antidiabetic, antiobesity, antihypertensive or appetite regulating drug is used.

In another embodiment the invention is concerned with the use according to above indications, wherein metformin is also used.

In another aspect the present invention is related to a method of treating a disorder of a patient where modulation of the activity of hormone-sensitive lipase is desired, the method comprising administering to a subject in need thereof a therapeutically effective amount of a compound according to the present invention or a pharmaceutically acceptable salt thereof.

In one embodiment the invention is concerned with a method of treating a disorder of a patient where lowering of the activity of hormone-sensitive lipase is desired, the method comprising administering to a subject in need thereof a therapeutically effective amount of a compound according to the invention or a pharmaceutically acceptable salt thereof.

In another embodiment the invention is concerned with the above methods wherein said administration is carried out by the oral, nasal, transdermal, pulmonal, or parenteral route.

In another embodiment the invention is concerned with the above methods wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

In another embodiment the invention is concerned with the above methods wherein the therapeutically effective amount of the compound is from about 0.05 to about 2000 mg, preferably from about 0.1 to about 500 mg and even more preferable from about 1.0 to about 100 mg of said compound per day.

In another embodiment the invention is concerned with the above methods wherein a further antidiabetic, antiobesity, antihypertensive or appetite regulating drug is administered to the patient.

In another embodiment the invention is concerned with above methods wherein metformin is also administered to the patient.

In another aspect the present invention relates to a process for the preparation of a compound according to formula I or its pharmaceutically acceptable salt, which comprises reacting the appropriate alcohol with the appropriate carbamoylating reagent in a solvent according to the reaction scheme P₁ 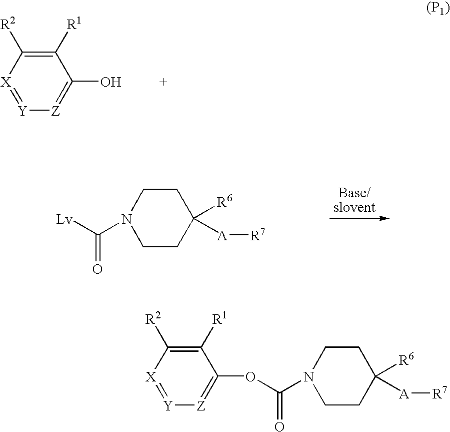
and isolating the disubstituted carbamate product.

In one embodiment the invention is concerned with the process P₁, wherein said carbamoylating reagent 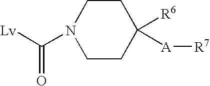
is selected from the group consisting of

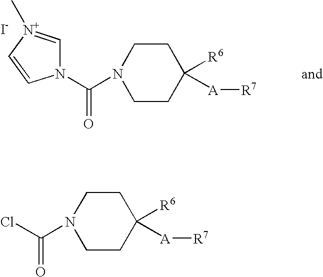

In another embodiment the invention is concerned with the process P₁, wherein said solvent is selected from the group consisting of tetrahydrofurane, dimethylformamide and N-methylpyrolidone.

In another embodiment the invention is concerned with the process P₁, wherein said base is selected from the group consisting of triethylamine, N,N-diisopropyl-N-ethylamine and DABCO.

In another embodiment the invention is concerned with a process for the preparation of a compound according to the invention, said process comprising the treatment of the appropriate amine with the appropriate acylating reagent in a solvent and in the presence of a base according to the reaction scheme P₂ 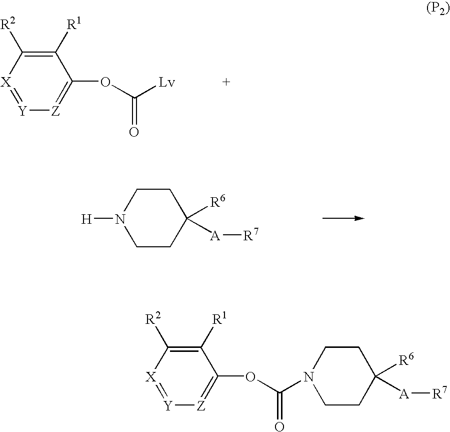
and isolating the disubstituted carbamate

In another embodiment the invention is concerned with the process P₂, wherein Lv is Cl.

In another embodiment the invention is concerned with the process P₂, wherein said solvent is selected from the group consisting of diethyl ether, tetrahydrofuran and dichloromethane.

In another embodiment the invention is concerned with the process P₂, wherein said base is selected from the group consisting of trimethylamine, triethylamine, ethyl-diisopropyl-amine and 1,4-diazabicyclo[2.2.2]octane.

In another embodiment the invention is concerned with te process P₂, wherein said base is present as a functionality in one or both of the substituents R⁶ and R⁷, thus forming a salt with the acid H-Lv.

In another embodiment the invention is concerned with a process for the preparation of a compound according to the invention, said process comprising the treatment of the appropriate alcohol (A′ is selected from OH or CH₂OH) with the appropriate hydroxy containing Mitzunobu reagent (HO—R⁷) or thiol containing Mitzunobu reagent (HS—R⁷) under standard mitzunobu conditions as described in reaction scheme P₃ or P₄: 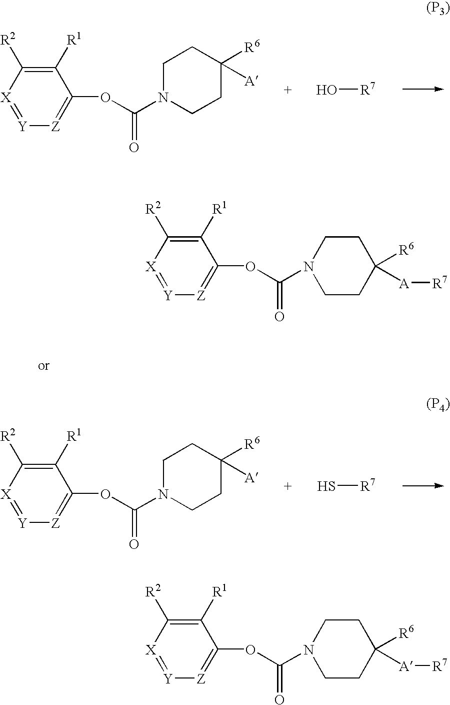
and isolating the disubstituted carbamate

The present invention also encompasses pharmaceutically acceptable salts of the present compounds. Such salts include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium, zinc, calcium salts and the like. Examples of amines and organic amines include ammonium, methylamine, dimethylamine, trimethylamine, ethylamine, diethylamine, propylamine, butylamine, tetramethylamine, ethanolamine, diethanolamine, triethanolamine, meglumine, ethylenediamine, choline, N,N′-dibenzylethylenediamine, N-benzylphenylethylamine, N-methyl-D-glucamine, guanidine and the like. Examples of cationic amino acids include lysine, arginine, histidine and the like.

The pharmaceutically acceptable salts are prepared by reacting the compound of formula I with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixture of solvents may be used. Organic bases like lysine, arginine, diethanolamine, choline, guandine and their derivatives etc. may also be used. Alternatively, acid addition salts wherever applicable are prepared by treatment with acids such as hydrochloric acid, hydrobromic acid, nitric acid, sulfuric acid, phosphoric acid, p-toluenesulphonic acid, methanesulfonic acid, acetic acid, citric acid, maleic acid salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane etc. Mixture of solvents may also be used.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, enzymatic resolution, resolving the diastereomeric salts formed with chiral acids such as mandelic acid, camphorsulfonic acid, tartaric acid, lactic acid, and the like wherever applicable or chiral bases such as brucine, (R)- or (S)-phenylethylamine, cinchona alkaloids and their derivatives and the like. Commonly used methods are compiled by Jaques et al in “Enantiomers, Racemates and Resolution” (Wiley Interscience, 1981). More specifically the compound of formula I may be converted to a 1:1 mixture of diastereomeric amides by treating with chiral amines, aminoacids, aminoalcohols derived from aminoacids; conventional reaction conditions may be employed to convert acid into an amide; the dia-stereomers may be separated either by fractional crystallization or chromatography and the stereoisomers of compound of formula I may be prepared by hydrolysing the pure diastereomeric amide.

Various polymorphs of compound of general formula I forming part of this invention may be prepared by crystallization of compound of formula I under different conditions. For example, using different solvents commonly used or their mixtures for recrystallization; crystallizations at different temperatures; various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffraction or such other techniques.

The invention also encompasses prodrugs of the present compounds, which on administration undergo chemical conversion by metabolic processes before becoming active pharmacological substances. In general, such prodrugs will be functional derivatives of the present compounds, which are readily convertible in vivo into the required compound of the formula I. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The invention also relates to pharmaceutical compositions comprising, as an active ingredient, at least one compound of the formula I or any optical or geometric isomer or tautomeric form thereof including mixtures of these or a pharmaceutically acceptable salt thereof together with one or more pharmaceutically acceptable carriers or diluents.

Furthermore, the invention relates to the use of compounds of the general formula I or their tautomeric forms, their stereoisomers, their polymorphs, their pharmaceutically acceptable salts or pharmaceutically acceptable solvates thereof for the preparation of a pharmaceutical composition for the treatment and/or prevention of disorders where a decreased level of plasma FFA is desirable, such as the conditions mentioned above.

In another aspect, the present invention relates to a method of treating and/or preventing type 2 diabetes, insulin resistance, metabolic syndrome X, impaired glucose tolerance, dyslipidemia and abnormalities of lipoprotein metabolism.

In a still further aspect, the present invention relates to the use of one or more compounds of the general formula I, or pharmaceutically acceptable salts thereof, for the preparation of a pharmaceutical composition for the treatment and/or prevention of type 2 diabetes, insulin resistance, metabolic syndrome X, impaired glucose tolerance, dyslipidemia and abnormalities of lipoprotein metabolism.

In a still further aspect, the present compounds are useful for the delaying or prevention of the progression from impaired glucose tolerance to type 2 diabetes.

In a still further aspect, the present compounds are useful for the delaying or prevention of the progression from non-insulin requiring type 2 diabetes to insulin requiring type 2 diabetes.

In another aspect, the present compounds reduce triglyceride levels and are accordingly useful for the treatment and/or prevention of ailments and disorders such as diabetes and/or obesity.

In still another aspect, the compounds of general formula I are useful for the treatment of hyperglycemia, elevated HbA_{1c} level, hyperinsulinemia, type 1.5 diabetes, latent autoimmune diabetes in adults, maturity onset diabetes, beta-cell apoptosis, hemochromatosis induced diabetes, impaired glucose tolerance, impaired fasting glucose, metabolic syndrome X, insulin resistance, impaired lipid tolerance, cystic fibrosis related diabetes, polycystic ovarian syndrome, and gestational diabetes.

In still another aspect, the compounds of general formula I are useful for the treatment of obesity, dyslipidemia, diabetic dyslipidemia, hyperlipidemia, hypertriglyceridemia, hyperlipoproteinemia, hypercholesterolemia, hypertension, essential hypertension, acute hypertensive emergency, arteriosclerosis, atherosclerosis, restenosis, intermittent claudication (atherosclerosis oblitterens), cardiovascular disease, cardiomyopathy, cardiac hypertrophy, left ventricular hypertrophy, coronary artery disease, early coronary artery disease, heart insufficiency, exercise tolerance, chronic heart failure, mild chronic heart failure, arrhythmia, cardiac dysrythmia, syncopy, heart attack, myocardial infarction, Q-wave myocardial infarction, stroke, acute coronary syndrome, angina pectoris, unstable angina, cardiac bypass reocclusion, diastolic dysfunction, systolic dysfunction, non-Q-wave cardiac necrosis, catabolic changes after surgery, acute pancreatitis, and irritable bowel syndrome

In still another aspect, the compounds of general formula I may be useful for the treatment of diabetic retinopathy, background retinopathy, preproliferative retinopathy, proliferative retinopathy, macular edema, cataracts, nephropathy, nephrotic syndrome, diabetic nephropathy, microalbuminuria, macroalbuminuria, neuropathy, diabetic neuropathy, distal symmetrical sensorimotor polyneuropathy, and diabetic autonomic neuropathy.

In still another aspect, the compounds of general formula I are useful for increasing the number of beta-cells in a patient, increasing the size of beta-cells in a patient or stimulating beta-ell proliferation, modulating beta-cell function and insulin secretion in a patient in need thereof, which method comprises administration of an effective amount of a compound of formula I to a patient in need thereof.

The compounds of the invention are also believed to be useful for reducing body weight in a patient in need thereof.

The compounds of the invention are also believed to be useful for weight neutral treatment of above mentioned diseases.

The compounds of the invention are also believed to be useful for redistributing fat in a patient in need thereof.

The compounds of the invention are also believed to be useful for redistributing central fat in a patient in need thereof.

The compounds of the invention are also believed to be useful for reducing or preventing central obesity.

The compounds of the invention are also believed to be useful for reducing postprandial serum lipid excursions.

The compounds of the invention are also believed to be useful for the treatment of fatty acid oxidation disorders such as MCAD.

In still another aspect, the compounds of general formula I are believed to be useful for the treatment of a disease, condition or disorder wherein cholesterol is a precursor. Such diseases, conditions or disorders may relate to testosterone, e.g. male contraception, excessive testosterone levels, PCOS and prostate cancer. They may also relate to cortisol or corticotropin, e.g. Cushing disease.

The compounds of the invention are also believed to be useful for the treatment of cancer. Thus, the compounds of the general formula I may be useful for the treatment of insulinoma (pancreatic islet cell tumors), e.g. malignant insulinomas and multiple insulinomas, adipose cell carcinomas, e.g. lipocarconoma.

The compounds of the invention are also believed to be useful for the treatment of phaechromocytoma and other diseases with increased catecholamine incretion. The compounds of the invention are also believed to be useful for the treatment of prostate cancer, e.g. adenocarcinoma.

In still another aspect, the compounds of general formula I may be useful for the treatment of hepatic steatosis.

In still another aspect, the compounds of general formula I may be useful for the treatment of cirrhosis.

In still another aspect, the compounds of general formula I may be useful for the treatment of AIDS or an AIDS related diseases, condition or disorders

In still another aspect, the compounds of general formula I may be useful for the treatment of lipodystrophy

In still another aspect, the compounds of general formula I may be useful for the treatment of lactic acidosis.

In yet another aspect, the compounds of the present invention are expected to be useful for the treatment of CNS diseases, conditions or disorders.

Thus, the compound of the present invention may be used for the treatment of Parkinsons disease, Alzheimers disease, ADHD (Attention Deficit Hyperactivity Disorder), feeding disorders such as bulimia and anorexia, depression, anxiety, cognitive memory disorders, age related cognitive decline, mild cognitive impairment and schizophrenia.

In yet another aspect, the compounds of the present invention may be useful for the treatment of inflammatory disorders, e.g. rheumatoid arthritis, psoriasis, systemic inflammatory response syndrome, sepsis and the like.

The present compounds may also be administered in combination with one or more further pharmacologically active substances eg., selected from antiobesity agents, antidiabetics, antihypertensive agents, agents for the treatment and/or prevention of complications resulting from or associated with diabetes and agents for the treatment and/or prevention of complications and disorders resulting from or associated with obesity.

Thus, in a further aspect of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators or TR β agonists.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

Suitable antidiabetics comprise insulin, exendin-4, GLP-1 (glucagon like peptide-1) and derivatives thereof such as those disclosed in WO 98/08871 to Novo Nordisk A/S, which is incorporated herein by reference as well as orally active hypoglycaemic agents.

The orally active hypoglycaemic agents preferably comprise sulphonylureas, biguanides, meglitinides, glucosidase inhibitors, glucagon antagonists such as those disclosed in WO 99/01423 to Novo Nordisk A/S and Agouron Pharmaceuticals, Inc., GLP-1 agonists, potassium channel openers such as those disclosed in WO 97/26265 and WO 99/03861 to Novo Nordisk A/S which are incorporated herein by reference, DPP-IV (dipeptidyl peptidase-IV) inhibitors, inhibitors of hepatic enzymes involved in stimulation of gluconeogenesis and/or glycogenolysis, glucose uptake modulators, compounds modifying the lipid metabolism such as antihyperlipidemic agents and antilipidemic agents as HMG CoA inhibitors (statins), compounds lowering food intake, RXR agonists and agents acting on the ATP-dependent potassium channel of the β-cells.

In one embodiment of the invention the present compounds are administered in combination with insulin.

In a further embodiment the present compounds are administered in combination with a sulphonylurea eg. tolbutamide, glibenclamide, glipizide or glicazide.

In another embodiment the present compounds are administered in combination with a biguanide eg. metformin.

In yet another embodiment the present compounds are administered in combination with a meglitinide eg. repaglinide or senaglinide.

In a further embodiment the present compounds are administered in combination with an α-glucosidase inhibitor eg. miglitol or acarbose.

In another embodiment the present compounds are administered in combination with an agent acting on the ATP-dependent potassium channel of the β-cells eg. tolbutamide, glibenclamide, glipizide, glicazide or repaglinide.

Furthermore, the present compounds may be administered in combination with nateglinide.

In still another embodiment the present compounds are administered in combination with an antihyperlipidemic agent or antilipidemic agent eg. cholestyramine, colestipol, clofibrate, gemfibrozil, lovastatin, pravastatin, simvastatin, probucol or dextrothyroxine.

In a further embodiment the present compounds are administered in combination with more than one of the above-mentioned compounds eg. in combination with a sulphonylurea and metformin, a sulphonylurea and acarbose, repaglinide and metformin, insulin and a sulphonylurea, insulin and metformin, insulin, insulin and lovastatin, etc.

Furthermore, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, alatriopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with one or more of the above-mentioned compounds and optionally one or more further pharmacologically active substances are considered to be within the scope of the present invention.

The present invention also relates to processes according to reaction schemes P₁ and P₂ for the preparation of the above said novel compounds, their derivatives, their analogs, their tautomeric forms, their stereoisomers, their polymorphs, their pharmaceutically acceptable salts or pharmaceutically acceptable solvates.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995. The compositions may appear in conventional forms, for example capsules, tablets, aerosols, solutions, suspensions or topical applications.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well-known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

The therapeutic dose of the compound will depend upon the frequency and mode of administration, the sex, age, weight and general condition of the subject treated, the nature and severity of the condition treated and any concomitant diseases to be treated and other factors evident to those skilled in the art. The formulations may conveniently be presented in unit dosage form by methods known to those skilled in the art. In one embodiment the composition in unit dosage form, comprises from about 0.05 to about 2000 mg, preferably from about 0.1 to about 500 mg of the compound of formula I pharmaceutically acceptable salt thereof.

In a still further embodiment the pharmaceutical composition is for oral, nasal, transdermal, pulmonal, or parenteral administration.

For parenteral routes, such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound of the invention contains a free base such salts are prepared in a conventional manner by treating a solution or suspension of a free base of the compound with a chemical equivalent of a pharmaceutically acceptable acid, for example, inorganic and organic acids. Representative examples are mentioned above. Physiologically acceptable salts of a compound with a hydroxy group include the anion of said compound in combination with a suitable cation such as sodium or ammonium ion.

For parenteral administration, solutions of the present compounds in sterile aqueous solution, aqueous propylene glycol or sesame or peanut oil may be employed. Such aqueous solutions should be suitable buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of suitable carriers are water, salt solutions, alcohols, polyethylene glycols, polyhydroxyethoxylated castor oil, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent may include any sustained release material known in the art, such as glyceryl monostearate or glyceryl distearate, alone or mixed with a wax. The formulations may also include wetting agents, emulsifying and suspending agents, preserving agents, sweetening agents or flavouring agents.

The pharmaceutical compositions formed by combining the compounds of the invention and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. These formulations may be in the form of powder or granules, as a solution or suspension in an aqueous or non-aqueous liquid, or as an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet which may be prepared by conventional tabletting techniques may contain:

| | |
|---|---|
| Core: | |
| Active compound (as free compound or salt thereof) | 5 mg |
| Colloidal silicon dioxide (Aerosil) | 1.5 mg |
| Cellulose, microcryst. (Avicel) | 70 mg |
| Modified cellulose gum (Ac-Di-Sol) | 7.5 mg |
| Magnesium stearate | q.s. |
| Coating: | |
| HPMC approx. | 9 mg |
| *Mywacett 9-40 T approx. | 0.9 mg |

*Acylated monoglyceride used as plasticizer for film coating.

The compounds of the invention may be administered to a patient which is a mammal, especially a human in need thereof. Such mammals include also animals, both domestic animals, e.g. household pets, and non-domestic animals such as wildlife.

In a further aspect of the invention the present compounds may be administered in combination with further pharmacologically active substances e.g. an antidiabetic or other pharmacologically active material, including other compounds for the treatment and/or prevention of insulin resistance and diseases, wherein insulin resistance is the pathophysiological mechanism.

Furthermore, the compounds according to the invention may be administered in combination with antiobesity agents or appetite regulating agents.

### EXAMPLES

General Methods

All reactions involving air-sensitive reagents were performed under nitrogen using syringe-septum cap techniques. The glassware was dried by heating with a heat-gun. MgSO₄ were used to dry solutions. Solvents were removed in vacuo by rotary evaporation. Melting points were recorded on a Büchi 535, Bruker AMX 400 and Bruker DRX 300 instruments were used to record ¹H NMR spectra at 400 and 300 MHz respectively with tetramethylsilane (TMS) as internal standard. Coupling constants (J) are given in Hz.

Materials

Test compounds were synthesized or when commercially available they were purchased from Aldrich, Specs, Maybridge or Bionet. For the synthesized compounds the procedure for synthesis and measured characteristics of the compound are stated in the example. All compounds for which no synthesis procedure is stated in the examples are commercially available and have been purchased, or were prepared by standard methods described in the literature.

The allyl esters e.g. (4-hydroxy-phenyl)-acetic acid allyl ester and 4-hydroxy-benzoic acid allyl ester were prepared according to the procedures described by Lee, J. C. (Synthetic Commun. 28 (11) 2021-2026 (1998).

1-Benzyl-methyl-(2-piperidin-4-yl-ethyl)-amine was prepared from 4-(2-oxo-ethyl)-piperidine-1-carboxylic acid tert butyl ester (prepared from 4-piperidineethanol) by a standard reductive amination protocol followed by an acidic N-BOC deprotecting step.

4-Hydroxymethyl-piperidine-1-carboxylic acid 4-(5-trifluoromethyl-pyridin-2-yloxy)-phenyl ester, 4-Hydroxy-piperidine-1-carboxylic acid 4-(5-trifluoromethyl-pyridin-2-yloxy)-phenyl ester and 4-(5-trifluoromethyl-pyridin-2-yloxy)-phenyl chloroformate were prepared as described in PCT/DK02/00852).

2-(2-piperidin-4-yl-ethyl)-1,2,3,4-tetrahydro-isoquinoline was prepared from 4-(2-oxoethyl)piperidine-1-carboxylic acid tert-butyl ester and 1,2,3,4-tetrahydroisoquinoline by a standard reductive amination protocol followed by an acidic N-BOC deprotecting step.

The following allyl esters were prepared form the corresponding phenol carboxylic acids by a reaction with allyl bromide applying CsF-celite in acetonitrile:

(3-hydroxy-phenyl)-acetic acid allyl ester,

(3-fluoro-4-hydroxy-phenyl)-acetic acid allyl ester,

3-(3-hydroxy-phenyl)-propionic acid allyl ester,

5-mercapto-[1,3,4]thiadiazol-2-ylsulfanyl)-acetic acid allyl ester,

2-mercapto-4-methyl-oxazol-5-yl)-acetic acid allyl ester,

(4-hydroxy-phenoxy)-acetic acid allyl ester, and

4-Hydroxy-phenyl)-acetic acid allyl ester.

### 4-(4-Allyloxycarbonylmethyl-phenoxy)-piperidine-1-carboxylic acid 4-(2-hydroxy-ethyl)-phenyl ester

a) 4-(4-Allyloxycarbonylmethyl-phenoxy)-piperidine-1-carboxylic acid tert-butyl ester was prepared by a classical mitzunobu reaction (DEAD, triphenylphosphine) of (4-Hydroxy-phenyl)-acetic acid allyl ester and tert-butoxycarbonyl-4-hydroxypiperidine.

b) [4-(Piperidin-4-yloxy)-phenyl]-acetic acid allyl ester was prepared by a HCL (ether) catalyzed deprotection of the piperidine: 4-(4-allyloxycarbonylmethyl-phenoxy)-piperidine-1-carboxylic acid tert-butyl ester.

c) 3-[4-(4-Allyloxycarbonylmethyl-phenoxy)-piperidine-1-carbonyl]-1-methyl-3H-imidazol-1-ium; iodide was prepared by a two step one pot reaction of first a 1,1′-carbonyldiimidazole (TEA in THF) condensation followed by a methylation (methyliodide) of the imidazole group.

d) 4-(4-Allyloxycarbonylmethyl-phenoxy)-piperidine-1-carboxylic acid 4-(2-hydroxy-ethyl)-phenyl ester was prepared by a carbamoylation reaction between 4-hydroxyphenethyl alcohol and 3-[4-(4-allyloxycarbonylmethyl-phenoxy)-piperidine-1-carbonyl]-1-methyl-3H-imidazol-1-ium; iodide (DIPEA, dichloromethane)

### 4-Hydroxy-piperidine-1-carboxylic acid 4-(3,3-dimethyl-butylcarbamoyl)-phenyl ester

a) N-(3,3-Dimethyl-butyl)-4-hydroxy-benzamide was prepared from 4-hydroxy-benzoic acid 2,5-dioxo-pyrrolidin-1-yl ester (prepared by standard literature procedures) which was condensed with 3,3-dimethylbytylamine (DIPEA, THF).

b) 4-(tert-Butyl-dimethyl-silanyloxy)-piperidine-1-carboxylic acid 4-(3,3-dimethyl-butylcarbamoyl)-phenyl ester was prepared from a condensation of N-(3,3-Dimethyl-butyl)-4-hydroxy-benzamide and 3-[4-(tert-Butyl-dimethyl-silanyloxy)-piperidine-1-carbonyl]-1-methyl-3H-imidazol-1-ium; iodide (DABCO, dichloromethane).

c) 4-Hydroxy-piperidine-1-carboxylic acid 4-(3,3-dimethyl-butylcarbamoyl)-phenyl ester was prepared from 4-(tert-butyl-dimethyl-silanyloxy)-piperidine-1-carboxylic acid 4-(3,3-dimethyl-butylcarbamoyl)-phenyl ester which was hydroxy deprotected with HCl

3-[4-(4-Allyloxycarbonylmethyl-phenoxy)-piperidine-1-carbonyl]-1-methyl-3H-imidazol-1-ium; iodide

a) 4-(4-Allyloxycarbonylmethyl-phenoxy)-piperidine-1-carboxylic acid tert-butyl ester was synthesized from (4-hydroxy-phenyl)-acetic acid allyl ester and tert-butoxycarbonyl-4-hydroxypiperidine by a standard mitzunobu protocol (triphenylphosphine, DEAD THF).

b) [4-(Piperidin-4-yloxy)-phenyl]-acetic acid allyl ester was synthesized by a HCl catalyzed deprotection of the piperidine: 4-(4-allyloxycarbonylmethyl-phenoxy)-piperidine-1-carboxylic acid tert-butyl ester.

c) 3-[4-(4-Allyloxycarbonylmethyl-phenoxy)-piperidine-1-carbonyl]-1-methyl-3H-imidazol-1-ium; iodide was synthesized from [4-(piperidin-4-yloxy)-phenyl]-acetic acid allyl ester and 1,1′-carbonyldiimidazole (TEA, THF). The imidazole intermediate was methylated with methyliodide in acetonitrile.

Thin layer chromatography was performed on Merck DC-Alufolien, silica gel 60 F₂₅₄ and components were visualized by UV₂₅₄. Flash chromatography was performed using silica gel Merck 60 size 0.04-0-063 mm and a Quad 12/25 flash system.

Preparative HPLC

The system consists of two Gilson 322 pumps and a Gilson manometric module 805. A Gilson 215 combined auto-injector and fraction collector performs injection and fraction collection. Detection is performed with a Gilson Diode array detector 170. A sample containing 25-100 mg of material dissolved in 0.5-2.0 ml of solvent (minimum water concentration: 10%).

Separation is performed on Waters Xterra, RP₁₈ 7 μm, columns 19 mm×150 mm, flow rate 15 ml/min (sample added with a flow rate of 5 ml/min for about 1 min). The most widely used gradient starts at 5% acetonitrile in water and ends after 14 min on 95% acetonitrile. This concentration is maintained for 6 min. The system is buffered with 0.05% TFA. In special cases the gradient is altered to fit the separation need. The pooled fractions are evaporated to dryness in vacuo.

HPLC-MS.

The following instrumentation was used:
Hewlett Packard series 1100 G1312A Bin Pump
Hewlett Packard series 1100 Column compartment
Hewlett Packard series 1100 G13 15A DAD diode array detector
Hewlett Packard series 1100 MSD

The instrument was controlled by HP Chemstation software.

The HPLC pump was connected to two eluent reservoirs containing:

A: 0.01% TFA in water

B: 0.01% TFA in acetonitrile

The analysis was performed at 40° C. by injecting an appropriate volume of the sample (preferably 1 μl) onto the column, which is eluted with a gradient of acetonitrile.

The HPLC conditions, detector settings and mass spectrometer settings which were used are as follows:

| | |
|---|---|
| Column | Waters Xterra MS C-18 × 3 mm id |
| Gradient | 10%-100% acetonitrile linear during 7.5 min |
| | at 1.0 ml/min |
| Detection | 210 nm (analogue output from DAD) |
| MS | Ionisation mode API-ES, Scan 100-1000 amu |
| | step 0.1 amu |

General Procedure 1

A solution of an alcohol (0.4 mmol), an appropriate thio or hydroxy mitzunobu reagent (e.g. phenol/thiophenol/N-hydroxyazole/azole) (0.33-0.4 mmol), and tributylphosphine (0.5-1.4 mmol) in THF (2-8 mL) was stirred under nitrogen at rt. in a flame dried reaction flask. ADDP (0.5-1.4 mmol) dissolved in THF (1.5-3 mL) was added and the reaction mixture was stirred at rt. for at least 16 h, sometimes filtered, evaporated to dryness and purified by preparative HPLC and/or flash chromatography.

General Procedure 2

A methyl or ethyl carboxylic acid ester (0.2 mmol) was dissolved in DMSO (1.5 ml) and an aqueous hydrochloric acid solution (2-3 N; 0.75 ml). The reaction mixture was heated to 70-95° C. for at least 2 h. Water (0.3 ml) was added to the evaporated or non-evaporated reaction mixture (DMSO solution) and the product mixture was purified by preparative HPLC.

General Procedure 3

An allylic ester (0.2 mmol), morpholine (0.67 mmol), Pd(PPh₃)₄ (0.02 mmol) in dry THF (5 mL) were stirred in darkness for 1 h at room temperature. For some reactions additional morpholine (0.67 mmol) and Pd(PPh₃)₄ (0.02 mmol) was added. The reaction mixture was evaporated to dryness, to give a crude product which was purified by preparative HPLC or flash chromatography.

General Procedure 4

An appropriate amine (1.0 eq.) and a base such as 1,4-diazabicyclo[2.2.2]octane (1.5-4.0 eq.) or diisopropylethylamine (1.2-4 eq) was dissolved in dichloromethane (0.01-0.2 mmol/ml concentration of the amine) or a mixture of dichloromethane and DMF in a dried reaction flask under nitrogen. The appropriate aryl chloroformate (1-1.3 eq.) (Prepared from the corresponding phenol by conventional methods) was added at room temperature. The reaction mixture was evaporated and purified by flash chromatography or preparative HPLC.

General Procedure 5

A solution of an alcohol (0.4 mmol), a phenol/thiophenol/N-hydroxyazole/azole or imide (0.4 mmol), diisopropylethylamin (0.44 mmol) and tributylphosphine (0.52 mmol) in THF (5 mL) was stirred under nitrogen at rt. 1,1′-(Azodicarbonyl)dipiperidine (ADDP, 0.52 mmol) dissolved in THF (5 mL) was added and the reaction mixture was stirred at rt for 16 h. The reaction mixture was filtered and the filtrate was evaporated to dryness, which gave a crude which was either purified by flash chromatography (Quad flash 25, EtOAc-heptane) or redissolved in MeCN and purified by preparative HPLC (Gilson).

General Procedure 6

A solution of an alcohol (0.4 mmol), a phenol/thiophenol/N-hydroxyazole/azole or imide (0.4 mmol), diisopropylethylamin (0.44 mmol) and solid supported triphenylphosphine (3 mmol/g, 1.2 mmol) in CH₂Cl₂ (2 mL) was stirred under nitrogen at rt. Di-tert-butylazodicarboxylate (DBAD, 1.2 mmol) dissolved in CH₂Cl₂ (1 mL) was added and the reaction mixture was stirred at rt for 16 h. TFA (0.5 mL) was added and the mixture was stirred for further 1 h at rt. Addition of EtOAc, filtration, followed by evaporation to dryness gave a crude which was either purified by flash chromatography (Quad flash 12, EtOAc-heptane) or redissolved in MeCN and purified by preparative HPLC (Gilson).

General Procedure 7

To a solution of the appropriate piperidine (0.45 mmol) and DIPEA (1.5 mmol) in dichloromethane (5 mL) in a nitrogen atmosphere was added the appropriate aryl chloroformate (0.75 mmol) at room temperature. The reaction mixture was stirred over night and evaporated to dryness. The crude product was either purified by flash chromatography (Quad flash 25, EtOAc-heptane) or redissolved in MeCN and purified by preparative HPLC (Gilson).

General Procedure 8

4-(4-Allyloxycarbonylmethyl-phenoxy)-piperidine-1-carboxylic acid 4-(2,5-dioxo-pyrrolidin-1-yloxycarbonyl)-phenyl ester (1.0 eq) was dissolved in tetrahydrofurane (0.1-0.2 mmol/ml concentration of allylester). Diisopropylethylamine (2.5-3.0 eq) was added together with an appropriate amine (2-2.4 eq). The reaction mixture was stirred for 5-20 hours evaporated and extracted four times with dichloromethane from 5% aqueous citric acid. The organic phase was dried and evaporated to give the crude product. The crude product was purified by preparative HPLC (Gilson).

General Procedure 9

4-(4-Allyloxycarbonylmethyl-phenoxy)-piperidine-1-carboxylic acid alkylcarbamoyl)-phenyl ester (1.0 eq), morpholine (3 eq.), tetrakis-(triphenylphosphin)-palladium (0.1 eq) was added to a reaction flask under nitrogen atmosphere. Tetrahydrofurane (0.05-0.15 mmol/ml concentration of allylester) was added and the reaction mixture was stirred for 1-5 days, evaporated to dryness and subjected to preparative HLC.

General Procedure 10

4-(4-Allyloxycarbonylmethyl-phenoxy)-piperidine-1-carboxylic acid 4-(2-hydroxy-ethyl)-phenyl ester (1.0 eq), tributylphosphine (1.5 eq), azadicarboxylic dipiperidide (ADDP) (1.5 eq), and an appropriate thio or hydroxy mitzunobu reagent (e.g. phenol/thiophenol/N-hydroxy or thioazole/azole) (1.0 eq), was added to a reaction flask under nitrogen. Tetrahydrofurane (0.05-0.2 mmol of 4-(4-Allyloxycarbonylmethyl-phenoxy)-piperidine-1-carboxylic acid 4-(2-hydroxy-ethyl)-phenyl ester). The reaction mixture was stirred or shaken for 1-4 days evaporated to dryness and purified by flash or preparative HPLC.

General Procedure 11

3-[4-(4-Allyloxycarbonylmethyl-phenoxy)-piperidine-1-carbonyl]-1-methyl-3H-imidazol-1-ium; iodide (1.0 eq), diisopropylethylamine (3 eq), a phenol (1.0 eq) and dichloromethane (0.05-1.5 mmol/ml of the 3-[4-(4-Allyloxycarbonylmethyl-phenoxy)-piperidine-1-carbonyl]-1-methyl-3H-imidazol-1-ium; iodide) was added to a reaction flask and stirred for at least 3 hours. The reaction mixture was evaporated and purified by prep. HPLC.

General Procedure 12

4-Hydroxy-piperidine-1-carboxylic acid 4-(3,3-dimethyl-butylcarbamoyl)-phenyl ester (1.0 eq), tributylphosphine (1.5 eq), azadicarboxylic dipiperidide (ADDP) (1.5 eq), and an appropriate thio or hydroxy mitzunobu reagent (e.g. phenol/thiophenol/N-hydroxy or thioazole/azole) (1.1 eq), was added to a reaction flask under nitrogen. Tetrahydrofurane (0.05-0.3 mmol/ml of 4-hydroxy-piperidine-1-carboxylic acid 4-(3,3-dimethyl-butylcarbamoyl)-phenyl ester) was added and the reaction mixture was stirred or shaken for 1-4 days. The reaction mixture was evaporated to dryness and purified by flash chromatography or preparative HPLC.

General Procedure 13

A solution of an alcohol (0.4 mmol), an appropriate thio or hydroxy mitzunobu reagent (e.g. phenol/thiophenol/N-hydroxyazole/azole) (0.33-0.4 mmol), and tributylphosphine (0.5-2.0 mmol) in THF (2-8 mL) was stirred under nitrogen at rt. in a flame dried reaction flask. ADDP (0.5-2.0 mmol) dissolved in THF (1.5-3 mL) was added and the reaction mixture was stirred at rt. for at least 16 h, sometimes filtered, evaporated to dryness and purified by preparative HPLC and/or flash chromatography.

Starting Materials/Intermediates

### 4-(Pyrazol-1-yloxy)-piperidine

Step A:

A solution of tert-butyl 4-hydroxypiperidine-1-carboxylate (6.04 g, 30 mmol), 1-hydroxy-pyrazole (2.52 g, 30 mmol) and tributylphosphine (9.74 mL, 39 mmol) in tetrahydrofuran (100 mL) was stirred under nitrogen at 0° C. A solution of 1,1′-(azodicarbonyl)dipiperidine (9.84 g, 39 mmol) in tetrahydrofuran (100 mL) was added and the mixture was stirred at room temperature for 16 hours, filtered and evaporated to dryness. The residue was triturated with dichloromethane (100 mL), filtered and evaporated to dryness yielding a crude yellow oil (18.5 g) containing 4-(pyrazol-1-yloxy)-piperidine-1-carboxylic acid tert-butyl ester, which was used without further purification in the following step.

¹H NMR (400 MHz, CDCl₃): δ 1.46 (s, 9H), 1.87 (m, 2H), 3.16 (m, 2H), 3.36 (m, 2H), 3.84 (m, 2H), 4.61 (m,1 H), 6.18 (t,1H), 7.27 (dd,1H), 7.31 (dd,1H); HPLC-MS (Method A): m/z=168 (M-Boc+H)⁺; Rt=3.60 min.

Step B:

The crude oil from step A was stirred in a mixture of dichloromethane (50 mL) and trifluoroacetic acid (50 mL) for about 60 hours and evaporated to dryness. The residue was partitioned between dichloromethane (50 mL) and 12.5 M hydrochloric acid (50 mL). The acidic aqueous phase was washed with dichloromethane (2×25 mL) followed by evaporation to dryness in vacuo. The residue was dissolved in 1N sodium hydroxide and extracted with dichloromethane (4×25 mL). The organic phase was dried over sodium sulphate and evaporated to dryness yielding 4-(pyrazol-1-yloxy)-piperidine (2.74 g, 34% yield) as a yellow oil.

¹H NMR (400 MHz, CDCl₃): δ 1.63 (m, 2H), 1.88 (s, 1H), 1.93 (m, 2H), 2.66 (m, 2H), 3.15 (m, 2H), 4.52 (m, 1H), 6.17 (t, 1H), 7.27 (dd, 1H), 7.31 (dd, 1H); HPLC-MS (Method A): m/z=168 (M+H)⁺, Rt=0.49 min.

The intermediate 4-hydroxy-piperidine-1-carboxylic acid 4-(4-trifluoromethyl-benzyl)-phenyl ester was prepared as described in PCT/DK02/00852, i.e. WO 03/51841)

4-Hydroxy-piperidine-1-carboxylic acid 4-(5-methyl-pyridin-2-ylmethyl)-phenyl ester, 3-[4-(tert-butyl-dimethyl-silanyloxy)-piperidine-1-carbonyl]-1-methyl-3H-imidazol-1-ium; iodide and were prepared as described in PCT/DK02/00852, i.e. WO 03/51841. 1-Hydroxypyrazole was prepared as described in Begtrup, Vedsø, J. Chem. Soc. Perkin Trans 1, 1995, 243. 1-Hydroxyimidazole was prepared as described in Eriksen et al., J. Org. Chem. 63, 1998, 12. 4-Hydroxy-piperidine-1-carboxylic acid 4-[2-(4,4-dimethyl-2,6-dioxo-piperidin-1-yl)-ethyl]-phenyl ester.

To a solution of 4-(2-Hydroxyethyl)phenol (25 mmol) and 4-diazabicyclo[2.2.2]octane (DABCO) (27 mmol) in CH₂Cl₂ (50 mL) was added 3-[4-(tert-butyl-dimethyl-silanyloxy)-piperidine-1-carbonyl]-1-methyl-3H-imidazol-1-ium; iodide (27 mmol). The reaction mixture was stirred for 16 hours at rt, filtered and the organic phase was washed with water three times, dried (MgSO₄) and evaporated to give 99% of 4-(tert-butyl-dimethyl-silanyloxy)-piperidine-1-carboxylic acid 4-(2-hydroxy-ethyl)-phenyl ester as a light yellow oil which was used without further purification.

4-(tert-Butyl-dimethyl-silanyloxy)-piperidine-1-carboxylic acid 4-(2-hydroxy-ethyl)-phenyl ester (5 mmol) 3,3-dimethylglutarimide (5 mmol), diisopropylethylamin (5.5 mmol) and tributylphosphine (6.5 mmol) in THF (25 mL) was stirred under nitrogen at rt. 1,1′-(Azodicarbonyl)dipiperidine (ADDP, 6.5 mmol) dissolved in THF (25 mL) was added and the reaction mixture was stirred at rt for 16 h. The reaction mixture was filtered, added water and extracted with EtOAc. The combined organic phases were dried and concentrated and purified by flash chromatography (Quad flash 40, EtOAc-heptane 1:3->1:0) to give 84% of 4-(tert-butyl-dimethyl-silanyloxy)-piperidine-1-carboxylic acid 4-[2-(4,4-dimethyl-2,6-dioxo-piperidin-1-yl)-ethyl]-phenyl ester as beige crystals. 4-(tert-Butyl-dimethyl-silanyloxy)-piperidine-1-carboxylic acid 4-[2-(4,4-dimethyl-2,6-dioxo-piperidin-1-yl)-ethyl]-phenyl ester (4 mmol) was deprotected by stirring with a 1.7 M solution of HCl in Et₂O (20 mL) in DCM (5 mL) for 16 h at rt. Evaporation to dryness followed by evaporation with MeCN produced 97% of the title compound as a solid.

¹H NMR (400MHz; CDCl₃): δ 1.06 (s, 6H), 1.54-1.63 (m, 2H), 1.90-1.98 (m, 2H), 2.24 (s, 4H), 2.78-2.83 (m, 2H), 3.18-3.37 (m, 2H), 3.90-4.06 (m, 5H), 7.01 (d, 2H), 7.24 (d, 2H); HPLC-MS: m/z=389.2 (M+1); Rₜ=3.01 min.

4-Benzyl piperidines 2-piperidin-4-ylmethyl-pyridine, 5-methyl-2-piperidin-4-ylmethyl-pyridine, 4-methyl-2-piperidin-4-ylmethyl-pyridine, (6-piperidin-4-ylmethyl-pyridin-2-yl)-methanol, 2-piperidin-4-ylmethyl-pyrimidine, 4-thiophen-3-ylmethyl-piperidine, 4-(4-cyanomethyl-benzyl)-piperidine-1-carboxylic acid tert-butyl ester and (4-piperidin-4-ylmethyl-phenyl)-acetic acid were prepared employing the pro-tocol described in Vice et al., J. Org. Chem. 66, 2001, 2487 using 2-bromo pyridine, 2-bromo-5methyl pyridine, 2-bromo-4-methyl pyridine, 2-bromo-6-formyl pyridine, 2-bromo pyrimidine, 3-bromo thiophene, 4-bromobenzyl cyanide or (4-bromophenyl)-acetic acid allyl ester respectively as aryl/heteroaryl bromides. 4-[4-(2H-Tetrazol-5-ylmethyl)-benzyl]-piperidine was prepared by treatment of 4-(4-cyanomethyl-benzyl)-piperidine-1-carboxylic acid tert-butyl ester with sodium azide using the protocol described by Meyer et al., Synthesis, 2003, 899. 4-[4-(1H-Tetrazol-5-ylmethyl)-phenoxy]-piperidine was prepared by treatment of 4-[4-(1H-tetrazol-5-ylmethyl)-phenoxy]-piperidine-1-carboxylic acid tert-butyl ester with DCM-TFA (1:1) at room temperature. 4-[4-(1H-Tetrazol-5-ylmethyl)-phenoxy]-piperidine-1-carboxylic acid tert-butyl ester was prepared in a similar fashion using 4-(4-cyanomethyl-phenoxy)-piperidine-1-carboxylic acid tert-butyl ester which was prepared from N-Boc protected 4-hydroxypiperidine and 4-hydroxybenzylpiperidine using the general procedure 5. 4-[4-(Trifluoromethyl)benzyl]phenyl chloroformate was prepared as follows: Ethyldiisopropylamine (0.7 mL, 4.02 mmol) was added to a solution of 4-(4-trifluoromethyl-benzyl)-phenol (1.0 g, 4.0 mmol) in 20% phosgene in toluene (3 ml, 5.5 mmol) with stirring at 0° C. The mixture was stirred in the melting ice bath for 3 h and evaporated to dryness. The residue was triturated with three portions of diethyl ether and the combined organic phase was evaporated to dryness to give 1.12 g (89%) of crude chloroformate, which was used in the following step without further purification. ¹H NMR (400 MHz, CDCl₃): δ 4.04 (s, 2 H), 7.15 (d, 2 H), 7.21 (d, 2 H), 7.28 (d, 2 H), 7.56 (d, 2 H).

### Example 1

### 4-[2-(Benzyl-methyl-amino)-ethyl]-piperidine-1-carboxylic acid 4-(5-trifluoromethyl-pyridin-2-yloxy)-phenyl ester

The title compound was prepared according to general procedure 4 applying the following starting materials: 4-(5-trifluoromethyl-pyridin-2-yloxy)-phenyl chloroformate and 1-benzyl-methyl-(2-piperidin-4-yl-ethyl)-amine. The crude product was subjected to flash chromatography (ethyl acetate/heptane, 1:1). HCl (g) in ether was added to a solution of the title product. The solvent was evaporated in vacuo and the title product dried under reduced pressure to give the hydrochloride. Yield: 190 mg; 53%. LC-MS: m/z: 514.1 (M+); Rt=3.83 min.

### Example 2

### 4-(Pyridin-2-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and Pyridine-2-thiol. Yield: 68 mg; 30%. LC-MS: m/z: 476.0 (M+); Rt=4.54 min.

### Example 3

### 4-(1-Pyridin-3-yl-1H-imidazol-2-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 1-Pyridin-3-yl-1H-imidazole-2-thiol. The crude product was subjected to flash chromatography (ethyl acetate). HCl (g) in ether was added to a solution of the title product. The solvent was evaporated in vacuo and the title product dried under reduced pressure to give the hydrochloride. Yield: 121 mg; 72%. LC-MS: m/z: 542.1 (M+); Rt=3.58 min.

### Example 4

### 4-(1-Methyl-1H-imidazol-2-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 1-Methyl-1H-imidazole-2-thiol. The crude product was filtered and subjected to flash chromatography (ethyl acetate). HCl (g) in ether was added to a solution of the title product. The solvent was evaporated in vacuo and the title compound dried under reduced pressure to give the hydrochloride. Yield: 161 mg; 61%. LC-MS: m/z: 479 (M+); Rt=3.35 min.

### Example 5

### 4-[4-(4-Methyl-piperazine-1-carbonyl)-phenoxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and (4-Hydroxy-phenyl)-(4-methyl-piperazin-1-yl)-methanone. The crude product was subjected to flash chromatography (ethyl acetate→dichloromethane/methanole/acetic acid, 80:8:2) after collection of the appropriate samples following preparative HPLC. HCl (g) in ether was added to a solution of the title product. The solvent was evaporated in vacuo and the title compound dried under reduced pressure to give the hydrochloride. Yield: 41 mg; 19 %. LC-MS: m/z: 585.3 (M+); Rt=3.48 min.

### Example 6

### 4-(1H-Imidazol-2-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 1H-Imidazole-2-thiol. The crude product was subjected to flash chromatography (dichloromethane/methanol/acetic acid, 80:8:2). HCl (g) in ether was added to a solution of the title product. The solvent was evaporated in vacuo to give the hydrochloride which was dried under reduced pressure. Yield: 147 mg; 74 %. LC-MS: m/z: 465.1 (M+); Rt=3.27 min.

### Example 7

### 4-(4-Piperazin-1-yl-phenoxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-piperazin-1-yl-phenol. HCl (g) in ether was added to a solution of the product. The solvent was evaporated in vacuo to give the hydrochloride. The hydrochloride was recrystallized from ethyl acetate and the title product dried under reduced pressure. Yield: 38 mg; 19 %. LC-MS: m/z: 543.1 (M+); Rt=3.60 min.

### Example 8

### 4-(Pyrazol-1-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and pyrazole-1-ol. The crude product was subjected to flash chromatography (ethyl acetate/heptane, 1:1) followed by preparative HPLC. The title product was dried under reduced pressure. Yield: 0.121 mg; 68%. LC-MS: m/z: 449.0 (M+), Rt=4.46 min.

### Example 9

### 4-(Imidazol-1-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and imidazole-1-ol. The crude product was subjected to flash chromatography (ethyl acetate/heptane, 1:3). HCl (g) in ether was added to a solution of the product. The solvent was evaporated in vacuo to give the hydrochloride which was dried under reduced pressure. Yield: 101 mg; 56 %. LC-MS: m/z: 449.0 (M+), Rt=2.91 min.

### Example 10

### 4-(Pyridin-3-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and pyridine-3-ol. The crude product was subjected to flash chromatography (ethyl acetate) followed by preparative HPLC. HCl (g) in ether was added to a solution of the product. The solvent was evaporated in vacuo to give the hydrochloride which was dried under reduced pressure. Yield: 61 mg; 33%. LC-MS: m/z: 460.1 (M+); Rt=3.36 min.

### Example 11

### 4-[4-(2-Dimethylamino-ethyl)-phenoxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-(2-dimethylamino-ethyl)-phenol. The crude product was subjected to flash chromatography (ethyl acetate/methanol, 9:1) followed by preparative HPLC. HCl (g) in ether was added to a solution of the product. The solvent was evaporated in vacuo to give the hydrochloride which was dried under reduced pressure. Yield: 69 mg; 30%. LC-MS: m/z: 530.2 (M+); Rt=3.44 min.

### Example 12

### 4-(5-Phenylsulfanyl-pyrazol-1-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 5-phenylsulfanyl-pyrazol-1ol. The crude product was subjected to flash chromatography (ethyl acetate/heptane, 1:5). The title product was dried under reduced pressure. Yield: 170 mg; 76 %. LC-MS: m/z: 557.1 (M+); Rt=5.51 min.

### Example 13

### 4-(4-Imidazol-1-yl-phenoxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-imidazol-1-yl-phenol. The crude product was subjected to flash chromatography (ethyl acetate) followed by preparative HPLC. HCl (g) in ether was added to a solution of the product and the title product was dried under reduced pressure. Yield: 62 mg; 27%. LC-MS: m/z: 552.1 (M+); Rt=3.34 min.

### Example 14

### 4-(Pyridin-2-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-imidazol-1-yl-phenol. The crude product was subjected to flash chromatography (ethyl acetate/heptane, 1:3). HCl (g) in ether was added to a solution of the product and the title product was dried under reduced pressure. Yield: 73 mg; 40%. LC-MS: m/z: 460.1 (M+); Rt=4.56 min.

### Example 15

### 4-(3-Pyridin-2-yl-pyrazol-1-yloxy)-piperidine-1-carboxylic acid 4-(5-trifluoomethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 3-pyridin-2-yl-pyrazol-1-ol. The crude product was subjected to flash chromatography (ethyl acetate/heptane, 3:1). HCl (g) in ether was added to a solution of the product and the title product was dried under reduced pressure. Yield: 99 mg; 44%. LC-MS: m/z: 526.2 (M+); Rt=4.01 min.

### Example 16

### 4-(2-Phenylsulfanyl-imidazol-1-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 2-phenylsulfanyl-imidazol-1-ol. The crude product was subjected to flash chromatography (ethyl acetate/heptane, 1:3). HCl (g) in ether was added to a solution of the product and the title product was dried under reduced pressure. Yield: 156 mg; 66%. LC-MS: m/z: 557.1 (M+); Rt=4.66 min.

### Example 17

### 4-(2-Chloro-imidazol-1-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 2-chloro-imidazol-1-ol. The crude product was subjected to flash chromatography (ethyl acetate/heptane, 1:1). HCl (g) in ether was added to a solution of the product and the title product was dried under reduced pressure. Yield: 143 mg; 69%. LC-MS: m/z: 483.1 (M+); Rt=4.42 min.

### Example 18

### 4-[2-(4-Methoxy-phenyl)-imidazol-1-yloxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 2-(4-methoxy-phenyl)-imidazol-1-ol. The crude product was subjected to flash chromatography (ethyl acetate/heptane, 1:1). HCl (g) in ether was added to a solution of the product and the title product was dried under reduced pressure. Yield: 99 mg; 42%. LC-MS: m/z: 555.1 (M+); Rt=3.53 min.

### Example 19

### 4-(2-Methyl-imidazol-1-yloxy)-piperidine-1-carboxylic acid 4-(5-trifluorometyhl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 2-methyl-imidazol-1-ol. The crude product was subjected to flash chromatography (ethyl acetate) followed by preparative HPLC. HCl (g) in ether was added to a solution of the product and the title product was dried under reduced pressure. Yield: 33 mg; 16%. LC-MS: m/z: 463.2 (M+); Rt=2.85 min.

### Example 20

### 4-(Pyridin-4-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and pyridine-4-thiol. The crude product was subjected to flash chromatography (ethyl acetate) followed by preparative HPLC. HCl (g) in ether was added to a solution of the product and the title product was dried under reduced pressure. Yield: 138 mg; 59%. LC-MS: m/z: 476.1 (M+); Rt=3.58 min.

### Example 21

### 4-(4-Pyridin-3-ylmethyl-phenoxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and pyridine-4-thiol. The crude product was subjected to flash chromatography (ethyl acetate) followed by preparative HPLC. HCl (g) in ether was added to a solution of the product and the title product was dried under reduced pressure. Yield: 24 mg; 11 %. LC-MS: m/z: 550.2 (M+); Rt=3.78 min.

### Example 22

### 4-[4-(4-Cyclopentyl-piperazin-1-yl)-phenoxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-(4-cyclopentyl-piperazin-1-yl)-phenol. The crude product was subjected to flash chromatography (ethyl acetate). The product was crystallized from DMSO, separated and HCl (g) in ether was added to an ethyl acetate solution of the product. The title product was dried under reduced pressure. Yield: 27 mg; 11%. LC-MS: m/z: 611.2 (M+); Rt=4.04 min.

### Example 23

### 4-(4-Pyridin-2-ylmethyl-phenoxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-pyridin-2-ylmethyl-phenol. The crude product was subjected to flash chromatography (ethyl acetate/heptane, (1:1)) followed by preparative HPLC. HCl (g) in ether was added to a solution of the product. The title product was dried under reduced pressure. Yield: 24 mg; 10%. LC-MS: m/z: 550.2 (M+); Rt=3.91 min.

### Example 24

### 4-[4-(5-Methyl-pyridin-2-ylmethyl)-phenoxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-(5-methyl-pyridin-2-ylmethyl)-phenol. The crude product was subjected to flash chromatography (ethyl acetate) followed by preparative HPLC. HCl (g) in ether was added to a solution of the product and the title product was dried under reduced pressure. Yield: 72 mg; 30%. LC-MS: m/z: 564.3 (M+); Rt=3.98 min.

### Example 25

### 4-(5-Pyridin-4-yl-[1,3,4]oxadiazol-2-ylsulfanyl)-piperidine-1-carboxylic acid 4-(5-trifluoromethyl-pyridin-2-yloxy)-phenyl ester

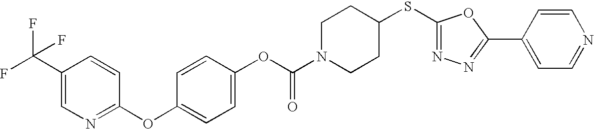

The title compound was prepared according to general procedure 1 applying the following starting materials 4-hydroxy-piperidine-1-carboxylic acid 4-(5-trifluoromethyl-pyridin-2-yloxy)-phenyl ester and 5-pyridin-4-yl-[1,3,4]oxadiazole-2-thiol. The crude product was subjected to flash chromatography (ethyl acetate, heptane, 2:1) followed by preparative HPLC. HCl (g) in ether was added to a solution of the product and the title product was dried under reduced pressure. Yield: 62 mg; 26 %. LC-MS: m/z: 544.0 (M+); Rt=4.22 min.

### Example 26

### 4-(5-Pyridin-4-yl-1H-[1,2,4]triazol-3-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 5-pyridin-4-yl-1H-[1,2,4]triazole-3-thiol. The crude product was subjected to flash chromatography (ethyl acetate, heptane, 3:1) followed by preparative HPLC. HCl (g) in ether was added to a solution of the product and the title product was dried under reduced pressure. Yield: 68 mg; 28%. LC-MS: m/z: 543.1 (M+); Rt=3.47 min.

### Example 27

### 4-(1-Methyl-1H-tetrazol-5-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 1-methyl-1H-tetrazole-5-thiol. The crude product was subjected to flash chromatography (ethyl acetate, heptane, 1:1). The title product was dried under reduced pressure. Yield: 153 mg; 80%. LC-MS: m/z: 481.0 (M+); Rt=4.34 min.

### Example 28

### 4-[4-(4-Ethoxycarbonyl-oxazol-2-yl)-phenoxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 2-(4-hydroxy-phenyl)-oxazole-4-carboxylic acid ethyl ester. The solvent was evaporated in vacuo to give the title product which was dried under reduced pressure. Yield: 76.3 mg; 32%. LC-MS: m/z: 620.1 (M+Na); Rt=5.31 min.

### Example 29

### 4-(4′-Methoxycarbonyl-biphenyl-4-yloxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4′-hydroxy-biphenyl-4-carboxylic acid methyl ester. The solvent was evaporated in vacuo to give the title product which was dried under reduced pressure. Yield: 69 mg; 29%. LC-MS: m/z: 593.1 (M+); Rt=5.77 min.

### Example 30

### 5-{1-[4-(5-Trifluoromethyl-pyridin-2-yloxy)-phenoxycarbonyl]-piperidin-4-yloxy)-1H-indole-2-carboxylic acid ethyl ester

The title compound was prepared according to general procedure 1 applying the following starting materials 4-hydroxy-piperidine-1-carboxylic acid 4-(5-trifluoromethyl-pyridin-2-yloxy)-phenyl ester and 5-hydroxy-1H-indole-2-carboxylic acid ethyl ester. The solvent was evaporated in vacuo to give the title product which was dried under reduced pressure. Yield: 76 mg; 33%. LC-MS: m/z: 570.2 (M+); Rt=5.37 min.

### Example 31

### -[4-(2-Ethoxycarbonyl-ethyl)-phenoxy]-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 3-(4-hydroxy-phenyl)-propionic acid ethyl ester. The crude product was subjected to flash chromatography (ethyl acetate) followed by preparative HPLC. The title product was dried under reduced pressure. Yield: 62 mg; 27%. LC-MS: m/z: 559.2 (M+); Rt=5.48 min. mg

### Example 32

### 4-(3-Ethoxycarbonyl-phenoxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 3-hydroxy-benzoic acid ethyl ester. The crude product was subjected to flash chromatography (ethyl acetate) followed by preparative HPLC. The title product was dried under reduced pressure. Yield: 65 mg; 31%. LC-MS: m/z: 531.1 (M+); Rt=5.48 min.

### Example 33

### 4-(4-Ethoxycarbonyl-1H-imidazol-2-ylsulfanyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 2-mercapto-1H-imidazole-4-carboxylic acids ethyl ester. The crude product was subjected to flash chromatography (ethyl acetate/heptane, 1:1). The title product was dried under reduced pressure. Yield: 184 mg; 86%. LC-MS: m/z: 559.2 (M+Na); Rt=4.03 min.

### Example 34

### 4-(4-Allyloxycarbonylmethyl-phenoxy)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and (4-hydroxy-phenyl)-acetic acid allyl ester. The crude product was subjected to flash chromatography (ethyl acetate/heptane, 1:1) followed by preparative HPLC. The title product was dried under reduced pressure. Yield: 36 mg; 16%. LC-MS: m/z: 557.2 (M+Na); Rt=5.36 min.

### Example 35

### 4-(4′-Carboxy-biphenyl-4-yloxy)-piperidine-1-carboxylic acid 4-(5-trifluoromethyl-pyridin-2-yloxy)-phenyl ester

The title compound was prepared according to general procedure 2 applying the following starting material: 4-(4′-Methoxycarbonyl-biphenyl-4-yloxy)-piperidine-1-carboxylic acid 4-(5-trifluoromethyl-pyridin-2-yloxy)-phenyl ester. The solvent was evaporated in vacuo to give the title product which was dried under reduced pressure. Yield: 14 mg; 10%. LC-MS: m/z: 579.1 (M+); Rt=5.40 min.

### Example 36

### 4-[4-(4-Carboxy-oxazol-2-yl)-phenoxy]-piperidine-1-carboxylic acid 4-(5-trifluoromethyl-pyridin-2-yloxy)-phenyl ester

The title compound was prepared according to general procedure 2 applying the following starting material: 4-(4′-Methoxycarbonyl-biphenyl-4-yloxy)-piperidine-1-carboxylic acid 4-(5-trifluoromethyl-pyridin-2-yloxy)-phenyl ester. The solvent was evaporated in vacuo to give the title product which was dried under reduced pressure. Yield: 6.7 mg; 6%. LC-MS: m/z: 570.1 (M+); Rt=5.45 min.

### Example 37

### 4-[4-(2-Carboxy-ethyl)-phenoxy]-piperidine-1-carboxylic acid 4-(5-trifluoromethyl-pyridin-2-yloxy)-phenyl ester

The title compound was prepared according to general procedure 2 applying the following starting material: 4-[4-(2-Ethoxycarbonyl-ethyl)-phenoxy]-piperidine-1-carboxylic acid 4-(5-trifluoromethyl-pyridin-2-yloxy)-phenyl ester. The solvent was evaporated in vacuo to give the title product which was dried under reduced pressure. Yield: 98 mg; 61 %. LC-MS: m/z: 531.1 (M+H); Rt=4.66 min.

### Example 38

### 4-(3-Carboxy-phenoxy)-piperidine-1-carboxylic acid 4-(5-trifluoromethyl-pyridin-2-yloxy)-phenyl ester

The title compound was prepared according to general procedure 2 applying the following starting material: 4-[4-(2-Ethoxycarbonyl-ethyl)-phenoxy]-piperidine-1-carboxylic acid 4-(5-trifluoromethyl-pyridin-2-yloxy)-phenyl ester. The solvent was evaporated in vacuo to give the title product which was dried under reduced pressure. Yield: 33 mg; 33%. LC-MS: m/z: 503.2 (M+H); Rt=4.48 min.

### Example 39

### 4-(4-Carboxy-phenoxy)-piperidine-1-carboxylic acid 4-(5-trifluoromethyl-pyridin-2-yloxy)-phenyl ester

The title compound was prepared according to general procedure 3 applying the following starting material: 4-(4-Allyloxycarbonyl-phenoxy)-piperidine-1-carboxylic acid 4-(5-trifluoromethyl-pyridin-2-yloxy)-phenyl ester. The crude product was subjected to flash chromatography (ethyl acetate/heptane/acetic acid, 5:5:0.5). The solvent was evaporated in vacuo to give the title product which was dried under reduced pressure. Yield: 92 mg; 91%. LC-MS: m/z: 503.0 (M+H); Rt=4.71 min.

### Example 40

### 4-(4-Carboxymethyl-phenoxy)-piperidine-1-carboxylic acid 4-(5-trifluoromethyl-pyridin-2-yloxy)-phenyl ester

The title compound was prepared according to general procedure 2 applying the following starting material: 4-(4-Methoxycarbonylmethyl-phenoxy)-piperidine-1-carboxylic acid 4-(5-trifluoromethyl-pyridin-2-yloxy)-phenyl ester. The crude product was subjected to flash chromatography (ethyl acetate/heptane/acetic acid, 5:5:0.5). The solvent was evaporated in vacuo to give the title product which was dried under reduced pressure. Yield: 105 mg; 100%. LC-MS: m/z: 517.1 (M+H); Rt=4.53 min.

### Example 41

### 4-(1H-imidazol-2-ylsulfanylmethyl)-piperidine-1-carboxylic acid 4-(5-trifluoromethyl-pyridin-2-yloxy)-phenyl ester

The title compound was prepared according to general procedure 1 applying the following starting materials 4-Hydroxymethyl-piperidine-1-carboxylic acid 4-(5-trifluoromethyl-pyridin-2-yloxy)-phenyl ester and 1H-imidazole-2-thiol. The crude product was subjected to flash chromatography (ethyl acetate/heptane, 1:1). The solvent was evaporated in vacuo to give the title product which was dried under reduced pressure. Yield: 103 mg; 54%. LC-MS: m/z: 479.0 (M+); Rt=3.78 min.

### Example 42

### 4-(4-Allyloxycarbonylmethyl-phenoxymethyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and (4-hydroxy-phenyl)-acetic acid allyl ester. The crude product was subjected to flash chromatography (ethyl acetate/heptane, 1:3). The solvent was evaporated in vacuo to give the title product which was dried under reduced pressure. Yield: 111 mg; 49%. LC-MS: m/z: 571.1 (M+); Rt=6.08 min.

### Example 43

### 4-(4-Allyloxycarbonyl-phenoxymethyl)-piperidine-1-carboxylic acid 4-(5-trifluoromethyl-pyridin-2-yloxy)-phenyl ester

The title compound was prepared according to general procedure 1 applying the following starting materials 4-hydroxy-piperidine-1-carboxylic acid 4-(5-trifluoromethyl-pyridin-2-yloxy)-phenyl ester and 4-hydroxy-benzoic acid allyl ester. The crude product was subjected to flash chromatography (ethyl acetate/heptane, 1:5). The solvent was evaporated in vacuo to give the title product which was dried under reduced pressure. Yield: 183 mg; 82%. LC-MS: m/z: 557.2 (M+); Rt=5.61 min.

### Example 44

### 4-(4-Carboxymethyl-phenoxymethyl)-piperidine-1-carboxylic acid 4-(5-trifluoromethyl-pyridin-2-yloxy)-phenyl ester

The title compound was prepared according to general procedure 3 applying the following starting material: 4-(4-Allyloxycarbonylmethyl-phenoxymethyl)-piperidine-1-carboxylic acid 4-(5-trifluoromethyl-pyridin-2-yloxy)-phenyl ester. The crude product was subjected to preparative HPLC. The solvent was evaporated in vacuo to give the title product which was dried under reduced pressure. Yield: 83.3 mg; 75%. LC-MS: m/z: 531.1 (M+H); Rt=5.46 min.

### Example 45

### 4-(4-Carboxy-phenoxymethyl)-piperidine-1-carboxylic acid 4-(5-trifluoromethyl-pyridin-2-yloxy)-phenyl ester

The title compound was prepared according to general procedure 3 applying the following starting material: 4-(4-Allyloxycarbonyl-phenoxymethyl)-piperidine-1-carboxylic acid 4-(5-trifluoromethyl-pyridin-2-yloxy)-phenyl ester. The crude product was subjected to flash chromatography (ethyl acetate/heptane/acetic acid, 5:5:0.05). The solvent was evaporated in vacuo to give the title product which was dried under reduced pressure. Yield: 90 mg; 87%. LC-MS: m/z: 517.1 (M+H); Rt=4.63 min.

### Example 46

### 4-(4-Allyloxycarbonyl-phenoxy)-piperidine-1-carboxylic acid 4-(4-trifluoromethyl-benzyl)-phenyl ester

A mixture of triphenylphosphine (177 mg, 0.67 mmol) and diethyl azodicarboxylate (106 μL, 0.67 mmol) in dry tetrahydrofuran (2 ml) was stirred at 0° C. under nitrogen for 15 min. Then a solution of 4-hydroxy-benzoic acid allyl ester (95 mg, 0.53 mmol) and 4-hydroxy-piperidine-1-carboxylic acid 4-(4-trifluoromethyl-benzyl)-phenyl ester (200 mg, 0.53 mmol) in dry tetrahydrofuran (1 ml) was added and the mixture was stirred for 1 h at room temperature. The mixture was evaporated to dryness and the residue was purified by flash column chromatography (SiO₂, dichloromethane) to give 124 mg (45%) of the pure allyl ester. ¹H NMR (400 MHz, CDCl₃): δ 1.91 (m, 2 H), 2.04 (m, 2 H), 3.73 (m, 4 H), 4.67 (m, 1 H), 4.80 (m, 2 H), 5.28 (m, 1 H), 5.40, (m, 1 H), 6.04 (m, 1 H), 6.95 (d, 2 H), 7.05 (d, 2 H), 7.16 (d, 2 H), 7.29 (d, 2 H), 7.53 (d, 2 H), 8.03 (d, 2 H); HPLC-MS: m/z =540(M+1)+, Rt=6.3 min.

### Example 47

### 4-(4-Carboxy-phenoxy)-piperidine-1-carboxylic acid 4-(4-trifluoromethyl-benzyl)-phenyl ester

A mixture of 4-(4-allyloxycarbonyl-phenoxy)-piperidine-1-carboxylic acid 4-(4-trifluoromethyl-benzyl)-phenyl ester (200 mg, 0.37 mmol), dimedone (83 mg, 0.59 mmol) and tetrakis(triphenylphosphine)palladium(0) (42 mg, 0.036 mmol) in dry tetrahydrofuran (10 mL) was stirred under nitrogen for 18 h at room temperature. The mixture was evaporated to dryness and the residue was purified by preparative HPLC (Gilson) followed by trituration with ethyl acetate giving 46 mg (25 %) of the title compound as white crystals. ¹H NMR (400 MHz, DMSO-d₆): δ 1.68 (br, 2 H), 2.03 (br, 2 H), 3.35 (br, 1 H), 3.48 (br, 1 H), 3.74 (br, 1 H), 3.87 (br, 1 H), 4.05 (s, 2 H), 4.77 (m, 1 H), 7.06 (d, 2 H), 7.08 (d, 2 H), 7.25 (d, 2 H), 7.47 (d, 2 H), 7.66 (d, 2 H), 7.89 (d, 2 H), 12.63 (br s,1 H); HPLC-MS: m/z =500 (M+1)+, Rt=5.2 min.

### b) 4-(4-Carboxy-phenoxy)-piperidine-1-carboxylic acid 4-(4-trifluoromethyl-benzyl)-phenyl ester

A mixture of 4-(4-allyloxycarbonyl-phenoxy)-piperidine-1-carboxylic acid 4-(4-trifluoromethyl-benzyl)-phenyl ester (200 mg, 0.37 mmol), dimedone (83 mg, 0.59 mmol) and tetrakis(triphenylphosphine)palladium(0) (42 mg, 0.036 mmol) in dry tetrahydrofuran (10 mL) was stirred under nitrogen for 18 h at room temperature. The mixture was evaporated to dryness and the residue was purified by preparative HPLC (Gilson) followed by trituration with ethyl acetate giving 46 mg (25 %) of the title compound as white crystals. ¹H NMR (400 MHz, DMSO-d₆): δ 1.68 (br, 2 H), 2.03 (br, 2 H), 3.35 (br, 1 H), 3.48 (br, 1 H), 3.74 (br, 1 H), 3.87 (br, 1 H), 4.05 (s, 2 H), 4.77 (m, 1 H), 7.06 (d, 2 H), 7.08 (d, 2 H), 7.25 (d, 2 H), 7.47 (d, 2 H), 7.66 (d, 2 H), 7.89 (d, 2 H), 12.63 (br s, 1 H); HPLC-MS: m/z =500 (M+1)+, Rt=5.2 min.

### Example 48

### General Procedure 5

### 4-(Pyrazol-1-yloxy)-piperidine-1-carboxylic acid 4-(5-methyl-pyridin-2-ylmethyl)-phenyl ester

The title compound (51%) was prepared as an oil using 4-hydroxy-piperidine-1-carboxylic acid 4-(5-methyl-pyridin-2-ylmethyl)-phenyl ester and 1-hydroxypyrazole. NMR (400 MHz; CDCl₃): δ 1.76-2.00 (m, 4H), 2.29 (s, 3H), 3.33-3.51 (m, 2H), 3.90-4.06 (m, 2H), 4.10 (s, 2H), 4.68-4.74 (m,1 H), 6.20 (t,1 H), 6.97-7.05 (m, 3H), 7.21-7.40 (m, 4H), 8.36 (d, 1H); HPLC-MS: m/z=393.1 (M+1); Rt=2.42 min.

### Example 49

### General Procedure 5

### 4-(Imidazol-1-yloxy)-piperidine-1-carboxylic acid 4-(6-methyl-pyridin-3-ylmethyl)-phenyl ester

The title compound (19%) was prepared as its TFA salt using 4-hydroxy-piperidine-1-carboxylic acid 4-(5-methyl-pyridin-2-ylmethyl)-phenyl ester and 1-hydroxyimidazole. HPLC-MS: m/z=393.1 (M+1); Rt=1.34 min.

### Example 50

### General Procedure 5

### 4-Phenoxy-piperidine-1-carboxylic acid 4-(6-methyl-pyridin-3-ylmethyl)-phenyl ester

The title compound (69%) was prepared as an oil using 4-hydroxy-piperidine-1-carboxylic acid 4-(5-methyl-pyridin-2-ylmethyl)-phenyl ester and phenol.

HPLC-MS: m/z=403.1 (M+1); Rₜ=3.34 min.

### Example 51

### General Procedure 5

### 4-(1H-Imidazol-2-ylsulfanyl)-piperidine-1-carboxylic acid 4-(5-methyl-pyridin-2-ylmethyl)-phenyl ester

The title compound (88%) was prepared as an oil using 4-hydroxy-piperidine-1-carboxylic acid 4-(5-methyl-pyridin-2-ylmethyl)-phenyl ester and 2-mercaptoimadazole.

HPLC-MS: m/z=409.1 (M+1); Rₜ=1.43 min.

### Example 52

### General Procedure 6

### 4-(4-Allyloxycarbonylmethyl-phenoxy)-piperidine-1-carboxylic acid 4-(5-methyl-pyridin-2-ylmethyl)-phenyl ester

The title compound (12%) was prepared as an oil using 4-hydroxy-piperidine-1-carboxylic acid 4-(5-methyl-pyridin-2-ylmethyl)-phenyl ester and (4-hydroxy-phenyl)-acetic acid allyl ester.

HPLC-MS: m/z=501.2 (M+1); Rₜ=4.22 min.

### Example 53

### 4-(4-Carboxymethyl-phenoxy)-piperidine-1-carboxylic acid 4-(5-methyl-pyridin-2-ylmethyl)-phenyl ester

4-(4-Allyloxycarbonylmethyl-phenoxy)-piperidine-1-carboxylic acid 4-(5-methyl-pyridin-2-ylmethyl)-phenyl ester (0.036 mmol), morpholine (0.12 mmol), Pd(PPh₃)₄ (0.0036 mmol) in dry THF (1 mL) were stirred in darkness for 1 h at room temperature. The reaction mixture was evaporated to dryness, to give a crude which was redissolved in MeCN and purified by preparative HPLC (Gilson). This gave the title compound as its TFA salt (78%) as an oil.

NMR (400MHz; CDCl₃): δ 1.82-2.05 (m, 4H), 2.48 (s, 3H), 3.54-3.88 (m, 4H), 3.58 (s, 2H), 4.39 (s, 2H), 4.52-4.57 (m,1H), 6.88 (d, 2H), 7.11 (d, 2H), 7.20 (d, 2H), 7.27 (d, 2H), 7.37 (d, 1H), 7.93 (dd, 1H), 8.69 (s, 1H), 9.90 (br s, 2H); HPLC-MS : m/z=461.0 (M+1); Rₜ=2.73 min.

### Example 54

### General Procedure 5

### 4-(1-Methyl-1H-imidazol-2-ylsulfanyl)-piperidine-1-carboxylic acid 4-[2-(4,4-dimethyl-2,6-dioxo-piperidin-1-yl)-ethyl]-phenyl ester

The title compound (65%) was prepared as its TFA salt using 4-hydroxy-piperidine-1-carboxylic acid 4-[2-(4,4-dimethyl-2,6-dioxo-piperidin-1-yl)-ethyl]-phenyl ester and 1-methyl-2-mercaptoimidazole.

¹H NMR (400 MHz; CDCl₃): δ 1.07 (s, 6H), 1.65-1.75 (m, 2H), 2.01-2.15 (m, 2H), 2.48 (s, 4H), 2.79-2.84 (m, 2H), 3.04-3.26 (m, 2H), 3.82 (s, 3H), 3.96-4.01 (m, 2H), 7.24 (d, 2H); HPLC-MS: m/z=485.2 (M+1); Rₜ=2.92 min.

### Example 55

### General Procedure 5

4-(4-Allyloxycarbonylmethyl-phenoxy)-piperidine-1-carboxylic acid 4-[2-(4,4-dimethyl-2,6-dioxo-piperidin-1-yl)-ethyl]-phenyl ester

The title compound (7%) was prepared as crystals using 4-hydroxy-piperidine-1-carboxylic acid 4-[2-(4,4-dimethyl-2,6-dioxo-piperidin-1-yl)-ethyl]-phenyl ester and (4-hydroxy-phenyl)-acetic acid allyl ester.

¹H NMR (400 MHz; CDCl₃): δ 1.04 (s, 6H), 1.83-2.03 (m, 4H), 2.48 (s, 4H), 2.79-2.84 (m, 2H), 3.52-3.89 (m, 4H), 3.60 (s, 2H), 4.51-4.56 (m,1H), 4.59 (dt,1H), 5.22 (dq, 1H), 5.28 (dq, 1H), 5.86-5.96 (m, 1H), 6.89 (d, 2H), 7.03 (d, 2H), 7.22 (d, 2H), 7.25 (d, 2H)l HPLC-MS: m/z=563.2 (M+1); Rₜ=5.21 min.

### Example 56

### General Procedure 5

### 4-(1H-Imidazol-2-ylsulfanyl)-piperidine-1-carboxylic acid 4-[2-(4,4-dimethyl-2,6-dioxo-piperidin-1-yl)-ethyl]-phenyl ester

The title compound (85%) was prepared as an oil using 4-hydroxy-piperidine-1-carboxylic acid 4-[2-(4,4-dimethyl-2,6-dioxo-piperidin-1-yl)-ethyl]-phenyl ester and 2-mercaptoimidazole.

¹H NMR (400 MHz; CDCl₃): δ 1.06 (s, 6H), 2.03 (br d, 2H), 2.47 (s, 4H), 2.77-2.82 (m, 2H), 3.04-3.25 (m, 2H), 3.45 (br s, 2H), 3.94-3.92 (m, 1H), 3.95-3.98 (m, 2H), 4.08-4.22 (m, 2H), 6.99 (d, 2H), 7.22 (d, 2H), 7.30 (s, 2H); HPLC-MS: m/z =471.3 (M+1); Rt=3.23 min.

### Example 57

### 4-(4-Carboxymethyl-phenoxy)-piperidine-1-carboxylic acid 4-[2-(4,4-dimethyl-2,6-dioxo-piperidin-1-yl)-ethyl]-phenyl ester

4-(4-Allyloxycarbonylmethyl-phenoxy)-piperidine-1-carboxylic acid 4-[2-(4,4-dimethyl-2,6-dioxo-piperidin-1-yl)-ethyl]-phenyl ester (0.025 mmol), morpholine (0.080 mmol), Pd(PPh₃)₄ (0.0025 mmol) in dry THF (1 mL) were stirred in darkness for 1 h at room temperature. The reaction mixture was evaporated to dryness, to give a crude which was redissolved in MeCN and purified by preparative HPLC (Gilson). This gave the title compound (70%) as yellow crystals.

¹H NMR (400 MHz; CDCl₃): δ 1.07 (s, 6H), 1.83-2.03 (m, 4H), 2.48 (s, 4H), 2.79-2.84 (m, 2H), 3.51-3.88 (m, 4H), 3.60 (s, 2H), 3.96-4.01 (m, 2H), 4.51-4.56 (m,1H), 6.90 (d, 2H), 7.03 (d, 2H), 7.22 (d, 2H), 7.24 (d, 2H); HPLC-MS: m/z=523.2 (M+1); Rt=4.79 min.

### Example 58

### General Procedure 5

### 4-(4-Allyloxycarbonyl-phenoxy)-piperidine-1-carboxylic acid 4-[2-(4,4-dimethyl-2,6-dioxo-piperidin-1-yl)-ethyl]-phenyl ester

The title compound (47%) was prepared as crystals using 4-hydroxy-piperidine-1-carboxylic acid 4-[2-(4,4-dimethyl-2,6-dioxo-piperidin-1-yl)-ethyl]-phenyl ester and 4-hydroxy-benzoic acid allyl ester. Triphenylphosphine and DEAD were used instead of tributylphosphine and ADDP.

¹H NMR (400 MHz; CDCl₃): δ 1.07 (s, 6H), 1.87-2.07 (m, 4H), 2.49 (s, 4H), 2.79-2.84 (m, 2H), 3.55-3.91 (m, 4H), 3.97-4.02 (m, 2H), 4.64-4.69 (m, 1H), 4.80 (dt, 1H), 5.29 (dq, 1H), 5.40 (dq, 1H), 5.99-6.09 (m, 1H), 6.95 (d, 2H), 7.03 (d, 2H), 7.25 (d, 2H), 8.03 (d, 2H); HPLC: m/z=549.3 (M+1); Rt=5.26 min.

### Example 59

### 4-(4-Carboxy-phenoxy)-piperidine-1-carboxylic acid 4-[2-(4,4-dimethyl-2,6-dioxo-piperidin-1-yl)-ethyl]-phenyl ester

4-(4-Allyloxycarbonyl-phenoxy)-piperidine-1-carboxylic acid 4-[2-(4,4-dimethyl-2,6-dioxo-piperidin-1-yl)-ethyl]-phenyl ester (0.11 mmol), morpholine (0.35 mmol), Pd(PPh₃)₄ (0.011 mmol) in dry THF (2 mL) were stirred in darkness for 16 h at room temperature. The reaction mixture was evaporated to dryness, to give a crude which was redissolved in MeCN and purified by preparative HPLC (Gilson). This gave the title compound (45%) as colorless crystals.

¹H NMR (400 MHz; CDCl₃): δ 1.06 (s, 6H), 1.89-2.08 (m, 4H), 2.50 (s, 4H), 2.80-2.85 (m, 2H), 3.57-3.90 (m, 4H), 3.99-4.04 (m, 2H), 4.67-4.72 (m,1 H), 6.98 (d, 2H), 7.04 (d, 2H), 7.26 (d, 2H), 8.08 (d, 2H); HPLC-MS: m/z=509.2 (M+1); Rt=3.99 min.

### Example 60

### General Procedure 5

### 4-(1H-[1,2,4]Triazol-3-ylsulfanyl)-piperidine-1-carboxylic acid 4-(5-methyl-pyridin-2-ylmethyl)-phenyl ester

The title compound (64%) was prepared as an oil using 4-hydroxy-piperidine-1-carboxylic acid 4-(5-methyl-pyridin-2-ylmethyl)-phenyl ester and 3-mercapto-1,2,4-triazole. Triphenylphosphine and DEAD were used instead of tributylphosphine and ADDP.

NMR (400 MHz; CDCl₃): δ 1.74 (br s, 2H), 2.14 (br d, 2H), 2.31 (s, 3H), 3.08-3.28 (m, 2H), 3.72-3.77 (m, 1H), 4.04-4.14 (m, 4H), 7.00 (d, 2H), 7.05 (d, 1H), 7.18 (dd, 1H), 8.02 (s, 1H), 8.38 (d, 1H); HPLC-MS: m/z=410.2 (M+1); Rₜ=2.04 min.

### Example 61

### 4-(Pyrazol-1-yloxy)-piperidine-1-carboxylic acid 5-benzoylamino-pyridin-2-yl ester

Phosgene (5 mL, 20% in toluene) was added to a stirred suspension of N-(6-hydroxy-pyridin-3-yl)-benzamide (0.32 g, 1.50 mmol) and triethylamine (0.23 mL, 1.65 mmol) in dichloromethane (10 mL). After stirring for 1 hour at room temperature, the solvent and excess phosgene were evaporated under reduced pressure. Dichloromethane (10 mL) was added to the residue, followed by 1,4-diazabicyclo[2.2.2]octane (168 mg, 1.50 mmol) and 4-(pyrazol-1-yloxy)-piperidine (0.25 g, 1.50 mmol). Stirring was continued for 1 hour at room temperature. The product was purified by flash column chromatography (SiO₂, gradient of 50-100% ethyl acetate in heptane) yielding the title compound (352 mg, 58% yield) as a white solid.

¹H NMR (300 MHz, CDCl₃): δ1.88 (m, 4H), 3.37 (m, 1H), 3.52 (m, 1H), 3.87 (m, 1H), 4.00 (m, 1H), 4.67 (m, 1H), 6.20 (t, 1H), 6.95 (d, 1H), 7.29 (dd, 1H), 7.32 (dd, 1H), 7.44 (m, 2H), 7.53 (m, 1H), 7.90 (d, 2H), 8.12 (dd, 1H), 8.39 (dd, 1H), 8.87 (s, 1H); HPLC-MS (Method A): m/z=430 (M+Na)⁺; Rt=3.14 min.

### Example 62

### 4-(Pyrazol-1-yloxy)-piperidine-1-carboxylic acid 4,4-dimethyl-2,6-dioxo-3,4,5,6-tetrahydro-2H-[1,3′]bipyridinyl-6′-yl ester

Phosgene (5 mL, 20% in toluene) was added to a stirred suspension of 6′-hydroxy-4,4-dimethyl-4,5-dihydro-3H-[1,3′]bipyridinyl-2,6-dione (0.35 g, 1.50 mmol) and triethylamine (0.23 mL, 1.65 mmol) in dichloromethane (10 mL). After stirring for 1 hour at room temperature, the solvent and excess phosgene were evaporated under reduced pressure. Dichloromethane (10 mL) was added to the residue, followed by 1,4-diazabicyclo[2.2.2]octane (168 mg, 1.50 mmol) and 4-(pyrazol-1-yloxy)-piperidine (0.50 g, 3.00 mmol). Stirring was continued for 1 hour at room temperature. The product was purified by flash column chromatography (SiO₂, gradient of 50-100% ethyl acetate in heptane) yielding the title compound (356 mg, 56% yield) as a white solid.

¹H NMR (300 MHz, CDCl₃): δ1.22 (s, 6H), 1.88 (m, 2H), 1.98 (m, 2H), 2.69 (s, 4H), 3.39-3.61 (m, 2H), 3.89-4.10 (m, 2H), 4.71 (m, 1H), 6.19 (t, 1H), 7.22 (d, 1H), 7.28 (m, 1H), 7.32 (m, 1H), 7.51 (dd, 1H), 8.09 (d, 1H); HPLC-MS (Method A): m/z=428 (M+H)⁺; Rt=3.21 min.

### Example 63

### General Procedure 5

### 4-(1-Methyl-1H-tetrazol-5-ylsulfanyl)-piperidine-1-carboxylic acid 4-(5-methyl-pyridin-2-ylmethyl)-phenyl ester

The title compound (25%) as it's TFA salt was prepared as an oil using 4-hydroxy-piperidine-1-carboxylic acid 4-(5-methyl-pyridin-2-ylmethyl)-phenyl ester and 1-methyl-5-mercaptotetrazole. HPLC-MS: m/z=425.2 (M+1); Rt=2.39 min.

### Example 64

### General Procedure 7

### 4-Pyridin-2-ylmethyl-piperidine-1-carboxylic acid 4-(5-trifluoromethyl-pyridin-2-yloxy)-phenyl ester

The title compound (78%) was prepared as an oil using 4-(5-trifluoromethyl-pyridin-2-yloxy)-phenyl chloroformate and 2-piperidin-4-ylmethyl-pyridine. HPLC-MS: m/z=458.0 (M+1); Rt=3.21 min.

### Example 65

### General Procedure 7

### 4-Pyridin-2-ylmethyl-piperidine-1-carboxylic acid 4-(4-trifluoromethyl-benzyl)-phenyl ester

The title compound (85%) was prepared as crystals using 4-[4-(trifluoromethyl)benzyl]phenyl chloroformate and 2-piperidin-4-ylmethyl-pyridine. HPLC-MS: m/z=455.2 (M+1); Rₜ=3.66 min.

### Example 66

### General Procedure 7

### 4-Pyrimidin-2-ylmethyl-piperidine-1-carboxylic acid 4-(5-trifluoromethyl-pyridin-2-yloxy)-phenyl ester

The title compound (37%) was prepared as an oil using 4-(5-trifluoromethyl-pyridin-2-yloxy)-phenyl chloroformate and 2-piperidin-4-ylmethyl-pyrimidine. HPLC-MS: m/z=459.1 (M+1); Rₜ=4.44 min.

### Example 67

### General Procedure 7

### 4-Pyrimidin-2-ylmethyl-piperidine-1-carboxylic acid 4-(4-trifluoromethyl-benzyl)-phenyl ester

The title compound (34%) was prepared as an oil using 4-[4-(trifluoromethyl)benzyl]phenyl chloroformate and 2-piperidin-4-ylmethyl-pyrimidine. HPLC-MS: m/z=456.1 (M+1); Rₜ=5.00 min.

### Example 68

### General Procedure 7

### 4-Thiophen-3-ylmethyl-piperidine-1-carboxylic acid 4-(5-trifluoromethyl-pyridin-2-yloxy)-phenyl ester

The title compound (39%) was prepared as an oil using 4-(5-trifluoromethyl-pyridin-2-yloxy)-phenyl chloroformate and 4-thiophen-3-ylmethyl-piperidine. HPLC-MS: m/z=463.0 (M+1); Rₜ=5.48 min.

### Example 69

### General Procedure 7

### 4-Thiophen-3-ylmethyl-piperidine-1-carboxylic acid 4-(4-trifluoromethyl-benzyl)-phenyl ester

The title compound (52%) was prepared as an oil using 4-[4-(trifluoromethyl)benzyl]phenyl chloroformate and 4-thiophen-3-ylmethyl-piperidine. HPLC-MS: m/z=460.1 (M+1); Rₜ=6.01 min.

### Example 70

### 4-Thiophen-3-ylmethyl-piperidine-1-carboxylic acid 4-(5-methyl-pyridin-2-ylmethyl)-phenyl ester

To a solution of 4-(5-methyl-pyridin-2-ylmethyl)phenol (1.0 mmol) and ethyidiisopropylamine (1.5 mmol) in CH₂Cl₂ (3 mL) at −30 ° C. was added trichloromethyl chloroformiate (1.5 mmol). The solution was stirred at −30 ° C. for 30 min and at reflux temperature for 1 h. The solution was evaporated to dryness and redissolved in CH₂Cl₂ (3 mL) and cooled to 0° C. before addition of 4-thiophen-3-ylmethyl-piperidine (1.0 mmol) and ethyidiisopropylamine (1.0 mmol). The solution was stirred at room temperature for 16 h evaporated to give the crude product which was purified by FC (Quad flash 40 CH₂Cl₂:Et₂O:Heptane:Et₃N 1:1:2:0.25->1:1:1:0.25) to give the title compound in 17% yield as a yellow oil. HPLC-MS: m/z=407.0 (M+1); Rₜ=3.49 min.

### Example 71

### General Procedure 7

### 4-(5-Methyl-pyridin-2-ylmethyl)-piperidine-1-carboxylic acid 4-(4-trifluoromethyl-benzyl)-phenyl ester

The title compound (75%) was prepared as crystals using 4-[4-(trifluoromethyl)benzyl]phenyl chloroformate and 5-methyl-2-piperidin-4-ylmethyl-pyridine. HPLC-MS: m/z=469.1 (M+1); Rₜ=3.79 min.

### Example 72

### General Procedure 7

### 4-(5-Methyl-pyridin-2-ylmethyl)-piperidine-1-carboxylic acid 4-(5-trifluoromethyl-pyridin-2-yloxy)-phenyl ester

The title compound (54%) was prepared as an oil using 4-(5-trifluoromethyl-pyridin-2-yloxy)-phenyl chloroformate and 5-methyl-2-piperidin-4-ylmethyl-pyridine. HPLC-MS: m/z=472.2 (M+1); Rₜ=3.35 min.

### Example 73

### 4-(4-Carboxymethyl-benzyl)-piperidine-1-carboxylic acid 4-(5-trifluoromethyl-pyridin-2-yloxy)-phenyl ester

To a solution of (4-piperidin-4-ylmethyl-phenyl)-acetic acid (0.33 mmol) in dichloromethane (5 mL) at 0° C. was added sodium bis(trimethylsilyl)amide (0.66 mmol, 1 M in THF) and stirring was continued for 30 min. The mixture was added 4-(5-trifluoromethyl-pyridin-2-yloxy)-phenyl chloroformate (0.66 mmol) and stirred at room temperature over night and evaporated to dryness. The crude product was redissolved in MeCN and purified by preparative HPLC (Gilson) to give 44% of the title compound as light yellow crystals. HPLC-MS: m/z=515.0 (M+1); Rₜ=4.75 min.

### Example 74

### 4-(4-Carboxymethyl-benzyl)-piperidine-1-carboxylic acid 4-(4-trifluoromethyl-benzyl)-phenyl ester

To a solution of (4-piperidin-4-ylmethyl-phenyl)-acetic acid (0.33 mmol) in dichloromethane (5 mL) at 0° C. was added sodium bis(trimethylsilyl)amide (0.66 mmol, 1M in THF) and stirring was continued for 30 min. The mixture was added 4-[4-(trifluoromethyl)benzyl]phenyl chloroformate (0.66 mmol) and stirred at room temperature over night and evaporated to dryness. The crude product was redissolved in MeCN and purified by preparative HPLC (Gilson) to give 30% of the title compound. HPLC-MS : m/z=512.2 (M+1); Rₜ=4.86 min.

### Example 75

### General Procedure 7

### 4-[4-(2H-Tetrazol-5-ylmethyl)-benzyl]-piperidine-1-carboxylic acid 4-(5-trifluoromethyl-pyridin-2-yloxy)-phenyl ester

The title compound (44%) was prepared as an oil using 4-(5-trifluoromethyl-pyridin-2-yloxy)-phenyl chloroformate and 4-[4-(2H-tetrazol-5-ylmethyl)-benzyl]-piperidine . HPLC-MS: m/z=539.1 (M+1)

### Example 76

### General Procedure 7

### 4-[4-(2H-Tetrazol-5-ylmethyl)-benzyl]-piperidine-l-carboxylic acid 4-(4-trifluoromethyl-benzyl)-phenyl ester

The title compound (35%) was prepared as an oil using 4-[4-(trifluoromethyl)benzyl]phenyl chloroformate and 4-[4-(2H-tetrazol-5-ylmethyl)-benzyl]-piperidine. NMR (400 MHz; CDCl₃): δ 1.11-1.32 (m, 2H), 1.68-1.75 (m, 3H), 2.45-2.60 (m, 2H), 2.74 (t, 1H), 2.91 (t, 1H), 3.97 (s, 2H), 4.10-4.30 (m, 4H), 7.00 (dd, 2H), 7.04-7.14 (m, 6H), 7.23 (d, 2H), 7.51 (d, 2H).

### Example 77

### General Procedure 7

### 4-(4-Methyl-pyridin-2-ylmethyl)-piperidine-1-carboxylic acid 4-(5-trifluoromethyl-pyridin-2-yloxy)-phenyl ester

The title compound (15%) was prepared as an oil using 4-(5-trifluoromethyl-pyridin-2-yloxy)-phenyl chloroformate and 4-methyl-2-piperidin-4-ylmethyl-pyridine. HPLC-MS : m/z=472.1 (M+1); Rₜ=3.36 min

### Example 78

### General Procedure 7

### 4-[4-(1H-Tetrazol-5-ylmethyl)-phenoxy]-piperidine-1-carboxylic acid 4-(5-trifluoromethyl-pyridin-2-yloxy)-phenyl ester

The title compound (4%) was prepared as an oil using 4-(5-trifluoromethyl-pyridin-2-yloxy)-phenyl chloroformate and 4-[4-(1H-tetrazol-5-ylmethyl)-phenoxy]-piperidine. HPLC-MS: m/z=541.2 (M+1); Rₜ=4.04 min

### Example 79

### General Procedure 7

### 4-(6-Hydroxymethyl-pyridin-2-ylmethyl)-piperidine-1-carboxylic acid 4-(5-trifluoromethyl-pyridin-2-yloxy)-phenyl ester

The title compound (11%) was prepared as an oil using 4-(5-trifluoromethyl-pyridin-2-yloxy)-phenyl chloroformate and (6-piperidin-4-ylmethyl-pyridin-2-yl)-methanol which was in situ silyl protected prior to addition by treatment with 1 equiv. trimethylsilyl chloride and 1 equiv. DIPEA. HPLC-MS: m/z=488.0 (M+1); Rₜ=3.21 min

### Example 80

### General Procedure 7

### 4-[4-(1H-Tetrazol-5-ylmethyl)-phenoxy]-piperidine-1-carboxylic acid 4-(4-trifluoromethyl-benzyl)-phenyl ester

The title compound (3%) was prepared as an oil using 4-[4-(trifluoromethyl)benzyl]phenyl chloroformate and 4-[4-(1H-tetrazol-5-ylmethyl)-phenoxy]-piperidine. HPLC-MS: m/z=538.2 (M+1); Rₜ=4.46 min

### Example 81

### General Procedure 7

### 4-(6-Hydroxymethyl-pyridin-2-ylmethyl)-piperidine-1-carboxylic acid 4-(4-trifluoromethyl-benzyl)-phenyl ester

The title compound (8%) was prepared as an oil using 4-[4-(trifluoromethyl)benzyl]phenyl chloroformate and (6-piperidin-4-ylmethyl-pyridin-2-yl)-methanol which was in situ silyl protected prior to addition by treatment with 1 equiv. trimethylsilyl chloride and 1 equiv. DIPEA. HPLC-MS: m/z=485.2 (M+1); Rₜ=3.62 min

### Example 82

### General Procedure 7

### 4-(4-Methyl-pyridin-2-ylmethyl)-piperidine-1-carboxylic acid 4-(4-trifluoromethyl-benzyl)-phenyl ester

The title compound (24%) was prepared as it's TFA salt using 4-[4-(trifluoromethyl)benzyl]phenyl chloroformate and 4-methyl-2-piperidin-4-ylmethyl-pyridine. HPLC-MS: m/z=469.1 (M+1); Rₜ=3.75 min

### Example 83

### 4-(4-Methyl-pyridin-2-ylmethyl)-piperidine-1-carboxylic acid 4-(5-methyl-pyridin-2-ylmethyl)-phenyl ester

To a solution of 4-(5-methyl-pyridin-2-ylmethyl)phenol (1.0 mmol) and ethyidiisopropylamine (1.5 mmol) in CH₂Cl₂ (3 mL) at −30° C. was added trichloromethyl chloroformiate (1.5 mmol). The solution was stirred at −30° C. for 30 min and at reflux temperature for 30 min. The solution was evaporated to dryness and redissolved in CH₂Cl₂ (3 mL) and cooled to 0° C. before addition of 4-methyl-2-piperidin-4-ylmethyl-pyridine (1.0 mmol) and ethyidiisopropylamine (1.0 mmol). The solution was stirred at room temperature for 16 h evaporated to give the crude product which was purified by preparative HPLC (Gilson) to give the title compound as it's TFA salt in 19% yield as an oil. HPLC-MS : m/z=416.2 (M+1); Rₜ=1.90 min.

### Example 84

### (Carboxymethyl-phenoxy)-piperidine-1-carboxylic acid 4-(3-methyl-butylcarbamoyl)-phenyl ester

The title compound was prepared according to general procedures 8 and 9 applying the following amine: Methyl-(3-methyl-butyl)-amine. LC-MS: m/z: 469.2 (M+); Rₜ=3.83 min.

### Example 85

### 4-(4-Carboxymethyl-phenoxy)-piperidine-1-carboxylic acid 4-phenethylcarbamoyl-phenyl ester

The title compound was prepared according to general procedures 8 and 9 applying the following amine: Phenethylamine. LC-MS: m/z: 503.2 (M+); Rt=3.82 min.

### Example 86

### 4-(4-Carboxymethyl-phenoxy)-piperidine-1-carboxylic acid 4-benzylcarbamoyl-phenyl ester

The title compound was prepared according to general procedure 8 and 9 applying the following amine: benzylamine. LC-MS: m/z: 489.2 (M+); Rt=3.72 min.

### Example 87

### 4-(4-Carboxymethyl-phenoxy)-piperidine-1-carboxylic acid 4-[(bicyclo[2.2.1 ]hept-2-ylmethyl)-carbamoyl]-phenyl ester

The title compound was prepared according to general procedures 8 and 9 applying the following amine: Bicyclo[2.2.1]hept-2-methylamine (C-bicyclo[2.2.1]hept-2-yl-methylamine). LC-MS: m/z: 507.5 (M+); Rt=3.95 min.

### Example 88

### 4-(4-Carboxymethyl-phenoxy)-piperidine-1-carboxylic acid 4-(cyclohexylmethyl-carbamoyl)-phenyl ester

The title compound was prepared according to general procedures 8 and 9 applying the following amine: Aminomethyl-cyclohexane (C-cyclohexyl-methylamine). LC-MS: m/z: 495.2 (M+); Rt=4.16 min.

The title compound was prepared according to general procedures 8 and 9 applying the following amine: 2-(4-fluoro-phenyl)-1,1-dimethyl-ethylamine. LC-MS: m/z: 549.3 (M+); Rt=4.41 min.

### Example 89

### 4-(4-Carboxymethyl-phenoxy)-piperidine-1-carboxylic acid 4-(3,3-dimethyl-butylcarbamoyl)-phenyl ester

The title compound was prepared according to general procedures 8 and 9 applying the following amine: 3,3-dimethylbutylamine. LC-MS: m/z: 483.3 (M+); Rt=4.03 min.

### Example 90

### 4-(4-Carboxymethyl-phenoxy)-piperidine-1-carboxylic acid 4-[(thiophen-2-ylmethyl)-carbamoyl]-phenyl ester

The title compound was prepared according to general procedures 8 and 9 applying the following amine: Thiophene-2-methylamine. LC-MS: m/z: 495.0 (M+); Rt=3.70 min.

### Example 91

### 4-(4-Carboxymethyl-phenoxy)-piperidine-1-carboxylic acid 4-(2-thiophen-2-yl-ethylcarbamoyl)-phenyl ester

The title compound was prepared according to general procedures 8 and 9 applying the following amine: 2-thiophen-2-ethylamine. LC-MS: m/z: 509.1 (M+); Rt=3.83 min.

### Example 92

### 4-(4-Carboxymethyl-phenoxy)-piperidine-1-carboxylic acid 4-(2-phenoxy-ethyl)-phenyl ester

The title compound was prepared according to general procedure 10 applying the following mitzunobu reagent: phenol. LC-MS: m/z: 510.1 (M+); Rt=4.95 min.

### Example 93

### 4-(4-Carboxymethyl-phenoxy)-piperidine-1-carboxylic acid 4-[2-(4-chloro-phenoxy)-ethyl]-phenyl ester

The title compound was prepared according to general procedure 10 applying the following mitzunobu reagent: 4-chlorphenol. LC-MS: m/z: 476.2 (M+); Rt=4.67 min.

### Example 94

### 4-(4-Carboxymethyl-phenoxy)-piperidine-1-carboxylic acid 4-[2-(1-methyl-1H-tetrazol-5-yloxy)-ethyl]-phenyl ester

The title compound was prepared according to general procedure 10 applying the following mitzunobu reagent: 1-methyl-1H-tetrazol-5-ol. LC-MS: m/z: 498.1 (M+); Rt=3.78 min.

### Example 95

### 4-(4-Carboxymethyl-phenoxy)-piperidine-1-carboxylic acid 4-[2-(3-chloro-phenoxy)-ethyl]-phenyl ester

The title compound was prepared according to general procedure 10 applying the following mitzunobu reagent: 3-chlorphenol. LC-MS: m/z: 51 0.1 (M+); Rt=4.97 min.

### Example 96

### 4-(4-Carboxymethyl-phenoxy)-piperidine-1-carboxylic acid 4-[2-(4-trifluoromethoxy-phenoxy)-ethyl]-phenyl ester

The title compound was prepared according to general procedure 10 applying the following mitzunobu reagent: 4-triflouromethoxy-phenol. LC-MS: m/z: 560.2 (M+); Rt=5.10 min.

### Example 97

### 4-(4-Carboxymethyl-phenoxy)-piperidine-1-carboxylic acid 4-[2-(4-[1,2,4]triazol-4-yl-phenoxy)-ethyl]-phenyl ester

The title compound was prepared according to general procedure 10 applying the following mitzunobu reagent: 4-[1,2,4]Triazol4-yl-phenol. LC-MS: m/z: 543.2 (M+); Rt=4.07 min.

### Example 98

### 4-(4-Carboxymethyl-phenoxy)-piperidine-1-carboxylic acid 4-(2-phenylsulfanyl-ethyl)-phenyl ester

The title compound was prepared according to general procedure 10 applying the following mitzunobu reagent: benzenethiol. LC-MS: m/z: 492.0 (M+); Rt=4.89 min.

### Example 99

### 4-(4-Carboxymethyl-phenoxy)-piperidine-1-carboxylic acid 4-[2-(2-chloro-phenoxy)-ethyl]-phenyl ester

The title compound was prepared according to general procedure 10 applying the following mitzunobu reagent: 2-chlorophenol. LC-MS: m/z: 510.0 (M+); Rt=4.88 min.

### Example 100

### 4-(4-Carboxymethyl-phenoxy)-piperidine-1-carboxylic acid 4-[2-(4-methyl-4H-[1,2,4]triazol-3-ylsulfanyl)-ethyl]-phenyl ester

The title compound was prepared according to general procedure 10 applying the following mitzunobu reagent: 4-methyl-[1,2,3]triazole-3-thiol. LC-MS: m/z: 497.0 (M+); Rt=3.66 min.

### Example 101

### 4-(4-Carboxymethyl-phenoxy)-piperidine-1-carboxylic acid 4-[2-(4-pyrrol-1-yl-phenoxy)-ethyl]-phenyl ester

The title compound was prepared according to general procedure 10 applying the following mitzunobu reagent: 4-pyrrol-1-yl-phenol. LC-MS: m/z: 541.1 (M+); Rt=4.97 min.

### Example 102

### 4-(4-Carboxymethyl-phenoxy)-piperidine-1-carboxylic acid 4-(3-chloro-5-trifluoromethyl-pyridin-2-ylmethyl)-phenyl ester

The title compound was prepared according to general procedure 11 applying the following phenol: 4-[(3-chloro-5-(trifluoromethyl)-2-pyridinyl)methyl]benzenol. LC-MS: m/z: 549.1 (M+); Rt=4.74 min.

### Example 103

### 4-(4-Carboxymethyl-phenoxy)-piperidine-1-carboxylic acid 4-(4-trifluoromethyl-phenoxy)-phenyl ester

The title compound was prepared according to general procedure 11 applying the following phenol: 4-[(4-trifluoromenthyl)phenoxy]phenol. LC-MS: m/z: 516.1 (M+); Rt=4.91 min.

### Example 104

### 4-(1H-Imidazol-2-ylsulfanyl)-piperidine-1-carboxylic acid 4-(3,3-dimethyl-butylcarbamoyl)-phenyl ester

The title compound was prepared according to general procedure 12 applying the following mitzunobu reagent: 1H-imidazole-2-thiol. LC-MS: m/z: 431.0 (M+); Rt=2.82 min.

### Example 105

### 4-(4-Carboxy-phenoxy)-piperidine-1-carboxylic acid 4-(3,3-dimethyl-butylcarbamoyl)-phenyl ester

a) 4-(4-Allyloxycarbonyl-phenoxy)-piperidine-1-carboxylic acid 4-(3,3-dimethyl-butylcarbamoyl)-phenyl ester was prepared according to general procedure 12 applying the following mitzunobu reagent: 4-Hydroxy-benzoic acid allyl ester.

b) 4-(4-Carboxy-phenoxy)-piperidine-1-carboxylic acid 4-(3,3-dimethyl-butylcarbamoyl)-phenyl ester was prepared by a standard deprotection of the allyl ester with tetrakis(triphenylphosphine) palladium and morpholine in THF. The reaction mixture was purified by flash chromatography dichloromethane/methanol/acetic acid (80:8:4) followed by preparative HPLC. LC-MS: m/z: 496.2 (M+); Rt=4.03 min.

### Example 106

### 4-(4-Carboxy-benzyl)-piperidine-1-carboxylic acid 4-(5-trifluoromethyl-pyridin-2-yloxy)-phenyl ester

a) 4-(4-Methoxycarbonyl-benzyl)-piperidine-1-carboxylic acid 4-(5-trifluoromethyl-pyridin-2-yloxy)-phenyl ester was prepared from 4-(4-methoxycarbonylbenzyl)piperidine hydrochloride and 4-(5-trifluoromethyl-pyridin-2-yloxy)-phenyl chloroformate (prepared from the respective phenol by condensation with a phosgene source) as described in general procedure 4.

b) The title product was prepared from 4-(4-methoxycarbonyl-benzyl)-piperidine-1-carboxylic acid 4-(5-trifluoromethyl-pyridin-2-yloxy)-phenyl ester by an analogous procedure to the one described in general procedure 2. LC-MS: m/z: 501.1 (M+); Rt=4.84 min.

### Example 107

### 4-(1H-Benzoimidazol-2-ylsulfanyl)-piperidine-1-carboxylic acid 4-(5-trifluoromethyl-pyridin-2-yloxy)-phenyl ester

4-Hydroxy-piperidine-1-carboxylic acid 4-(5-trifluoromethyl-pyridin-2-yloxy)-phenyl ester and 2-mercaptobenzimidazole was condensed as described in general procedure 1. LC-MS: m/z: 515.1 (M+); Rt=3.63 min.

### Example 108

### 4-(4-Carboxy-1H-imidazol-2-ylsulfanyl)-piperidine-1-carboxylic acid 4-(5-trifluoromethyl-pyridin-2-yloxy)-phenyl ester

a) 4-(4-Ethoxycarbonyl-1H-imidazol-2-ylsulfanyl)-piperidine-1-carboxylic acid 4-(5-trifluoromethyl-pyridin-2-yloxy)-phenyl ester was prepared from 4-ethoxycarbonylimidazole-2-thiol and 4-Hydroxy-piperidine-1-carboxylic acid 4-(5-trifluoromethyl-pyridin-2-yloxy)-phenyl ester applying general procedure 1.

b) The title product was prepared from 4-(4-ethoxycarbonyl-1H-imidazol-2-ylsulfanyl)-piperidine-1-carboxylic acid 4-(5-trifluoromethyl-pyridin-2-yloxy)-phenyl ester applying general procedure 2. LC-MS: m/z: 509.1 (M+); Rt=3.26 min.

### Example 109

### 6-{1-[4-(5-Trifluoromethyl-pyridin-2-yloxy)-phenoxycarbonyl]-piperidin-4-yloxy}-nicotinic acid

a) 6-{1-[4-(5-Trifluoromethyl-pyridin-2-yloxy)-phenoxycarbonyl]-piperidin-4-yloxy)-nicotinic acid allyl ester was prepared from 6-hydroxy-nicotinic acid allylic ester and 4-Hydroxy-piperidine-1-carboxylic acid 4-(5-trifluoromethyl-pyridin-2-yloxy)-phenyl ester applying general procedure 1.

b) The title product was prepared from 6-{1-[4-(5-trifluoromethyl-pyridin-2-yloxy)-phenoxycarbonyl]-piperidin-4-yloxy}-nicotinic acid allyl ester applying general procedure 3. LC-MS: m/z: 504.1 (M+); Rt=4.36 min.

### Example 110

### 4-(3-Carboxymethyl-phenoxy)-piperidine-1-carboxylic acid 4-(5-trifluoromethyl-pyridin-2-yloxy)-phenyl ester

a) 4-(3-Allyloxycarbonylmethyl-phenoxy)-piperidine-1-carboxylic acid 4-(5-trifluoromethyl-pyridin-2-yloxy)-phenyl ester was prepared from (3-hydroxy-phenyl)-acetic acid allyl ester and 4-Hydroxy-piperidine-1-carboxylic acid 4-(5-trifluoromethyl-pyridin-2-yloxy)-phenyl ester applying general procedure 1.

b) The title product was prepared from 4-(3-allyloxycarbonylmethyl-phenoxy)-piperidine-1-carboxylic acid 4-(5-trifluoromethyl-pyridin-2-yloxy)-phenyl ester applying general procedure 3. LC-MS: m/z: 517.0 (M+); Rt=4.56 min.

### Example 111

### 4-(4-Carboxymethyl-2-fluoro-phenoxy)-piperidine-1-carboxylic acid 4-(5-trifluoromethyl-pyridin-2-yloxy)-phenyl ester

a) 4-(4-Allyloxycarbonylmethyl-2-fluoro-phenoxy)-piperidine-1-carboxylic acid 4-(5-trifluoromethyl-pyridin-2-yloxy)-phenyl ester was prepared from (3-fluoro-4-hydroxy-phenyl)-acetic acid allyl ester and 4-Hydroxy-piperidine-1-carboxylic acid 4-(5-trifluoromethyl-pyridin-2-yloxy)-phenyl ester applying general procedure 1.

b) The title product was prepared from 4-(4-allyloxycarbonylmethyl-2-fluoro-phenoxy)-piperidine-1-carboxylic acid 4-(5-trifluoromethyl-pyridin-2-yloxy)-phenyl ester applying general procedure 3. LC-MS: m/z: 535.0 (M+); Rt=4.58 min.

### Example 112

### 4-[3-(2-Carboxy-ethyl)-phenoxy]-piperidine-1-carboxylic acid 4-(5-trifluoromethyl-pyridin-2-yloxy)-phenyl ester

a) 4-[3-(2-Allyloxycarbonyl-ethyl)-phenoxy]-piperidine-1-carboxylic acid 4-(5-trifluoromethyl-pyridin-2-yloxy)-phenyl ester was prepared from 3-(3-hydroxy-phenyl)-propionic acid allyl ester and 4-hydroxy-piperidine-1-carboxylic acid 4-(5-trifluoromethyl-pyridin-2-yloxy)-phenyl ester applying general procedure 1.

b) The title product was prepared from 4-[3-(2-allyloxycarbonyl-ethyl)-phenoxy]-piperidine-1-carboxylic acid 4-(5-trifluoromethyl-pyridin-2-yloxy)-phenyl ester applying general procedure 3. LC-MS: m/z: 531.1 (M+); Rt=4.71 min.

### Example 113

### 4-(5-Carboxymethylsulfanyl-[1,3,4]thiadiazol-2-ylsulfanyl)-piperidine-1-carboxylic acid 4-(5-trifluoromethyl-pyridin-2-yloxy)-phenyl ester

a) 4-(5-Allyloxycarbonylmethylsulfanyl-[1,3,4]thiadiazol-2-ylsulfanyl)-piperidine-1-carboxylic acid 4-(5-trifluoromethyl-pyridin-2-uloxy)-phenyl ester was prepared from 5-mercapto-[1,3,4]thiadiazol-2-ylsulfanyl)-acetic acid allyl ester and 4-hydroxy-piperidine-1-carboxylic acid 4-(5-trifluoromethyl-pyridin-2-yloxy)-phenyl ester applying general procedure 1.

b) The title product was prepared from 4-(5-allyloxycarbonylmethylsulfanyl-[1,3,4]thiadiazol-2-ylsulfanyl)-piperidine-1-carboxylic acid 4-(5-trifluoromethyl-pyridin-2-uloxy)-phenyl ester applying general procedure 3. LC-MS: m/z: 573.0 (M+); Rt=4.63 min.

### Example 114

### 4-(5-carboxymethyl-4-methyl-oxazol-2-ylsulfanyl)-piperidine-1-carboxylic acid 4-(5-trifluoromethyl-pyridin-2-yloxy)-phenyl ester

a) 4-(5-Allyloxycarbonylmethyl-4-methyl-oxazol-2-ylsulfanyl)-piperidine-1-carboxylic acid 4-(5-trifluoromethyl-pyridin-2-yloxy)-phenyl ester was prepared from 2-mercapto-4-methyl-oxazol-5-yl)-acetic acid allyl ester and 4-hydroxy-piperidine-1-carboxylic acid 4-(5-trifluoromethyl-pyridin-2-yloxy)-phenyl ester applying general procedure 1.

b) The title product was prepared from 4-(5-allyloxycarbonylmethyl-4-methyl-oxazol-2-ylsulfanyl)-piperidine-1-carboxylic acid 4-(5-trifluoromethyl-pyridin-2-yloxy)-phenyl ester applying general procedure 3. LC-MS: m/z: 576.0 (M+39); Rt=4.37 min.

### Example 115

### 4-(4-Carboxymethoxy-phenoxy)-piperidine-1-carboxylic acid 4-(5-trifluoromethyl-pyridin-2-yloxy)-phenyl ester

a) 4-(4-Allyloxycarbonylmethoxy-phenoxy)-piperidine-1-carboxylic acid 4-(5-trifluoromethyl-pyridin-2-yloxy)-phenyl ester was prepared from (4-hydroxy-phenoxy)-acetic acid allyl ester and 4-hydroxy-piperidine-1-carboxylic acid 4-(5-trifluoromethyl-pyridin-2-yloxy)-phenyl ester applying general procedure 1.

b) The title product was prepared from 4-(4-allyloxycarbonylmethoxy-phenoxy)-piperidine-1-carboxylic acid 4-(5-trifluoromethyl-pyridin-2-yloxy)-phenyl ester applying general procedure 3. LC-MS: m/z: 533.1 (M+); Rt=4.48 min.

### Example 116

### 4-(4-Carboxymethyl-phenoxy)-piperidine-1-carboxylic acid 4-(3-trifluoromethyl-phenoxy)-phenyl ester

a) 4-(3-Trifluoromethyl-phenoxy)-phenol was prepared from [3-(4-benzyloxy-phenoxy)-phenyl]-triflouromethyl hydroquinone monobenzylether and 3-chlorophenylboronic acid (Cu(II)acetate, TEA in dichloromethane).

b) 4-(4-Allyloxycarbonylmethyl-phenoxy)-piperidine-1-carboxylic acid 4-(3-trifluoromethyl-phenoxy)-phenyl ester was prepared by a condensation between 3-[4-(4-allyloxycarbonylmethyl-phenoxy)-piperidine-1-carbonyl]-1-methyl-3H-imidazol-1-ium; iodide and 4-(3-trifluoromethyl-phenoxy)-phenol

c) The title product was prepared from 4-(4-allyloxycarbonylmethyl-phenoxy)-piperidine-1-carboxylic acid 4-(3-trifluoromethyl-phenoxy)-phenyl ester applying general procedure 3. LC-MS: m/z: 516.1 (M+); Rt=4.90 min.

### Example 117

### 4-(4-Carboxymethyl-phenoxy)-piperidine-1-carboxylic acid 4-(5-chloro-pyridin-2-yloxy)-phenyl ester

a) 4-(5-Chloro-pyridin-2-yloxy)-phenol was prepared from 2,5-dichloropyridin and hydroquinone (KOH in DMSO, 90° C. 1 h.).

b) 4-(4-Allyloxycarbonylmethyl-phenoxy)-piperidine-1-carboxylic acid 4-(5-chloro-pyridin-2-yloxy)-phenyl ester was prepared by a condensation between 3-[4-(4-allyloxycarbonylmethyl-phenoxy)-piperidine-1-carbonyl]-1-methyl-3H-imidazol-1-ium; iodide and 4-(5-chloro-pyridin-2-yloxy)-phenol

c) The title product was prepared from 4-(4-allyloxycarbonylmethyl-phenoxy)-piperidine-1-carboxylic acid 4-(5-chloro-pyridin-2-yloxy)-phenyl ester applying general procedure 3. LC-MS: m/z: 483.1 (M+); Rt=4.37 min.

### Example 118

### 4-(4-Carboxymethyl-phenoxy)-piperidine-1-carboxylic acid 4-(3-chloro-5-trifluoromethyl-pyridin-2-yloxy)-phenyl ester

a) 4-(3-Chloro-5-trifluoromethyl-pyridin-2-yloxy)-phenol was prepared from 2,3-dichloro-5-(trifluoromethyl)pyridine and hydroquinone (KOH in DMSO).

b) 4-(4-Allyloxycarbonylmethyl-phenoxy)-piperidine-1-carboxylic acid 4-(3-chloro-5-trifluoromethyl-pyridin-2-yloxy)-phenyl ester was prepared by a condensation between 3-[4-(4-allyloxycarbonylmethyl-phenoxy)-piperidine-1-carbonyl]-1-methyl-3H-imidazol-1-ium; iodide and 4-(3-chloro-5-trifluoromethyl-pyridin-2-yloxy)-phenol.

c) The title product was prepared from 4-(4-allyloxycarbonylmethyl-phenoxy)-piperidine-1-carboxylic acid 4-(3-chloro-5-trifluoromethyl-pyridin-2-yloxy)-phenyl ester applying general procedure 3. LC-MS: m/z: 551.1 (M+); Rt=4.86 min.

### Example 119

### 4-(4-Carboxymethyl-phenoxy)-piperidine-1-carboxylic acid 4-p-tolyloxy-phenyl ester

a) 4-(4-Allyloxycarbonylmethyl-phenoxy)-piperidine-1-carboxylic acid 4-hydroxy-phenyl ester was prepared by a condensation between 3-[4-(4-allyloxycarbonylmethyl-phenoxy)-piperidine-1-carbonyl]-1-methyl-3H-imidazol-1-ium; iodide and hydroq uinone (DI PEA, THF).

b) 4-(4-Allyloxycarbonylmethyl-phenoxy)-piperidine-1-carboxylic acid 4-p-tolyloxy-phenyl ester was prepared from 4-(4-allyloxycarbonylmethyl-phenoxy)-piperidine-1-carboxylic acid 4-hydroxy-phenyl ester and 4-methylbenzenboronic acid (Cu(II)acetate, TEA in dichloromethane).

c) The title product was prepared from 4-(4-allyloxycarbonylmethyl-phenoxy)-piperidine-1-carboxylic acid 4-p-tolyloxy-phenyl ester applying general procedure 3. LC-MS: m/z: 462.1 (M+); Rt=4.83 min.

### Example 120

### 4-(Pyridin-2-ylsulfanylmethyl)-piperidine-1-carboxylic acid 4-(5-trifluoromethyl-pyridin-2-yloxy)-phenyl ester

The title compound was prepared according to general procedure 13 from 4-hydroxymethyl-piperidine-1-carboxylic acid 4-(5-trifluoromethyl-pyridin-2-yloxy)-phenyl ester and 2-mercaptopyridine. Purification by prep. HPLC. LC-MS: m/z: 490.2 (M+); Rt=4.64 min.

### Example 121

### 4-(Pyridin-2-yloxymethyl)-piperidine-1-carboxylic acid 4-(5-trifluoromethyl-pyridin-2-yloxy)-phenyl ester

The title compound was prepared according to general procedure 13 from 4-hydroxymethyl-piperidine-1-carboxylic acid 4-(5-trifluoromethyl-pyridin-2-yloxy)-phenyl ester and 2-hydroxypyridine. Purification by prep. HPLC. LC-MS: m/z: 474.1 (M+); Rt=4.47 min.

### Example 122

### 4-(1H-Imidazol-2-ylsulfanyl)-piperidine-1-carboxylic acid 4-(5-methylsulfanyl-pyridin-2-yloxy)-phenyl ester

The title compound was prepared according to general procedure 13 from 4-Hydroxy-piperidine-1-carboxylic acid 4-(5-methylsulfanyl-pyridin-2-yloxy)-phenyl ester and 2-mercaptoimidazole.

4-Hydroxy-piperidine-1-carboxylic acid 4-(5-methylsulfanyl-pyridin-2-yloxy)-phenyl ester was prepared from 5-bromo-2-[4-(tert-butyl-dimethyl-silanyloxy)-phenoxy]-pyridine via 4-(5-Methylsulfanyl-pyridin-2-yloxy)-phenol. Purification by prep. HPLC. LC-MS: m/z: 465.1 (M+23); Rt=2.90 min.

### Example 123

### 4-(1H-Tetrazol-5-ylsulfanyl)-piperidine-1-carboxylic acid 4-(5-trifluoromethyl-pyridin-2-yloxy)-phenyl ester

### a) 4-Thiocyanatopiperidine-1-carboxylic acid 4-(5-trifluoromethyl-pyridin-2-yloxy)-phenyl ester

Diethyl azodicarboxylate (824 μL, 5.23 mmol) was added to a mixture of triphenylphosphine (1.37 g, 5.23 mmol) in dry acetonitrile (20 ml) with stirring at 0° C. under nitrogen. Then ammonium thiocyanate (398 mg, 5.23 mmol) was added at room temperature followed by a solution of 4-hydroxypiperidine-1-carboxylic acid 4-(5-trifluoromethyl-pyridin-2-yloxy)-phenyl ester (1.0 g, 2.62 mmol) in 10 mL of dry acetonitril and the mixture was stirred overnight. The solvents were evaporated to dryness and the residue was purified by preparative HPLC (Gilson) to give 175 mg (16%) of almost pure thiocyanate. ¹H NMR (400 MHz, CDCl₃): δ 1.83-1.98 (br, 2 H), 2.15-2.28 (m, 2 H), 3.05-3.32 (m, 2 H), 3.41-3.50 (m, 1 H), 4.20-4.35 (m, 2 H), 7.01 (d, J=8.59 Hz, 1 H), 7.10-7.22 (m, 4 H), 7.90 (dd, J=8.59, 2.53 Hz, 1 H), 8.43 (br s, 1 H); HPLC-MS: m/z=424(M+1)⁺, Rt=4.2 min.

### b) 4-(1H-Tetrazol-5-ylsulfanyl)-piperidine-1-carboxylic acid 4-(5-trifluoromethyl-pyridin-2-yloxy)-phenyl ester

A mixture of 4-thiocyanatopiperidine-1-carboxylic acid 4-(5-trifluoromethyl-pyridin-2-yloxy)-phenyl ester (175 mg, 0.41 mmol), sodium azide (30 mg, 0.46 mmol) and zinc bromide (93 mg, 0.41 mmol) in 3 mL of water was heated in a microwave oven at 160° C. for 60 min. The solvent was decanted and the residue was taken up in acetonitrile/TFA (99:1) and purified by preparative HPLC (Gilson) to give 12 mg (6%) of the title compound as a colorless oil. ¹H NMR (400 MHz, CDCl₃): δ 1.74-1.91 (m, 2 H), 2.19-2.34 (m, 2 H), 3.14-3.41 (m, 2 H), 3.97-4.06 (m, 1 H), 4.10-4.27 (m, 2 H), 7.02 (d, J=8.59 Hz, 1 H), 7.11-7.19 (m, 4 H), 7.93 (dd, J=8.59, 2.53 Hz, 1 H), 8.43 (s, 1 H); HPLC-MS: m/z=467 (M+1)⁺, Rt=3.7 min.

### Example 124

### 4-(4-Carboxymethyl-phenoxy)-piperidine-1-carboxylic acid 4-(4-trifluoromethyl-benzyl)-phenyl ester

### a) 4-(4-Methoxycarbonylmethyl-phenoxy)-piperidine-1-carboxylic acid tert-butyl ester

A solution of tert-butyl 4-hydroxypiperidine-1-carboxylate (55.0 g, 273 mmol) and diethyl azodicarboxylate (43.0 mL, 273 mmol) in dry tetrahydrofuran (250 ml) was added during 2 h to a stirred solution of methyl (4-hydroxyphenyl)acetate (36.3 g, 218 mmol) and triphenylphosphine (71.7 g, 273 mmol) in dry tetrahydrofuran (300 ml) at 0° C. under nitrogen. The mixture was stirred over night at room temperature and then evaporated to approximately half of its volume. Some solid triphenylphosphine oxide was removed by trituration with diethyl ether (175 mL). The filtrate was evaporated to dryness and the trituration was repeated with ethyl acetate/heptane (1:1). After filtration, the filtrate was evaporated to dryness and the residue was purified by flash column chromatography (SiO₂, heptane/ethyl acetate (7:3)) to give 54.4g (71%) of the pure product as a colourless oil. ¹H NMR (400 MHz, CDCl₃): δ 1.47 (s, 9 H), 1.69-1.78 (m, 2 H), 1.85-1.94 (m, 2 H), 3.29-3.37 (m, 2 H), 3.56 (s, 2 H), 3.65-3.73 (m, 2 H), 3.68 (s, 3 H), 4.40-4.47 (m, 1 H), 6.86 (d, J=8.59 Hz, 2 H), 7.18 (d, J=8.59 Hz, 2 H); HPLC: m/z=372(M+23)⁺, 250 ((M−100)+1)⁺, Rt=4.5 min.

### b) [4-(Piperidin-4-yloxy)phenyl]acetic acid

A mixture of 4-(4-methoxycarbonylmethyl-phenoxy)-piperidine-1-carboxylic acid tert-butyl ester (23.4 g, 67.1 mmol) in 6N hydrochloric acid (200 mL) was heated at 95° C. with stirring over night. The cooled solution was evaporated to dryness to give a quantitative yield (20.5 g) of the crude deprotected product as its hydrochloride. ¹H NMR (400 MHz, DMSO-d₆): δ 1.78-1.89 (m, 2 H), 2.05-2.15 (m, 2 H), 2.98-3.10 (m, 2 H), 3.14-3.25 (m, 2 H), 3.49 (s, 2 H), 4.58-4.66 (m, 1 H), 6.94 (d, J=8.59 Hz, 2 H), 7.18 (d, J=8.59 Hz, 2 H), 9.13 (br s, 1 H), 9.19 (br s, 1 H); HPLC-MS: m/z=236 (M+1)⁺, Rt=1.3 min.

To a stirred solution of the crude hydrochloride (20.5 g) in water (100 mL) was added a solution of 10N sodium hydroxide (ca. 15 mL) until the precipitated free acid intermediate was dissolved at pH 11. The solution was evaporated to dryness to give 23.2 g of almost pure sodium [4-(piperidin-4-yloxy)phenyl]acetate (as a mixture with sodium chloride). ¹H NMR (400 MHz, DMSO-d₆) δ 1.32-1.46 (m, 2 H), 1.83-1.91 (m, 2 H), 2.51-2.57 (m, 2 H), 2 H), 3.15 (s, 2 H), 4.23-4.33 (m, 1 H), 6.76 (d, J=8.59 Hz, 2 H), 7.11 (d, J=8.59 Hz, 2 H).

### c) 4-(4-Carboxymethyl-phenoxy)-piperidine-1-carboxylic acid 4-(4-trifluoromethyl-benzyl)-phenyl ester

A solution of 4-[4-(trifluoromethyl)benzyl]phenyl chloroformate (294 mg, 0.93 mmol) in dichloromethane (5 mL) was added during 5 min to a stirred suspension of sodium [4-(piperidin-4-yloxy)phenyl]acetate/sodium chloride mixture from above (241 mg) in dichloromethane (5 mL) at 0° C. under nitrogen. The mixture was stirred for 3 h in the melting ice bath. The mixture was evaporated to dryness and the residue was purified by flash column chromatography (SiO₂, ethyl acetate/heptane (1:1)) to give 173 mg of the pure product as a colourless oil. ¹H NMR (400 MHz, CDCl₃) δ 1.80-2.05 (m, 4 H), 3.49-3.68 (m, 2 H), 3.56 (s, 2 H), 3.68-3.91 (m, 2 H), 4.01 (s, 2 H), 4.49-4.56 (m, 1 H), 6.88 (d, J=8.59 Hz, 2 H), ca. 6.9 (very br s, 1 H), 7.04 (d, J=8.59 Hz, 2 H), 7.14 (d, J=8.59 Hz, 2 H), 7.20; HPLC-MS: m/z =514 (M+1)⁺, Rt=4.9 min.

### Example 125

### 4-(1H-Imidazol-2-ylsulfanyl)-piperidine-1-carboxylic acid 4-(4-trifluoromethyl-benzyl)-phenyl ester

To a stirred solution of 4-hydroxy-piperidine-1-carboxylic acid 4-(4-trifluoromethyl-benzyl)-phenyl ester (379 mg, 1.0 mmol) and 1H-imidazole-2-thiol (100 mg, 1.0 mmol) in dry tetrahydrofuran (10 mL) was added 1,1′-(azodicarbonyl)dipiperidine (378 mg, 1.5 mmol) at room temperature under nitrogen in a Schlenk tube. Tributylphosphine (303 mg, 1.5 mmol) was added to the yellow solution to give a voluminous grey suspension which was stirred at room temperature for 3 days. The resulting white suspension was filtered and the filtrate was evaporated to dryness. The residue was triturated with dichloromethane to give 229 mg (50%) of almost pure title compound as the free base. ¹H NMR (400 MHz, DMSO-d₆) δ 1.39-1.62 (m, 2 H), 1.89-2.01 (m, 2 H), 2.94-3.26 (m, 2 H), 3.38-3.48 (m, 1 H), 3.81-4.09 (m, 2 H), 4.04 (s, 2 H), 6.99 (br s, 1 H), 7.03 (d, J=8.08 Hz, 2 H), 7.21 (br s, 1 H), 7.24 (d, J=8.08 Hz, 2 H), 7.46 (d, J=8.08 Hz, 2 H), 7.65 (d, J=8.08 Hz, 2 H), 12.39 (br s,1 H); HPLC-MS: m/z=462 (M+1)⁺, Rt=3.82 min.

The free base (225 mg, 0.48 mmol) was dissolved in boiling ethanol (25 mL) and filtered. To the cooled filtrate was added a solution of hydrogen chloride in diethyl ether (3.2 M, 20 mL) and the solution was evaporated to dryness. The residue was triturated with petroleum benzene to give 208 mg (44%) of the title compound as its hydrochloride. ¹H NMR (400 MHz, CDCl₃) δ 1.51-1.74 (m, 2 H), 1.95-2.10 (m, 2 H), 3.04-3.38 (m, 2 H), 3.99 (s, 2 H), 4.05-4.28 (m, 3 H), 7.01 (d, J=8.08 Hz, 2 H), 7.14 (d, J=8.08 Hz, 2 H), 7.27 (d, J=8.08 Hz, 2 H), 7.27 (s, 2 H), 7.52 (d, J=8.08 Hz, 2 H), 15.03 (br s, 2 H).

### Example 126

### 4-(2-Bromo-4-carboxymethyl-phenoxy)-piperidine-1-carboxylic acid 4-(5-trifluoromethyl-pyridin-2-yloxy)-phenyl ester

### a) [3-Bromo-4-(piperidin-4-yloxy)-phenyl]-acetic acid

Bromine (0.15 mL, 2.9 mmol) was added to a stirred suspension of 4-(4-methoxycarbonylmethyl-phenoxy)-piperidine-1-carboxylic acid tert-butyl ester (0.5 g, 1.43 mmol) in acetic acid (5 mL) at room temperature. Then the mixture was heated at reflux for 15 min and evaporated to dryness to give the almost pure [3-bromo-4-(piperidin-4-yloxy)-phenyl]-acetic acid methyl ester. HPLC-MS: m/z=328/330(M+1)⁺, Rt=2.1 min.

The crude methyl ester was hydrolysed to the acid by heating a suspension of the ester in 6N hydrochloric acid (5 mL) at 95° C. over night. The solution was evaporated to dryness and further dried by evaporation from toluene to give the hydrochloride of [3-bromo-4-(piperidin-4-yloxy)-phenyl]-acetic acid. ¹H NMR (400 MHz, DMSO-d₆) δ 1.84-1.94 (m, 2 H), 2.04-2.14 (m, 2 H), 3.06-3.14 (m, 2 H), 3.15-3.24 (m, 2 H), 3.54 (s, 2 H), 4.72-4.78 (m, 1 H), 7.17 (d, J=8.59 Hz, 1 H), 7.23 (dd, J=8.59, 2.02 Hz, 1 H), 7.51 (d, J=2.02 Hz, 1 H), 8.87 (br s, 1 H), 8.95 (br s,1 H), 12.4 (very broad s, 1 H); HPLC-MS: m/z=314/316(M+1)⁺, Rt=1.8 min.

The hydrochloride was dissolved in water (20 mL) and 1 N sodium hydroxide (2.86 mL) was added to the solution which was stirred for 30 min and evaporated to dryness. The residue was dried in vacuo over sicapent to give 610 mg of almost pure sodium [3-bromo-4-(piperidin-4-yloxy)phenyl]acetate (as a mixture with sodium chloride)

### b) 4-(2-Bromo-4-carboxymethyl-phenoxy)-piperidine-1-carboxylic acid 4-(5-trifluoromethyl-pyridin-2-yloxy)-phenyl ester

A solution of 4-(5-trifluoromethyl-pyridin-2-yloxy)-phenyl chloroformate (454 mg, 1.43 mmol) in dichloromethane (10 mL) was added during 5 min to a stirred suspension of sodium [3-bromo-4-(piperidin-4-yloxy)phenyl]acetate/sodium chloride mixture from above (600 mg) in dichloromethane (40 mL) at 0° C. under nitrogen. The mixture was stirred for 30 min at 0° C. and for 5 days at room temperature. After filtration the filtrate was evaporated to dryness and the residue was purified by flash column chromatography (SiO₂, dichloromethane→dichloromethane/acetic acid/ethyl acetate (10:1:1)) to give 280 mg (33%) of the pure product as a colourless oil. ¹H NMR (400 MHz, CDCl₃) δ 1.98 (s, 4 H), 3.58 (s, 2 H), 3.67-3.77 (m, 2 H), 3.77-3.87 (m, 2 H), 4.67 (m, 1 H), 6.90 (d, J=8.59 Hz, 1 H), 7.00 (d, J=8.59 Hz, 1 H), 7.09-7.21 (m, 5 H), 7.51 (d, J=2.02 Hz, 1 H), 7.89 (dd, J=9.10, 2.53 Hz, 1 H), 8.44 (br s, 1 H); HPLC-MS: m/z=595/597 (M+1)⁺, Rt=4.8 min.

Pharmaceutical Methods

Compounds of formula I may be evaluated in vitro for their efficacy and potency to inhibit HSL, and such evaluation may be performed as described below.

Assays

Hormone-Sensitive Lipase (HSL)

Materials:

The Hormone-sensitive lipase was provided by Dr. Cecilia Holm, from Lund University Sweden or produced and purified by Novo Nordisk (NN) using the reagents and protocols used by Dr. Holm. The substrates used are: ³H-labeled triolein (TO) from Amersham, Buckinghamshire, U.K. cat No. TRA191; 5-20 Ci/mmol dissolved in toluene, triolein (Sigma, Cat. No. T-1740), fluorochrome-labeled triacylglyceride (cis-octadec-9-enoic acid 2-[12-(7-nitrobenzo[1,2,5]oxadiazol-4-ylamino)dodecanoyloxy]-1-cis-octadec-9-enoyloxymethyl-ethyl ester) prepared by Novo Nordisk (NN) by conventional methods, and 1,3-(di[³H]-stearin), 2-(PEG-Biotin)glycerol prepared in collaboration with Amersham Pharmacia Biotech, UK and described in WO 01/073442. Phosphatidyl choline (PC) and phosphatidyl inositol (PI) are from Sigma (St Luis Mo. cat. Nos. P-3556 and P-5954 respectively). All other reagents are of commercial grade and obtained from various commercial sources.

Methods.

3190.1: Assay for determination of percent inhibition of hormone sensitive lipase by compound at 10 μM sample concentration.

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor receptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 70 μL PED-BSA buffer) is pre-incubated for 30 min at 25° C. before addition of substrate (100 μL). Amount of formed product is measured after 120 min incubation at 37° C.

Results are given as percent activity relative to a non-inhibited sample (no compound).

3190.2: Assay for determination of IC₅₀ value for the inhibition of hormone sensitive lipase by compound. Standard concentrations of compound are 100 μM and 5-fold dilutions (initial concentration corresponding to 10 μM final concentration and 5-fold).

A lipid emulsion with fluorochrome-labeled triacylglyceride and phospholipid is used as substrate with a standard concentration of highly purified HSL (12 μg/mL initial concentration corresponding to 600 ng/mL final concentration). BSA is added as product acceptor. The transfer of the fluorochrome from the lipid phase to the water (BSA) phase changes the fluorescent properties of the fluorochrome. The changes can be monitored on a fluorimeter with an excitation wavelength of 450 nm and an emission wavelength of 545 nm.

Compound and HSL (20 μL compound, 10 μL enzyme and 70 μL PED-BSA buffer) is pre-incubated for 30 min at 25° C. before addition of substrate (100 μL). Amount of formed product is measured after 120 min incubation at 37° C.

Results are given as IC₅₀ values after 4PL fit of obtained activity data.

Results

With these methods the compounds of the examples are found to be inhibitors of HSL:

TABLE 1
Inhibition of HSL by compounds of the examples according to above
assay 3190.1 (% activity relative to non-inhibited sample).

| | | Test 3190.1 |
|---|---|---|
| Example | | HSL_FL |
| No | Compound | % ACTIVITY |
| 3 | 4-(1-Pyridin-3-yl-1H-imidazol-2-ylsulfanyl)-piperidine-1- | 5 |
| | carboxylic acid 4-(5-trifluoromethyl-pyridin-2-yloxy)-phenyl ester | |
| 85 | 4-(4-Carboxymethyl-phenoxy)-piperidine-1- | 5 |
| | carboxylic acid 4-phenethylcarbamoyl-phenyl ester | |
| 92 | 4-(4-Carboxymethyl-phenoxy)-piperidine-1-carboxylic acid | 3 |
| | 4-(2-phenoxy-ethyl)-phenyl ester | |
| 94 | 4-(4-Carboxymethyl-phenoxy)-piperidine-1-carboxylic acid | 6 |
| | 4-[2-(1-methyl-1H-tetrazol-5-yloxy)-ethyl]-phenyl ester | |
| 102 | 4-(4-Carboxymethyl-phenoxy)-piperidine-1-carboxylic acid 4- | 11 |
| | (3-chloro-5-trifluoromethyl-pyridin-2-ylmethyl)-phenyl ester | |
| 103 | 4-(4-Carboxymethyl-phenoxy)-piperidine-1-carboxylic acid 4- | 6 |
| | (4-trifluoromethyl-phenoxy)-phenyl ester | |
| 97 | 4-(4-Carboxymethyl-phenoxy)-piperidine-1-carboxylic acid 4- | 15 |
| | [2-(4-[1,2,4]triazol-4-yl-phenoxy)-ethyl]-phenyl ester | |
| 35 | 4-(4′-Carboxy-biphenyl-4-yloxy)-piperidine-1-carboxylic acid 4- | 14 |
| | (5-trifluoromethyl-pyridin-2-yloxy)-phenyl ester | |
| 36 | 4-[4-(4-Carboxy-oxazol-2-yl)-phenoxy]-piperidine-1-carboxylic | 8 |
| | acid 4-(5-trifluoromethyl-pyridin-2-yloxy)-phenyl ester | |
| 105 | 4-(4-Carboxy-phenoxy)-piperidine-1-carboxylic acid 4-(3,3-dimethyl- | 11 |
| | butylcarbamoyl)-phenyl ester | |
| 88 | 4-(4-Carboxymethyl-phenoxy)-piperidine-1-carboxylic acid 4- | 3 |
| | (cyclohexylmethyl-carbamoyl)-phenyl ester | |
| 10 | 4-(Pyridin-3-yloxy)-piperidine-1-carboxylic acid 4-(5- | 1 |
| | trifluoromethyl-pyridin-2-yloxy)-phenyl ester | |
| 12 | 4-(5-Phenylsulfanyl-pyrazol-1-yloxy)-piperidine-1-carboxylic acid | 11 |
| | 4-(5-trifluoromethyl-pyridin-2-yloxy)-phenyl ester | |
| 37 | 4-[4-(2-Carboxy-ethyl)-phenoxy]-piperidine-1-carboxylic acid | 1 |
| | 4-(5-trifluoromethyl-pyridin-2-yloxy)-phenylester | |
| 39 | 4-(4-Carboxy-phenoxy)-piperidine-1-carboxylic acid | 5 |
| | 4-(5-trifluoromethyl-pyridin-2-yloxy)-phenyl ester | |
| 22 | 4-[4-(4-Cyclopentyl-piperazin-1-yl)-phenoxy]-piperidine-1- | 4 |
| | carboxylic acid 4-(5-trifluoromethyl-pyridin-2-yloxy)-phenyl ester | |
| 44 | 4-(4-Carboxymethyl-phenoxymethyl)-piperidine-1-carboxylic acid | 5 |
| | 4-(5-trifluoromethyl-pyridin-2-yloxy)-phenyl ester | |
| 1 | 4-[2-(Benzyl-methyl-amino)-ethyl]-piperidine-1-carboxylic acid | 1 |
| | 4-(5-trifluoromethyl-pyridin-2-yloxy)-phenyl ester | |
| 110 | 4-(3-Carboxymethyl-phenoxy)-piperidine-1-carboxylic acid | 20 |
| | 4-(5-trifluoromethyl-pyridin-2-yloxy)-phenyl ester | |
| 112 | 4-[3-(2-Carboxy-ethyl)-phenoxy]-piperidine-1-carboxylic acid | 14 |
| | 4-(5-trifluoromethyl-pyridin-2-yloxy)-phenyl ester | |
| 91 | 4-(4-Carboxymethyl-phenoxy)-piperidine-1-carboxylic acid | 10 |
| | 4-(2-thiophen-2-yl-ethylcarbamoyl)-phenyl ester | |
| 117 | 4-(4-Carboxymethyl-phenoxy)-piperidine-1-carboxylic acid | 8 |
| | 4-(5-chloro-pyridin-2-yloxy)-phenyl ester | |
| 48 | 4-(Pyrazol-1-yloxy)-piperidine-1-carboxylic acid | 1 |
| | 4-(5-methyl-pyridin-2-ylmethyl)-phenyl ester | |
| 50 | 4-Phenoxy-piperidine-1-carboxylic acid | 3 |
| | 4-(6-methyl-pyridin-3-ylmethyl)-phenyl ester | |
| 55 | 4-(4-Allyloxycarbonylmethyl-phenoxy)-piperidine-1-carboxylic acid | 1 |
| | 4-[2-(4,4-dimethyl-2,6-dioxo-piperidin-1-yl)-ethyl]-phenyl ester | |
| 59 | 4-(4-Carboxy-phenoxy)-piperidine-1-carboxylic acid | 31 |
| | 4-[2-(4,4-dimethyl-2,6-dioxo-piperidin-1-yl)-ethyl]-phenyl ester | |
| 67 | 4-Pyrimidin-2-ylmethyl-piperidine-1-carboxylic acid | 4 |
| | 4-(4-trifluoromethyl-benzyl)-phenyl ester | |
| 68 | 4-Thiophen-3-ylmethyl-piperidine-1-carboxylic acid | 3 |
| | 4-(5-trifluoromethyl-pyridin-2-yloxy)-phenyl ester | |
| 71 | 4-(5-Methyl-pyridin-2-ylmethyl)-piperidine-1-carboxylic acid | 9 |
| | 4-(4-trifluoromethyl-benzyl)-phenyl ester | |
| 74 | 4-(4-Carboxymethyl-benzyl)-piperidine-1-carboxylic acid | 3 |
| | 4-(4-trifluoromethyl-benzyl)-phenyl ester | |
| 61 | 4-(Pyrazol-1-yloxy)-piperidine-1-carboxylic acid | 15 |
| | 5-benzoylamino-pyridin-2-yl ester | |
| 31 | 4-[4-(2-Ethoxycarbonyl-ethyl)-phenoxy]-piperidine-1- | 0 |
| | carboxylic acid 4-(5-trifluoromethyl-pyridin-2-yloxy)-phenyl ester | |

1. A compound of the general formula (I): 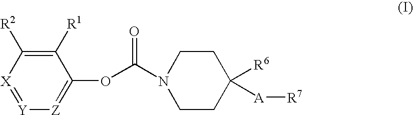
wherein
R¹ and R² are independently selected from hydrogen, hydroxy, sulfanyl, amino, halogen, sulfo, C₁₋₆-alkyl, C₁₋₆-alkoxy, C₂₋₆-alkenyl, aryl, heteroaryl, C₃₋₄-heterocyclyl and C₃₋₁₀-cycloalkyl wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆alkyl, C₁₋₆-alkoxy, C₂₋₆-alkenyl, aryl, heteroaryl, C₃₋₄-heterocyclyl, and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₄-heterocyclyl, and C₃₋₁₀-cycloalkyl;
X is N or C—R³; Y is N or C—R⁴; Z is N or C—R⁵;
R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₄-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆-alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
R⁶ is hydrogen or fluor;
A is selected from —O—, —S—, —S(═O)—, —S(═O)₂—, —CH₂, —CH₂O—, —CH₂S—, —CH₂CH₂—N(R⁸)—, —CH₂CHF—N(R⁸)—, —CH₂CF₂—N(R⁸)—, and —CHFCH₂—N(R⁸)—;
R⁸ is selected from hydrogen, C₁₋₆-alkyl, C₂₋₄-alkenyl, aryl, heteroaryl, C₃₋₈-heterocyclyl and C₃₋₁₀-cycloalkyl, wherein each of C₁₋₆alkyl, C₂₋₄-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆alkyl, C₂₋₄-alkenyl, aryl, heteroaryl, C₃₋₈-heterocyclyl and C₃₋₁₀-cycloalkyl, wherein each of hydroxy, sulfanyl, amino, sulfo, C₁₋₆alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
R⁷ is selected from aryl or heteroaryl, which may optionally be substituted with one or more substituents selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy;
with the proviso that X, Y and Z are not all CH;
and with the proviso that said compound is not
1-Piperidinecarboxylic acid, 4-(phenylmethyl)-, 4-nitrophenyl ester,
4-Benzylpiperidine-1-carboxylic acid 4-(5-trifluoromethylpyridin-2-yloxy)phenyl ester,
1-Piperidinecarboxylic acid, 4-[[4-[(dimethylamino)methyl]phenyl]methyl]-, 4-methylphenyl ester, or
1-Piperidinecarboxylic acid, 4-[[4-(1-piperidinylmethyl)phenyl]methyl]-, 4-chlorophenyl ester;
as well as diastereomers, enantiomers or tautomeric forms thereof, mixtures of these, pharmaceutically acceptable salts thereof, pharmaceutically acceptable solvates thereof, or polymorphs.

2. A compound according to claim 1, wherein A is —CH₂—.

3. A compound according to claim 1, wherein R¹ and R² are independently selected from hydrogen, hydroxy, sulfanyl, amino, halogen, sulfo, C₁₋₆alkyl, C₁₋₆alkoxy, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₁₋₆alkoxy, C₂₋₆-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

4. A compound according to claim 1, wherein R³, R⁴ and R⁵ are independently selected from hydrogen, hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₄-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl.

5. A compound according to claim 1, wherein R¹ is hydrogen.

6. A compound according to claim 1, wherein R² is hydrogen.

7. A compound according to claim 1, wherein R¹ is hydrogen and R² is hydrogen.

8. A compound according to claim 1, wherein R² is selected from the group consisting of

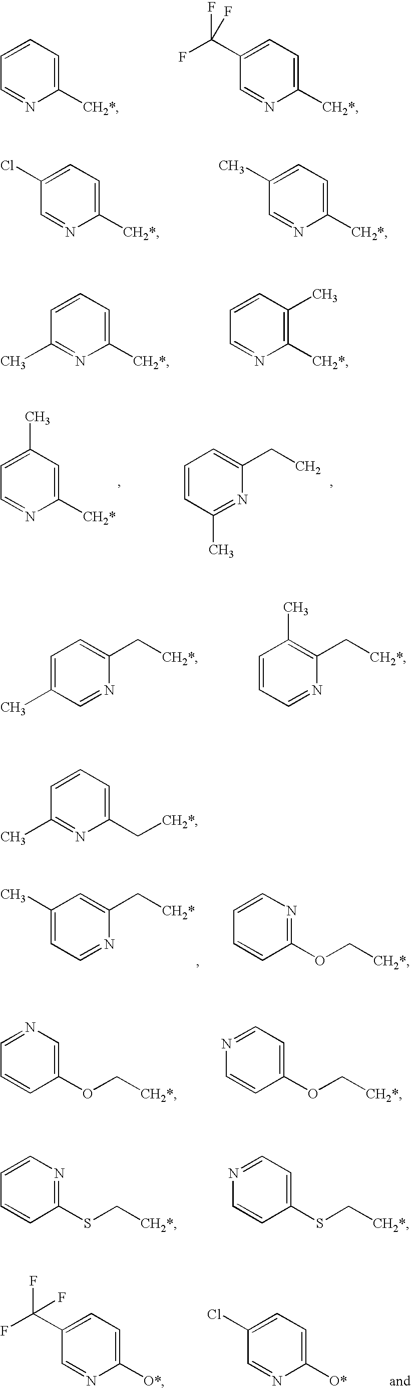

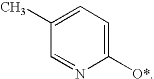

9. A compound according to claim 1, wherein R² is selected from the group consisting of 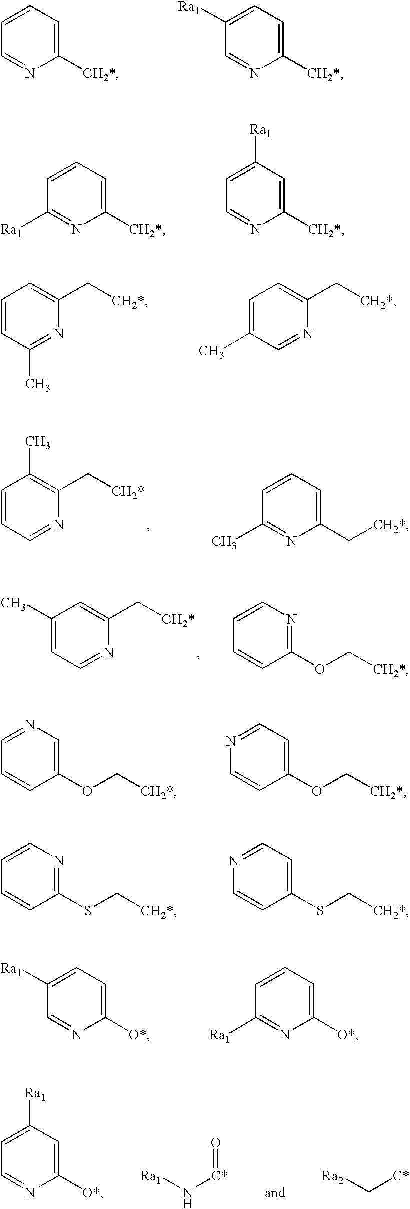
wherein Ra1 is selected from hydroxy, sulfanyl, oxo, halogen, amino, sulfo, perhalomethyl, perhalomethoxy, C₁₋₆alkyl, C₁₋₆-alkoxy, C₂₋₆alkenyl, aryl, heteroaryl, C₃₋₈-heterocyclyl, and C₃₋₁₀-cycloalkyl;
wherein Ra2 is selected from C₁₋₆-alkoxy, wherein C₁₋₆-alkoxy may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

10. A compound according to claim 1, wherein R² is selected from the group consisting of 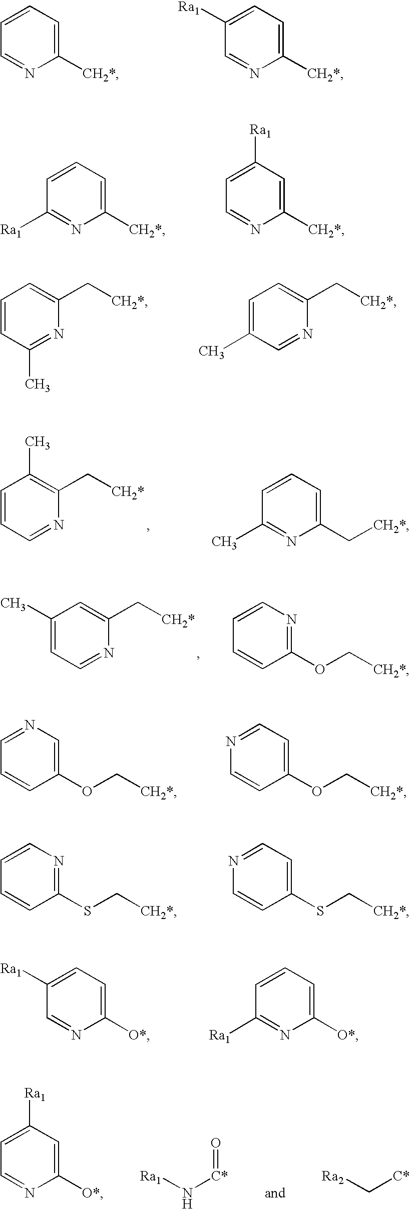
wherein Ra1 is selected from sulfanyl, halogen, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl;
wherein Ra2 is selected from C₁₋₆-alkoxy, wherein C₁₋₆-alkoxy may optionally be substituted with one or more substituents independently selected from hydroxy, sulfanyl, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

11. A compound according to claim 1, wherein R² is selected from the group consisting of 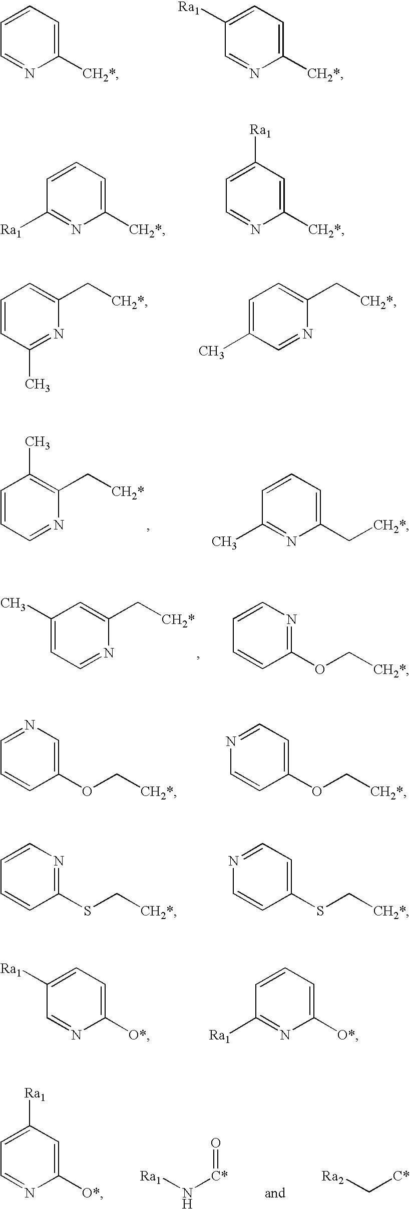
wherein Ra1 is selected from halogen, perhalomethyl, C₁₋₆-alkyl, C₁₋₆-alkoxy, C₂₋₆-alkenyl, heteroaryl, C₃₋₈-heterocyclyl, and C₃₋₁₀-cycloalkyl;
wherein Ra2 is selected from C₁₋₆-alkoxy, wherein C₁₋₆-alkoxy may optionally be substituted with one or more substituents independently selected from hydroxy, halogen, amino, perhalomethyl, C₁₋₆-alkyl, C₁₋₆-alkoxy, C₂₋₆-alkenyl, heteroaryl, C₃₋₄-heterocyclyl, and C₃₋₁₀-cycloalkyl.

12. A compound according to claim 1, wherein R² is selected from the group consisting of 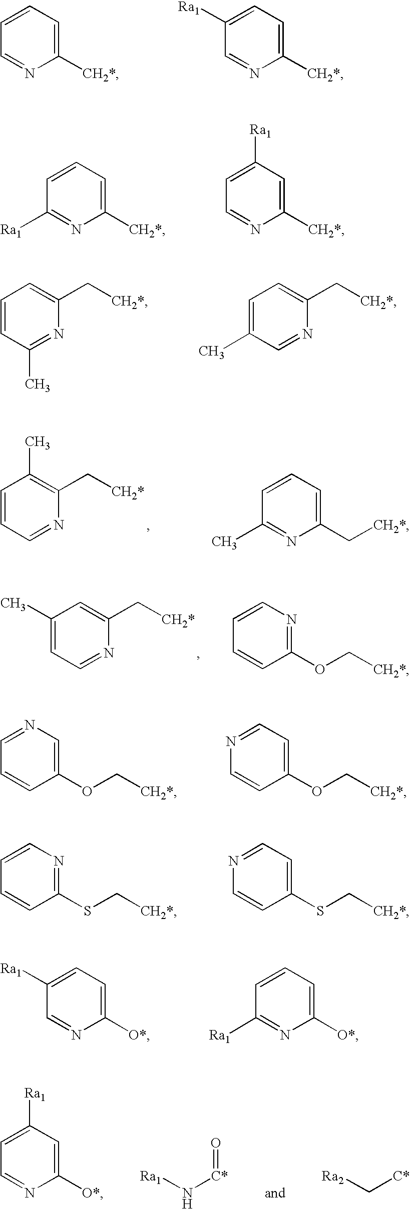
wherein Ra1 is selected from halogen, perhalomethyl, C₁₋₆-alkyl, C₁₋₆-alkoxy, heteroaryl, and C₃₋₈-heterocyclyl;
wherein Ra2 is selected from C₁₋₃-alkoxy, wherein C₁₋₃-alkoxy may optionally be substituted with one or more substituents independently selected from hydroxy, halogen, amino, C₁₋₆-alkyl, C₁₋₆-alkoxy, heteroaryl and C₃₋₈-heterocyclyl.

13. A compound according to claim 1, wherein R² is selected from the group consisting of 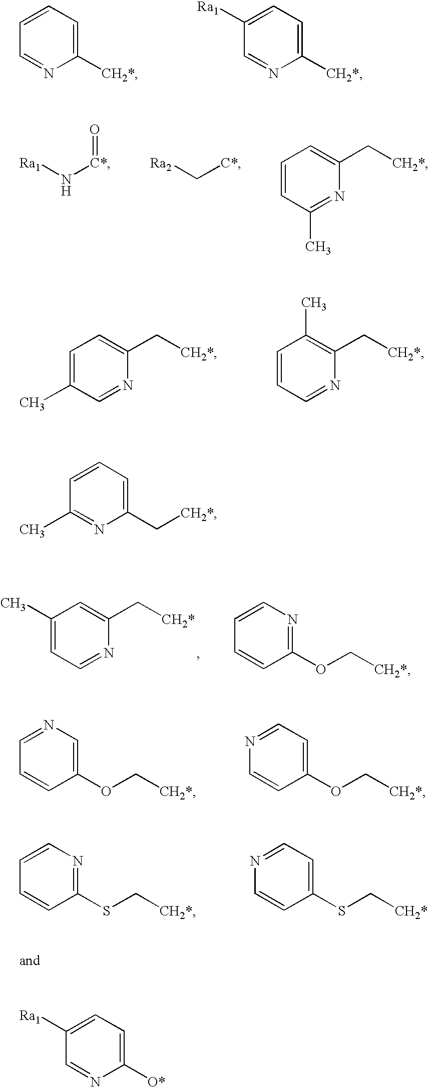
wherein Ra1 is selected from halogen, perhalomethyl, C₁₋₆-alkyl, C₁₋₆-alkoxy, heteroaryl, and C₃₋₈-heterocyclyl;
wherein Ra2 is selected from C₁₋₃-alkoxy, wherein C₁₋₃-alkoxy may optionally be substituted with one or more substituents independently selected from hydroxy, halogen, amino, C₁₋₆-alkyl, C₁₋₆-alkoxy, heteroaryl and C₃₋₈-heterocyclyl.

14. A compound according to claim 1, wherein R² is selected from the group consisting of

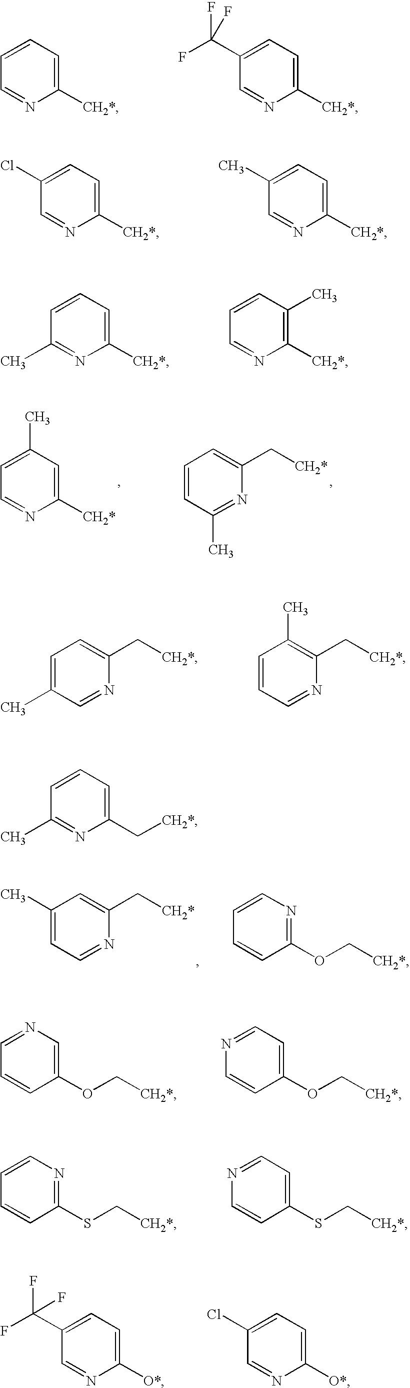

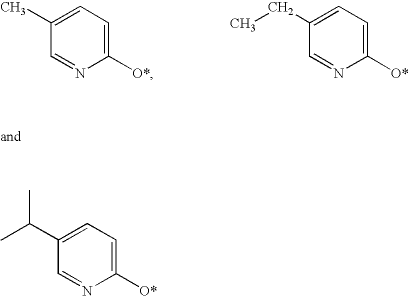

15. A compound according to claim 1, wherein R² is selected from the group consisting of

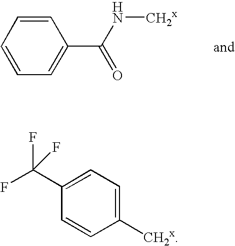

16. A compound according to claim 1, wherein R² is selected from the group consisting of

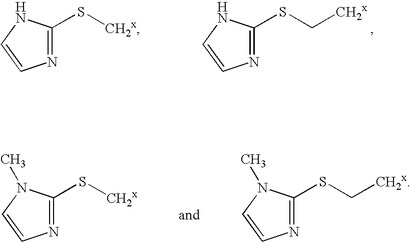

17. A compound according to claim 1, wherein R² is selected from the group consisting of

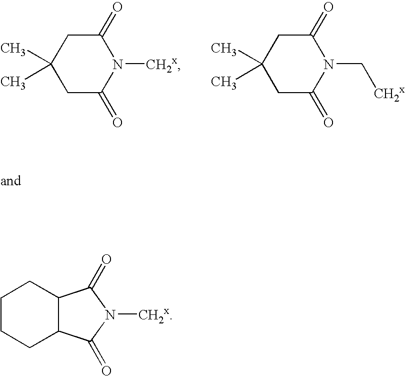

18. A compound according to claim 1, wherein X is N.

19. A compound according to claim 1, wherein X is CH.

20. A compound according to claim 1, wherein X is C—R³.

21. A compound according to claim 20, wherein R³ is selected from the group consisting of

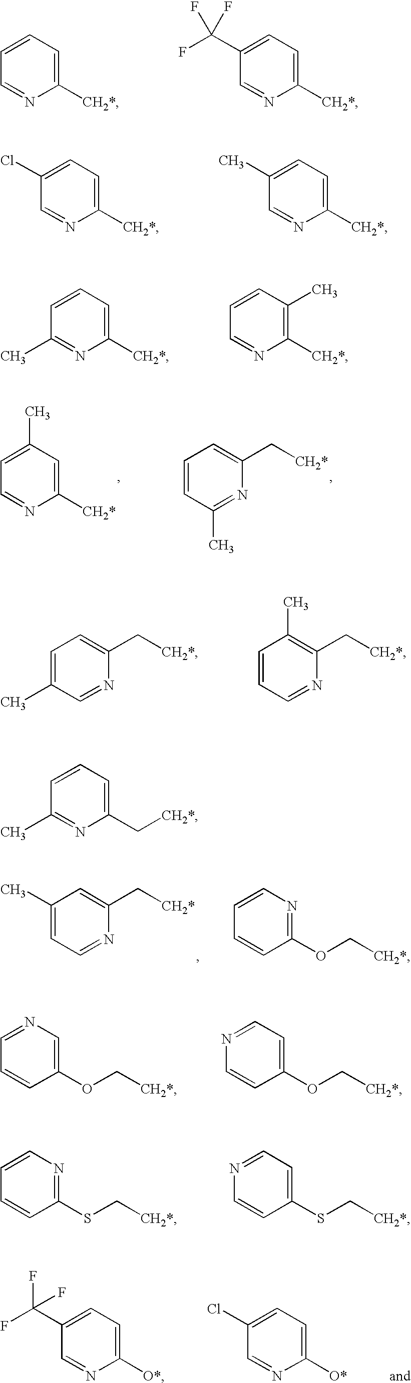

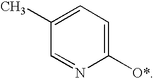

22. A compound according to claim 20, wherein R³ is selected from the group consisting of 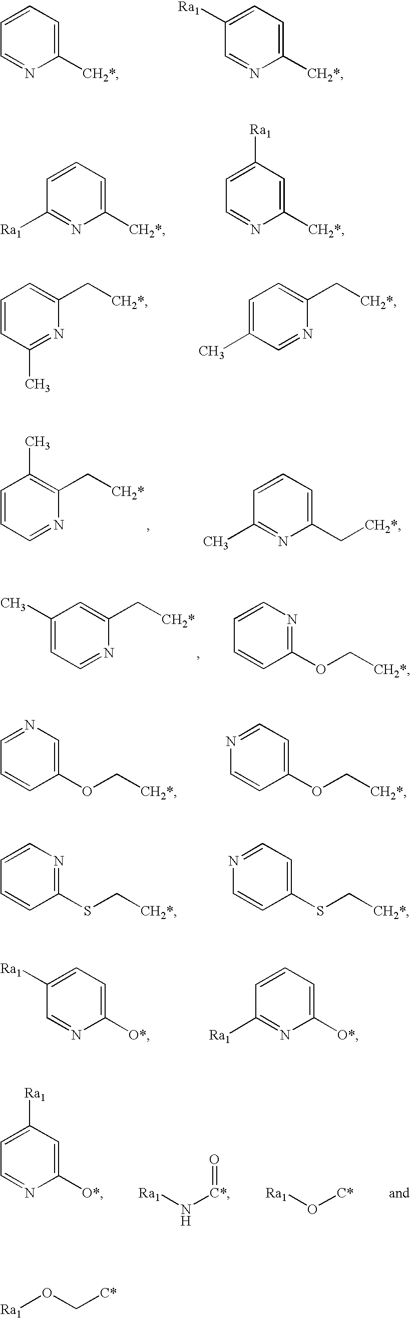
wherein Ra1 is selected from hydroxy, sulfanyl, oxo, halogen, amino, sulfo, C₁₋₆alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆alkyl, C₂₋₄-alkenyl, aryl, heteroaryl, C₃₋₈-heterocyclyl and C₃₋₁₀-cycloalkyl may optionally be substituted with one or more substituents independently selected from hydroxy, sulfanyl, oxo, halogen, amino, sulfo, perhalomethyl, perhalomethoxy, C₁₋₆-alkyl, C₁₋₆-alkoxy, C₂₋₆-alkenyl, aryl, heteroaryl, C₃₋₈-heterocyclyl, and C₃₋₁₀-cycloalkyl.

23. A compound according to claim 20, wherein R³ is selected from the group consisting of 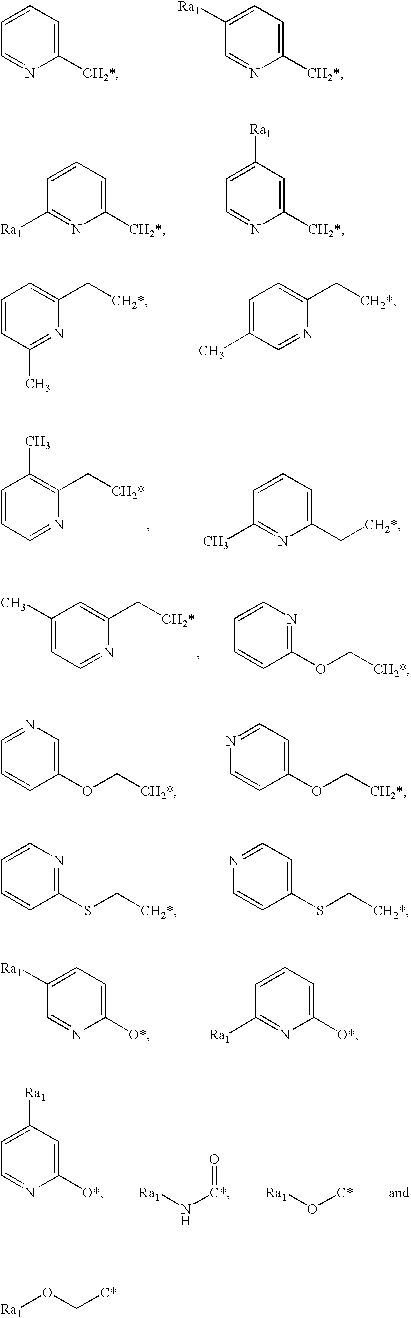
wherein Ra1 is selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, halogen, amino, perhalomethyl, perhalomethoxy, C₁₋₆-alkyl, C₁₋₆-alkoxy, aryl, heteroaryl, C₃₋₈-heterocyclyl, and C₃₋₁₀-cycloalkyl.

24. A compound according to claim 20, wherein R³ is selected from the group consisting of 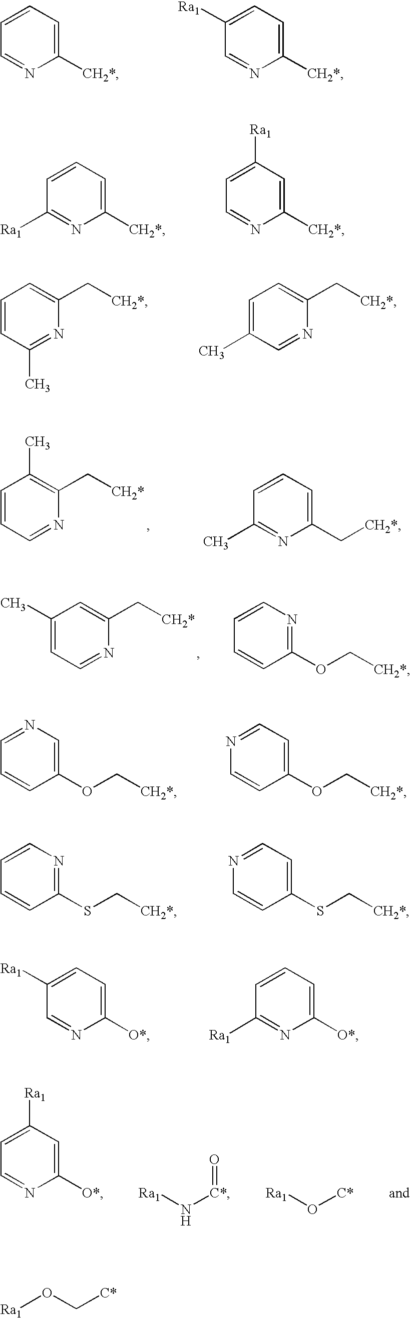
wherein Ra1 is selected from sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₈-heterocyclyl, and C₃₋₇-cycloalkyl.

25. A compound according to claim 20, wherein R³ is selected from the group consisting of 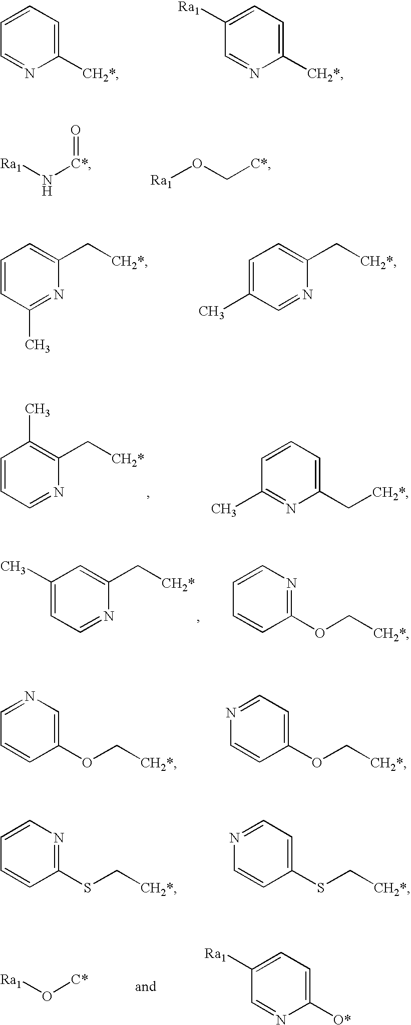
wherein Ra1 is selected from sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of sulfanyl, sulfo, C₁₋₆-alkyl, C₂₋₆-alkenyl, aryl, heteroaryl, C₃₋₈-heterocyclyl and C₃₋₁₀-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₈-heterocyclyl, and C₃₋₇-cycloalkyl.

26. A compound according to claim 20, wherein R³ is selected from the group consisting of 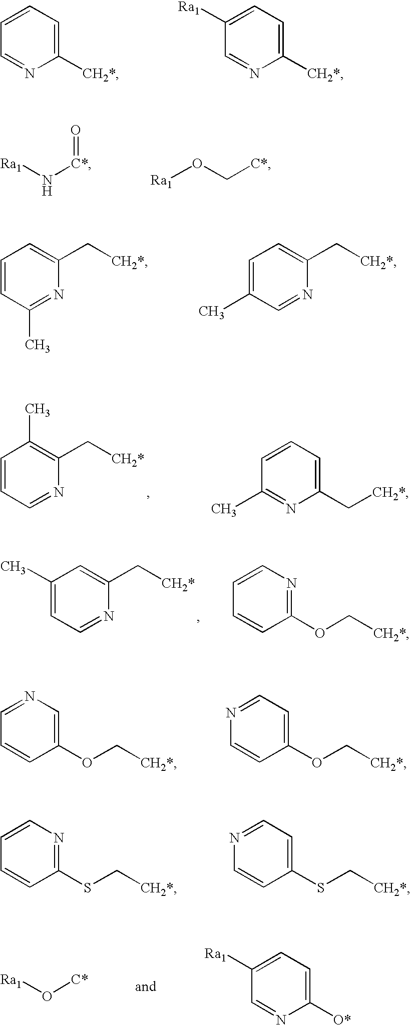
wherein Ra1 is selected from sulfanyl, halogen, C₁₋₆alkyl, aryl, heteroaryl, C₃₋₈-heterocyclyl and C₃₋₇-cycloalkyl, wherein each of sulfanyl, C₁₋₆alkyl, aryl, heteroaryl, C₃₋₈-heterocyclyl and C₃₋₇-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₈-heterocyclyl, and C₃₋₇-cycloalkyl.

27. A compound according to claim 20, wherein R³ is selected from the group consisting of 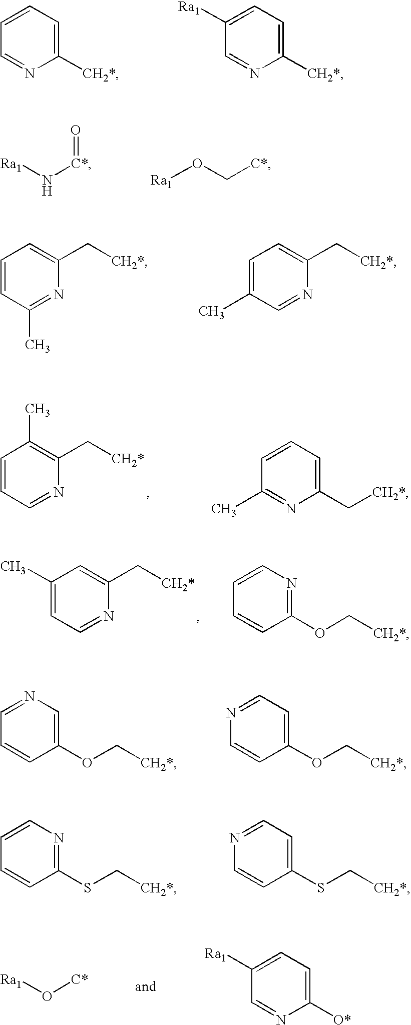
wherein Ra1 is selected from halogen, C₁₋₆alkyl, aryl, heteroaryl, C₃₋₆-heterocyclyl and C₃₋₄-cycloalkyl, wherein each of C₁₋₆-alkyl, aryl, heteroaryl, C₃₋₆-heterocyclyl and C₃₋₄-cycloalkyl may optionally be substituted with one or more substituents independently selected from hydroxy, halogen, amino, perhalomethyl, perhalomethoxy, C₁₋₄-alkyl, C₁₋₄-alkoxy, aryl, heteroaryl, C₃₋₆-heterocyclyl, and C₃₋₆-cycloalkyl.

28. A compound according to claim 20, wherein R³ is selected from the group consisting of 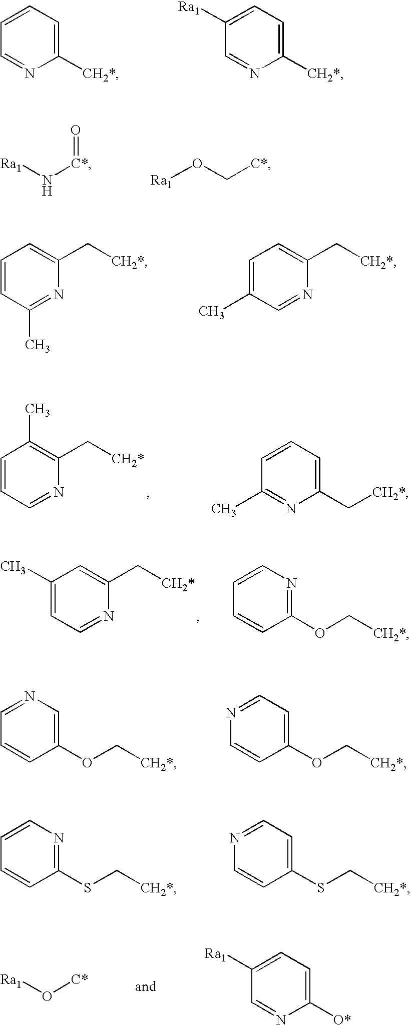
wherein Ra1 is selected from halogen, C₁₋₆-alkyl, aryl, heteroaryl, C₃₋₆-heterocyclyl and C₃₋₄-cycloalkyl, wherein each of C₁₋₆-alkyl, aryl, heteroaryl may optionally be substituted with one or more substituents independently selected from hydroxy, halogen, perhalomethyl, perhalomethoxy, C₁₋₄-alkyl, C₁₋₄-alkoxy, C₃₋₆-heterocyclyl, and C₃₋₆-cycloalkyl.

29. A compound according to claim 1, wherein R³ is selected from the group consisting of

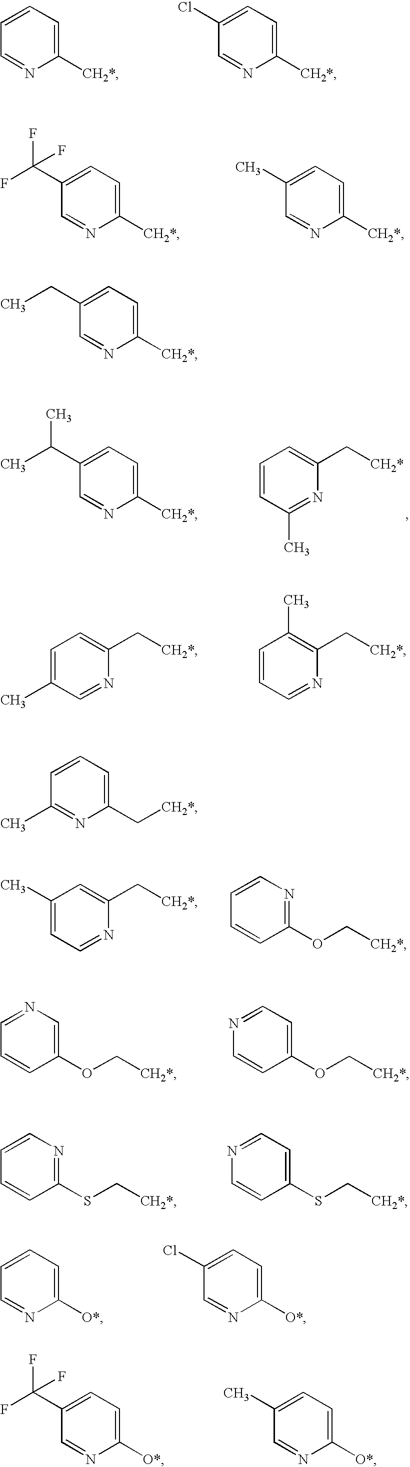

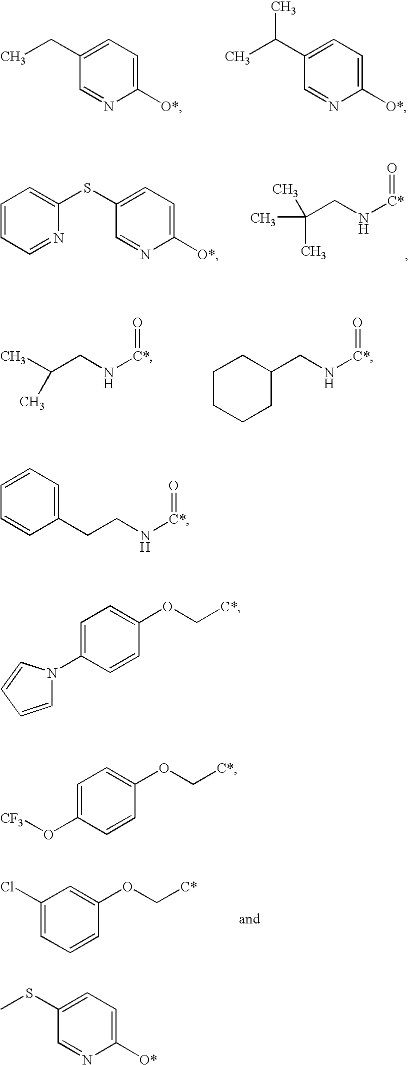

30. A compound according to claim 1, wherein R³ is selected from the group consisting of

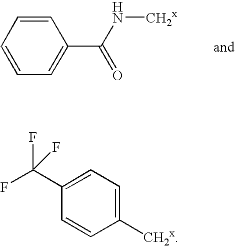

31. A compound according to claim 1, wherein R³ is selected from the group consisting of

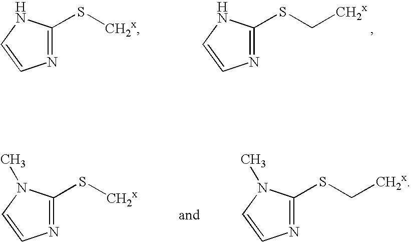

32. A compound according to claim 1, wherein R³ is selected from the group consisting of

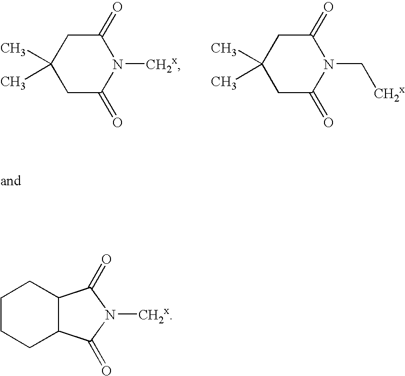

33. A compound according to claim 1, wherein R³ is selected from the group consisting of 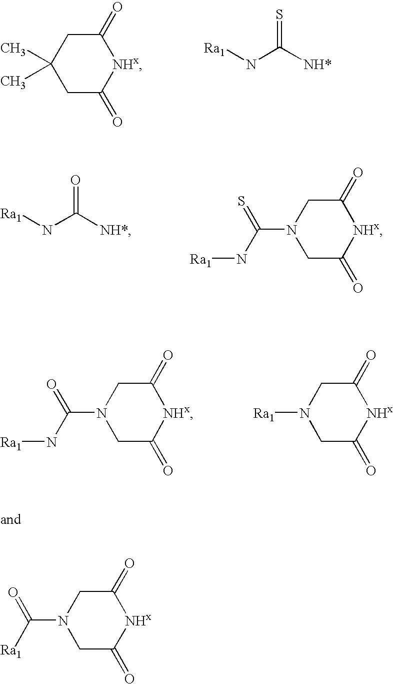
wherein Ra₁ is selected from C₁₋₆-alkyl, aryl, heteroaryl, C₃₋₆-heterocyclyl and C₃₋₇-cycloalkyl.

34. A compound according to claim 1, wherein Z is N.

35. A compound according to claim 1, wherein Z is C—R⁵.

36. A compound according to claim 1, wherein Z is CH.

37. A compound according to claim 1, wherein Y is N.

38. A compound according to claim 1, wherein Y is CH.

39. A compound according to claim 1, wherein Y is C—R⁴.

40. A compound according to claim 1, wherein only one of X, Y and Z is N.

41. A compound according to claim 1, wherein X is C—R³, Y is C—R⁴ and Z is C—R⁵.

42. A compound according to claim 1, wherein Y and Z are both CH.

43. A compound according to claim 42, wherein X is C—R³.

44. A compound according to claim 43, wherein R³ is selected from the group consisting of

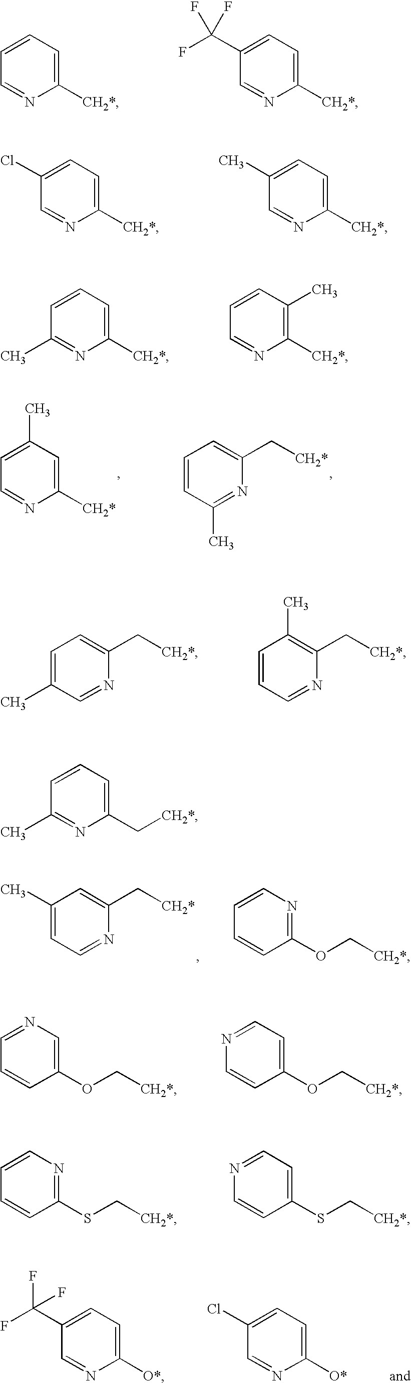

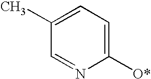

45. A compound according to claim 1, wherein R⁶ is H.

46. A compound according to claim 1, wherein —R⁷ is selected from the group consisting of

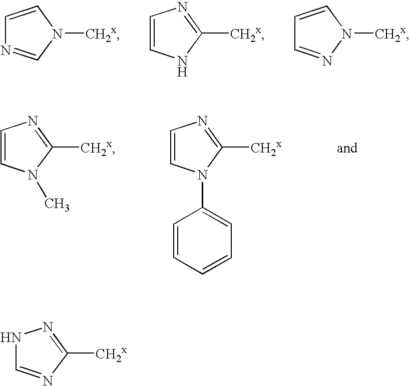

47. A compound according to claim 1, wherein —R⁷ is selected from the group consisting of

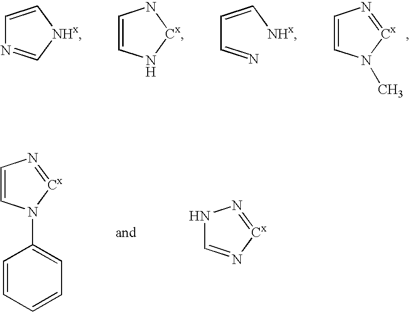

48. A compound according to claim 1, wherein —R⁷ is selected from the group consisting of 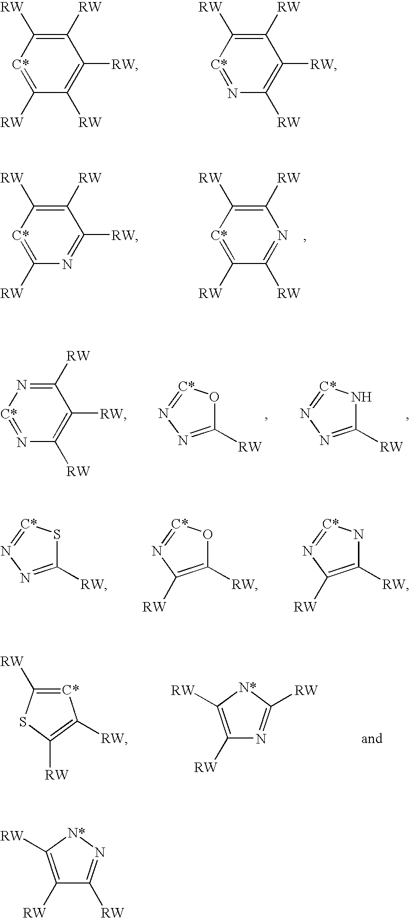
wherein, RW is selected from hydroxy, sulfanyl, halogen, amino, sulfo, C₁₋₆-alkyl, C₂₋₄-alkenyl, aryl, heteroaryl, C₃₋₈-heterocyclyl and C₃₋₁₀-cycloalkyl, wherein each of hydroxy, sulfanyl, amino, sulfo, C₁₋₆alkyl, C₂₋₄-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆alkyl, C₂₋₄-alkenyl, aryl, heteroaryl, C₃₋₈-heterocyclyl and C₃₋₁₀-cycloalkyl, wherein each of hydroxy, sulfanyl, amino, sulfo, C₁₋₆-alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, C₁₋₆-alkyl, perhalomethyl and perhalomethoxy.

49. A compound according to claim 1, wherein —R⁷ is selected from the group consisting of 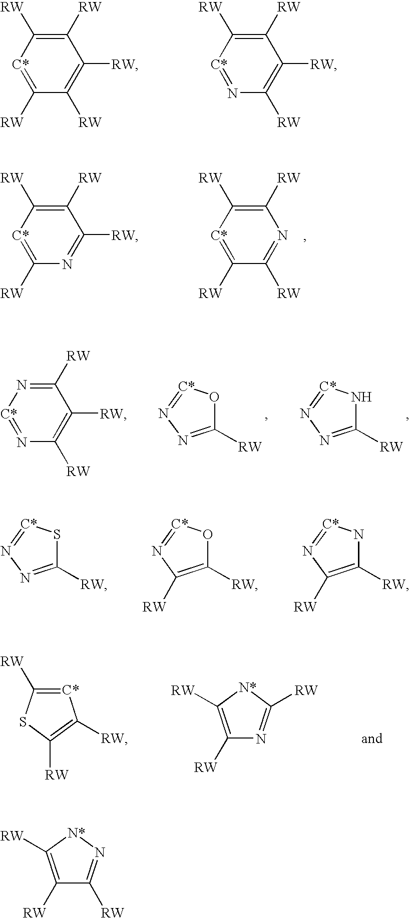
wherein, RW is selected from hydroxy, sulfanyl, halogen, amino, sulfo, C₁₋₆-alkyl, C₂₋₆-alkenyl, aryl, heteroaryl, C₃₋₈-heterocyclyl and C₃₋₁₀-cycloalkyl, wherein each of hydroxy, sulfanyl, amino, sulfo, C₁₋₆alkyl, C₂₋₆-alkenyl, aryl, heteroaryl, C₃₋₈-heterocyclyl and C₃₋₁₀-cycloalkyl is optionally substituted with one or more substituents independently selected from hydroxy, sulfanyl, oxo, halogen, amino, sulfo, C₁₋₆-alkyl, C₂₋₆-alkenyl, aryl, heteroaryl, C₃₋₈-heterocyclyl and C₃₋₁₀-cycloalkyl.

50. A compound according to claim 1, wherein —R⁷ is selected from the group consisting of 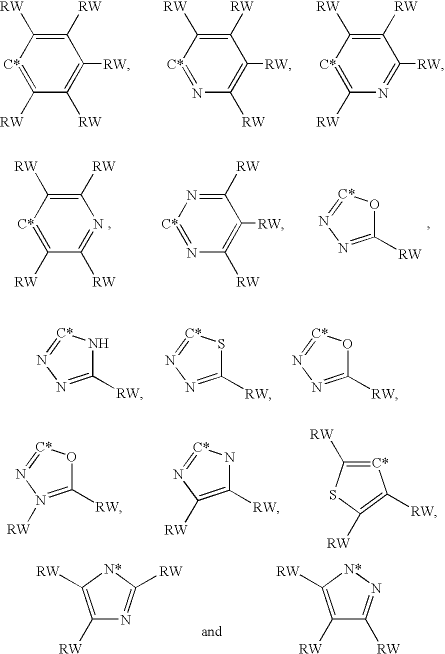
wherein, RW is selected from hydroxy, sulfanyl, halogen, amino, sulfo, C₁₋₆-alkyl, C₂₋₄-alkenyl, aryl, heteroaryl, C₃₋₈-heterocyclyl and C₃₋₁₀-cycloalkyl.

51. A compound according to claim 1, wherein —R⁷ is selected from the group consisting of

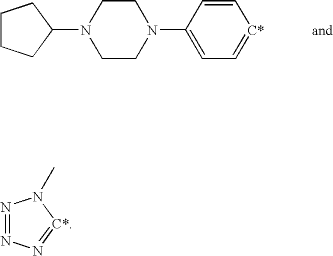

52. A compound according to claim 1, wherein —R⁷ is selected from the group consisting of

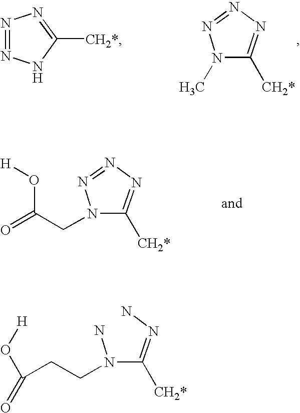

53. A compound according to claim 1, wherein —R⁷ is selected from the group consisting of

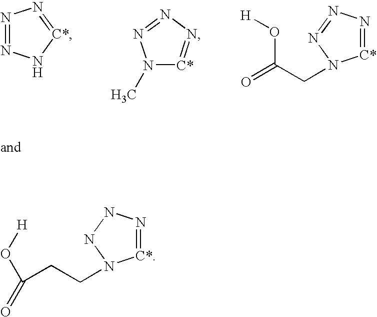

54. A compound according to claim 1, wherein —R⁷ is selected from the group consisting of

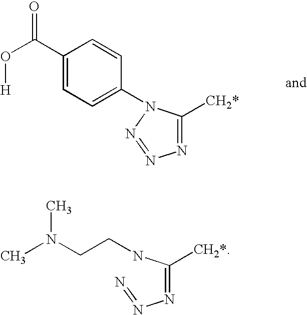

55. A compound according to claim 1, wherein —R⁷ is selected from the group consisting of

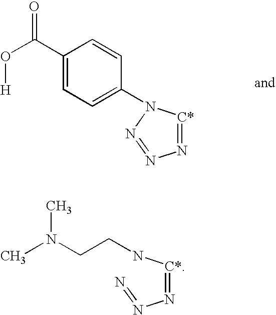

56. A compound according to claim 1, wherein —R⁷ is selected from the group consisting of

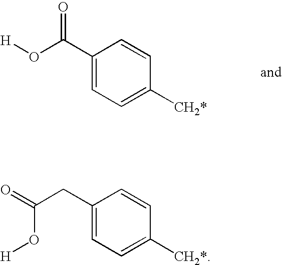

57. A compound according to claim 1, wherein —R⁷ is selected from the group consisting of

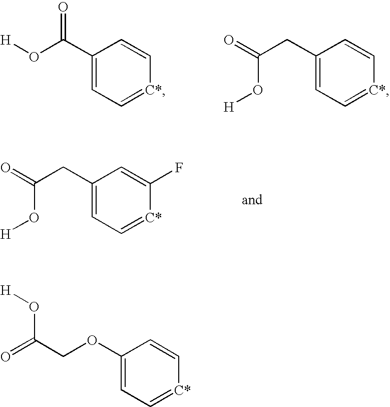

58. A compound according to claim 1, wherein —R⁷ is selected from the group consisting of

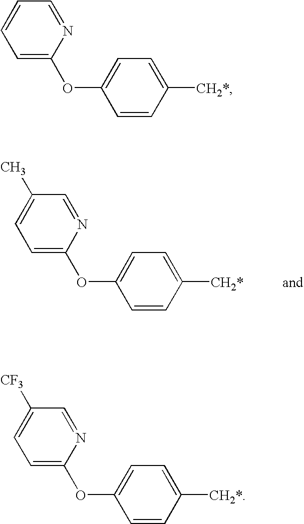

59. A compound according to claim 1, wherein —R⁷ is selected from the group consisting of

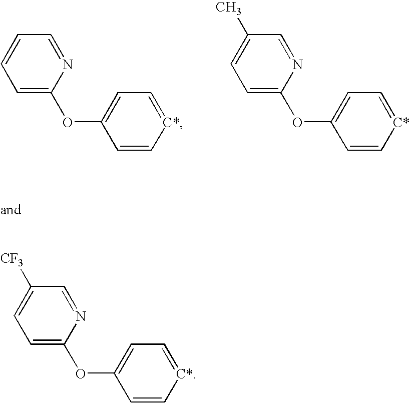

60. A compound according to claim 1, wherein —R⁷ is selected from the group consisting of

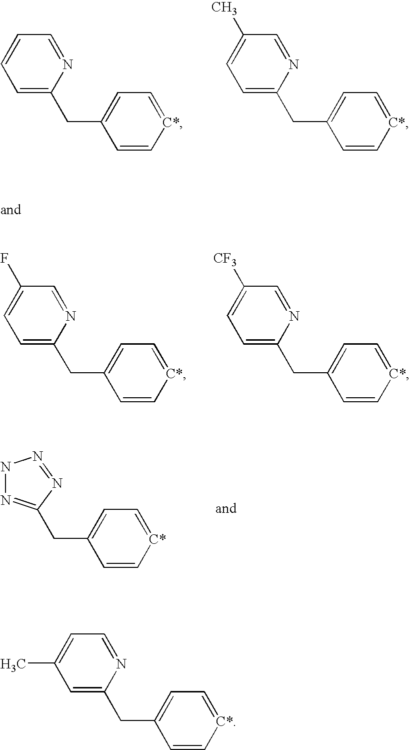

61. A compound according to claim 1, wherein A is —O—.

62. A compound according to claim 1, wherein A is —S—, —S(═O)— or —S(═O)₂—.

63. A compound according to claim 1, wherein A is —S—.

64. A compound according to claim 1, wherein A is —CH₂O— or —CH₂S—.

65. A compound according to claim 1, wherein A is selected from —CH₂CH₂—N(R⁸)—, —CH₂CHF—N(R⁸)—, —CH₂CF₂—N(R⁸)—, or —CHFCH₂—N(R⁸)—.

66. A compound according to claim 65, wherein R⁸ is selected from hydrogen, C₁₋₆alkyl, C₂₋₆-alkenyl, C₁₋₆alkyl which has been substituted with one or more halogens, or C₂₋₆-alkenyl which has been substituted with one or more halogens.

67. A compound according to claim 1, where the compound is selected from the group consisting of
4-[2-(Benzyl-methyl-amino)-ethyl]-piperidine-1-carboxylic acid 4-(5-trifluoromethyl-pyridin-2-yloxy)-phenyl ester,
4-(Pyridin-2-ylsulfanyl)-piperidine-1-carboxylic acid 4-(5-trifluoromethyl-pyridin-2-yloxy)-phenyl ester,
4-(1-Pyridin-3-yl-1H-imidazol-2-ylsulfanyl)-piperidine-1-carboxylic acid 4-(5-trifluoromethyl-pyridin-2-yloxy)-phenyl ester,
4-(1-Methyl-1H-imidazol-2-ylsulfanyl)-piperidine-1-carboxylic acid 4-(5-trifluoromethyl-pyridin-2-yloxy)-phenyl ester,
4-[4-(4-Methyl-piperazine-1-carbonyl)-phenoxy]-piperidine-1-carboxylic acid 4-(5-trifluoromethyl-pyridin-2-yloxy)-phenyl ester,
4-(1H-Imidazol-2-ylsulfanyl)-piperidine-1-carboxylic acid 4-(5-trifluoromethyl-pyridin-2-yloxy)-phenyl ester,
4-(4-Piperazin-1-yl-phenoxy)-piperidine-1-carboxylic acid 4-(5-trifluoromethyl-pyridin-2-yloxy)-phenyl ester,
4-(Pyrazol-1-yloxy)-piperidine-1-carboxylic acid 4-(5-trifluoromethyl-pyridin-2-yloxy)-phenyl ester,
4-(Imidazol-1-yloxy)-piperidine-1-carboxylic acid 4-(5-trifluoromethyl-pyridin-2-yloxy)-phenyl ester,
4-(Pyridin-3-yloxy)-piperidine-1-carboxylic acid 4-(5-trifluoromethyl-pyridin-2-yloxy)-phenyl ester,
4-[4-(2-Dimethylamino-ethyl)-phenoxy]-piperidine-1-carboxylic acid 4-(5-trifluoromethyl-pyridin-2-yloxy)-phenyl ester,
4-(5-Phenylsulfanyl-pyrazol-1-yloxy)-piperidine-1-carboxylic acid 4-(5-trifluoromethyl-pyridin-2-yloxy)-phenyl ester,
4-(4-Imidazol-1-yl-phenoxy)-piperidine-1-carboxylic acid 4-(5-trifluoromethyl-pyridin-2-yloxy)-phenyl ester,
4-(Pyridin-2-yloxy)-piperidine-1-carboxylic acid 4-(5-trifluoromethyl-pyridin-2-yloxy)-phenyl ester,
4-(3-Pyridin-2-yl-pyrazol-1-yloxy)-piperidine-1-carboxylic acid 4-(5-trifluoomethyl-pyridin-2-yloxy)-phenyl ester,
4-(2-Phenylsulfanyl-imidazol-1-yloxy)-piperidine-1-carboxylic acid 4-(5-trifluoromethyl-pyridin-2-yloxy)-phenyl ester,
4-(2-Chloro-imidazol-1-yloxy)-piperidine-1-carboxylic acid 4-(5-trifluoromethyl-pyridin-2-yloxy)-phenyl ester,
4-[2-(4-Methoxy-phenyl)-imidazol-1-yloxy]-piperidine-1-carboxylic acid 4-(5-trifluoromethyl-pyridin-2-yloxy)-phenyl ester,
4-(2-Methyl-imidazol-1-yloxy)-piperidine-1-carboxylic acid 4-(5-trifluorometyhl-pyridin-2-yloxy)-phenyl ester,
4-(Pyridin-4-ylsulfanyl)-piperidine-1-carboxylic acid 4-(5-trifluoromethyl-pyridin-2-yloxy)-phenyl ester,
4-(4-Pyridin-3-ylmethyl-phenoxy)-piperidine-1-carboxylic acid 4-(5-trifluoromethyl-pyridin-2-yloxy)-phenyl ester,
4-[4-(4-Cyclopentyl-piperazin-1-yl)-phenoxy]-piperidine-1-carboxylic acid 4-(5-trifluoromethyl-pyridin-2-yloxy)-phenyl ester,
4-(4-Pyridin-2-ylmethyl-phenoxy)-piperidine-1-carboxylic acid 4-(5-trifluoromethyl-pyridin-2-yloxy)-phenyl ester,
4-[4-(5-Methyl-pyridin-2-ylmethyl)-phenoxy]-piperidine-1-carboxylic acid 4-(5-trifluoromethyl-pyridin-2-yloxy)-phenyl ester,
4-(5-Pyridin-4-yl-[1,3,4]oxadiazol-2-ylsulfanyl)-piperidine-1-carboxylic acid 4-(5-trifluoromethyl-pyridin-2-yloxy)-phenyl ester,
4-(5-Pyridin-4-yl-1H-[1,2,4]triazol-3-ylsulfanyl)-piperidine-1-carboxylic acid 4-(5-trifluoromethyl-pyridin-2-yloxy)-phenyl ester,
4-(1-Methyl-1H-tetrazol-5-ylsulfanyl)-piperidine-1-carboxylic acid 4-(5-trifluoromethyl-pyridin-2-yloxy)-phenyl ester,
4-[4-(4-Ethoxycarbonyl-oxazol-2-yl)-phenoxy]-piperidine-1-carboxylic acid 4-(5-trifluoromethyl-pyridin-2-yloxy)-phenyl ester,
4-(4′-Methoxycarbonyl-biphenyl-4-yloxy)-piperidine-1-carboxylic acid 4-(5-trifluoromethyl-pyridin-2-yloxy)-phenyl ester,
5-{1-[4-(5-Trifluoromethyl-pyridin-2-yloxy)-phenoxycarbonyl]-piperidin-4-yloxy)-1H-indole-2-carboxylic acid ethyl ester,
4-[4-(2-Ethoxycarbonyl-ethyl)-phenoxy]-piperidine-1-carboxylic acid 4-(5-trifluoromethyl-pyridin-2-yloxy)-phenyl ester,
4-(3-Ethoxycarbonyl-phenoxy)-piperidine-1-carboxylic acid 4-(5-trifluoromethyl-pyridin-2-yloxy)-phenyl ester,
4-(4-Ethoxycarbonyl-1H-imidazol-2-ylsulfanyl)-piperidine-1-carboxylic acid 4-(5-trifluoromethyl-pyridin-2-yloxy)-phenyl ester,
4-(4-Allyloxycarbonylmethyl-phenoxy)-piperidine-1-carboxylic acid 4-(5-trifluoromethyl-pyridin-2-yloxy)-phenyl ester,
4-(4′-Carboxy-biphenyl-4-yloxy)-piperidine-1-carboxylic acid 4-(5-trifluoromethyl-pyridin-2-yloxy)-phenyl ester,
4-[4-(4-Carboxy-oxazol-2-yl)-phenoxy]-piperidine-1-carboxylic acid 4-(5-trifluoromethyl-pyridin-2-yloxy)-phenyl ester,
4-[4-(2-Carboxy-ethyl)-phenoxy]-piperidine-1-carboxylic acid 4-(5-trifluoromethyl-pyridin-2-yloxy)-phenyl ester,
4-(3-Carboxy-phenoxy)-piperidine-1-carboxylic acid 4-(5-trifluoromethyl-pyridin-2-yloxy)-phenyl ester,
4-(4-Carboxy-phenoxy)-piperidine-1-carboxylic acid 4-(5-trifluoromethyl-pyridin-2-yloxy)-phenyl ester,
4-(4-Carboxymethyl-phenoxy)-piperidine-1-carboxylic acid 4-(5-trifluoromethyl-pyridin-2-yloxy)-phenyl ester,
4-(1H-imidazol-2-ylsulfanylmethyl)-piperidine-1-carboxylic acid 4-(5-trifluoromethyl-pyridin-2-yloxy)-phenyl ester,
4-(4-Allyloxycarbonylmethyl-phenoxymethyl)-piperidine-1-carboxylic acid 4-(5-trifluoromethyl-pyridin-2-yloxy)-phenyl ester,
4-(4-Allyloxycarbonyl-phenoxymethyl)-piperidine-1-carboxylic acid 4-(5-trifluoromethyl-pyridin-2-yloxy)-phenyl ester,
4-(4-Carboxymethyl-phenoxymethyl)-piperidine-1-carboxylic acid 4-(5-trifluoromethyl-pyridin-2-yloxy)-phenyl ester,
4-(4-Carboxy-phenoxymethyl)-piperidine-1-carboxylic acid 4-(5-trifluoromethyl-pyridin-2-yloxy)-phenyl ester,
4-(4-Allyloxycarbonyl-phenoxy)-piperidine-1-carboxylic acid 4-(4-trifluoromethyl-benzyl)-phenyl ester,
4-(4-Carboxy-phenoxy)-piperidine-1-carboxylic acid 4-(4-trifluoromethyl-benzyl)-phenyl ester,
4-(Pyrazol-1-yloxy)-piperidine-1-carboxylic acid 4-(5-methyl-pyridin-2-ylmethyl)-phenyl ester,
4-(Imidazol-1-yloxy)-piperidine-1-carboxylic acid 4-(6-methyl-pyridin-3-ylmethyl)-phenyl ester,
4-Phenoxy-piperidine-1-carboxylic acid 4-(6-methyl-pyridin-3-ylmethyl)-phenyl ester,
4-(1H-Imidazol-2-ylsulfanyl)-piperidine-1-carboxylic acid 4-(5-methyl-pyridin-2-ylmethyl)-phenyl ester,
4-(4-Allyloxycarbonylmethyl-phenoxy)-piperidine-1-carboxylic acid 4-(5-methyl-pyridin-2-ylmethyl)-phenyl ester,
4-(4-Carboxymethyl-phenoxy)-piperidine-1-carboxylic acid 4-(5-methyl-pyridin-2-ylmethyl)-phenyl ester,
4-(1-Methyl-1H-imidazol-2-ylsulfanyl)-piperidine-1-carboxylic acid 4-[2-(4,4-dimethyl-2-piperidin-1-yl)-ethyl]-phenyl ester,
4-(4-Allyloxycarbonylmethyl-phenoxy)-piperidine-1-carboxylic acid 4-[2-(4,4-dimethyl-2,6-dioxo-piperidin-1-yl)-ethyl]-phenyl ester,
4-(1H-Imidazol-2-ylsulfanyl)-piperidine-1-carboxylic acid 4-[2-(4,4-dimethyl-2,6-dioxo-piperidin-1-yl)-ethyl]-phenyl ester, 4-(4-Carboxymethyl-phenoxy)-piperidine-1-carboxylic acid 4-[2-(4,4-dimethyl-2,6-dioxo-piperidin-1-yl)-ethyl]-phenyl ester,
4-(4-Allyloxycarbonyl-phenoxy)-piperidine-1-carboxylic acid 4-[2-(4,4-dimethyl-2,6-dioxo-piperidin-1-yl)-ethyl]-phenyl ester,
4-(4-Carboxy-phenoxy)-piperidine-1-carboxylic acid 4-[2-(4,4-dimethyl-2,6-dioxo-piperidin-1-yl)-ethyl]-phenyl ester,
4-(1H-[1,2,4]Triazol-3-ylsulfanyl)-piperidine-1-carboxylic acid 4-(5-methyl-pyridin-2-ylmethyl)-phenyl ester,4-(Pyrazol-1-yloxy)-piperidine-1-carboxylic acid 5-benzoylamino-pyridin-2-yl ester,
4-(Pyrazol-1-yloxy)-piperidine-1-carboxylic acid 4,4-dimethyl-2,6-dioxo-3,4,5,6-tetrahydro-2H-[1,3′]bipyridinyl-6′-yl ester,
4-[2-(3,4-Dihydro-1H-isoquinolin-2-yl)-ethyl]-piperidine-1-carboxylic acid 4-(5-trifluoromethyl-pyridin-2-yloxy)-phenyl ester,
4-(2-Piperidin-1-yl-ethyl)-piperidine-1-carboxylic acid 4-(5-trifluoromethyl-pyridin-2-yloxy)-phenyl ester,
4-(1H-Benzoimidazol-2-ylsulfanyl)-piperidine-1-carboxylic acid 4-(5-trifluoromethyl-pyridin-2-yloxy)-phenyl ester,
4-(Thiazolo[4,5-b]pyridin-2-ylsulfanyl)-piperidine-1-carboxylic acid 4-(5-trifluoromethyl-pyridin-2-yloxy)-phenyl ester, and
4-(7H-Purin-6-ylsulfanyl)-piperidine-1-carboxylic acid 4-(5-trifluoromethyl-pyridin-2-yloxy)-phenyl ester.

86. A compound according to claim 1, where the compound is selected from the group consisting of
4-(1-Methyl-1H-tetrazol-5-ylsulfanyl)-piperidine-1-carboxylic acid 4-(5-methyl-pyridin-2-ylmethyl)-phenyl ester,
4-[1-(2-Dimethylamino-ethyl)-1H-tetrazol-5-ylsulfanyl]-piperidine-1-carboxylic acid 4-(5-methyl-pyridin-2-ylmethyl)-phenyl ester,
4-(Imidazol-1-yloxy)-piperidine-1-carboxylic acid 4-[2-(4,4-dimethyl-2,6-dioxo-piperidin-1-yl)-ethyl]-phenyl ester,
4-(Pyrazol-1-yloxy)-piperidine-1-carboxylic acid 4-[2-(4,4-dimethyl-2,6-dioxo-piperidin-1-yl)-ethyl]-phenyl ester,
4-(1H-[1,2,4]Triazol-3-ylsulfanyl)-piperidine-1-carboxylic acid 4-(5-trifluoromethyl-pyridin-2-yloxy)-phenyl ester,
4-[1-(4-Carboxy-phenyl)-1H-tetrazol-5-ylsulfanyl]-piperidine-1-carboxylic acid 4-(5-trifluoromethyl-pyridin-2-yloxy)-phenyl ester,
4-[4-(Pyridin-2-yloxy)-phenoxy]-piperidine-1-carboxylic acid 4-(5-trifluoromethyl-pyridin-2-yloxy)-phenyl ester,
4-[4-(5-Methyl-pyridin-2-yloxy)-phenoxy]-piperidine-1-carboxylic acid 4-(5-trifluoromethyl-pyridin-2-yloxy)-phenyl ester,
4-(1-Methyl-1H-imidazol-2-ylsulfanylmethyl)-piperidine-1-carboxylic acid 4-(5-trifluoromethyl-pyridin-2-yloxy)-phenyl ester,
4-(Imidazol-1-yloxymethyl)-piperidine-1-carboxylic acid 4-(5-trifluoromethyl-pyridin-2-yloxy)-phenyl ester,
4-[4-(Pyridin-2-yloxy)-phenoxymethyl]-piperidine-1-carboxylic acid 4-(5-trifluoromethyl-pyridin-2-yloxy)-phenyl ester,
4-[4-(5-Methyl-pyridin-2-yloxy)-phenoxymethyl]-piperidine-1-carboxylic acid 4-(5-trifluoromethyl-pyridin-2-yloxy)-phenyl ester,
4-(1-Methyl-1H-tetrazol-5-ylsulfanylmethyl)-piperidine-1-carboxylic acid 4-(5-trifluoromethyl-pyridin-2-yloxy)-phenyl ester,
4-[1-(2-Dimethylamino-ethyl)-1H-tetrazol-5-ylsulfanyl]-piperidine-1-carboxylic acid 4-(5-trifluoromethyl-pyridin-2-yloxy)-phenyl ester,
4-[1-(2-Carboxy-ethyl)-1H-tetrazol-5-ylsulfanyl]-piperidine-1-carboxylic acid 4-(5-trifluoromethyl-pyridin-2-yloxy)-phenyl ester,
4-(1-Carboxymethyl-1H-tetrazol-5-ylsulfanyl)-piperidine-1-carboxylic acid 4-(5-trifluoromethyl-pyridin-2-yloxy)-phenyl ester,
4-(Pyrazol-1-yloxymethyl)-piperidine-1-carboxylic acid 4-(5-trifluoromethyl-pyridin-2-yloxy)-phenyl ester,
4-(1H-[1,2,4]Triazol-3-ylsulfanylmethyl)-piperidine-1-carboxylic acid 4-(5-trifluoromethyl-pyridin-2-yloxy)-phenyl ester,
4-(1H-[1,2,4]Triazol-3-ylsulfanyl)-piperidine-1-carboxylic acid 4-(5-chloro-pyridin-2-yloxy)-phenyl ester,
4-(1H-Imidazol-2-ylsulfanyl)-piperidine-1-carboxylic acid 4-(5-chloro-pyridin-2-yloxy)-phenyl ester,
4-(1-Methyl-1H-imidazol-2-ylsulfanyl)-piperidine-1-carboxylic acid 4-(5-chloro-pyridin-2-yloxy)-phenyl ester,
4-(1-Methyl-1H-tetrazol-5-ylsulfanyl)-piperidine-1-carboxylic acid 4-(5-chloro-pyridin-2-yloxy)-phenyl ester,
4-(Imidazol-1-yloxy)-piperidine-1-carboxylic acid 4-(5-chloro-pyridin-2-yloxy)-phenyl ester,
4-[1-(4-Carboxy-phenyl)-1H-tetrazol-5-ylsulfanyl]-piperidine-1-carboxylic acid 4-(5-chloro-pyridin-2-yloxy)-phenyl ester,
4-(4-Carboxymethyl-phenoxy)-piperidine-1-carboxylic acid 4-(5-chloro-pyridin-2-yloxy)-phenyl ester,
4-(Pyrazol-1-yloxy)-piperidine-1-carboxylic acid 4-(5-chloro-pyridin-2-yloxy)-phenyl ester,
4-(4-Carboxy-phenoxy)-piperidine-1-carboxylic acid 4-(5-chloro-pyridin-2-yloxy)-phenyl ester,
4-[4-(Pyridin-2-yloxy)-phenoxy]-piperidine-1-carboxylic acid 4-(5-chloro-pyridin-2-yloxy)-phenyl ester,
4-[4-(5-Methyl-pyridin-2-yloxy)-phenoxy]-piperidine-1-carboxylic acid 4-(5-chloro-pyridin-2-yloxy)-phenyl ester,
4-(4-Carboxy-phenoxymethyl)-piperidine-1-carboxylic acid 4-(5-chloro-pyridin-2-yloxy)-phenyl ester,
4-(4-Carboxymethyl-phenoxymethyl)-piperidine-1-carboxylic acid 4-(5-chloro-pyridin-2-yloxy)-phenyl ester,
4-(1H-Imidazol-2-ylsulfanylmethyl)-piperidine-1-carboxylic acid 4-(5-chloro-pyridin-2-yloxy)-phenyl ester,
4-(1-Methyl-1H-imidazol-2-ylsulfanylmethyl)-piperidine-1-carboxylic acid 4-(5-chloro-pyridin-2-yloxy)-phenyl ester,
4-(Imidazol-1-yloxymethyl)-piperidine-1-carboxylic acid 4-(5-chloro-pyridin-2-yloxy)-phenyl ester,
4-[4-(Pyridin-2-yloxy)-phenoxymethyl]-piperidine-1-carboxylic acid 4-(5-chloro-pyridin-2-yloxy)-phenyl ester,
4-[4-(5-Methyl-pyridin-2-yloxy)-phenoxymethyl]-piperidine-1-carboxylic acid 4-(5-chloro-pyridin-2-yloxy)-phenyl ester,
4-(1-Methyl-1H-tetrazol-5-ylsulfanylmethyl)-piperidine-1-carboxylic acid 4-(5-chloro-pyridin-2-yloxy)-phenyl ester,
4-[1-(2-Dimethylamino-ethyl)-1H-tetrazol-5-ylsulfanyl]-piperidine-1-carboxylic acid 4-(5-chloro-pyridin-2-yloxy)-phenyl ester,
4-[1-(2-Carboxy-ethyl)-1H-tetrazol-5-ylsulfanyl]-piperidine-1-carboxylic acid 4-(5-chloro-pyridin-2-yloxy)-phenyl ester,
4-(1-Carboxymethyl-1H-tetrazol-5-ylsulfanyl)-piperidine-1-carboxylic acid 4-(5-chloro-pyridin-2-yloxy)-phenyl ester,
4-(Pyrazol-1-yloxymethyl)-piperidine-1-carboxylic acid 4-(5-chloro-pyridin-2-yloxy)-phenyl ester,
4-(1H-[1,2,4]Triazol-3-ylsulfanylmethyl)-piperidine-1-carboxylic acid 4-(5-chloro-pyridin-2-yloxy)-phenyl ester,
4-[2-(Benzyl-methyl-amino)-ethyl]-piperidine-1-carboxylic acid 4-(5-chloro-pyridin-2-yloxy)-phenyl ester,
4-(1H-[1,2,4]Triazol-3-ylsulfanyl)-piperidine-1-carboxylic acid 4-(3,3-dimethyl-butylcarbamoyl)-phenyl ester,
4-(1H-Imidazol-2-ylsulfanyl)-piperidine-1-carboxylic acid 4-(3,3-dimethyl-butylcarbamoyl)-phenyl ester,
4-(1-Methyl-1H-imidazol-2-ylsulfanyl)-piperidine-1-carboxylic acid 4-(3,3-dimethyl-butylcarbamoyl)-phenyl ester,
4-(1-Methyl-1H-tetrazol-5-ylsulfanyl)-piperidine-1-carboxylic acid 4-(3,3-dimethyl-butylcarbamoyl)-phenyl ester,
4-(Imidazol-1-yloxy)-piperidine-1-carboxylic acid 4-(3,3-dimethyl-butylcarbamoyl)-phenyl ester,
4-[1-(4-Carboxy-phenyl)-1H-tetrazol-5-ylsulfanyl]-piperidine-1-carboxylic acid 4-(3,3-dimethyl-butylcarbamoyl)-phenyl ester,
4-(4-Carboxymethyl-phenoxy)-piperidine-1-carboxylic acid 4-(3,3-dimethyl-butylcarbamoyl)-phenyl ester,
4-(Pyrazol-1-yloxy)-piperidine-1-carboxylic acid 4-(3,3-dimethyl-butylcarbamoyl)-phenyl ester,
4-(4-Carboxy-phenoxy)-piperidine-1-carboxylic acid 4-(3,3-dimethyl-butylcarbamoyl)-phenyl ester,
4-[4-(Pyridin-2-yloxy)-phenoxy]-piperidine-1-carboxylic acid 4-(3,3-dimethyl-butylcarbamoyl)-phenyl ester,
4-[4-(5-Methyl-pyridin-2-yloxy)-phenoxy]-piperidine-1-carboxylic acid 4-(3,3-dimethyl-butylcarbamoyl)-phenyl ester,
4-(4-Carboxy-phenoxymethyl)-piperidine-1-carboxylic acid 4-(3,3-dimethyl-butylcarbamoyl)-phenyl ester,
4-(4-Carboxymethyl-phenoxymethyl)-piperidine-1-carboxylic acid 4-(3,3-dimethyl-butylcarbamoyl)-phenyl ester,
4-(1H-Imidazol-2-ylsulfanylmethyl)-piperidine-1-carboxylic acid 4-(3,3-dimethyl-butylcarbamoyl)-phenyl ester,
4-(1-Methyl-1H-imidazol-2-ylsulfanylmethyl)-piperidine-1-carboxylic acid 4-(3,3-dimethyl-butylcarbamoyl)-phenyl ester,
4-(Imidazol-1-yloxymethyl)-piperidine-1-carboxylic acid 4-(3,3-dimethyl-butylcarbamoyl)-phenyl ester,
4-[4-(Pyridin-2-yloxy)-phenoxymethyl]-piperidine-1-carboxylic acid 4-(3,3-dimethyl-butylcarbamoyl)-phenyl ester,
4-[4-(5-Methyl-pyridin-2-yloxy)-phenoxymethyl]-piperidine-1-carboxylic acid 4-(3,3-dimethyl-butylcarbamoyl)-phenyl ester,
4-(1-Methyl-1H-tetrazol-5-ylsulfanylmethyl)-piperidine-1-carboxylic acid 4-(3,3-dimethyl-butylcarbamoyl)-phenyl ester,
4-[1-(2-Dimethylamino-ethyl)-1H-tetrazol-5-ylsulfanyl]-piperidine-1-carboxylic acid 4-(3,3-dimethyl-butylcarbamoyl)-phenyl ester,
4-[1-(2-Carboxy-ethyl)-1H-tetrazol-5-ylsulfanyl]-piperidine-1-carboxylic acid 4-(3,3-dimethyl-butylcarbamoyl)-phenyl ester,
4-(1-Carboxymethyl-1H-tetrazol-5-ylsulfanyl)-piperidine-1-carboxylic acid 4-(3,3-dimethyl-butylcarbamoyl)-phenyl ester,
4-(Pyrazol-1-yloxymethyl)-piperidine-1-carboxylic acid 4-(3,3-dimethyl-butylcarbamoyl)-phenyl ester,
4-(1H-[1,2,4]Triazol-3-ylsulfanylmethyl)-piperidine-1-carboxylic acid 4-(3,3-dimethyl-butylcarbamoyl)-phenyl ester,
4-[2-(Benzyl-methyl-amino)-ethyl]-piperidine-1-carboxylic acid 4-(3,3-dimethyl-butylcarbamoyl)-phenyl ester,
4-(1-Methyl-1H-imidazol-2-ylsulfanyl)-piperidine-1-carboxylic acid 4-(5-methyl-pyridin-2-ylmethyl)-phenyl ester,
4-[1-(4-Carboxy-phenyl)-1H-tetrazol-5-ylsulfanyl]-piperidine-1-carboxylic acid 4-(5-methyl-pyridin-2-ylmethyl)-phenyl ester,
4-(4-Carboxy-phenoxy)-piperidine-1-carboxylic acid 4-(5-methyl-pyridin-2-ylmethyl)-phenyl ester,
4-[4-(Pyridin-2-yloxy)-phenoxy]-piperidine-1-carboxylic acid 4-(5-methyl-pyridin-2-ylmethyl)-phenyl ester,
4-[4-(5-Methyl-pyridin-2-yloxy)-phenoxy]-piperidine-1-carboxylic acid 4-(5-methyl-pyridin-2-ylmethyl)-phenyl ester,
4-(4-Carboxy-phenoxymethyl)-piperidine-1-carboxylic acid 4-(5-methyl-pyridin-2-ylmethyl)-phenyl ester,
4-(4-Carboxymethyl-phenoxymethyl)-piperidine-1-carboxylic acid 4-(5-methyl-pyridin-2-ylmethyl)-phenyl ester,
4-(1H-Imidazol-2-ylsulfanylmethyl)-piperidine-1-carboxylic acid 4-(5-methyl-pyridin-2-ylmethyl)-phenyl ester,
4-(1-Methyl-1H-imidazol-2-ylsulfanylmethyl)-piperidine-l -carboxylic acid 4-(5-methyl-pyridin-2-ylmethyl)-phenyl ester,
4-(Imidazol-1-yloxymethyl)-piperidine-1-carboxylic acid 4-(5-methyl-pyridin-2-ylmethyl)-phenyl ester,
4-[4-(Pyridin-2-yloxy)-phenoxymethyl]-piperidine-1-carboxylic acid 4-(5-methyl-pyridin-2-ylmethyl)-phenyl ester,
4-[4-(5-Methyl-pyridin-2-yloxy)-phenoxymethyl]-piperidine-1-carboxylic acid 4-(5-methyl-pyridin-2-ylmethyl)-phenyl ester,
4-(1-Methyl-1H-tetrazol-5-ylsulfanylmethyl)-piperidine-1-carboxylic acid 4-(5-methyl-pyridin-2-ylmethyl)-phenyl ester,
4-[1-(2-Carboxy-ethyl)-1H-tetrazol-5-ylsulfanyl]-piperidine-1-carboxylic acid 4-(5-methyl-pyridin-2-ylmethyl)-phenyl ester,
4-(1-Carboxymethyl-1H-tetrazol-5-ylsulfanyl)-piperidine-1-carboxylic acid 4-(5-methyl-pyridin-2-ylmethyl)-phenyl ester,
4-(Pyrazol-1-yloxymethyl)-piperidine-1-carboxylic acid 4-(5-methyl-pyridin-2-ylmethyl)-phenyl ester,
4-(1H-[1,2,4]Triazol-3-ylsulfanylmethyl)-piperidine-1-carboxylic acid 4-(5-methyl-pyridin-2-ylmethyl)-phenyl ester,
4-[2-(Benzyl-methyl-amino)-ethyl]-piperidine-1-carboxylic acid 4-(5-methyl-pyridin-2-ylmethyl)-phenyl ester,
4-(1H-[1,2,4]Triazol-3-ylsulfanyl)-piperidine-1-carboxylic acid 4-(4-trifluoromethyl-benzyl)-phenyl ester,
4-(1H-Imidazol-2-ylsulfanyl)-piperidine-1-carboxylic acid 4-(4-trifluoromethyl-benzyl)-phenyl ester,
4-(1-Methyl-1H-imidazol-2-ylsulfanyl)-piperidine-1-carboxylic acid 4-(4-trifluoromethyl-benzyl)-phenyl ester,
4-(1-Methyl-1H-tetrazol-5-ylsulfanyl)-piperidine-1-carboxylic acid 4-(4-trifluoromethyl-benzyl)-phenyl ester,
4-(Imidazol-1-yloxy)-piperidine-1-carboxylic acid 4-(4-trifluoromethyl-benzyl)-phenyl ester,
4-[1-(4-Carboxy-phenyl)-1H-tetrazol-5-ylsulfanyl]-piperidine-1-carboxylic acid 4-(4-trifluoromethyl-benzyl)-phenyl ester,
4-(4-Carboxymethyl-phenoxy)-piperidine-1-carboxylic acid 4-(4-trifluoromethyl-benzyl)-phenyl ester,
4-(Pyrazol-1-yloxy)-piperidine-1-carboxylic acid 4-(4-trifluoromethyl-benzyl)-phenyl ester,
4-[4-(Pyridin-2-yloxy)-phenoxy]-piperidine-1-carboxylic acid 4-(4-trifluoromethyl-benzyl)-phenyl ester,
4-[4-(5-Methyl-pyridin-2-yloxy)-phenoxy]-piperidine-1-carboxylic acid 4-(4-trifluoromethyl-benzyl)-phenyl ester,
4-(4-Carboxy-phenoxymethyl)-piperidine-1-carboxylic acid 4-(4-trifluoromethyl-benzyl)-phenyl ester,
4-(4-Carboxymethyl-phenoxymethyl)-piperidine-1-carboxylic acid 4-(4-trifluoromethyl-benzyl)-phenyl ester,
4-(1H-Imidazol-2-ylsulfanylmethyl)-piperidine-1-carboxylic acid 4-(4-trifluoromethyl-benzyl)-phenyl ester,
4-(1-Methyl-1H-imidazol-2-ylsulfanylmethyl)-piperidine-1-carboxylic acid 4-(4-trifluoromethyl-benzyl)-phenyl ester,
4-(Imidazol-1-yloxymethyl)-piperidine-1-carboxylic acid 4-(4-trifluoromethyl-benzyl)-phenyl ester,
4-[4-(Pyridin-2-yloxy)-phenoxymethyl]-piperidine-1-carboxylic acid 4-(4-trifluoromethyl-benzyl)-phenyl ester,
4-[4-(5-Methyl-pyridin-2-yloxy)-phenoxymethyl]-piperidine-1-carboxylic acid 4-(4-trifluoromethyl-benzyl)-phenyl ester,
4-(1-Methyl-1H-tetrazol-5-ylsulfanylmethyl)-piperidine-1-carboxylic acid 4-(4-trifluoromethyl-benzyl)-phenyl ester,
4-[1-(2-Dimethylamino-ethyl)-1H-tetrazol-5-ylsulfanyl]-piperidine-1-carboxylic acid 4-(4-trifluoromethyl-benzyl)-phenyl ester,
4-[1-(2-Carboxy-ethyl)-1H-tetrazol-5-ylsulfanyl]-piperidine-1-carboxylic acid 4-(4-trifluoromethyl-benzyl)-phenyl ester,
4-(1-Carboxymethyl-1H-tetrazol-5-ylsulfanyl)-piperidine-1-carboxylic acid 4-(4-trifluoromethyl-benzyl)-phenyl ester,
4-(Pyrazol-1-yloxymethyl)-piperidine-1-carboxylic acid 4-(4-trifluoromethyl-benzyl)-phenyl ester,
4-(1H-[1,2,4]Triazol-3-ylsulfanylmethyl)-piperidine-1-carboxylic acid 4-(4-trifluoromethyl-benzyl)-phenyl ester,
4-[2-(Benzyl-methyl-amino)-ethyl]-piperidine-1-carboxylic acid 4-(4-trifluoromethyl-benzyl)-phenyl ester,
4-(1H-[1,2,4]Triazol-3-ylsulfanyl)-piperidine-1-carboxylic acid 4,4-dimethyl-2,6-dioxo-3,4,5,6-tetrahydro-2H-[1,3′]bipyridinyl-6′-yl ester,
4-(1H-Imidazol-2-ylsulfanyl)-piperidine-1-carboxylic acid 4,4-dimethyl-2,6-dioxo-3,4,5,6-tetrahydro-2H-[1,3′]bipyridinyl-6′-yl ester,
4-(1-Methyl-1H-imidazol-2-ylsulfanyl)-piperidine-1-carboxylic acid 4,4-dimethyl-2,6-dioxo-3,4,5,6-tetrahydro-2H-[1,3′]bipyridinyl-6′-yl ester,
4-(1-Methyl-1H-tetrazol-5-ylsulfanyl)-piperidine-1-carboxylic acid 4,4-dimethyl-2,6-dioxo-3,4,5,6-tetrahydro-2H-[1,3′]bipyridinyl-6′-yl ester,
4-(Imidazol-1-yloxy)-piperidine-1-carboxylic acid 4,4-dimethyl-2,6-dioxo-3,4,5,6-tetrahydro-2H-[1,3′]bipyridinyl-6′-yl ester,
4-[1-(4-Carboxy-phenyl)-1H-tetrazol-5-ylsulfanyl]-piperidine-1-carboxylic acid 4,4-dimethyl-2,6-dioxo-3,4,5,6-tetrahydro-2H-[1,3′]bipyridinyl-6′-yl ester,
4-(4-Carboxymethyl-phenoxy)-piperidine-1-carboxylic acid 4,4-dimethyl-2,6-dioxo-3,4,5,6-tetrahydro-2H-[1,3′]bipyridinyl-6′-yl ester,
4-(4-Carboxy-phenoxy)-piperidine-1-carboxylic acid 4,4-dimethyl-2,6-dioxo-3,4,5,6-tetrahydro-2H-[1,3′]bipyridinyl-6′-yl ester,
4-[4-(Pyridin-2-yloxy)-phenoxy]-piperidine-1-carboxylic acid 4,4-dimethyl-2,6-dioxo-3,4,5,6-tetrahydro-2H-[1,3′]bipyridinyl-6′-yl ester,
4-[4-(5-Methyl-pyridin-2-yloxy)-phenoxy]-piperidine-1-carboxylic acid 4,4-dimethyl-2,6-dioxo-3,4,5,6-tetrahydro-2H-[1,3′]bipyridinyl-6′-yl ester,
4-(4-Carboxy-phenoxymethyl)-piperidine-1-carboxylic acid 4,4-dimethyl-2,6-dioxo-3,4,5,6-tetrahydro-2H-[1,3′]bipyridinyl-6′-yl ester,
4-(4-Carboxymethyl-phenoxymethyl)-piperidine-1-carboxylic acid 4,4-dimethyl-2,6-dioxo-3,4,5,6-tetrahydro-2H-[1,3′]bipyridinyl-6′-yl ester,
4-(1H-Imidazol-2-ylsulfanylmethyl)-piperidine-1-carboxylic acid 4,4-dimethyl-2,6-dioxo-3,4,5,6-tetrahydro-2H-[1,3′]bipyridinyl-6′-yl ester,
4-(1-Methyl-1H-imidazol-2-ylsulfanylmethyl)-piperidine-1-carboxylic acid 4,4-dimethyl-2,6-dioxo-3,4,5,6-tetrahydro-2H-[1,3′]bipyridinyl-6′-yl ester,
4-(Imidazol-1-yloxymethyl)-piperidine-1-carboxylic acid 4,4-dimethyl-2,6-dioxo-3,4,5,6-tetrahydro-2H-[1,3′]bipyridinyl-6′-yl ester,
4-[4-(Pyridin-2-yloxy)-phenoxymethyl]-piperidine-1-carboxylic acid 4,4-dimethyl-2,6-dioxo-3,4,5,6-tetrahydro-2H-[1,3′]bipyridinyl-6′-yl ester,
4-[4-(5-Methyl-pyridin-2-yloxy)-phenoxymethyl]-piperidine-1-carboxylic acid 4,4-dimethyl-2,6-dioxo-3,4,5,6-tetrahydro-2H-[1,3′]bipyridinyl-6′-yl ester,
4-(1-Methyl-1H-tetrazol-5-ylsulfanylmethyl)-piperidine-1-carboxylic acid 4,4-dimethyl-2,6-dioxo-3,4,5,6-tetrahydro-2H-[1,3′]bipyridinyl-6′-yl ester,
4-[1-(2-Dimethylamino-ethyl)-1H-tetrazol-5-ylsulfanyl]-piperidine-1-carboxylic acid 4,4-dimethyl-2,6-dioxo-3,4,5,6-tetrahydro-2H-[1,3′]bipyridinyl-6′-yl ester,
4-[1-(2-Carboxy-ethyl)-1H-tetrazol-5-ylsulfanyl]-piperidine-1-carboxylic acid 4,4-dimethyl-2,6-dioxo-3,4,5,6-tetrahydro-2H-[1,3′]bipyridinyl-6′-yl ester,
4-(1-Carboxymethyl-1H-tetrazol-5-ylsulfanyl)-piperidine-1-carboxylic acid 4,4-dimethyl-2,6-dioxo-3,4,5,6-tetrahydro-2H-[1,3′]bipyridinyl-6′-yl ester,
4-(Pyrazol-1-yloxymethyl)-piperidine-1-carboxylic acid 4,4-dimethyl-2,6-dioxo-3,4,5,6-tetrahydro-2H-[1,3′]bipyridinyl-6′-yl ester,
4-(1H-[1,2,4]Triazol-3-ylsulfanylmethyl)-piperidine-1-carboxylic acid 4,4-dimethyl-2,6-dioxo-3,4,5,6-tetrahydro-2H-[1,3′]bipyridinyl-6′-yl ester,
4-[2-(Benzyl-methyl-amino)-ethyl]-piperidine-1-carboxylic acid 4,4-dimethyl-2,6-dioxo-3,4,5,6-tetrahydro-2H-[1,3′]bipyridinyl-6′-yl ester,
4-(1H-[1,2,4]Triazol-3-ylsulfanyl)-piperidine-1-carboxylic acid 5-[2-(4,4-dimethyl-2,6-dioxo-piperidin-1-yl)-ethyl]-pyridin-2-yl ester,
4-(1H-Imidazol-2-ylsulfanyl)-piperidine-1-carboxylic acid 5-[2-(4,4-dimethyl-2,6-dioxo-piperidin-1-yl)-ethyl]-pyridin-2-yl ester,
4-(1-Methyl-1H-imidazol-2-ylsulfanyl)-piperidine-1-carboxylic acid 5-[2-(4,4-dimethyl-2,6-dioxo-piperidin-1-yl)-ethyl]-pyridin-2-yl ester,
4-(1-Methyl-1H-tetrazol-5-ylsulfanyl)-piperidine-1-carboxylic acid 5-[2-(4,4-dimethyl-2,6-dioxo-piperidin-1-yl)-ethyl]-pyridin-2-yl ester,
4-(Imidazol-1-yloxy)-piperidine-1-carboxylic acid 5-[2-(4,4-dimethyl-2,6-dioxo-piperidin-1-yl)-ethyl]-pyridin-2-yl ester,
4-[1-(4-Carboxy-phenyl)-1H-tetrazol-5-ylsulfanyl]-piperidine-1-carboxylic acid 5-[2-(4,4-dimethyl-2,6-dioxo-piperidin-1-yl)-ethyl]-pyridin-2-yl ester,
4-(4-Carboxymethyl-phenoxy)-piperidine-1-carboxylic acid 5-[2-(4,4-dimethyl-2,6-dioxo-piperidin-1-yl)-ethyl]-pyridin-2-yl ester,
4-(Pyrazol-1-yloxy)-piperidine-1-carboxylic acid 5-[2-(4,4-dimethyl-2,6-dioxo-piperidin-1-yl)-ethyl]-pyridin-2-yl ester,
4-(4-Carboxy-phenoxy)-piperidine-1-carboxylic acid 5-[2-(4,4-dimethyl-2,6-dioxo-piperidin-1-yl)-ethyl]-pyridin-2-yl ester,
4-[4-(Pyridin-2-yloxy)-phenoxy]-piperidine-1-carboxylic acid 5-[2-(4,4-dimethyl-2,6-dioxo-piperidin-1-yl)-ethyl]-pyridin-2-yl ester,
4-[4-(5-Methyl-pyridin-2-yloxy)-phenoxy]-piperidine-1-carboxylic acid 5-[2-(4,4-dimethyl-2,6-dioxo-piperidin-1-yl)-ethyl]-pyridin-2-yl ester,
4-(4-Carboxy-phenoxymethyl)-piperidine-1-carboxylic acid 5-[2-(4,4-dimethyl-2,6-dioxo-piperidin-1-yl)-ethyl]-pyridin-2-yl ester,
4-(4-Carboxymethyl-phenoxymethyl)-piperidine-1-carboxylic acid 5-[2-(4,4-dimethyl-2,6-dioxo-piperidin-1-yl)-ethyl]-pyridin-2-yl ester,
4-(1H-Imidazol-2-ylsulfanylmethyl)-piperidine-1-carboxylic acid 5-[2-(4,4-dimethyl-2,6-dioxo-piperidin-1-yl)-ethyl]-pyridin-2-yl ester,
4-(1-Methyl-1H-imidazol-2-ylsulfanylmethyl)-piperidine-1-carboxylic acid 5-[2-(4,4-dimethyl-2,6-dioxo-piperidin-1-yl)-ethyl]-pyridin-2-yl ester,
4-(Imidazol-1-yloxymethyl)-piperidine-1-carboxylic acid 5-[2-(4,4-dimethyl-2,6-dioxo-piperidin-1-yl)-ethyl]-pyridin-2-yl ester,
4-[4-(Pyridin-2-yloxy)-phenoxymethyl]-piperidine-1-carboxylic acid 5-[2-(4,4-dimethyl-2,6-dioxo-piperidin-1-yl)-ethyl]-pyridin-2-yl ester,
4-[4-(5-Methyl-pyridin-2-yloxy)-phenoxymethyl]-piperidine-i-carboxylic acid 5-[2-(4,4-dimethyl-2,6-dioxo-piperidin-1-yl)-ethyl]-pyridin-2-yl ester,
4-(1-Methyl-1H-tetrazol-5-ylsulfanylmethyl)-piperidine-1-carboxylic acid 5-[2-(4,4-dimethyl-2,6-dioxo-piperidin-1-yl)-ethyl]-pyridin-2-yl ester,
4-[1-(2-Dimethylamino-ethyl)-1H-tetrazol-5-ylsulfanyl]-piperidine-1-carboxylic acid 5-[2-(4,4-dimethyl-2,6-dioxo-piperidin-1-yl)-ethyl]-pyridin-2-yl ester,
4-[1-(2-Carboxy-ethyl)-1H-tetrazol-5-ylsulfanyl]-piperidine-1-carboxylic acid 5-[2-(4,4-dimethyl-2,6-dioxo-piperidin-1-yl)-ethyl]-pyridin-2-yl ester,
4-(1-Carboxymethyl-1H-tetrazol-5-ylsulfanyl)-piperidine-1-carboxylic acid 5-[2-(4,4-dimethyl-2,6-dioxo-piperidin-1-yl)-ethyl]-pyridin-2-yl ester,
4-(Pyrazol-1-yloxymethyl)-piperidine-1-carboxylic acid 5-[2-(4,4-dimethyl-2,6-dioxo-piperidin-1-yl)-ethyl]-pyridin-2-yl ester,
4-(1H-[1,2,4]Triazol-3-ylsulfanylmethyl)-piperidine-1-carboxylic acid 5-[2-(4,4-dimethyl-2,6-dioxo-piperidin-1-yl)-ethyl]-pyridin-2-yl ester,
4-[2-(Benzyl-methyl-amino)-ethyl]-piperidine-1-carboxylic acid 5-[2-(4,4-dimethyl-2,6-dioxo-piperidin-1-yl)-ethyl]-pyridin-2-yl ester,
4-(1H-[1,2,4]Triazol-3-ylsulfanyl)-piperidine-1-carboxylic acid 5-benzoylamino-pyridin-2-yl ester,
4-(1H-Imidazol-2-ylsulfanyl)-piperidine-1-carboxylic acid 5-benzoylamino-pyridin-2-yl ester,
4-(1-Methyl-1H-imidazol-2-ylsulfanyl)-piperidine-1-carboxylic acid 5-benzoylamino-pyridin-2-yl ester,
4-(1-Methyl-1H-tetrazol-5-ylsulfanyl)-piperidine-1-carboxylic acid 5-benzoylamino-pyridin-2-yl ester,
4-(Imidazol-1-yloxy)-piperidine-1-carboxylic acid 5-benzoylamino-pyridin-2-yl ester,
4-[1-(4-Carboxy-phenyl)-1H-tetrazol-5-ylsulfanyl]-piperidine-1-carboxylic acid 5-benzoylamino-pyridin-2-yl ester,
4-(4-Carboxymethyl-phenoxy)-piperidine-1-carboxylic acid 5-benzoylamino-pyridin-2-yl ester,
4-(4-Carboxy-phenoxy)-piperidine-1-carboxylic acid 5-benzoylamino-pyridin-2-yl ester,
4-[4-(Pyridin-2-yloxy)-phenoxy]-piperidine-1-carboxylic acid 5-benzoylamino-pyridin-2-yl ester,
4-[4-(5-Methyl-pyridin-2-yloxy)-phenoxy]-piperidine-1-carboxylic acid 5-benzoylamino-pyridin-2-yl ester,
4-(4-Carboxy-phenoxymethyl)-piperidine-1-carboxylic acid 5-benzoylamino-pyridin-2-yl ester,
4-(4-Carboxymethyl-phenoxymethyl)-piperidine-1-carboxylic acid 5-benzoylamino-pyridin-2-yl ester,
4-(1H-Imidazol-2-ylsulfanylmethyl)-piperidine-1-carboxylic acid 5-benzoylamino-pyridin-2-yl ester,
4-(1-Methyl-1H-imidazol-2-ylsulfanylmethyl)-piperidine-1-carboxylic acid 5-benzoylamino-pyridin-2-yl ester,
4-(Imidazol-1-yloxymethyl)-piperidine-1-carboxylic acid 5-benzoylamino-pyridin-2-yl ester,
4-[4-(Pyridin-2-yloxy)-phenoxymethyl]-piperidine-1-carboxylic acid 5-benzoylamino-pyridin-2-yl ester,
4-[4-(5-Methyl-pyridin-2-yloxy)-phenoxymethyl]-piperidine-1-carboxylic acid 5-benzoylamino-pyridin-2-yl ester,
4-(1-Methyl-1H-tetrazol-5-ylsulfanylmethyl)-piperidine-1-carboxylic acid 5-benzoylamino-pyridin-2-yl ester,
4-[1-(2-Dimethylamino-ethyl)-1H-tetrazol-5-ylsulfanyl]-piperidine-1-carboxylic acid 5-benzoylamino-pyridin-2-yl ester,
4-[1-(2-Carboxy-ethyl)-1H-tetrazol-5-ylsulfanyl]-piperidine-1-carboxylic acid 5-benzoylamino-pyridin-2-yl ester,
4-(1-Carboxymethyl-1H-tetrazol-5-ylsulfanyl)-piperidine-1-carboxylic acid 5-benzoylamino-pyridin-2-yl ester,
4-(Pyrazol-1-yloxymethyl)-piperidine-1-carboxylic acid 5-benzoylamino-pyridin-2-yl ester,
4-(1H-[1,2,4]Triazol-3-ylsulfanylmethyl)-piperidine-1-carboxylic acid 5-benzoylamino-pyridin-2-yl ester,
4-[2-(Benzyl-methyl-amino)-ethyl]-piperidine-1-carboxylic acid 5-benzoylamino-pyridin-2-yl ester,
4-(1H-[1,2,4]Triazol-3-ylsulfanyl)-piperidine-1-carboxylic acid 5-(3-tert-butyl-thioureido)-pyridin-2-yl ester,
4-(1H-Imidazol-2-ylsulfanyl)-piperidine-1-carboxylic acid 5-(3-tert-butyl-thioureido)-pyridin-2-yl ester,
4-(1-Methyl-1H-imidazol-2-ylsulfanyl)-piperidine-1-carboxylic acid 5-(3-tert-butyl-thioureido)-pyridin-2-yl ester,
4-(1-Methyl-1H-tetrazol-5-ylsulfanyl)-piperidine-1-carboxylic acid 5-(3-tert-butyl-thioureido)-pyridin-2-yl ester,
4-(Imidazol-1-yloxy)-piperidine-1-carboxylic acid 5-(3-tert-butyl-thioureido)-pyridin-2-yl ester,
4-[1-(4-Carboxy-phenyl)-1H-tetrazol-5-ylsulfanyl]-piperidine-1-carboxylic acid 5-(3-tert-butyl-thioureido)-pyridin-2-yl ester,
4-(4-Carboxymethyl-phenoxy)-piperidine-1-carboxylic acid 5-(3-tert-butyl-thioureido)-pyridin-2-yl ester,
4-(Pyrazol-1-yloxy)-piperidine-1-carboxylic acid 5-(3-tert-butyl-thioureido)-pyridin-2-yl ester,
4-(4-Carboxy-phenoxy)-piperidine-1-carboxylic acid 5-(3-tert-butyl-thioureido)-pyridin-2-yl ester,
4-[4-(Pyridin-2-yloxy)-phenoxy]-piperidine-1-carboxylic acid 5-(3-tert-butyl-thioureido)-pyridin-2-yl ester,
4-[4-(5-Methyl-pyridin-2-yloxy)-phenoxy]-piperidine-1-carboxylic acid 5-(3-tert-butyl-thioureido)-pyridin-2-yl ester,
4-(4-Carboxy-phenoxymethyl)-piperidine-1-carboxylic acid 5-(3-tert-butyl-thioureido)-pyridin-2-yl ester,
4-(4-Carboxymethyl-phenoxymethyl)-piperidine-1-carboxylic acid 5-(3-tert-butyl-thioureido)-pyridin-2-yl ester,
4-(1H-Imidazol-2-ylsulfanylmethyl)-piperidine-1-carboxylic acid 5-(3-tert-butyl-thioureido)-pyridin-2-yl ester,
4-(1-Methyl-1H-imidazol-2-ylsulfanylmethyl)-piperidine-1-carboxylic acid 5-(3-tert-butyl-thioureido)-pyridin-2-yl ester,
4-(Imidazol-1-yloxymethyl)-piperidine-1-carboxylic acid 5-(3-tert-butyl-thioureido)-pyridin-2-yl ester,
4-[4-(Pyridin-2-yloxy)-phenoxymethyl]-piperidine-1-carboxylic acid 5-(3-tert-butyl-thioureido)-pyridin-2-yl ester,
4-[4-(5-Methyl-pyridin-2-yloxy)-phenoxymethyl]-piperidine-1-carboxylic acid 5-(3-tert-butyl-thioureido)-pyridin-2-yl ester,
4-(1-Methyl-1H-tetrazol-5-ylsulfanylmethyl)-piperidine-1-carboxylic acid 5-(3-tert-butyl-thioureido)-pyridin-2-yl ester,
4-[1-(2-Dimethylamino-ethyl)-1H-tetrazol-5-ylsulfanyl]-piperidine-1-carboxylic acid 5-(3-tert-butyl-thioureido)-pyridin-2-yl ester,
4-[1-(2-Carboxy-ethyl)-1H-tetrazol-5-ylsulfanyl]-piperidine-1-carboxylic acid 5-(3-tert-butyl-thioureido)-pyridin-2-yl ester,
4-(1-Carboxymethyl-1H-tetrazol-5-ylsulfanyl)-piperidine-1-carboxylic acid 5-(3-tert-butyl-thioureido)-pyridin-2-yl ester,
4-(Pyrazol-1-yloxymethyl)-piperidine-1-carboxylic acid 5-(3-tert-butyl-thioureido)-pyridin-2-yl ester,
4-(1H-[1,2,4]Triazol-3-ylsulfanylmethyl)-piperidine-1-carboxylic acid 5-(3-tert-butyl-thioureido)-pyridin-2-yl ester,
4-[2-(Benzyl-methyl-amino)-ethyl]-piperidine-1-carboxylic acid 5-(3-tert-butyl-thioureido)-pyridin-2-yl ester,
4-(1H-[1,2,4]Triazol-3-ylsulfanyl)-piperidine-1-carboxylic acid 4-[2-(pyridin-4-ylsulfanyl)-ethyl]-phenyl ester,
4-(1H-Imidazol-2-ylsulfanyl)-piperidine-1-carboxylic acid 4-[2-(pyridin-4-ylsulfanyl)-ethyl]-phenyl ester,
4-(1-Methyl-1H-imidazol-2-ylsulfanyl)-piperidine-1-carboxylic acid 4-[2-(pyridin-4-ylsulfanyl)-ethyl]-phenyl ester,
4-(1-Methyl-1H-tetrazol-5-ylsulfanyl)-piperidine-1-carboxylic acid 4-[2-(pyridin-4-ylsulfanyl)-ethyl]-phenyl ester,
4-(Imidazol-1-yloxy)-piperidine-1-carboxylic acid 4-[2-(pyridin-4-ylsulfanyl)-ethyl]-phenyl ester,
4-[1-(4-Carboxy-phenyl)-1H-tetrazol-5-ylsulfanyl]-piperidine-1-carboxylic acid 4-[2-(pyridin-4-ylsulfanyl)-ethyl]-phenyl ester,
4-(4-Carboxymethyl-phenoxy)-piperidine-1-carboxylic acid 4-[2-(pyridin-4-ylsulfanyl)-ethyl]-phenyl ester,
4-(Pyrazol-1-yloxy)-piperidine-1-carboxylic acid 4-[2-(pyridin4-ylsulfanyl)-ethyl]-phenyl ester,
4-(4-Carboxy-phenoxy)-piperidine-1-carboxylic acid 4-[2-(pyridin-4-ylsulfanyl)-ethyl]-phenyl ester,
4-[4-(Pyridin-2-yloxy)-phenoxy]-piperidine-1-carboxylic acid 4-[2-(pyridin-4-ylsulfanyl)-ethyl]-phenyl ester,
4-[4-(5-Methyl-pyridin-2-yloxy)-phenoxy]-piperidine-1-carboxylic acid 4-[2-(pyridin-4-ylsulfanyl)-ethyl]-phenyl ester,
4-(4-Carboxy-phenoxymethyl)-piperidine-1-carboxylic acid 4-[2-(pyridin-4-ylsulfanyl)-ethyl]-phenyl ester,
4-(4-Carboxymethyl-phenoxymethyl)-piperidine-1-carboxylic acid 4-[2-(pyridin-4-ylsulfanyl)-ethyl]-phenyl ester,
4-(1H-Imidazol-2-ylsulfanylmethyl)-piperidine-1-carboxylic acid 4-[2-(pyridin-4-ylsulfanyl)-ethyl]-phenyl ester,
4-(1-Methyl-1H-imidazol-2-ylsulfanylmethyl)-piperidine-1-carboxylic acid 4-[2-(pyridin-4-ylsulfanyl)-ethyl]-phenyl ester,
4-(Imidazol-1-yloxymethyl)-piperidine-1-carboxylic acid 4-[2-(pyridin-4-ylsulfanyl)-ethyl]-phenyl ester,
4-[4-(Pyridin-2-yloxy)-phenoxymethyl]-piperidine-1-carboxylic acid 4-[2-(pyridin-4-ylsulfanyl)-ethyl]-phenyl ester,
4-[4-(5-Methyl-pyridin-2-yloxy)-phenoxymethyl]-piperidine-1-carboxylic acid 4-[2-(pyridin-4-ylsulfanyl)-ethyl]-phenyl ester,
4-(1-Methyl-1H-tetrazol-5-ylsulfanylmethyl)-piperidine-1-carboxylic acid 4-[2-(pyridin-4-ylsulfanyl)-ethyl]-phenyl ester,
4-[1-(2-Dimethylamino-ethyl)-1H-tetrazol-5-ylsulfanyl]-piperidine-1-carboxylic acid 4-[2-(pyridin-4-ylsulfanyl)-ethyl]-phenyl ester,
4-[1-(2-Carboxy-ethyl)-1H-tetrazol-5-ylsulfanyl]-piperidine-1-carboxylic acid 4-[2-(pyridin-4-ylsulfanyl)-ethyl]-phenyl ester,
4-(1-Carboxymethyl-1H-tetrazol-5-ylsulfanyl)-piperidine-1-carboxylic acid 4-[2-(pyridin-4-ylsulfanyl)-ethyl]-phenyl ester,
4-(Pyrazol-1-yloxymethyl)-piperidine-1-carboxylic acid 4-[2-(pyridin-4-ylsulfanyl)-ethyl]-phenyl ester,
4-(1H-[1,2,4]Triazol-3-ylsulfanylmethyl)-piperidine-1-carboxylic acid 4-[2-(pyridin-4-ylsulfanyl)-ethyl]-phenyl ester,
4-[2-(Benzyl-methyl-amino)-ethyl]-piperidine-1-carboxylic acid 4-[2-(pyridin-4-ylsulfanyl)-ethyl]-phenyl ester,
4-(1H-[1,2,4]Triazol-3-ylsulfanyl)-piperidine-1-carboxylic acid 4-(1-methyl-1H-imidazol-2-ylsulfanylmethyl)-phenyl ester,
4-(1H-Imidazol-2-ylsulfanyl)-piperidine-1-carboxylic acid 4-(1-methyl-1H-imidazol-2-ylsulfanylmethyl)-phenyl ester,
4-(1-Methyl-1H-imidazol-2-ylsulfanyl)-piperidine-1-carboxylic acid 4-(1-methyl-1H-imidazol-2-ylsulfanylmethyl)-phenyl ester,
4-(1-Methyl-1H-tetrazol-5-ylsulfanyl)-piperidine-1-carboxylic acid 4-(1-methyl-1H-imidazol-2-ylsulfanylmethyl)-phenyl ester,
4-(Imidazol-1-yloxy)-piperidine-1-carboxylic acid 4-(1-methyl-1H-imidazol-2-ylsulfanylmethyl)-phenyl ester,
4-[1-(4-Carboxy-phenyl)-1H-tetrazol-5-ylsulfanyl]-piperidine-1-carboxylic acid 4-(1-methyl-1H-imidazol-2-ylsulfanylmethyl)-phenyl ester,
4-(4-Carboxymethyl-phenoxy)-piperidine-1-carboxylic acid 4-(1-methyl-1H-imidazol-2-ylsulfanylmethyl)-phenyl ester,
4-(Pyrazol-1-yloxy)-piperidine-1-carboxylic acid 4-(1-methyl-1H-imidazol-2-ylsulfanylmethyl)-phenyl ester,
4-(4-Carboxy-phenoxy)-piperidine-1-carboxylic acid 4-(1-methyl-1H-imidazol-2-ylsulfanylmethyl)-phenyl ester,
4-[4-(Pyridin-2-yloxy)-phenoxy]-piperidine-1-carboxylic acid 4-(1-methyl-1H-imidazol-2-ylsulfanylmethyl)-phenyl ester,
4-[4-(5-Methyl-pyridin-2-yloxy)-phenoxy]-piperidine-1-carboxylic acid 4-(1-methyl-1H-imidazol-2-ylsulfanylmethyl)-phenyl ester,
4-(4-Carboxy-phenoxymethyl)-piperidine-1-carboxylic acid 4-(1-methyl-1H-imidazol-2-ylsulfanylmethyl)-phenyl ester,
4-(4-Carboxymethyl-phenoxymethyl)-piperidine-1-carboxylic acid 4-(1-methyl-1H-imidazol-2-ylsulfanylmethyl)-phenyl ester,
4-(1H-Imidazol-2-ylsulfanylmethyl)-piperidine-1-carboxylic acid 4-(1-methyl-1H-imidazol-2-ylsulfanylmethyl)-phenyl ester,
4-(1-Methyl-1H-imidazol-2-ylsulfanylmethyl)-piperidine-1-carboxylic acid 4-(1-methyl-1H-imidazol-2-ylsulfanylmethyl)-phenyl ester,
4-(Imidazol-1-yloxymethyl)-piperidine-1-carboxylic acid 4-(1-methyl-1H-imidazol-2-ylsulfanylmethyl)-phenyl ester,
4-[4-(Pyridin-2-yloxy)-phenoxymethyl]-piperidine-1-carboxylic acid 4-(1-methyl-1H-imidazol-2-ylsulfanylmethyl)-phenyl ester,
4-[4-(5-Methyl-pyridin-2-yloxy)-phenoxymethyl]-piperidine-1-carboxylic acid 4-(1-methyl-1H-imidazol-2-ylsulfanylmethyl)-phenyl ester,
4-(1-Methyl-1H-tetrazol-5-ylsulfanylmethyl)-piperidine-1-carboxylic acid 4-(1-methyl-1H-imidazol-2-ylsulfanylmethyl)-phenyl ester,
4-[1-(2-Dimethylamino-ethyl)-1H-tetrazol-5-ylsulfanyl]-piperidine-1-carboxylic acid 4-(1-methyl-1H-imidazol-2-ylsulfanylmethyl)-phenyl ester,
4-[1-(2-Carboxy-ethyl)-1H-tetrazol-5-ylsulfanyl]-piperidine-1-carboxylic acid 4-(1-methyl-1H-imidazol-2-ylsulfanylmethyl)-phenyl ester,
4-(1-Carboxymethyl-1H-tetrazol-5-ylsulfanyl)-piperidine-1-carboxylic acid 4-(1-methyl-1H-imidazol-2-ylsulfanylmethyl)-phenyl ester,
4-(Pyrazol-1-yloxymethyl)-piperidine-1-carboxylic acid 4-(1-methyl-1H-imidazol-2-ylsulfanylmethyl)-phenyl ester,
4-(1H-[1,2,4]Triazol-3-ylsulfanylmethyl)-piperidine-1-carboxylic acid 4-(l1-methyl-1H-imidazol-2-ylsulfanylmethyl)-phenyl ester,
4-[2-(Benzyl-methyl-amino)-ethyl]-piperidine-1-carboxylic acid 4-(1-methyl-1H-imidazol-2-ylsulfanylmethyl)-phenyl ester,
4-(1H-[1,2,4]Triazol-3-ylsulfanyl)-piperidine-1-carboxylic acid 4-benzoylamino-phenyl ester,
4-(1H-Imidazol-2-ylsulfanyl)-piperidine-1-carboxylic acid 4-benzoylamino-phenyl ester,
4-(1-Methyl-1H-imidazol-2-ylsulfanyl)-piperidine-1-carboxylic acid 4-benzoylamino-phenyl ester,
4-(1-Methyl-1H-tetrazol-5-ylsulfanyl)-piperidine-1-carboxylic acid 4-benzoylamino-phenyl ester,
4-(Imidazol-1-yloxy)-piperidine-1-carboxylic acid 4-benzoylamino-phenyl ester,
4-[1-(4-Carboxy-phenyl)-1H-tetrazol-5-ylsulfanyl]-piperidine-1-carboxylic acid 4-benzoylamino-phenyl ester,
4-(4-Carboxymethyl-phenoxy)-piperidine-1-carboxylic acid 4-benzoylamino-phenyl ester,
4-(Pyrazol-1-yloxy)-piperidine-1-carboxylic acid 4-benzoylamino-phenyl ester,
4-(4-Carboxy-phenoxy)-piperidine-1-carboxylic acid 4-benzoylamino-phenyl ester,
4-[4-(Pyridin-2-yloxy)-phenoxy]-piperidine-1-carboxylic acid 4-benzoylamino-phenyl ester,
4-[4-(5-Methyl-pyridin-2-yloxy)-phenoxy]-piperidine-1-carboxylic acid 4-benzoylamino-phenyl ester,
4-(4-Carboxy-phenoxymethyl)-piperidine-1-carboxylic acid 4-benzoylamino-phenyl ester,
4-(4-Carboxymethyl-phenoxymethyl)-piperidine-1-carboxylic acid 4-benzoylamino-phenyl ester,
4-(1H-Imidazol-2-ylsulfanylmethyl)-piperidine-1-carboxylic acid 4-benzoylamino-phenyl ester,
4-(1-Methyl-1H-imidazol-2-ylsulfanylmethyl)-piperidine-1-carboxylic acid 4-benzoylamino-phenyl ester,
4-(Imidazol-1-yloxymethyl)-piperidine-1-carboxylic acid -4-benzoylamino-phenyl ester,
4-[4-(Pyridin-2-yloxy)-phenoxymethyl]-piperidine-1-carboxylic acid 4-benzoylamino-phenyl ester,
4-[4-(5-Methyl-pyridin-2-yloxy)-phenoxymethyl]-piperidine-1-carboxylic acid 4-benzoylamino-phenyl ester,
4-(1-Methyl-1H-tetrazol-5-ylsulfanylmethyl)-piperidine-1-carboxylic acid 4-benzoylamino-phenyl ester,
4-[1-(2-Dimethylamino-ethyl)-1H-tetrazol-5-ylsulfanyl]-piperidine-1-carboxylic acid 4-benzoylamino-phenyl ester,
4-[1-(2-Carboxy-ethyl)-1H-tetrazol-5-ylsulfanyl]-piperidine-1-carboxylic acid 4-benzoylamino-phenyl ester,
4-(1-Carboxymethyl-1H-tetrazol-5-ylsulfanyl)-piperidine-1-carboxylic acid 4-benzoylamino-phenyl ester,
4-(Pyrazol-1-yloxymethyl)-piperidine-1-carboxylic acid 4-benzoylamino-phenyl ester,
4-(1H-[1,2,4]Triazol-3-ylsulfanylmethyl)-piperidine-1-carboxylic acid 4-benzoylamino-phenyl ester,
4-[2-(Benzyl-methyl-amino)-ethyl]-piperidine-1-carboxylic acid 4-benzoylamino-phenyl ester,
4-(1H-[1,2,4]Triazol-3-ylsulfanyl)-piperidine-1-carboxylic acid 5-(1,3-dioxo-octahydro-isoindol-2-yl)-pyridin-2-yl ester,
4-(1H-Imidazol-2-ylsulfanyl)-piperidine-1-carboxylic acid 5-(1,3-dioxo-octahydro-isoindol-2-yl)-pyridin-2-yl ester,
4-(1-Methyl-1H-imidazol-2-ylsulfanyl)-piperidine-1-carboxylic acid 5-(1,3-dioxo-octahydro-isoindol-2-yl)-pyridin-2-yl ester,
4-(1-Methyl-1H-tetrazol-5-ylsulfanyl)-piperidine-1-carboxylic acid 5-(1,3-dioxo-octahydro-isoindol-2-yl)-pyridin-2-yl ester,
4-(Imidazol-1-yloxy)-piperidine-1-carboxylic acid 5-(1,3-dioxo-octahydro-isoindol-2-yl)-pyridin-2-yl ester,
4-[1-(4-Carboxy-phenyl)-1H-tetrazol-5-ylsulfanyl]-piperidine-1-carboxylic acid 5-(1,3-dioxo-octahydro-isoindol-2-yl)-pyridin-2-yl ester,
4-(4-Carboxymethyl-phenoxy)-piperidine-1-carboxylic acid 5-(1,3-dioxo-octahydro-isoindol-2-yl)-pyridin-2-yl ester,
4-(Pyrazol-1-yloxy)-piperidine-1-carboxylic acid 5-(1,3-dioxo-octahydro-isoindol-2-yl)-pyridin-2-yl ester,
4-(4-Carboxy-phenoxy)-piperidine-1-carboxylic acid 5-(1,3-dioxo-octahydro-isoindol-2-yl)-pyridin-2-yl ester,
4-[4-(Pyridin-2-yloxy)-phenoxy]-piperidine-1-carboxylic acid 5-(1,3-dioxo-octahydro-isoindol-2-yl)-pyridin-2-yl ester,
4-[4-(5-Methyl-pyridin-2-yloxy)-phenoxy]-piperidine-1-carboxylic acid 5-(1,3-dioxo-octahydro-isoindol-2-yl)-pyridin-2-yl ester,
4-(4-Carboxy-phenoxymethyl)-piperidine-1-carboxylic acid 5-(1,3-dioxo-octahydro-isoindol-2-yl)-pyridin-2-yl ester,
4-(4-Carboxymethyl-phenoxymethyl)-piperidine-1-carboxylic acid 5-(1,3-dioxo-octahydro-isoindol-2-yl)-pyridin-2-yl ester,
4-(1H-Imidazol-2-ylsulfanylmethyl)-piperidine-1-carboxylic acid 5-(1,3-dioxo-octahydro-isoindol-2-yl)-pyridin-2-yl ester,
4-(1-Methyl-1H-imidazol-2-ylsulfanylmethyl)-piperidine-1-carboxylic acid 5-(1,3-dioxo-octahydro-isoindol-2-yl)-pyridin-2-yl ester,
4-(Imidazol-1-yloxymethyl)-piperidine-1-carboxylic acid 5-(1,3-dioxo-octahydro-isoindol-2-yl)-pyridin-2-yl ester,
4-[4-(Pyridin-2-yloxy)-phenoxymethyl]-piperidine-1-carboxylic acid 5-(1,3-dioxo-octahydro-isoindol-2-yl)-pyridin-2-yl ester,
4-[4-(5-Methyl-pyridin-2-yloxy)-phenoxymethyl]-piperidine-1-carboxylic acid 5-(1,3-dioxo-octahydro-isoindol-2-yl)-pyridin-2-yl ester,
4-(1-Methyl-1H-tetrazol-5-ylsulfanylmethyl)-piperidine-1-carboxylic acid 5-(1,3-dioxo-octahydro-isoindol-2-yl)-pyridin-2-yl ester,
4-[1-(2-Dimethylamino-ethyl)-1H-tetrazol-5-ylsulfanyl]-piperidine-1-carboxylic acid 5-(1,3-dioxo-octahydro-isoindol-2-yl)-pyridin-2-yl ester,
4-[1-(2-Carboxy-ethyl)-1H-tetrazol-5-ylsulfanyl]-piperidine-1-carboxylic acid 5-(1,3-dioxo-octahydro-isoindol-2-yl)-pyridin-2-yl ester,
4-(1-Carboxymethyl-1H-tetrazol-5-ylsulfanyl)-piperidine-1-carboxylic acid 5-(1,3-dioxo-octahydro-isoindol-2-yl)-pyridin-2-yl ester,
4-(Pyrazol-1-yloxymethyl)-piperidine-1-carboxylic acid 5-(1,3-dioxo-octahydro-isoindol-2-yl)-pyridin-2-yl ester,
4-(1H-[1,2,4]Triazol-3-ylsulfanylmethyl)-piperidine-1-carboxylic acid 5-(1,3-dioxo-octahydro-isoindol-2-yl)-pyridin-2-yl ester,
4-[2-(Benzyl-methyl-amino)-ethyl]-piperidine-1-carboxylic acid 5-(1,3-dioxo-octahydro-isoindol-2-yl)-pyridin-2-yl ester,
4-(1H-[1,2,4]Triazol-3-ylsulfanyl)-piperidine-1-carboxylic acid quinolin-7-yl ester,
4-(1H-Imidazol-2-ylsulfanyl)-piperidine-1-carboxylic acid quinolin-7-yl ester,
4-(1-Methyl-1H-imidazol-2-ylsulfanyl)-piperidine-1-carboxylic acid quinolin-7-yl ester,
4-(1-Methyl-1H-tetrazol-5-ylsulfanyl)-piperidine-1-carboxylic acid quinolin-7-yl ester,
4-(Imidazol-1-yloxy)-piperidine-1-carboxylic acid quinolin-7-yl ester,
4-[1-(4-Carboxy-phenyl)-1H-tetrazol-5-ylsulfanyl]-piperidine-1-carboxylic acid quinolin-7-yl ester,
4-(4-Carboxymethyl-phenoxy)-piperidine-1-carboxylic acid quinolin-7-yl ester,
4-(Pyrazol-1-yloxy)-piperidine-1-carboxylic acid quinolin-7-yl ester,
4-(4-Carboxy-phenoxy)-piperidine-1-carboxylic acid quinolin-7-yl ester,
4-[4-(Pyridin-2-yloxy)-phenoxy]-piperidine-1-carboxylic acid quinolin-7-yl ester,
4-[4-(5-Methyl-pyridin-2-yloxy)-phenoxy]-piperidine-1-carboxylic acid quinolin-7-yl ester,
4-(4-Carboxy-phenoxymethyl)-piperidine-1-carboxylic acid quinolin-7-yl ester,
4-(4-Carboxymethyl-phenoxymethyl)-piperidine-1-carboxylic acid quinolin-7-yl ester,
4-(1H-Imidazol-2-ylsulfanylmethyl)-piperidine-1-carboxylic acid quinolin-7-yl ester,
4-(1-Methyl-1H-imidazol-2-ylsulfanylmethyl)-piperidine-1-carboxylic acid quinolin-7-yl ester,
4-(Imidazol-1-yloxymethyl)-piperidine-1-carboxylic acid quinolin-7-yl ester,
4-[4-(Pyridin-2-yloxy)-phenoxymethyl]-piperidine-1-carboxylic acid quinolin-7-yl ester,
4-[4-(5-Methyl-pyridin-2-yloxy)-phenoxymethyl]-piperidine-1-carboxylic acid quinolin-7-yl ester,
4-(1-Methyl-1H-tetrazol-5-ylsulfanylmethyl)-piperidine-1-carboxylic acid quinolin-7-yl ester,
4-[1-(2-Dimethylamino-ethyl)-1H-tetrazol-5-ylsulfanyl]-piperidine-1-carboxylic acid quinolin-7-yl ester,
4-[1-(2-Carboxy-ethyl)-1H-tetrazol-5-ylsulfanyl]-piperidine-1-carboxylic acid quinolin-7-yl ester,
4-(1-Carboxymethyl-1H-tetrazol-5-ylsulfanyl)-piperidine-1-carboxylic acid quinolin-7-yl ester,
4-(Pyrazol-1-yloxymethyl)-piperidine-1-carboxylic acid quinolin-7-yl ester,
4-(1H-[1,2,4]Triazol-3-ylsulfanylmethyl)-piperidine-1-carboxylic acid quinolin-7-yl ester,
4-[2-(Benzyl-methyl-amino)-ethyl]-piperidine-1-carboxylic acid quinolin-7-yl ester,
4-(1H-[1,2,4]Triazol-3-ylsulfanyl)-piperidine-1-carboxylic acid 4-(1,1,3,3-tetramethyl-butylcarbamoyl)-phenyl ester,
4-(1H-Imidazol-2-ylsulfanyl)-piperidine-1-carboxylic acid 4-(1,1,3,3-tetramethyl-butylcarbamoyl)-phenyl ester,
4-(1-Methyl-1H-imidazol-2-ylsulfanyl)-piperidine-1-carboxylic acid 4-(1,1,3,3-tetramethyl-butylcarbamoyl)-phenyl ester,
4-(1-Methyl-1H-tetrazol-5-ylsulfanyl)-piperidine-1-carboxylic acid 4-(1,1,3,3-tetramethyl-butylcarbamoyl)-phenyl ester,
4-(Imidazol-1-yloxy)-piperidine-1-carboxylic acid 4-(1,1,3,3-tetramethyl-butylcarbamoyl)-phenyl ester,
4-[1-(4-Carboxy-phenyl)-1H-tetrazol-5-ylsulfanyl]-piperidine-1-carboxylic acid 4-(1,1,3,3-tetramethyl-butylcarbamoyl)-phenyl ester,
4-(4-Carboxymethyl-phenoxy)-piperidine-1-carboxylic acid 4-(1,1,3,3-tetramethyl-butylcarbamoyl)-phenyl ester,
4-(Pyrazol-1-yloxy)-piperidine-1-carboxylic acid 4-(1,1,3,3-tetramethyl-butylcarbamoyl)-phenyl ester,
4-(4-Carboxy-phenoxy)-piperidine-1-carboxylic acid 4-(1,1,3,3-tetramethyl-butylcarbamoyl)-phenyl ester,
4-[4-(Pyridin-2-yloxy)-phenoxy]-piperidine-1-carboxylic acid 4-(1,1,3,3-tetramethyl-butylcarbamoyl)-phenyl ester,
4-[4-(5-Methyl-pyridin-2-yloxy)-phenoxy]-piperidine-1-carboxylic acid 4-(1,1,3,3-tetramethyl-butylcarbamoyl)-phenyl ester,
4-(4-Carboxy-phenoxymethyl)-piperidine-1-carboxylic acid 4-(1,1,3,3-tetramethyl-butylcarbamoyl)-phenyl ester,
4-(4-Carboxymethyl-phenoxymethyl)-piperidine-1-carboxylic acid 4-(1,1,3,3-tetramethyl-butylcarbamoyl)-phenyl ester,
4-(1H-Imidazol-2-ylsulfanylmethyl)-piperidine-1-carboxylic acid 4-(1,1,3,3-tetramethyl-butylcarbamoyl)-phenyl ester,
4-(1-Methyl-1H-imidazol-2-ylsulfanylmethyl)-piperidine-1-carboxylic acid 4-(1,1,3,3-tetramethyl-butylcarbamoyl)-phenyl ester,
4-(Imidazol-1-yloxymethyl)-piperidine-1-carboxylic acid 4-(1,1,3,3-tetramethyl-butylcarbamoyl)-phenyl ester,
4-[4-(Pyridin-2-yloxy)-phenoxymethyl]-piperidine-1-carboxylic acid 4-(1,1,3,3-tetramethyl-butylcarbamoyl)-phenyl ester,
4-[4-(5-Methyl-pyridin-2-yloxy)-phenoxymethyl]-piperidine-1-carboxylic acid 4-(1,1,3,3-tetramethyl-butylcarbamoyl)-phenyl ester,
4-(1-Methyl-1H-tetrazol-5-ylsulfanylmethyl)-piperidine-1-carboxylic acid 4-(1,1,3,3-tetramethyl-butylcarbamoyl)-phenyl ester,
4-[1-(2-Dimethylamino-ethyl)-1H-tetrazol-5-ylsulfanyl]-piperidine-1-carboxylic acid 4-(1,1,3,3-tetramethyl-butylcarbamoyl)-phenyl ester,
4-[1-(2-Carboxy-ethyl)-1H-tetrazol-5-ylsulfanyl]-piperidine-1-carboxylic acid 4-(1,1,3,3-tetramethyl-butylcarbamoyl)-phenyl ester,
4-(1-Carboxymethyl-1H-tetrazol-5-ylsulfanyl)-piperidine-1-carboxylic acid 4-(1,1,3,3-tetramethyl-butylcarbamoyl)-phenyl ester,
4-(Pyrazol-1-yloxymethyl)-piperidine-1-carboxylic acid 4-(1,1,3,3-tetramethyl-butylcarbamoyl)-phenyl ester,
4-(1H-[1,2,4]Triazol-3-ylsulfanylmethyl)-piperidine-1-carboxylic acid 4-(1,1,3,3-tetramethyl-butylcarbamoyl)-phenyl ester,
4-[2-(Benzyl-methyl-amino)-ethyl]-piperidine-1-carboxylic acid 4-(1,1,3,3-tetramethyl-butylcarbamoyl)-phenyl ester,
4-(1H-[1,2,4]Triazol-3-ylsulfanyl)-piperidine-1-carboxylic acid 4-(cyclohexanecarbonyl-amino)-phenyl ester,
4-(1H-Imidazol-2-ylsulfanyl)-piperidine-1-carboxylic acid 4-(cyclohexanecarbonyl-amino)-phenyl ester,
4-(1-Methyl-1H-imidazol-2-ylsulfanyl)-piperidine-1-carboxylic acid 4-(cyclohexanecarbonyl-amino)-phenyl ester,
4-(1-Methyl-1H-tetrazol-5-ylsulfanyl)-piperidine-1-carboxylic acid 4-(cyclohexanecarbonyl-amino)-phenyl ester,
4-(Imidazol-1-yloxy)-piperidine-1-carboxylic acid 4-(cyclohexanecarbonyl-amino)-phenyl ester,
4-[1-(4-Carboxy-phenyl)-1H-tetrazol-5-ylsulfanyl]-piperidine-1-carboxylic acid 4-(cyclohexanecarbonyl-amino)-phenyl ester,
4-(4-Carboxymethyl-phenoxy)-piperidine-1-carboxylic acid 4-(cyclohexanecarbonyl-amino)-phenyl ester,
4-(Pyrazol-1-yloxy)-piperidine-1-carboxylic acid 4-(cyclohexanecarbonyl-amino)-phenyl ester,
4-(4-Carboxy-phenoxy)-piperidine-1-carboxylic acid 4-(cyclohexanecarbonyl-amino)-phenyl ester,
4-[4-(Pyridin-2-yloxy)-phenoxy]-piperidine-1-carboxylic acid 4-(cyclohexanecarbonyl-amino)-phenyl ester,
4-[4-(5-Methyl-pyridin-2-yloxy)-phenoxy]-piperidine-1-carboxylic acid 4-(cyclohexanecarbonyl-amino)-phenyl ester,
4-(4-Carboxy-phenoxymethyl)-piperidine-1-carboxylic acid 4-(cyclohexanecarbonyl-amino)-phenyl ester,
4-(4-Carboxymethyl-phenoxymethyl)-piperidine-1-carboxylic acid 4-(cyclohexanecarbonyl-amino)-phenyl ester,
4-(1H-Imidazol-2-ylsulfanylmethyl)-piperidine-1-carboxylic acid 4-(cyclohexanecarbonyl-amino)-phenyl ester,
4-(1-Methyl-1H-imidazol-2-ylsulfanylmethyl)-piperidine-1-carboxylic acid 4-(cyclohexanecarbonyl-amino)-phenyl ester,
4-(Imidazol-1-yloxymethyl)-piperidine-1-carboxylic acid 4-(cyclohexanecarbonyl-amino)-phenyl ester,
4-[4-(Pyridin-2-yloxy)-phenoxymethyl]-piperidine-1-carboxylic acid 4-(cyclohexanecarbonyl-amino)-phenyl ester,
4-[4-(5-Methyl-pyridin-2-yloxy)-phenoxymethyl]-piperidine-1-carboxylic acid 4-(cyclohexanecarbonyl-amino)-phenyl ester,
4-(1-Methyl-1H-tetrazol-5-ylsulfanylmethyl)-piperidine-1-carboxylic acid 4-(cyclohexanecarbonyl-amino)-phenyl ester,
4-[1-(2-Dimethylamino-ethyl)-1H-tetrazol-5-ylsulfanyl]-piperidine-1-carboxylic acid 4-(cyclohexanecarbonyl-amino)-phenyl ester,
4-[1-(2-Carboxy-ethyl)-1H-tetrazol-5-ylsulfanyl]-piperidine-1-carboxylic acid 4-(cyclohexanecarbonyl-amino)-phenyl ester,
4-(1-Carboxymethyl-1H-tetrazol-5-ylsulfanyl)-piperidine-1-carboxylic acid 4-(cyclohexanecarbonyl-amino)-phenyl ester,
4-(Pyrazol-1-yloxymethyl)-piperidine-1-carboxylic acid 4-(cyclohexanecarbonyl-amino)-phenyl ester,
4-(1H-[1,2,4]Triazol-3-ylsulfanylmethyl)-piperidine-1-carboxylic acid 4-(cyclohexanecarbonyl-amino)-phenyl ester,
4-[2-(Benzyl-methyl-amino)-ethyl]-piperidine-1-carboxylic acid 4-(cyclohexanecarbonyl-amino)-phenyl ester,
4-(1H-[1,2,4]Triazol-3-ylsulfanyl)-piperidine-1-carboxylic acid 4-[2-(4,4-dimethyl-2,6-dioxo-piperidin-1-yl)-ethyl]-phenyl ester,
4-(1-Methyl-1H-tetrazol-5-ylsulfanyl)-piperidine-1-carboxylic acid 4-[2-(4,4-dimethyl-2,6-dioxo-piperidin-1-yl)-ethyl]-phenyl ester,
4-[1-(4-Carboxy-phenyl)-1H-tetrazol-5-ylsulfanyl]-piperidine-1-carboxylic acid 4-[2-(4,4-dimethyl-2,6-dioxo-piperidin-1-yl)-ethyl]-phenyl ester,
4-[4-(Pyridin-2-yloxy)-phenoxy]-piperidine-1-carboxylic acid 4-[2-(4,4-dimethyl-2,6-dioxo-piperidin-1-yl)-ethyl]-phenyl ester,
4-[4-(5-Methyl-pyridin-2-yloxy)-phenoxy]-piperidine-1-carboxylic acid 4-[2-(4,4-dimethyl-2,6-dioxo-piperidin-1-yl)-ethyl]-phenyl ester,
4-(4-Carboxy-phenoxymethyl)-piperidine-1-carboxylic acid 4-[2-(4,4-dimethyl-2,6-dioxo-piperidin-1-yl)-ethyl]-phenyl ester,
4-(4-Carboxymethyl-phenoxymethyl)-piperidine-1-carboxylic acid 4-[2-(4,4-dimethyl-2,6-dioxo-piperidin-1-yl)-ethyl]-phenyl ester,
4-(1H-Imidazol-2-ylsulfanylmethyl)-piperidine-1-carboxylic acid 4-[2-(4,4-dimethyl-2,6-dioxo-piperidin-1-yl)-ethyl]-phenyl ester,
4-(1-Methyl-1H-imidazol-2-ylsulfanylmethyl)-piperidine-1-carboxylic acid 4-[2-(4,4-dimethyl-2,6-dioxo-piperidin-1-yl)-ethyl]-phenyl ester,
4-(Imidazol-1-yloxymethyl)-piperidine-1-carboxylic acid 4-[2-(4,4-dimethyl-2,6-dioxo-piperidin-1-yl)-ethyl]-phenyl ester,
4-[4-(Pyridin-2-yloxy)-phenoxymethyl]-piperidine-1-carboxylic acid 4-[2-(4,4-dimethyl-2,6-dioxo-piperidin-1-yl)-ethyl]-phenyl ester,
4-[4-( 5-Methyl-pyridin-2-yloxy)-phenoxymethyl]-piperidine-1-carboxylic acid 4-[2-(4,4-dimethyl-2,6-dioxo-piperidin-1-yl)-ethyl]-phenyl ester,
4-(1-Methyl-1H-tetrazol-5-ylsulfanylmethyl)-piperidine-1-carboxylic acid 4-[2-(4,4-dimethyl-2,6-dioxo-piperidin-1-yl)-ethyl]-phenyl ester,
4-[1-(2-Dimethylamino-ethyl)-1H-tetrazol-5-ylsulfanyl]-piperidine-1-carboxylic acid 4-[2-(4,4-dimethyl-2,6-dioxo-piperidin-1-yl)-ethyl]-phenyl ester,
4-[1-(2-Carboxy-ethyl)-1H-tetrazol-5-ylsulfanyl]-piperidine-1-carboxylic acid 4-[2-(4,4-dimethyl-2,6-dioxo-piperidin-1-yl)-ethyl]-phenyl ester,
4-(1-Carboxymethyl-1H-tetrazol-5-ylsulfanyl)-piperidine-1-carboxylic acid 4-[2-(4,4-dimethyl-2,6-dioxo-piperidin-1-yl)-ethyl]-phenyl ester,
4-(Pyrazol-1-yloxymethyl)-piperidine-1-carboxylic acid 4-[2-(4,4-dimethyl-2,6-dioxo-piperidin-1-yl)-ethyl]-phenyl ester,
4-(1H-[1,2,4]Triazol-3-ylsulfanylmethyl)-piperidine-1-carboxylic acid 4-[2-(4,4-dimethyl-2,6-dioxo-piperidin-1-yl)-ethyl]-phenyl ester,
4-[2-(Benzyl-methyl-amino)-ethyl]-piperidine-1-carboxylic acid 4-[2-(4,4-dimethyl-2,6-dioxo-piperidin-1-yl)-ethyl]-phenyl ester,
4-(1H-[1,2,4]Triazol-3-ylsulfanyl)-piperidine-1-carboxylic acid 4-(6-methyl-pyridin-2-ylmethyl)-phenyl ester,
4-(1H-Imidazol-2-ylsulfanyl)-piperidine-1-carboxylic acid 4-(6-methyl-pyridin-2-ylmethyl)-phenyl ester,
4-(1-Methyl-1H-imidazol-2-ylsulfanyl)-piperidine-1-carboxylic acid 4-(6-methyl-pyridin-2-ylmethyl)-phenyl ester,
4-(1-Methyl-1H-tetrazol-5-ylsulfanyl)-piperidine-1-carboxylic acid 4-(6-methyl-pyridin-2-ylmethyl)-phenyl ester,
4-(Imidazol-1-yloxy)-piperidine-1-carboxylic acid 4-(6-methyl-pyridin-2-ylmethyl)-phenyl ester,
4-[1-(4-Carboxy-phenyl)-1H-tetrazol-5-ylsulfanyl]-piperidine-1-carboxylic acid 4-(6-methyl-pyridin-2-ylmethyl)-phenyl ester,
4-(4-Carboxymethyl-phenoxy)-piperidine-1-carboxylic acid 4-(6-methyl-pyridin-2-ylmethyl)-phenyl ester,
4-(Pyrazol-1-yloxy)-piperidine-1-carboxylic acid 4-(6-methyl-pyridin-2-ylmethyl)-phenyl ester,
4-(4-Carboxy-phenoxy)-piperidine-1-carboxylic acid 4-(6-methyl-pyridin-2-ylmethyl)-phenyl ester,
4-[4-(Pyridin-2-yloxy)-phenoxy]-piperidine-1-carboxylic acid 4-(6-methyl-pyridin-2-ylmethyl)-phenyl ester,
4-[4-(5-Methyl-pyridin-2-yloxy)-phenoxy]-piperidine-1-carboxylic acid 4-(6-methyl-pyridin-2-ylmethyl)-phenyl ester,
4-(4-Carboxy-phenoxymethyl)-piperidine-1-carboxylic acid 4-(6-methyl-pyridin-2-ylmethyl)-phenyl ester,
4-(4-Carboxymethyl-phenoxymethyl)-piperidine-1-carboxylic acid 4-(6-methyl-pyridin-2-ylmethyl)-phenyl ester,
4-(1H-Imidazol-2-ylsulfanylmethyl)-piperidine-1-carboxylic acid 4-(6-methyl-pyridin-2-ylmethyl)-phenyl ester,
4-(1-Methyl-1H-imidazol-2-ylsulfanylmethyl)-piperidine-1-carboxylic acid 4-(6-methyl-pyridin-2-ylmethyl)-phenyl ester,
4-(Imidazol-1-yloxymethyl)-piperidine-1-carboxylic acid 4-(6-methyl-pyridin-2-ylmethyl)-phenyl ester,
4-[4-(Pyridin-2-yloxy)-phenoxymethyl]-piperidine-1-carboxylic acid 4-(6-methyl-pyridin-2-ylmethyl)-phenyl ester,
4-[4-(5-Methyl-pyridin-2-yloxy)-phenoxymethyl]-piperidine-1-carboxylic acid 4-(6-methyl-pyridin-2-ylmethyl)-phenyl ester,
4-(1-Methyl-1H-tetrazol-5-ylsulfanylmethyl)-piperidine-1-carboxylic acid 4-(6-methyl-pyridin-2-ylmethyl)-phenyl ester,
4-[1-(2-Dimethylamino-ethyl)-1H-tetrazol-5-ylsulfanyl]-piperidine-1-carboxylic acid 4-(6-methyl-pyridin-2-ylmethyl)-phenyl ester,
4-[1-(2-Carboxy-ethyl)-1H-tetrazol-5-ylsulfanyl]-piperidine-1-carboxylic acid 4-(6-methyl-pyridin-2-ylmethyl)-phenyl ester,
4-(1-Carboxymethyl-1H-tetrazol-5-ylsulfanyl)-piperidine-1-carboxylic acid 4-(6-methyl-pyridin-2-ylmethyl)-phenyl ester,
4-(Pyrazol-1-yloxymethyl)-piperidine-1-carboxylic acid 4-(6-methyl-pyridin-2-ylmethyl)-phenyl ester,
4-(1H-[1,2,4]Triazol-3-ylsulfanylmethyl)-piperidine-1-carboxylic acid 4-(6-methyl-pyridin-2-ylmethyl)-phenyl ester,
4-[2-(Benzyl-methyl-amino)-ethyl]-piperidine-1-carboxylic acid 4-(6-methyl-pyridin-2-ylmethyl)-phenyl ester,
4-(1H-[1,2,4]Triazol-3-ylsulfanyl)-piperidine-1-carboxylic acid 4-(1H-imidazol-2-ylsulfanylmethyl)-phenyl ester,
4-(1H-Imidazol-2-ylsulfanyl)-piperidine-1-carboxylic acid 4-(1H-imidazol-2-ylsulfanylmethyl)-phenyl ester,
4-(1-Methyl-1H-imidazol-2-ylsulfanyl)-piperidine-1-carboxylic acid 4-(1H-imidazol-2-ylsulfanylmethyl)-phenyl ester,
4-(1-Methyl-1H-tetrazol-5-ylsulfanyl)-piperidine-1-carboxylic acid 4-(1H-imidazol-2-ylsulfanylmethyl)-phenyl ester,
4-(Imidazol-1-yloxy)-piperidine-1-carboxylic acid 4-(1H-imidazol-2-ylsulfanylmethyl)-phenyl ester,
4-[1-(4-Carboxy-phenyl)-1H-tetrazol-5-ylsulfanyl]-piperidine-1-carboxylic acid 4-(1H-imidazol-2-ylsulfanylmethyl)-phenyl ester,
4-(4-Carboxymethyl-phenoxy)-piperidine-1-carboxylic acid 4-(1H-imidazol-2-ylsulfanylmethyl)-phenyl ester,
4-(Pyrazol-1-yloxy)-piperidine-1-carboxylic acid 4-(1H-imidazol-2-ylsulfanylmethyl)-phenyl ester,
4-(4-Carboxy-phenoxy)-piperidine-1-carboxylic acid 4-(1H-imidazol-2-ylsulfanylmethyl)-phenyl ester,
4-[4-(Pyridin-2-yloxy)-phenoxy]-piperidine-1-carboxylic acid 4-(1H-imidazol-2-ylsulfanylmethyl)-phenyl ester,
4-[4-(5-Methyl-pyridin-2-yloxy)-phenoxy]-piperidine-1-carboxylic acid 4-(1H-imidazol-2-ylsulfanylmethyl)-phenyl ester,
4-(4-Carboxy-phenoxymethyl)-piperidine-1-carboxylic acid 4-(1H-imidazol-2-ylsulfanylmethyl)-phenyl ester,
4-(4-Carboxymethyl-phenoxymethyl)-piperidine-1-carboxylic acid 4-(1H-imidazol-2-ylsulfanylmethyl)-phenyl ester,
4-(1H-Imidazol-2-ylsulfanylmethyl)-piperidine-1-carboxylic acid 4-(1H-imidazol-2-ylsulfanylmethyl)-phenyl ester,
4-(1-Methyl-1H-imidazol-2-ylsulfanylmethyl)-piperidine-1-carboxylic acid 4-(1H-imidazol-2-ylsulfanylmethyl)-phenyl ester,
4-(Imidazol-1-yloxymethyl)-piperidine-1-carboxylic acid 4-(1H-imidazol-2-ylsulfanylmethyl)-phenyl ester,
4-[4-(Pyridin-2-yloxy)-phenoxymethyl]-piperidine-1-carboxylic acid 4-(1H-imidazol-2-ylsulfanylmethyl)-phenyl ester,
4-[4-(5-Methyl-pyridin-2-yloxy)-phenoxymethyl]-piperidine-1-carboxylic acid 4-(1H-imidazol-2-ylsulfanylmethyl)-phenyl ester,
4-(1-Methyl-1H-tetrazol-5-ylsulfanylmethyl)-piperidine-1-carboxylic acid 4-(1H-imidazol-2-ylsulfanylmethyl)-phenyl ester,
4-[1-(2-Dimethylamino-ethyl)-1H-tetrazol-5-ylsulfanyl]-piperidine-1-carboxylic acid 4-(1H-imidazol-2-ylsulfanylmethyl)-phenyl ester,
4-[1-(2-Carboxy-ethyl)-1H-tetrazol-5-ylsulfanyl]-piperidine-1-carboxylic acid 4-(1H-imidazol-2-ylsulfanylmethyl)-phenyl ester,
4-(1-Carboxymethyl-1H-tetrazol-5-ylsulfanyl)-piperidine-1-carboxylic acid 4-(1H-imidazol-2-ylsulfanylmethyl)-phenyl ester,
4-(Pyrazol-1-yloxymethyl)-piperidine-1-carboxylic acid 4-(1H-imidazol-2-ylsulfanylmethyl)-phenyl ester,
4-(1H-[1,2,4]Triazol-3-ylsulfanylmethyl)-piperidine-1-carboxylic acid 4-(1H-imidazol-2-ylsulfanylmethyl)-phenyl ester,
4-[2-(Benzyl-methyl-amino)-ethyl]-piperidine-1-carboxylic acid 4-(1H-imidazol-2-ylsulfanylmethyl)-phenyl ester,
4-(1H-[1,2,4]Triazol-3-ylsulfanyl)-piperidine-1-carboxylic acid 4-[2-(1H-imidazol-ylsulfanyl)-ethyl]-phenyl ester,
4-(1H-Imidazol-2-ylsulfanyl)-piperidine-1-carboxylic acid 4-[2-(1H-imidazol-2-ylsulfanyl)-ethyl]-phenyl ester,
4-(1-Methyl-1H-imidazol-2-ylsulfanyl)-piperidine-1-carboxylic acid 4-[2-(1H-imidazol-2-ylsulfanyl)-ethyl]-phenyl ester,
4-(1-Methyl-1H-tetrazol-5-ylsulfanyl)-piperidine-1-carboxylic acid 4-[2-(1H-imidazol-2-ylsulfanyl)-ethyl]-phenyl ester,
4-(Imidazol-1-yloxy)-piperidine-1-carboxylic acid 4-[2-(1H-imidazol-2-ylsulfanyl)-ethyl]-phenyl ester,
4-[1-(4-Carboxy-phenyl)-1H-tetrazol-5-ylsulfanyl]-piperidine-1-carboxylic acid 4-[2-(1H-imidazol-2-ylsulfanyl)-ethyl]-phenyl ester,
4-(4-Carboxymethyl-phenoxy)-piperidine-1-carboxylic acid 4-[2-(1H-imidazol-2-ylsulfanyl)-ethyl]-phenyl ester,
4-(Pyrazol-1-yloxy)-piperidine-1-carboxylic acid 4-[2-(1H-imidazol-2-ylsulfanyl)-ethyl]-phenyl ester,
4-(4-Carboxy-phenoxy)-piperidine-1-carboxylic acid 4-[2-(1H-imidazol-2-ylsulfanyl)-ethyl]-phenyl ester,
4-[4-(Pyridin-2-yloxy)-phenoxy]-piperidine-1-carboxylic acid 4-[2-(1H-imidazol-2-ylsulfanyl)-ethyl]-phenyl ester,
4-[4-(5-Methyl-pyridin-2-yloxy)-phenoxy]-piperidine-1-carboxylic acid 4-[2-(1H-imidazol-2-ylsulfanyl)-ethyl]-phenyl ester,
4-(4-Carboxy-phenoxymethyl)-piperidine-1-carboxylic acid 4-[2-(1H-imidazol-2-ylsulfanyl)-ethyl]-phenyl ester,
4-(4-Carboxymethyl-phenoxymethyl)-piperidine-1-carboxylic acid 4-[2-(1H-imidazol-2-ylsulfanyl)-ethyl]-phenyl ester,
4-(1H-Imidazol-2-ylsulfanylmethyl)-piperidine-1-carboxylic acid 4-[2-(1H-imidazol-2-ylsulfanyl)-ethyl]-phenyl ester,
4-(1-Methyl-1H-imidazol-2-ylsulfanylmethyl)-piperidine-1-carboxylic acid 4-[2-(1H-imidazol-2-ylsulfanyl)-ethyl]-phenyl ester,
4-(Imidazol-1-yloxymethyl)-piperidine-1-carboxylic acid 4-[2-(1H-imidazol-2-ylsulfanyl)-ethyl]-phenyl ester,
4-[4-(Pyridin-2-yloxy)-phenoxymethyl]-piperidine-1-carboxylic acid 4-[2-(1H-imidazol-2-ylsulfanyl)-ethyl]-phenyl ester,
4-[4-(5-Methyl-pyridin-2-yloxy)-phenoxymethyl]-piperidine-1-carboxylic acid 4-[2-(1H-imidazol-2-ylsulfanyl)-ethyl]-phenyl ester,
4-(1-Methyl-1H-tetrazol-5-ylsulfanylmethyl)-piperidine-1-carboxylic acid 4-[2-(1H-imidazol-2-ylsulfanyl)-ethyl]-phenyl ester,
4-[1-(2-Dimethylamino-ethyl)-1H-tetrazol-5-ylsulfanyl]-piperidine-1-carboxylic acid 4-[2-(1H-imidazol-2-ylsulfanyl)-ethyl]-phenyl ester,
4-[1-(2-Carboxy-ethyl)-1H-tetrazol-5-ylsulfanyl]-piperidine-1-carboxylic acid 4-[2-(1H-imidazol-2-ylsulfanyl)-ethyl]-phenyl ester,
4-(1-Carboxymethyl-1H-tetrazol-5-ylsulfanyl)-piperidine-1-carboxylic acid 4-[2-(1H-imidazol-2-ylsulfanyl)-ethyl]-phenyl ester,
4-(Pyrazol-1-yloxymethyl)-piperidine-1-carboxylic acid 4-[2-(i H-imidazol-2-ylsulfanyl)-ethyl]-phenyl ester,
4-(1H-[1,2,4]Triazol-3-ylsulfanylmethyl)-piperidine-1-carboxylic acid 4-[2-(1H-imidazol-2-ylsulfanyl)-ethyl]-phenyl ester,
4-[2-(Benzyl-methyl-amino)-ethyl]-piperidine-1-carboxylic acid 4-[2-(1H-imidazol-2-ylsulfanyl)-ethyl]-phenyl ester,
4-(1H-[1,2,4]Triazol-3-ylsulfanyl)-piperidine-1-carboxylic acid 6-(4-isopropyl-5-oxo-2-thioxo-imidazolidin-1-yl)-pyridin-3-yl ester,
4-(1H-Imidazol-2-ylsulfanyl)-piperidine-1-carboxylic acid 6-(4-isopropyl-5-oxo-2-thioxo-imidazolidin-1-yl)-pyridin-3-yl ester,
4-(1-Methyl-1H-imidazol-2-ylsulfanyl)-piperidine-1-carboxylic acid 6-(4-isopropyl-5-oxo-2-thioxo-imidazolidin-1-yl)-pyridin-3-yl ester,
4-(1-Methyl-1H-tetrazol-5-ylsulfanyl)-piperidine-1-carboxylic acid 6-(4-isopropyl-5-oxo-2-thioxo-imidazolidin-1-yl)-pyridin-3-yl ester,
4-(Imidazol-1-yloxy)-piperidine-1-carboxylic acid 6-(4-isopropyl-5-oxo-2-thioxo-imidazolidin-1-yl)-pyridin-3-yl ester,
4-[1-(4-Carboxy-phenyl)-1H-tetrazol-5-ylsulfanyl]-piperidine-1-carboxylic acid 6-(4-isopropyl-5-oxo-2-thioxo-imidazolidin-1-yl)-pyridin-3-yl ester,
4-(4-Carboxymethyl-phenoxy)-piperidine-1-carboxylic acid 6-(4-isopropyl-5-oxo-2-thioxo-imidazolidin-1-yl)-pyridin-3-yl ester,
4-(Pyrazol-1-yloxy)-piperidine-1-carboxylic acid 6-(4-isopropyl-5-oxo-2-thioxo-imidazolidin-1-yl)-pyridin-3-yl ester,
4-(4-Carboxy-phenoxy)-piperidine-1-carboxylic acid 6-(4-isopropyl-5-oxo-2-thioxo-imidazolidin-1-yl)-pyridin-3-yl ester,
4-[4-(Pyridin-2-yloxy)-phenoxy]-piperidine-1-carboxylic acid 6-(4-isopropyl-5-oxo-2-thioxo-imidazolidin-1-yl)-pyridin-3-yl ester,
4-[4-(5-Methyl-pyridin-2-yloxy)-phenoxy]-piperidine-i-carboxylic acid 6-(4-isopropyl-5-oxo-2-thioxo-imidazolidin-1-yl)-pyridin-3-yl ester,
4-(4-Carboxy-phenoxymethyl)-piperidine-1-carboxylic acid 6-(4-isopropyl-5-oxo-2-thioxo-imidazolidin-1-yl)-pyridin-3-yl ester,
4-(4-Carboxymethyl-phenoxymethyl)-piperidine-1-carboxylic acid 6-(4-isopropyl-5-oxo-2-thioxo-imidazolidin-1-yl)-pyridin-3-yl ester,
4-(1H-Imidazol-2-ylsulfanylmethyl)-piperidine-1-carboxylic acid 6-(4-isopropyl-5-oxo-2-thioxo-imidazolidin-1-yl)-pyridin-3-yl ester,
4-(1-Methyl-1H-imidazol-2-ylsulfanylmethyl)-piperidine-1-carboxylic acid 6-(4-isopropyl-5-oxo-2-thioxo-imidazolidin-1-yl)-pyridin-3-yl ester,
4-(Imidazol-1-yloxymethyl)-piperidine-1-carboxylic acid 6-(4-isopropyl-5-oxo-2-thioxo-imidazolidin-1-yl)-pyridin-3-yl ester,
4-[4-(Pyridin-2-yloxy)-phenoxymethyl]-piperidine-1-carboxylic acid 6-(4-isopropyl-5-oxo-2-thioxo-imidazolidin-1-yl)-pyridin-3-yl ester,
4-[4-(5-Methyl-pyridin-2-yloxy)-phenoxymethyl]-piperidine-1-carboxylic acid 6-(4-isopropyl-5-oxo-2-thioxo-imidazolidin-1-yl)-pyridin-3-yl ester,
4-(1-Methyl-1H-tetrazol-5-ylsulfanylmethyl)-piperidine-1-carboxylic acid 6-(4-isopropyl-5-oxo-2-thioxo-imidazolidin-1-yl)-pyridin-3-yl ester,
4-[1-(2-Dimethylamino-ethyl)-1H-tetrazol-5-ylsulfanyl]-piperidine-1-carboxylic acid 6-(4-isopropyl-5-oxo-2-thioxo-imidazolidin-1-yl)-pyridin-3-yl ester,
4-[1-(2-Carboxy-ethyl)-1H-tetrazol-5-ylsulfanyl]-piperidine-1-carboxylic acid 6-(4-isopropyl-5-oxo-2-thioxo-imidazolidin-1-yl)-pyridin-3-yl ester,
4-(1-Carboxymethyl-1H-tetrazol-5-ylsulfanyl)-piperidine-1-carboxylic acid 6-(4-isopropyl-5-oxo-2-thioxo-imidazolidin-1-yl)-pyridin-3-yl ester,
4-(Pyrazol-1-yloxymethyl)-piperidine-1-carboxylic acid 6-(4-isopropyl-5-oxo-2-thioxo-imidazolidin-1-yl)-pyridin-3-yl ester,
4-(1H-[1,2,4]Triazol-3-ylsulfanylmethyl)-piperidine-1-carboxylic acid 6-(4-isopropyl-5-oxo-2-thioxo-imidazolidin-1-yl)-pyridin-3-yl ester,
4-[2-(Benzyl-methyl-amino)-ethyl]-piperidine-1-carboxylic acid 6-(4-isopropyl-5-oxo-2-thioxo-imidazolidin-1-yl)-pyridin-3-yl ester,
4-(1H-[1,2,4]Triazol-3-ylsulfanyl)-piperidine-1-carboxylic acid 6-(4-morpholin-4-ylmethyl-benzoylamino)-pyridin-3-yl ester,
4-(1H-Imidazol-2-ylsulfanyl)-piperidine-1-carboxylic acid 6-(4-morpholin-4-ylmethyl-benzoylamino)-pyridin-3-yl ester,
4-(1-Methyl-1H-imidazol-2-ylsulfanyl)-piperidine-1-carboxylic acid 6-(4-morpholin-4-ylmethyl-benzoylamino)-pyridin-3-yl ester,
4-(1-Methyl-1H-tetrazol-5-ylsulfanyl)-piperidine-1-carboxylic acid 6-(4-morpholin-4-ylmethyl-benzoylamino)-pyridin-3-yl ester,
4-(Imidazol-1-yloxy)-piperidine-1-carboxylic acid 6-(4-morpholin-4-ylmethyl-benzoylamino)-pyridin-3-yl ester,
4-[1-(4-Carboxy-phenyl)-1H-tetrazol-5-ylsulfanyl]-piperidine-1-carboxylic acid 6-(4-morpholin-4-ylmethyl-benzoylamino)-pyridin-3-yl ester,
4-(4-Carboxymethyl-phenoxy)-piperidine-1-carboxylic acid 6-(4-morpholin-4-ylmethyl-benzoylamino)-pyridin-3-yl ester,
4-(Pyrazol-1-yloxy)-piperidine-1-carboxylic acid 6-(4-morpholin-4-ylmethyl-benzoylamino)-pyridin-3-yl ester,
4-(4-Carboxy-phenoxy)-piperidine-1-carboxylic acid 6-(4-morpholin-4-ylmethyl-benzoylamino)-pyridin-3-yl ester,
4-[4-(Pyridin-2-yloxy)-phenoxy]-piperidine-1-carboxylic acid 6-(4-morpholin-4-ylmethyl-benzoylamino)-pyridin-3-yl ester,
4-[4-(5-Methyl-pyridin-2-yloxy)-phenoxy]-piperidine-1-carboxylic acid 6-(4-morpholin-4-ylmethyl-benzoylamino)-pyridin-3-yl ester,
4-(4-Carboxy-phenoxymethyl)-piperidine-1-carboxylic acid 6-(4-morpholin-4-ylmethyl-benzoylamino)-pyridin-3-yl ester,
4-(4-Carboxymethyl-phenoxymethyl)-piperidine-1-carboxylic acid 6-(4-morpholin-4-ylmethyl-benzoylamino)-pyridin-3-yl ester,
4-(1H-Imidazol-2-ylsulfanylmethyl)-piperidine-1-carboxylic acid 6-(4-morpholin-4-ylmethyl-benzoylamino)-pyridin-3-yl ester,
4-(1-Methyl-1H-imidazol-2-ylsulfanylmethyl)-piperidine-1-carboxylic acid 6-(4-morpholin-4-ylmethyl-benzoylamino)-pyridin-3-yl ester,
4-(Imidazol-1-yloxymethyl)-piperidine-1-carboxylic acid 6-(4-morpholin-4-ylmethyl-benzoylamino)-pyridin-3-yl ester,
4-[4-(Pyridin-2-yloxy)-phenoxymethyl]-piperidine-1-carboxylic acid 6-(4-morpholin-4-ylmethyl-benzoylamino)-pyridin-3-yl ester,
4-[4-(5-Methyl-pyridin-2-yloxy)-phenoxymethyl]-piperidine-1-carboxylic acid 6-(4-morpholin-4-ylmethyl-benzoylamino)-pyridin-3-yl ester,
4-(1-Methyl-1H-tetrazol-5-ylsulfanylmethyl)-piperidine-1-carboxylic acid 6-(4-morpholin-4-ylmethyl-benzoylamino)-pyridin-3-yl ester,
4-[1-(2-Dimethylamino-ethyl)-1H-tetrazol-5-ylsulfanyl]-piperidine-1-carboxylic acid 6-(4-morpholin-4-ylmethyl-benzoylamino)-pyridin-3-yl ester,
4-[1-(2-Carboxy-ethyl)-1H-tetrazol-5-ylsulfanyl]-piperidine-1-carboxylic acid 6-(4-morpholin-4-ylmethyl-benzoylamino)-pyridin-3-yl ester,
4-(1-Carboxymethyl-1H-tetrazol-5-ylsulfanyl)-piperidine-1-carboxylic acid 6-(4-morpholin-4-ylmethyl-benzoylamino)-pyridin-3-yl ester,
4-(Pyrazol-1-yloxymethyl)-piperidine-1-carboxylic acid 6-(4-morpholin-4-ylmethyl-benzoylamino)-pyridin-3-yl ester,
4-(1H-[1,2,4]Triazol-3-ylsulfanylmethyl)-piperidine-1-carboxylic acid 6-(4-morpholin-4-ylmethyl-benzoylamino)-pyridin-3-yl ester,
4-[2-(Benzyl-methyl-amino)-ethyl]-piperidine-1-carboxylic acid 6-(4-morpholin-4-ylmethyl-benzoylamino)-pyridin-3-yl ester,
4-(1H-[1,2,4]Triazol-3-ylsulfanyl)-piperidine-1-carboxylic acid 4-(4-morpholin-4-ylmethyl-benzoylamino)-phenyl ester,
4-(1H-Imidazol-2-ylsulfanyl)-piperidine-1-carboxylic acid 4-(4-morpholin-4-ylmethyl-benzoylamino)-phenyl ester,
4-(1-Methyl-1H-imidazol-2-ylsulfanyl)-piperidine-1-carboxylic acid 4-(4-morpholin-4-ylmethyl-benzoylamino)-phenyl ester,
4-(1-Methyl-1H-tetrazol-5-ylsulfanyl)-piperidine-1-carboxylic acid 4-(4-morpholin-4-ylmethyl-benzoylamino)-phenyl ester,
4-(Imidazol-1-yloxy)-piperidine-1-carboxylic acid 4-(4-morpholin-4-ylmethyl-benzoylamino)-phenyl ester,
4-[1-(4-Carboxy-phenyl)-1H-tetrazol-5-ylsulfanyl]-piperidine-1-carboxylic acid 4-(4-morpholin-4-ylmethyl-benzoylamino)-phenyl ester,
4-(4-Carboxymethyl-phenoxy)-piperidine-1-carboxylic acid 4-(4-morpholin-4-ylmethyl-benzoylamino)-phenyl ester,
4-(Pyrazol-1-yloxy)-piperidine-1-carboxylic acid 4-(4-morpholin-4-ylmethyl-benzoylamino)-phenyl ester,
4-(4-Carboxy-phenoxy)-piperidine-1-carboxylic acid 4-(4-morpholin-4-ylmethyl-benzoylamino)-phenyl ester,
4-[4-(Pyridin-2-yloxy)-phenoxy]-piperidine-1-carboxylic acid 4-(4-morpholin-4-ylmethyl-benzoylamino)-phenyl ester,
4-[4-(5-Methyl-pyridin-2-yloxy)-phenoxy]-piperidine-1-carboxylic acid 4-(4-morpholin-4-ylmethyl-benzoylamino)-phenyl ester,
4-(4-Carboxy-phenoxymethyl)-piperidine-1-carboxylic acid 4-(4-morpholin-4-ylmethyl-benzoylamino)-phenyl ester,
4-(4-Carboxymethyl-phenoxymethyl)-piperidine-1-carboxylic acid 4-(4-morpholin-4-ylmethyl-benzoylamino)-phenyl ester,
4-(1H-Imidazol-2-ylsulfanylmethyl)-piperidine-1-carboxylic acid 4-(4-morpholin-4-ylmethyl-benzoylamino)-phenyl ester,
4-(1-Methyl-1H-imidazol-2-ylsulfanylmethyl)-piperidine-1-carboxylic acid 4-(4-morpholin-4-ylmethyl-benzoylamino)-phenyl ester,
4-(Imidazol-1-yloxymethyl)-piperidine-1-carboxylic acid 4-(4-morpholin-4-ylmethyl-benzoylamino)-phenyl ester,
4-[4-(Pyridin-2-yloxy)-phenoxymethyl]-piperidine-1-carboxylic acid 4-(4-morpholin-4-ylmethyl-benzoylamino)-phenyl ester,
4-[4-(5-Methyl-pyridin-2-yloxy)-phenoxymethyl]-piperidine-1-carboxylic acid 4-(4-morpholin-4-ylmethyl-benzoylamino)-phenyl ester,
4-(1-Methyl-1H-tetrazol-5-ylsulfanylmethyl)-piperidine-1-carboxylic acid 4-(4-morpholin-4-ylmethyl-benzoylamino)-phenyl ester,
4-[1-(2-Dimethylamino-ethyl)-1H-tetrazol-5-ylsulfanyl]-piperidine-1-carboxylic acid 4-(4-morpholin-4-ylmethyl-benzoylamino)-phenyl ester,
4-[1-(2-Carboxy-ethyl)-1H-tetrazol-5-ylsulfanyl]-piperidine-1-carboxylic acid 4-(4-morpholin-4-ylmethyl-benzoylamino)-phenyl ester,
4-(1-Carboxymethyl-1H-tetrazol-5-ylsulfanyl)-piperidine-1-carboxylic acid 4-(4-morpholin-4-ylmethyl-benzoylamino)-phenyl ester,
4-(Pyrazol-1-yloxymethyl)-piperidine-1-carboxylic acid 4-(4-morpholin-4-ylmethyl-benzoylamino)-phenyl ester,
4-(1H-[1,2,4]Triazol-3-ylsulfanylmethyl)-piperidine-1-carboxylic acid 4-(4-morpholin-4-ylmethyl-benzoylamino)-phenyl ester,
4-[2-(Benzyl-methyl-amino)-ethyl]-piperidine-1-carboxylic acid 4-(4-morpholin-4-ylmethyl-benzoylamino)-phenyl ester,
4-(1H-[1,2,4]Triazol-3-ylsulfanyl)-piperidine-1-carboxylic acid 6-(4-piperidin-1-ylmethyl-benzoylamino)-pyridin-3-yl ester,
4-(1H-Imidazol-2-ylsulfanyl)-piperidine-1-carboxylic acid 6-(4-piperidin-1-ylmethyl-benzoylamino)-pyridin-3-yl ester,
4-(1-Methyl-1H-imidazol-2-ylsulfanyl)-piperidine-1-carboxylic acid 6-(4-piperidin-1-ylmethyl-benzoylamino)-pyridin-3-yl ester,
4-(1-Methyl-1H-tetrazol-5-ylsulfanyl)-piperidine-1-carboxylic acid 6-(4-piperidin-1-ylmethyl-benzoylamino)-pyridin-3-yl ester,
4-(Imidazol-1-yloxy)-piperidine-1-carboxylic acid 6-(4-piperidin-1-ylmethyl-benzoylamino)-pyridin-3-yl ester,
4-[1-(4-Carboxy-phenyl)-1H-tetrazol-5-ylsulfanyl]-piperidine-1-carboxylic acid 6-(4-piperidin-1-ylmethyl-benzoylamino)-pyridin-3-yl ester,
4-(4-Carboxymethyl-phenoxy)-piperidine-1-carboxylic acid 6-(4-piperidin-1-ylmethyl-benzoylamino)-pyridin-3-yl ester,
4-( Pyrazol-1-yloxy)-piperidine-1-carboxylic acid 6-(4-piperidin-1-ylmethyl-benzoylamino)-pyridin-3-yl ester,
4-(4-Carboxy-phenoxy)-piperidine-1-carboxylic acid 6-(4-piperidin-1-ylmethyl-benzoylamino)-pyridin-3-yl ester,
4-[4-(Pyridin-2-yloxy)-phenoxy]-piperidine-1-carboxylic acid 6-(4-piperidin-1-ylmethyl-benzoylamino)-pyridin-3-yl ester,
4-[4-(5-Methyl-pyridin-2-yloxy)-phenoxy]-piperidine-1-carboxylic acid 6-(4-piperidin-1-ylmethyl-benzoylamino)-pyridin-3-yl ester,
4-(4-Carboxy-phenoxymethyl)-piperidine-1-carboxylic acid 6-(4-piperidin-1-ylmethyl-benzoylamino)-pyridin-3-yl ester,
4-(4-Carboxymethyl-phenoxymethyl)-piperidine-1-carboxylic acid 6-(4-piperidin-1-ylmethyl-benzoylamino)-pyridin-3-yl ester,
4-(1H-Imidazol-2-ylsulfanylmethyl)-piperidine-1-carboxylic acid 6-(4-piperidin-1-ylmethyl-benzoylamino)-pyridin-3-yl ester,
4-(1-Methyl-1H-imidazol-2-ylsulfanylmethyl)-piperidine-1-carboxylic acid 6-(4-piperidin-1-ylmethyl-benzoylamino)-pyridin-3-yl ester,
4-(Imidazol-1-yloxymethyl)-piperidine-1-carboxylic acid 6-(4-piperidin-1-ylmethyl-benzoylamino)-pyridin-3-yl ester,
4-[4-(Pyridin-2-yloxy)-phenoxymethyl]-piperidine-1-carboxylic acid 6-(4-piperidin-1-ylmethyl-benzoylamino)-pyridin-3-yl ester,
4-[4-(5-Methyl-pyridin-2-yloxy)-phenoxymethyl]-piperidine-1-carboxylic acid 6-(4-piperidin-1-ylmethyl-benzoylamino)-pyridin-3-yl ester,
4-(1-Methyl-1H-tetrazol-5-ylsulfanylmethyl)-piperidine-1-carboxylic acid 6-(4-piperidin-1-ylmethyl-benzoylamino)-pyridin-3-yl ester,
4-[1-(2-Dimethylamino-ethyl)-1H-tetrazol-5-ylsulfanyl]-piperidine-1-carboxylic acid 6-(4-piperidin-1-ylmethyl-benzoylamino)-pyridin-3-yl ester,
4-[1-(2-Carboxy-ethyl)-1H-tetrazol-5-ylsulfanyl]-piperidine-1-carboxylic acid 6-(4-piperidin-1-ylmethyl-benzoylamino)-pyridin-3-yl ester,
4-(1-Carboxymethyl-1H-tetrazol-5-ylsulfanyl)-piperidine-1-carboxylic acid 6-(4-piperidin-1-ylmethyl-benzoylamino)-pyridin-3-yl ester,
4-(Pyrazol-1-yloxymethyl)-piperidine-1-carboxylic acid 6-(4-piperidin-1-ylmethyl-benzoylamino)-pyridin-3-yl ester,
4-(1H-[1,2,4]Triazol-3-ylsulfanylmethyl)-piperidine-1-carboxylic acid 6-(4-piperidin-1-ylmethyl-benzoylamino)-pyridin-3-yl ester,
4-[2-(Benzyl-methyl-amino)-ethyl]-piperidine-1-carboxylic acid 6-(4-piperidin-1-ylmethyl-benzoylamino)-pyridin-3-yl ester,
4-(1H-[1,2,4]Triazol-3-ylsulfanyl)-piperidine-1-carboxylic acid 4-(4-piperidin-1-ylmethyl-benzoylamino)-phenyl ester,
4-(1H-Imidazol-2-ylsulfanyl)-piperidine-1-carboxylic acid 4-(4-piperidin-1-ylmethyl-benzoylamino)-phenyl ester,
4-(1-Methyl-1H-imidazol-2-ylsulfanyl)-piperidine-1-carboxylic acid 4-(4-piperidin-1-ylmethyl-benzoylamino)-phenyl ester,
4-(1-Methyl-1H-tetrazol-5-ylsulfanyl)-piperidine-1-carboxylic acid 4-(4-piperidin-1-ylmethyl-benzoylamino)-phenyl ester,
4-(Imidazol-1-yloxy)-piperidine-1-carboxylic acid 4-(4-piperidin-1-ylmethyl-benzoylamino)-phenyl ester,
4-[1-(4-Carboxy-phenyl)-1H-tetrazol-5-ylsulfanyl]-piperidine-1-carboxylic acid 4-(4-piperidin-1-ylmethyl-benzoylamino)-phenyl ester,
4-(4-Carboxymethyl-phenoxy)-piperidine-1-carboxylic acid 4-(4-piperidin-1-ylmethyl-benzoylamino)-phenyl ester,
4-(Pyrazol-1-yloxy)-piperidine-1-carboxylic acid 4-(4-piperidin-1-ylmethyl-benzoylamino)-phenyl ester,
4-(4-Carboxy-phenoxy)-piperidine-1-carboxylic acid 4-(4-piperidin-1-ylmethyl-benzoylamino)-phenyl ester,
4-[4-(Pyridin-2-yloxy)-phenoxy]-piperidine-1-carboxylic acid 4-(4-piperidin-1-ylmethyl-benzoylamino)-phenyl ester,
4-[4-(5-Methyl-pyridin-2-yloxy)-phenoxy]-piperidine-1-carboxylic acid 4-(4-piperidin-1-ylmethyl-benzoylamino)-phenyl ester,
4-(4-Carboxy-phenoxymethyl)-piperidine-1-carboxylic acid 4-(4-piperidin-1-ylmethyl-benzoylamino)-phenyl ester,
4-(4-Carboxymethyl-phenoxymethyl)-piperidine-1-carboxylic acid 4-(4-piperidin-1-ylmethyl-benzoylamino)-phenyl ester,
4-(1H-Imidazol-2-ylsulfanylmethyl)-piperidine-1-carboxylic acid 4-(4-piperidin-1-ylmethyl-benzoylamino)-phenyl ester,
4-(1-Methyl-1H-imidazol-2-ylsulfanylmethyl)-piperidine-1-carboxylic acid 4-(4-piperidin-1-ylmethyl-benzoylamino)-phenyl ester,
4-(Imidazol-1-yloxymethyl)-piperidine-1-carboxylic acid 4-(4-piperidin-1-ylmethyl-benzoylamino)-phenyl ester,
4-[4-(Pyridin-2-yloxy)-phenoxymethyl]-piperidine-1-carboxylic acid 4-(4-piperidin-1-ylmethyl-benzoylamino)-phenyl ester,
4-[4-(5-Methyl-pyridin-2-yloxy)-phenoxymethyl]-piperidine-1-carboxylic acid 4-(4-piperidin-1-ylmethyl-benzoylamino)-phenyl ester,
4-(1-Methyl-1H-tetrazol-5-ylsulfanylmethyl)-piperidine-1-carboxylic acid 4-(4-piperidin-1-ylmethyl-benzoylamino)-phenyl ester,
4-[1-(2-Dimethylamino-ethyl)-1H-tetrazol-5-ylsulfanyl]-piperidine-1-carboxylic acid 4-(4-piperidin-1-ylmethyl-benzoylamino)-phenyl ester,
4-[1-(2-Carboxy-ethyl)-1H-tetrazol-5-ylsulfanyl]-piperidine-1-carboxylic acid 4-(4-piperidin-1-ylmethyl-benzoylamino)-phenyl ester,
4-(1-Carboxymethyl-1H-tetrazol-5-ylsulfanyl)-piperidine-1-carboxylic acid 4-(4-piperidin-1-ylmethyl-benzoylamino)-phenyl ester,
4-(Pyrazol-1-yloxymethyl)-piperidine-1-carboxylic acid 4-(4-piperidin-1-ylmethyl-benzoylamino)-phenyl ester,
4-(1H-[1,2,4]Triazol-3-ylsulfanylmethyl)-piperidine-1-carboxylic acid 4-(4-piperidin-1-ylmethyl-benzoylamino)-phenyl ester,
4-[2-(Benzyl-methyl-amino)-ethyl]-piperidine-1-carboxylic acid 4-(4-piperidin-1-ylmethyl-benzoylamino)-phenyl ester,
4-(1H-[1,2,4]Triazol-3-ylsulfanyl)-piperidine-1-carboxylic acid 5-(4-piperidin-1-ylmethyl-benzoylamino)-pyridin-2-yl ester,
4-(1H-Imidazol-2-ylsulfanyl)-piperidine-1-carboxylic acid 5-(4-piperidin-1-ylmethyl-benzoylamino)-pyridin-2-yl ester,
4-(1-Methyl-1H-imidazol-2-ylsulfanyl)-piperidine-1-carboxylic acid 5-(4-piperidin-1-ylmethyl-benzoylamino)-pyridin-2-yl ester,
4-(1-Methyl-1H-tetrazol-5-ylsulfanyl)-piperidine-1-carboxylic acid 5-(4-piperidin-1-ylmethyl-benzoylamino)-pyridin-2-yl ester,
4-(Imidazol-1-yloxy)-piperidine-1-carboxylic acid 5-(4-piperidin-1-ylmethyl-benzoylamino)-pyridin-2-yl ester,
4-[1-(4-Carboxy-phenyl)-1H-tetrazol-5-ylsulfanyl]-piperidine-1-carboxylic acid 5-(4-piperidin-1-ylmethyl-benzoylamino)-pyridin-2-yl ester,
4-(4-Carboxymethyl-phenoxy)-piperidine-1-carboxylic acid 5-(4-piperidin-1-ylmethyl-benzoylamino)-pyridin-2-yl ester,
4-(Pyrazol-1-yloxy)-piperidine-1-carboxylic acid 5-(4-piperidin-1-ylmethyl-benzoylamino)-pyridin-2-yl ester,
4-(4-Carboxy-phenoxy)-piperidine-1-carboxylic acid 5-(4-piperidin-1-ylmethyl-benzoylamino)-pyridin-2-yl ester,
4-[4-(Pyridin-2-yloxy)-phenoxy]-piperidine-1-carboxylic acid 5-(4-piperidin-1-ylmethyl-benzoylamino)-pyridin-2-yl ester,
4-[4-(5-Methyl-pyridin-2-yloxy)-phenoxy]-piperidine-1-carboxylic acid 5-(4-piperidin-1-ylmethyl-benzoylamino)-pyridin-2-yl ester,
4-(4-Carboxy-phenoxymethyl)-piperidine-1-carboxylic acid 5-(4-piperidin-1-ylmethyl-benzoylamino)-pyridin-2-yl ester,
4-(4-Carboxymethyl-phenoxymethyl)-piperidine-1-carboxylic acid 5-(4-piperidin-1-ylmethyl-benzoylamino)-pyridin-2-yl ester,
4-(1H-Imidazol-2-ylsulfanylmethyl)-piperidine-1-carboxylic acid 5-(4-piperidin-1-ylmethyl-benzoylamino)-pyridin-2-yl ester,
4-(1-Methyl-1H-imidazol-2-ylsulfanylmethyl)-piperidine-1-carboxylic acid 5-(4-piperidin-1-ylmethyl-benzoylamino)-pyridin-2-yl ester,
4-(Imidazol-1-yloxymethyl)-piperidine-1-carboxylic acid 5-(4-piperidin-1-ylmethyl-benzoylamino)-pyridin-2-yl ester,
4-[4-(Pyridin-2-yloxy)-phenoxymethyl]-piperidine-1-carboxylic acid 5-(4-piperidin-1-ylmethyl-benzoylamino)-pyridin-2-yl ester,
4-[4-(5-Methyl-pyridin-2-yloxy)-phenoxymethyl]-piperidine-1-carboxylic acid 5-(4-piperidin-1-ylmethyl-benzoylamino)-pyridin-2-yl ester,
4-(1-Methyl-1H-tetrazol-5-ylsulfanylmethyl)-piperidine-1-carboxylic acid 5-(4-piperidin-1-ylmethyl-benzoylamino)-pyridin-2-yl ester,
4-[1-(2-Dimethylamino-ethyl)-1H-tetrazol-5-ylsulfanyl]-piperidine-1-carboxylic acid 5-(4-piperidin-1-ylmethyl-benzoylamino)-pyridin-2-yl ester,
4-[1-(2-Carboxy-ethyl)-1H-tetrazol-5-ylsulfanyl]-piperidine-1-carboxylic acid 5-(4-piperdin-1-ylmethyl-benzoylamino)-pyridin-2-yl ester,
4-(1-Carboxymethyl-1H-tetrazol-5-ylsulfanyl)-piperidine-1-carboxylic acid 5-(4-piperidin-1-ylmethyl-benzoylamino)-pyridin-2-yl ester,
4-( Pyrazol-1-yloxymethyl)-piperidine-1-carboxylic acid 5-(4-piperidin-1-ylmethyl-benzoylamino)-pyridin-2-yl ester,
4-(1H-[1,2,4]Triazol-3-ylsulfanylmethyl)-piperidine-1-carboxylic acid 5-(4-piperidin-1-ylmethyl-benzoylamino)-pyridin-2-yl ester,
4-[2-(Benzyl-methyl-amino)-ethyl]-piperidine-1-carboxylic acid 5-(4-piperidin-1-ylmethyl-benzoylamino)-pyridin-2-yl ester,
4-(1H-[1,2,4]Triazol-3-ylsulfanyl)-piperidine-1-carboxylic acid 5-[4-(2-ethyl-piperidin-1-ylmethyl)-benzoylamino]-pyridin-2-yl ester,
4-(1H-Imidazol-2-ylsulfanyl)-piperidine-1-carboxylic acid 5-[4-(2-ethyl-piperidin-1-ylmethyl)-benzoylamino]-pyridin-2-yl ester,
4-(1-Methyl-1H-imidazol-2-ylsulfanyl)-piperidine-1-carboxylic acid 5-[4-(2-ethyl-piperidin-1-ylmethyl)-benzoylamino]-pyridin-2-yl ester,
4-(1-Methyl-1H-tetrazol-5-ylsulfanyl)-piperidine-1-carboxylic acid 5-[4-(2-ethyl-piperidin-1-ylmethyl)-benzoylamino]-pyridin-2-yl ester,
4-(Imidazol-1-yloxy)-piperidine-1-carboxylic acid 5-[4-(2-ethyl-piperidin-1-ylmethyl)-benzoylamino]-pyridin-2-yl ester,
4-[1-(4-Carboxy-phenyl)-1H-tetrazol-5-ylsulfanyl]-piperidine-1-carboxylic acid 5-[4-(2-ethyl-piperidin-1-ylmethyl)-benzoylamino]-pyridin-2-yl ester,
4-(4-Carboxymethyl-phenoxy)-piperidine-1-carboxylic acid 5-[4-(2-ethyl-piperidin-1-ylmethyl)-benzoylamino]-pyridin-2-yl ester,
4-(Pyrazol-1-yloxy)-piperidine-1-carboxylic acid 5-[4-(2-ethyl-piperidin-1-ylmethyl)-benzoylamino]-pyridin-2-yl ester,
4-(4-Carboxy-phenoxy)-piperidine-1-carboxylic acid 5-[4-(2-ethyl-piperidin-1-ylmethyl)-benzoylamino]-pyridin-2-yl ester,
4-[4-(Pyridin-2-yloxy)-phenoxy]-piperidine-1-carboxylic acid 5-[4-(2-ethyl-piperidin-1-ylmethyl)-benzoylamino]-pyridin-2-yl ester,
4-[4-(5-Methyl-pyridin-2-yloxy)-phenoxy]-piperidine-1-carboxylic acid 5-[4-(2-ethyl-piperidin-1-ylmethyl)-benzoylamino]-pyridin-2-yl ester,
4-(4-Carboxy-phenoxymethyl)-piperidine-1-carboxylic acid 5-[4-(2-ethyl-piperidin-1-ylmethyl)-benzoylamino]-pyridin-2-yl ester,
4-(4-Carboxymethyl-phenoxymethyl)-piperidine-1-carboxylic acid 5-[4-(2-ethyl-piperidin-1-ylmethyl)-benzoylamino]-pyridin-2-yl ester,
4-(1H-Imidazol-2-ylsulfanylmethyl)-piperidine-1-carboxylic acid 5-[4-(2-ethyl-piperidin-1-ylmethyl)-benzoylamino]-pyridin-2-yl ester,
4-(1-Methyl-1H-imidazol-2-ylsulfanylmethyl)-piperidine-1-carboxylic acid 5-[4-(2-ethyl-piperidin-1-ylmethyl)-benzoylamino]-pyridin-2-yl ester,
4-(Imidazol-1-yloxymethyl)-piperidine-1-carboxylic acid 5-[4-(2-ethyl-piperidin-1-ylmethyl)-benzoylamino]-pyridin-2-yl ester,
4-[4-(Pyridin-2-yloxy)-phenoxymethyl]-piperidine-1-carboxylic acid 5-[4-(2-ethyl-piperidin-1-ylmethyl)-benzoylamino]-pyridin-2-yl ester,
4-[4-(5-Methyl-pyridin-2-yloxy)-phenoxymethyl]-piperidine-1-carboxylic acid 5-[4-(2-ethyl-piperidin-1-ylmethyl)-benzoylamino]-pyridin-2-yl ester,
4-(1-Methyl-1H-tetrazol-5-ylsulfanylmethyl)-piperidine-1-carboxylic acid 5-[4-(2-ethyl-piperidin-1-ylmethyl)-benzoylamino]-pyridin-2-yl ester,
4-[1-(2-Dimethylamino-ethyl)-1H-tetrazol-5-ylsulfanyl]-piperidine-1-carboxylic acid 5-[4-(2-ethyl-piperidin-1-ylmethyl)-benzoylamino]-pyridin-2-yl ester,
4-[1-(2-Carboxy-ethyl)-1H-tetrazol-5-ylsulfanyl]-piperidine-1-carboxylic acid 5-[4-(2-ethyl-piperidin-1-ylmethyl)-benzoylamino]-pyridin-2-yl ester,
4-(1-Carboxymethyl-1H-tetrazol-5-ylsulfanyl)-piperidine-1-carboxylic acid 5-[4-(2-ethyl-piperidin-1-ylmethyl)-benzoylamino]-pyridin-2-yl ester
4-( Pyrazol-1-yloxymethyl)-piperidine-1-carboxylic acid 5-[4-(2-ethyl-piperidin-1-ylmethyl)-benzoylamino]-pyridin-2-yl ester,
4-(1H-[1,2,4]Triazol-3-ylsulfanylmethyl)-piperidine-1-carboxylic acid 5-[4-(2-ethyl-piperidin-1-ylmethyl)-benzoylamino]-pyridin-2-yl ester,
4-[2-(Benzyl-methyl-amino)-ethyl]-piperidine-1-carboxylic acid 5-[4-(2-ethyl-piperidin-1-ylmethyl)-benzoylamino]-pyridin-2-yl ester,
4-(1H-[1,2,4]Triazol-3-ylsulfanyl)-piperidine-1-carboxylic acid 5-[4-(4-methyl-piperidin-1-ylmethyl)-benzoylamino]-pyridin-2-yl ester,
4-(1H-Imidazol-2-ylsulfanyl)-piperidine-1-carboxylic acid 5-[4-(4-methyl-piperidin-1-ylmethyl)-benzoylamino]-pyridin-2-yl ester,
4-(1-Methyl-1H-imidazol-2-ylsulfanyl)-piperidine-1-carboxylic acid 5-[4-(4-methyl-piperidin-1-ylmethyl)-benzoylamino]-pyridin-2-yl ester,
4-(1-Methyl-1H-tetrazol-5-ylsulfanyl)-piperidine-1-carboxylic acid 5-[4-(4-methyl-piperidin-1-ylmethyl)-benzoylamino]-pyridin-2-yl ester,
4-(Imidazol-1-yloxy)-piperidine-1-carboxylic acid 5-[4-(4-methyl-piperidin-1-ylmethyl)-benzoylamino]-pyridin-2-yl ester,
4-[1-(4-Carboxy-phenyl)-1H-tetrazol-5-ylsulfanyl]-piperidine-1-carboxylic acid 5-[4-(4-methyl-piperidin-1-ylmethyl)-benzoylamino]-pyridin-2-yl ester,
4-(4-Carboxymethyl-phenoxy)-piperidine-1-carboxylic acid 5-[4-(4-methyl-piperidin-1-ylmethyl)-benzoylamino]-pyridin-2-yl ester,
4-( Pyrazol-1-yloxy)-piperidine-1-carboxylic acid 5-[4-(4-methyl-piperidin-1-ylmethyl)-benzoylamino]-pyridin-2-yl ester,
4-(4-Carboxy-phenoxy)-piperidine-1-carboxylic acid 5-[4-(4-methyl-piperidin-1-ylmethyl)-benzoylamino]-pyridin-2-yl ester,
4-[4-(Pyridin-2-yloxy)-phenoxy]-piperidine-1-carboxylic acid 5-[4-(4-methyl-piperidin-1-ylmethyl)-benzoylamino]-pyridin-2-yl ester,
4-[4-(5-Methyl-pyridin-2-yloxy)-phenoxy]-piperidine-1-carboxylic acid 5-[4-(4-methyl-piperidin-1-ylmethyl)-benzoylamino]-pyridin-2-yl ester,
4-(4-Carboxy-phenoxymethyl)-piperidine-1-carboxylic acid 5-[4-(4-methyl-piperidin-1-ylmethyl)-benzoylamino]-pyridin-2-yl ester,
4-(4-Carboxymethyl-phenoxymethyl)-piperidine-1-carboxylic acid 5-[4-(4-methyl-piperidin-1-ylmethyl)-benzoylamino]-pyridin-2-yl ester,
4-(1H-Imidazol-2-ylsulfanylmethyl)-piperidine-1-carboxylic acid 5-[4-(4-methyl-piperidin-1-ylmethyl)-benzoylamino]-pyridin-2-yl ester,
4-(1-Methyl-1H-imidazol-2-ylsulfanylmethyl)-piperidine-1-carboxylic acid 5-[4-(4-methyl-piperidin-1-ylmethyl)-benzoylamino]-pyridin-2-yl ester,
4-(Imidazol-1-yloxymethyl)-piperidine-1-carboxylic acid 5-[4-(4-methyl-piperidin-1-ylmethyl)-benzoylamino]-pyridin-2-yl ester,
4-[4-(Pyridin-2-yloxy)-phenoxymethyl]-piperidine-1-carboxylic acid 5-[4-(4-methyl-piperidin-1-ylmethyl)-benzoylamino]-pyridin-2-yl ester,
4-[4-(5-Methyl-pyridin-2-yloxy)-phenoxymethyl]-piperidine-1-carboxylic acid 5-[4-(4-methyl-piperidin-1-ylmethyl)-benzoylamino]-pyridin-2-yl ester,
4-(1-Methyl-1H-tetrazol-5-ylsulfanylmethyl)-piperidine-1-carboxylic acid 5-[4-(4-methyl-piperidin-1-ylmethyl)-benzoylamino]-pyridin-2-yl ester,
4-[1-(2-Dimethylamino-ethyl)-1H-tetrazol-5-ylsulfanyl]-piperidine-1-carboxylic acid 5-[4-(4-methyl-piperidin-1-ylmethyl)-benzoylamino]-pyridin-2-yl ester,
4-[1-(2-Carboxy-ethyl)-1H-tetrazol-5-ylsulfanyl]-piperidine-1-carboxylic acid 5-[4-(4-methyl-piperidin-1-ylmethyl)-benzoylamino]-pyridin-2-yl ester,
4-(1-Carboxymethyl-1H-tetrazol-5-ylsulfanyl)-piperidine-1-carboxylic acid 5-[4-(4-methyl-piperidin-1-ylmethyl)-benzoylamino]-pyridin-2-yl ester,
4-(Pyrazol-1-yloxymethyl)-piperidine-1-carboxylic acid 5-[4-(4-methyl-piperidin-1-ylmethyl)-benzoylamino]-pyridin-2-yl ester,
4-(1H-[1,2,4]Triazol-3-ylsulfanylmethyl)-piperidine-1-carboxylic acid 5-[4-(4-methyl-piperidin-1-ylmethyl)-benzoylamino]-pyridin-2-yl ester,
4-[2-(Benzyl-methyl-amino)-ethyl]-piperidine-1-carboxylic acid 5-[4-(4-methyl-piperidin-1-ylmethyl)-benzoylamino]-pyridin-2-yl ester,
4-(1H-[1,2,4]Triazol-3-ylsulfanyl)-piperidine-1-carboxylic acid 5-[4-(3-methyl-piperidin-1-ylmethyl)-benzoylamino]-pyridin-2-yl ester,
4-(1H-Imidazol-2-ylsulfanyl)-piperidine-1-carboxylic acid 5-[4-(3-methyl-piperidin-1-ylmethyl)-benzoylamino]-pyridin-2-yl ester,
4-(1-Methyl-1H-imidazol-2-ylsulfanyl)-piperidine-1-carboxylic acid 5-[4-(3-methyl-piperidin-1-ylmethyl)-benzoylamino]-pyridin-2-yl ester,
4-(1-Methyl-1H-tetrazol-5-ylsulfanyl)-piperidine-1-carboxylic acid 5-[4-(3-methyl-piperidin-1-ylmethyl)-benzoylamino]-pyridin-2-yl ester,
4-(Imidazol-1-yloxy)-piperidine-1-carboxylic acid 5-[4-(3-methyl-piperidin-1-ylmethyl)-benzoylamino]-pyridin-2-yl ester,
4-[1-(4-Carboxy-phenyl)-1H-tetrazol-5-ylsulfanyl]-piperidine-1-carboxylic acid 5-[4-(3-methyl-piperidin-1-ylmethyl)-benzoylamino]-pyridin-2-yl ester,
4-(4-Carboxymethyl-phenoxy)-piperidine-1-carboxylic acid 5-[4-(3-methyl-piperidin-1-ylmethyl)-benzoylamino]-pyridin-2-yl ester,
4-(Pyrazol-1-yloxy)-piperidine-1-carboxylic acid 5-[4-(3-methyl-piperidin-1-ylmethyl)-benzoylamino]-pyridin-2-yl ester,
4-(4-Carboxy-phenoxy)-piperidine-1-carboxylic acid 5-[4-(3-methyl-piperidin-1-ylmethyl)-benzoylamino]-pyridin-2-yl ester,
4-[4-(Pyridin-2-yloxy)-phenoxy]-piperidine-1-carboxylic acid 5-[4-(3-methyl-piperidin-1-ylmethyl)-benzoylamino]-pyridin-2-yl ester,
4-[4-(5-Methyl-pyridin-2-yloxy)-phenoxy]-piperidine-1-carboxylic acid 5-[4-(3-methyl-piperidin-1-ylmethyl)-benzoylamino]-pyridin-2-yl ester,
4-(4-Carboxy-phenoxymethyl)-piperidine-1-carboxylic acid 5-[4-(3-methyl-piperidin-1-ylmethyl)-benzoylamino]-pyridin-2-yl ester,
4-(4-Carboxymethyl-phenoxymethyl)-piperidine-1-carboxylic acid 5-[4-(3-methyl-piperidin-1-ylmethyl)-benzoylamino]-pyridin-2-yl ester,
4-(1H-Imidazol-2-ylsulfanylmethyl)-piperidine-1-carboxylic acid 5-[4-(3-methyl-piperidin-1-ylmethyl)-benzoylamino]-pyridin-2-yl ester,
4-(1-Methyl-1H-imidazol-2-ylsulfanylmethyl)-piperidine-1-carboxylic acid 5-[4-(3-methyl-piperidin-1-ylmethyl)-benzoylamino]-pyridin-2-yl ester,
4-(Imidazol-1-yloxymethyl)-piperidine-1-carboxylic acid 5-[4-(3-methyl-piperidin-1-ylmethyl)-benzoylamino]-pyridin-2-yl ester,
4-[4-(Pyridin-2-yloxy)-phenoxymethyl]-piperidine-1-carboxylic acid 5-[4-(3-methyl-piperidin-1-ylmethyl)-benzoylamino]-pyridin-2-yl ester,
4-[4-(5-Methyl-pyridin-2-yloxy)-phenoxymethyl]-piperidine-1-carboxylic acid 5-[4-(3-methyl-piperidin-1-ylmethyl)-benzoylamino]-pyridin-2-yl ester,
4-(1-Methyl-1H-tetrazol-5-ylsulfanylmethyl)-piperidine-1-carboxylic acid 5-[4-(3-methyl-piperidin-1-ylmethyl)-benzoylamino]-pyridin-2-yl ester,
4-[1-(2-Dimethylamino-ethyl)-1H-tetrazol-5-ylsulfanyl]-piperidine-1-carboxylic acid 5-[4-(3-methyl-piperidin-1-ylmethyl)-benzoylamino]-pyridin-2-yl ester,
4-[1-(2-Carboxy-ethyl)-1H-tetrazol-5-ylsulfanyl]-piperidine-1-carboxylic acid 5-[4-(3-methyl-piperidin-1-ylmethyl)-benzoylamino]-pyridin-2-yl ester,
4-(1-Carboxymethyl-1H-tetrazol-5-ylsulfanyl)-piperidine-1-carboxylic acid 5-[4-(3-methyl-piperidin-1-ylmethyl)-benzoylamino]-pyridin-2-yl ester,
4-(Pyrazol-1-yloxymethyl)-piperidine-1-carboxylic acid 5-[4-(3-methyl-piperidin-1-ylmethyl)-benzoylamino]-pyridin-2-yl ester,
4-(1H-[1,2,4]Triazol-3-ylsulfanylmethyl)-piperidine-1-carboxylic acid 5-[4-(3-methyl-piperidin-1-ylmethyl)-benzoylamino]-pyridin-2-yl ester,
4-[2-(Benzyl-methyl-amino)-ethyl]-piperidine-1-carboxylic acid 5-[4-(3-methyl-piperidin-1-ylmethyl)-benzoylamino]-pyridin-2-yl ester,
4-(1H-[1,2,4]Triazol-3-ylsulfanyl)-piperidine-1-carboxylic acid 5-[4-(2-methyl-piperidin-1-ylmethyl)-benzoylamino]-pyridin-2-yl ester,
4-(1H-Imidazol-2-ylsulfanyl)-piperidine-1-carboxylic acid 5-[4-(2-methyl-piperidin-1-ylmethyl)-benzoylamino]-pyridin-2-yl ester,
4-(1-Methyl-1H-imidazol-2-ylsulfanyl)-piperidine-1-carboxylic acid 5-[4-(2-methyl-piperidin-1-ylmethyl)-benzoylamino]-pyridin-2-yl ester,
4-(1-Methyl-1H-tetrazol-5-ylsulfanyl)-piperidine-1-carboxylic acid 5-[4-(2-methyl-piperidin-1-ylmethyl)-benzoylamino]-pyridin-2-yl ester,
4-(Imidazol-1-yloxy)-piperidine-1-carboxylic acid 5-[4-(2-methyl-piperidin-1-ylmethyl)-benzoylamino]-pyridin-2-yl ester,
4-[1-(4-Carboxy-phenyl)-1H-tetrazol-5-ylsulfanyl]-piperidine-1-carboxylic acid 5-[4-(2-methyl-piperidin-1-ylmethyl)-benzoylamino]-pyridin-2-yl ester,
4-(4-Carboxymethyl-phenoxy)-piperidine-1-carboxylic acid 5-[4-(2-methyl-piperidin-1-ylmethyl)-benzoylamino]-pyridin-2-yl ester,
4-(Pyrazol-1-yloxy)-piperidine-1-carboxylic acid 5-[4-(2-methyl-piperidin-1-ylmethyl)-benzoylamino]-pyridin-2-yl ester,
4-(4-Carboxy-phenoxy)-piperidine-1-carboxylic acid 5-[4-(2-methyl-piperidin-1-ylmethyl)-benzoylamino]-pyridin-2-yl ester,
4-[4-(Pyridin-2-yloxy)-phenoxy]-piperidine-1-carboxylic acid 5-[4-(2-methyl-piperidin-1-ylmethyl)-benzoylamino]-pyridin-2-yl ester,
4-[4-(5-Methyl-pyridin-2-yloxy)-phenoxy]-piperidine-1-carboxylic acid 5-[4-(2-methyl-piperidin-1-ylmethyl)-benzoylamino]-pyridin-2-yl ester,
4-(4-Carboxy-phenoxymethyl)-piperidine-1-carboxylic acid 5-[4-(2-methyl-piperidin-1-ylmethyl)-benzoylamino]-pyridin-2-yl ester,
4-(4-Carboxymethyl-phenoxymethyl)-piperidine-1-carboxylic acid 5-[4-(2-methyl-piperidin-1-ylmethyl)-benzoylamino]-pyridin-2-yl ester,
4-(1H-Imidazol-2-ylsulfanylmethyl)-piperidine-1-carboxylic acid 5-[4-(2-methyl-piperidin-1-ylmethyl)-benzoylamino]-pyridin-2-yl ester,
4-(1-Methyl-1H-imidazol-2-ylsulfanylmethyl)-piperidine-1-carboxylic acid 5-[4-(2-methyl-piperidin-1-ylmethyl)-benzoylamino]-pyridin-2-yl ester,
4-(Imidazol-1-yloxymethyl)-piperidine-1-carboxylic acid 5-[4-(2-methyl-piperidin-1-ylmethyl)-benzoylamino]-pyridin-2-yl ester,
4-[4-(Pyridin-2-yloxy)-phenoxymethyl]-piperidine-1-carboxylic acid 5-[4-(2-methyl-piperidin-1-ylmethyl)-benzoylamino]-pyridin-2-yl ester,
4-[4-(5-Methyl-pyridin-2-yloxy)-phenoxymethyl]-piperidine-1-carboxylic acid 5-[4-(2-methyl-piperidin-1-ylmethyl)-benzoylamino]-pyridin-2-yl ester,
4-(1-Methyl-1H-tetrazol-5-ylsulfanylmethyl)-piperidine-1-carboxylic acid 5-[4-(2-methyl-piperidin-1-ylmethyl)-benzoylamino]-pyridin-2-yl ester,
4-[1-(2-Dimethylamino-ethyl)-1H-tetrazol-5-ylsulfanyl]-piperidine-1-carboxylic acid 5-[4-(2-methyl-piperidin-1-ylmethyl)-benzoylamino]-pyridin-2-yl ester,
4-[1-(2-Carboxy-ethyl)-1H-tetrazol-5-ylsulfanyl]-piperidine-1-carboxylic acid 5-[4-(2-methyl-piperidin-1-ylmethyl)-benzoylamino]-pyridin-2-yl ester,
4-(1-Carboxymethyl-1H-tetrazol-5-ylsulfanyl)-piperidine-1-carboxylic acid 5-[4-(2-methyl-piperidin-1-ylmethyl)-benzoylamino]-pyridin-2-yl ester,
4-(Pyrazol-1-yloxymethyl)-piperidine-1-carboxylic acid 5-[4-(2-methyl-piperidin-1-ylmethyl)-benzoylamino]-pyridin-2-yl ester,
4-(1H-[1,2,4]Triazol-3-ylsulfanylmethyl)-piperidine-1-carboxylic acid 5-[4-(2-methyl-piperidin-1-ylmethyl)-benzoylamino]-pyridin-2-yl ester,
4-[2-(Benzyl-methyl-amino)-ethyl]-piperidine-1-carboxylic acid 5-[4-(2-methyl-piperidin-1-ylmethyl)-benzoylamino]-pyridin-2-yl ester,
4-(1H-[1,2,4]Triazol-3-ylsulfanyl)-piperidine-1-carboxylic acid 4-(5-trifluoromethyl-pyridin-2-ylmethyl)-phenyl ester,
4-(1H-Imidazol-2-ylsulfanyl)-piperidine-1-carboxylic acid 4-(5-trifluoromethyl-pyridin-2-ylmethyl)-phenyl ester,
4-(1-Methyl-1H-imidazol-2-ylsulfanyl)-piperidine-1-carboxylic acid 4-(5-trifluoromethyl-pyridin-2-ylmethyl)-phenyl ester,
4-(1-Methyl-1H-tetrazol-5-ylsulfanyl)-piperidine-1-carboxylic acid 4-(5-trifluoromethyl-pyridin-2-ylmethyl)-phenyl ester,
4-(Imidazol-1-yloxy)-piperidine-1-carboxylic acid 4-(5-trifluoromethyl-pyridin-2-ylmethyl)-phenyl ester,
4-[1-(4-Carboxy-phenyl)-1H-tetrazol-5-ylsulfanyl]-piperidine-1-carboxylic acid 4-(5-trifluoromethyl-pyridin-2-ylmethyl)-phenyl ester,
4-(4-Carboxymethyl-phenoxy)-piperidine-1-carboxylic acid 4-(5-trifluoromethyl-pyridin-2-ylmethyl)-phenyl ester,
4-(Pyrazol-1-yloxy)-piperidine-1-carboxylic acid 4-(5-trifluoromethyl-pyridin-2-ylmethyl)-phenyl ester,
4-(4-Carboxy-phenoxy)-piperidine-1-carboxylic acid 4-(5-trifluoromethyl-pyridin-2-ylmethyl)-phenyl ester,
4-[4-(Pyridin-2-yloxy)-phenoxy]-piperidine-1-carboxylic acid 4-(5-trifluoromethyl-pyridin-2-ylmethyl)-phenyl ester,
4-[4-(5-Methyl-pyridin-2-yloxy)-phenoxy]-piperidine-1-carboxylic acid 4-(5-trifluoromethyl-pyridin-2-ylmethyl)-phenyl ester,
4-(4-Carboxy-phenoxymethyl)-piperidine-1-carboxylic acid 4-(5-trifluoromethyl-pyridin-2-ylmethyl)-phenyl ester,
4-(4-Carboxymethyl-phenoxymethyl)-piperidine-1-carboxylic acid 4-(5-trifluoromethyl-pyridin-2-ylmethyl)-phenyl ester,
4-(1H-Imidazol-2-ylsulfanylmethyl)-piperidine-1-carboxylic acid 4-(5-trifluoromethyl-pyridin-2-ylmethyl)-phenyl ester,
4-(1-Methyl-1H-imidazol-2-ylsulfanylmethyl)-piperidine-1-carboxylic acid 4-(5-trifluoromethyl-pyridin-2-ylmethyl)-phenyl ester,
4-(Imidazol-1-yloxymethyl)-piperidine-1-carboxylic acid 4-(5-trifluoromethyl-pyridin-2-ylmethyl)-phenyl ester,
4-[4-(Pyridin-2-yloxy)-phenoxymethyl]-piperidine-1-carboxylic acid 4-(5-trifluoromethyl-pyridin-2-ylmethyl)-phenyl ester,
4-[4-(5-Methyl-pyridin-2-yloxy)-phenoxymethyl]-piperidine-1-carboxylic acid 4-(5-trifluoromethyl-pyridin-2-ylmethyl)-phenyl ester,
4-(1-Methyl-1H-tetrazol-5-ylsulfanylmethyl)-piperidine-1-carboxylic acid 4-(5-trifluoromethyl-pyridin-2-ylmethyl)-phenyl ester,
4-[1-(2-Dimethylamino-ethyl)-1H-tetrazol-5-ylsulfanyl]-piperidine-1-carboxylic acid 4-(5-trifluoromethyl-pyridin-2-ylmethyl)-phenyl ester,
4-[1-(2-Carboxy-ethyl)-1H-tetrazol-5-ylsulfanyl]-piperidine-1-carboxylic acid 4-(5-trifluoromethyl-pyridin-2-ylmethyl)-phenyl ester,
4-(1-Carboxymethyl-1H-tetrazol-5-ylsulfanyl)-piperidine-1-carboxylic acid 4-(5-trifluoromethyl-pyridin-2-ylmethyl)-phenyl ester,
4-(Pyrazol-1-yloxymethyl)-piperidine-1-carboxylic acid 4-(5-trifluoromethyl-pyridin-2-ylmethyl)-phenyl ester,
4-(1H-[1,2,4]Triazol-3-ylsulfanylmethyl)-piperidine-1-carboxylic acid 4-(5-trifluoromethyl-pyridin-2-ylmethyl)-phenyl ester,
4-[2-(Benzyl-methyl-amino)-ethyl]-piperidine-1-carboxylic acid 4-(5-trifluoromethyl-pyridin-2-ylmethyl)-phenyl ester,
4-(1H-[1,2,4]Triazol-3-ylsulfanyl)-piperidine-1-carboxylic acid 4-(4-methyl-pyridin-2-ylmethyl)-phenyl ester,
4-(1H-Imidazol-2-ylsulfanyl)-piperidine-1-carboxylic acid 4-(4-methyl-pyridin-2-ylmethyl)-phenyl ester,
4-(1-Methyl-1H-imidazol-2-ylsulfanyl)-piperidine-1-carboxylic acid 4-(4-methyl-pyridin-2-ylmethyl)-phenyl ester,
4-(1-Methyl-1H-tetrazol-5-ylsulfanyl)-piperidine-1-carboxylic acid 4-(4-methyl-pyridin-2-ylmethyl)-phenyl ester,
4-(Imidazol-1-yloxy)-piperidine-1-carboxylic acid 4-(4-methyl-pyridin-2-ylmethyl)-phenyl ester,
4-[1-(4-Carboxy-phenyl)-1H-tetrazol-5-ylsulfanyl]-piperidine-1-carboxylic acid 4-(4-methyl-pyridin-2-ylmethyl)-phenyl ester,
4-(4-Carboxymethyl-phenoxy)-piperidine-1-carboxylic acid 4-(4-methyl-pyridin-2-ylmethyl)-phenyl ester,
4-(Pyrazol-1-yloxy)-piperidine-1-carboxylic acid 4-(4-methyl-pyridin-2-ylmethyl)-phenyl ester,
4-(4-Carboxy-phenoxy)-piperidine-1-carboxylic acid 4-(4-methyl-pyridin-2-ylmethyl)-phenyl ester,
4-[4-(Pyridin-2-yloxy)-phenoxy]-piperidine-1-carboxylic acid 4-(4-methyl-pyridin-2-ylmethyl)-phenyl ester,
4-[4-(5-Methyl-pyridin-2-yloxy)-phenoxy]-piperidine-1-carboxylic acid 4-(4-methyl-pyridin-2-ylmethyl)-phenyl ester,
4-(4-Carboxy-phenoxymethyl)-piperidine-1-carboxylic acid 4-(4-methyl-pyridin-2-ylmethyl)-phenyl ester,
4-(4-Carboxymethyl-phenoxymethyl)-piperidine-1-carboxylic acid 4-(4-methyl-pyridin-2-ylmethyl)-phenyl ester,
4-(1H-Imidazol-2-ylsulfanylmethyl)-piperidine-1-carboxylic acid 4-(4-methyl-pyridin-2-ylmethyl)-phenyl ester,
4-(1-Methyl-1H-imidazol-2-ylsulfanylmethyl)-piperidine-1-carboxylic acid 4-(4-methyl-pyridin-2-ylmethyl)-phenyl ester,
4-(Imidazol-1-yloxymethyl)-piperidine-1-carboxylic acid 4-(4-methyl-pyridin-2-ylmethyl)-phenyl ester,
4-[4-(Pyridin-2-yloxy)-phenoxymethyl]-piperidine-1-carboxylic acid 4-(4-methyl-pyridin-2-ylmethyl)-phenyl ester,
4-[4-(5-Methyl-pyridin-2-yloxy)-phenoxymethyl]-piperidine-1-carboxylic acid 4-(4-methyl-pyridin-2-ylmethyl)-phenyl ester,
4-(1-Methyl-1H-tetrazol-5-ylsulfanylmethyl)-piperidine-1-carboxylic acid 4-(4-methyl-pyridin-2-ylmethyl)-phenyl ester,
4-[1-(2-Dimethylamino-ethyl)-1H-tetrazol-5-ylsulfanyl]-piperidine-1-carboxylic acid 4-(4-methyl-pyridin-2-ylmethyl)-phenyl ester,
4-[1-(2-Carboxy-ethyl)-1H-tetrazol-5-ylsulfanyl]-piperidine-1-carboxylic acid 4-(4-methyl-pyridin-2-ylmethyl)-phenyl ester,
4-(1-Carboxymethyl-1H-tetrazol-5-ylsulfanyl)-piperidine-1-carboxylic acid 4-(4-methyl-pyridin-2-ylmethyl)-phenyl ester,
4-(Pyrazol-1-yloxymethyl)-piperidine-1-carboxylic acid 4-(4-methyl-pyridin-2-ylmethyl)-phenyl ester,
4-(1H-[1,2,4]Triazol-3-ylsulfanylmethyl)-piperidine-1-carboxylic acid 4-(4-methyl-pyridin-2-ylmethyl)-phenyl ester,
4-[2-(Benzyl-methyl-amino)-ethyl]-piperidine-1-carboxylic acid 4-(4-methyl-pyridin-2-ylmethyl)-phenyl ester,
4-(1H-[1,2,4]Triazol-3-ylsulfanyl)-piperidine-1-carboxylic acid 4-pyridin-2-ylmethyl-phenyl ester,
4-(1H-Imidazol-2-ylsulfanyl)-piperidine-1-carboxylic acid 4-pyridin-2-ylmethyl-phenyl ester,
4-(1-Methyl-1H-imidazol-2-ylsulfanyl)-piperidine-1-carboxylic acid 4-pyridin-2-ylmethyl-phenyl ester,
4-(1-Methyl-1H-tetrazol-5-ylsulfanyl)-piperidine-1-carboxylic acid 4-pyridin-2-ylmethyl-phenyl ester,
4-(Imidazol-1-yloxy)-piperidine-1-carboxylic acid 4-pyridin-2-ylmethyl-phenyl ester,
4-[1-(4-Carboxy-phenyl)-1H-tetrazol-5-ylsulfanyl]-piperidine-1-carboxylic acid 4-pyridin-2-ylmethyl-phenyl ester,
4-(4-Carboxymethyl-phenoxy)-piperidine-1-carboxylic acid 4-pyridin-2-ylmethyl-phenyl ester,
4-(Pyrazol-1-yloxy)-piperidine-1-carboxylic acid 4-pyridin-2-ylmethyl-phenyl ester,
4-(4-Carboxy-phenoxy)-piperidine-1-carboxylic acid 4-pyridin-2-ylmethyl-phenyl ester,
4-[4-(Pyridin-2-yloxy)-phenoxy]-piperidine-1-carboxylic acid 4-pyridin-2-ylmethyl-phenyl ester,
4-[4-(5-Methyl-pyridin-2-yloxy)-phenoxy]-piperidine-1-carboxylic acid 4-pyridin-2-ylmethyl-phenyl ester,
4-(4-Carboxy-phenoxymethyl)-piperidine-1-carboxylic acid 4-pyridin-2-ylmethyl-phenyl ester,
4-(4-Carboxymethyl-phenoxymethyl)-piperidine-1-carboxylic acid 4-pyridin-2-ylmethyl-phenyl ester,
4-(1H-Imidazol-2-ylsulfanylmethyl)-piperidine-1-carboxylic acid 4-pyridin-2-ylmethyl-phenyl ester,
4-(1-Methyl-1H-imidazol-2-ylsulfanylmethyl)-piperidine-1-carboxylic acid 4-pyridin-2-ylmethyl-phenyl ester,
4-(Imidazol-1-yloxymethyl)-piperidine-1-carboxylic acid 4-pyridin-2-ylmethyl-phenyl ester,
4-[4-(Pyridin-2-yloxy)-phenoxymethyl]-piperidine-1-carboxylic acid 4-pyridin-2-ylmethyl-phenyl ester,
4-[4-(5-Methyl-pyridin-2-yloxy)-phenoxymethyl]-piperidine-1-carboxylic acid 4-pyridin-2-ylmethyl-phenyl ester,
4-(1-Methyl-1H-tetrazol-5-ylsulfanylmethyl)-piperidine-1-carboxylic acid 4-pyridin-2-ylmethyl-phenyl ester,
4-(1-(2-Dimethylamino-ethyl)-1H-tetrazol-5-ylsulfanyl]-piperidine-1-carboxylic acid 4-pyridin-2-ylmethyl-phenyl ester,
4-[1-(2-Carboxy-ethyl)-1H-tetrazol-5-ylsulfanyl]-piperidine-1-carboxylic acid 4-pyridin-2-ylmethyl-phenyl ester,
4-(1-Carboxymethyl-1H-tetrazol-5-ylsulfanyl)-piperidine-1-carboxylic acid 4-pyridin-2-ylmethyl-phenyl ester,
4-(Pyrazol-1-yloxymethyl)-piperidine-1-carboxylic acid 4-pyridin-2-ylmethyl-phenyl ester,
4-(1H-[1,2,4]Triazol-3-ylsulfanylmethyl)-piperidine-1-carboxylic acid 4-pyridin-2-ylmethyl-phenyl ester,
4-[2-(Benzyl-methyl-amino)-ethyl]-piperidine-1-carboxylic acid 4-pyridin-2-ylmethyl-phenyl ester,
4-(1H-[1,2,4]Triazol-3-ylsulfanyl)-piperidine-1-carboxylic acid 4-(3-methyl-pyridin-2-ylmethyl)-phenyl ester,
4-(1H-Imidazol-2-ylsulfanyl)-piperidine-1-carboxylic acid 4-(3-methyl-pyridin-2-ylmethyl)-phenyl ester,
4-(1-Methyl-1H-imidazol-2-ylsulfanyl)-piperidine-1-carboxylic acid 4-(3-methyl-pyridin-2-ylmethyl)-phenyl ester,
4-(1-Methyl-1H-tetrazol-5-ylsulfanyl)-piperidine-1-carboxylic acid 4-(3-methyl-pyridin-2-ylmethyl)-phenyl ester,
4-(Imidazol-1-yloxy)-piperidine-1-carboxylic acid 4-(3-methyl-pyridin-2-ylmethyl)-phenyl ester,
4-[1-(4-Carboxy-phenyl)-1H-tetrazol-5-ylsulfanyl]-piperidine-1-carboxylic acid 4-(3-methyl-pyridin-2-ylmethyl)-phenyl ester,
4-(4-Carboxymethyl-phenoxy)-piperidine-1-carboxylic acid 4-(3-methyl-pyridin-2-ylmethyl)-phenyl ester,
4-(Pyrazol-1-yloxy)-piperidine-1-carboxylic acid 4-(3-methyl-pyridin-2-ylmethyl)-phenyl ester,
4-(4-Carboxy-phenoxy)-piperidine-1-carboxylic acid 4-(3-methyl-pyridin-2-ylmethyl)-phenyl ester,
4-[4-(Pyridin-2-yloxy)-phenoxy]-piperidine-1-carboxylic acid 4-(3-methyl-pyridin-2-ylmethyl)-phenyl ester,
4-[4-(5-Methyl-pyridin-2-yloxy)-phenoxy]-piperidine-1-carboxylic acid 4-(3-methyl-pyridin-2-ylmethyl)-phenyl ester,
4-(4-Carboxy-phenoxymethyl)-piperidine-1-carboxylic acid 4-(3-methyl-pyridin-2-ylmethyl)-phenyl ester,
4-(4-Carboxymethyl-phenoxymethyl)-piperidine-1-carboxylic acid 4-(3-methyl-pyridin-2-ylmethyl)-phenyl ester,
4-(1H-Imidazol-2-ylsulfanylmethyl)-piperidine-1-carboxylic acid 4-(3-methyl-pyridin-2-ylmethyl)-phenyl ester,
4-(1-Methyl-1H-imidazol-2-ylsulfanylmethyl)-piperidine-1-carboxylic acid 4-(3-methyl-pyridin-2-ylmethyl)-phenyl ester,
4-(Imidazol-1-yloxymethyl)-piperidine-1-carboxylic acid 4-(3-methyl-pyridin-2-ylmethyl)-phenyl ester,
4-[4-(Pyridin-2-yloxy)-phenoxymethyl]-piperidine-1-carboxylic acid 4-(3-methyl-pyridin-2-ylmethyl)-phenyl ester,
4-[4-(5-Methyl-pyridin-2-yloxy)-phenoxymethyl]-piperidine-1-carboxylic acid 4-(3-methyl-pyridin-2-ylmethyl)-phenyl ester,
4-(1-Methyl-1H-tetrazol-5-ylsulfanylmethyl)-piperidine-1-carboxylic acid 4-(3-methyl-pyridin-2-ylmethyl)-phenyl ester,
4-[1-(2-Dimethylamino-ethyl)-1H-tetrazol-5-ylsulfanyl]-piperidine-1-carboxylic acid 4-(3-methyl-pyridin-2-ylmethyl)-phenyl ester,
4-[1-(2-Carboxy-ethyl)-1H-tetrazol-5-ylsulfanyl]-piperidine-1-carboxylic acid 4-(3-methyl-pyridin-2-ylmethyl)-phenyl ester,
4-(1-Carboxymethyl-1H-tetrazol-5-ylsulfanyl)-piperidine-1-carboxylic acid 4-(3-methyl-pyridin-2-ylmethyl)-phenyl ester,
4-(Pyrazol-1-yloxymethyl)-piperidine-1-carboxylic acid 4-(3-methyl-pyridin-2-ylmethyl)-phenyl ester,
4-(1H-[1,2,4]Triazol-3-ylsulfanylmethyl)-piperidine-1-carboxylic acid 4-(3-methyl-pyridin-2-ylmethyl)-phenyl ester,
4-[2-(Benzyl-methyl-amino)-ethyl]-piperidine-1-carboxylic acid 4-(3-methyl-pyridin-2-ylmethyl)-phenyl ester,
4-(1H-[1,2,4]Triazol-3-ylsulfanyl)-piperidine-1-carboxylic acid 4-[2-(1-methyl-1H-imidazol-2-ylsulfanyl)-ethyl]-phenyl ester,
4-(1H-Imidazol-2-ylsulfanyl)-piperidine-1-carboxylic acid 4-[2-(1-methyl-1H-imidazol-2 ylsulfanyl)-ethyl]-phenyl ester,
4-(1-Methyl-1H-imidazol-2-ylsulfanyl)-piperidine-1-carboxylic acid 4-[2-(1-methyl-1H-imidazol-2-ylsulfanyl)-ethyl]-phenyl ester,
4-(1-Methyl-1H-tetrazol-5-ylsulfanyl)-piperidine-1-carboxylic acid 4-[2-(1-methyl-1H-imidazol-2-ylsulfanyl)-ethyl]-phenyl ester,
4-(Imidazol-1-yloxy)-piperidine-1-carboxylic acid 4-[2-(1-methyl-1H-imidazol-2-ylsulfanyl ethyl]-phenyl ester,
4-[1-(4-Carboxy-phenyl)-1H-tetrazol-5-ylsulfanyl]-piperidine-1-carboxylic acid 4-[2-(1-methyl-1H-imidazol-2-ylsulfanyl)-ethyl]-phenyl ester,
4-(4-Carboxymethyl-phenoxy)-piperidine-1-carboxylic acid 4-[2-(1-methyl-1H-imidazol-2-ylsulfanyl)-ethyl]-phenyl ester,
4-(Pyrazol-1-yloxy)-piperidine-1-carboxylic acid 4-[2-(1-methyl-1H-imidazol-2-ylsulfanyl)-ethyl]-phenyl ester,
4-(4-Carboxy-phenoxy)-piperidine-1-carboxylic acid 4-[2-(1-methyl-1H-imidazol-2-ylsulfanyl)-ethyl]-phenyl ester,
4-[4-(Pyridin-2-yloxy)-phenoxy]-piperidine-1-carboxylic acid 4-[2-(1-methyl-1H-imidazol-2-ylsulfanyl)-ethyl]-phenyl ester,
4-[4-(5-Methyl-pyridin-2-yloxy)-phenoxy]-piperidine-1-carboxylic acid 4-[2-(1-methyl-1H-imidazol-2-ylsulfanyl)-ethyl]-phenyl ester,
4-(4-Carboxy-phenoxymethyl)-piperidine-1-carboxylic acid 4-[2-(1-methyl-1H-imidazol-2-ylsulfanyl)-ethyl]-phenyl ester,
4-(4-Carboxymethyl-phenoxymethyl)-piperidine-1-carboxylic acid 4-[2-(1-methyl-1H-imidazol-2-ylsulfanyl)-ethyl]-phenyl ester,
4-(1H-Imidazol-2-ylsulfanylmethyl)-piperidine-1-carboxylic acid 4-[2-(1-methyl-1H-imidazol-2-ylsulfanyl)-ethyl]-phenyl ester,
4-(1-Methyl-1H-imidazol-2-ylsulfanylmethyl)-piperidine-1-carboxylic acid 4-[2-(1-methyl-1H-imidazol-2-ylsulfanyl)-ethyl]-phenyl ester,
4-(Imidazol-1-yloxymethyl)-piperidine-1-carboxylic acid 4-[2-(1-methyl-1H-imidazol-2-ylsulfanyl)-ethyl]-phenyl ester,
4-[4-(Pyridin-2-yloxy)-phenoxymethyl]-piperidine-1-carboxylic acid 4-[2-(1-methyl-1H-imidazol-2-ylsulfanyl)-ethyl]-phenyl ester,
4-[4-(5-Methyl-pyridin-2-yloxy)-phenoxymethyl]-piperidine-1-carboxylic acid 4-[2-(1-methyl-1H-imidazol-2-ylsulfanyl)-ethyl]-phenyl ester,
4-(1-Methyl-1H-tetrazol-5-ylsulfanylmethyl)-piperidine-1-carboxylic acid 4-[2-(1-methyl-1H-imidazol-2-ylsulfanyl)-ethyl]-phenyl ester,
4-[1-(2-Dimethylamino-ethyl)-1H-tetrazol-5-ylsulfanyl]-piperidine-1-carboxylic acid 4-[2-(1-methyl-1H-imidazol-2-ylsulfanyl)-ethyl]-phenyl ester,
4-[1-(2-Carboxy-ethyl)-1H-tetrazol-5-ylsulfanyl]-piperidine-1-carboxylic acid 4-[2-(1-methyl-1H-imidazol-2-ylsulfanyl)-ethyl]-phenyl ester,
4-(1-Carboxymethyl-1H-tetrazol-5-ylsulfanyl)-piperidine-1-carboxylic acid 4-[2-(1-methyl-1H-imidazol-2-ylsulfanyl)-ethyl]-phenyl ester,
4-(Pyrazol-1-yloxymethyl)-piperidine-1-carboxylic acid 4-[2-(1-methyl-1H-imidazol-2-ylsulfanyl)-ethyl]-phenyl ester,
4-(1H-[1,2,4]Triazol-3-ylsulfanylmethyl)-piperidine-1-carboxylic acid 4-[2-(1-methyl-imidazol-2-ylsulfanyl)-ethyl]-phenyl ester,
4-[2-(Benzyl-methyl-amino)-ethyl]-piperidine-1-carboxylic acid 4-[2-(1-methyl-1H-imidazol-2-ylsulfanyl)-ethyl]-phenyl ester,
4-(1H-[1,2,4]Triazol-3-ylsulfanyl)-piperidine-1-carboxylic acid 4-[2-(pyridin-2-ylsulfanyl)-ethyl]-phenyl ester,
4-(1H-Imidazol-2-ylsulfanyl)-piperidine-1-carboxylic acid 4-[2-(pyridin-2-ylsulfanyl)-ethyl]-phenyl ester,
4-(1-Methyl-1H-imidazol-2-ylsulfanyl)-piperidine-1-carboxylic acid 4-[2-(pyridin-2-ylsulfanyl)-ethyl]-phenyl ester,
4-(1-Methyl-1H-tetrazol-5-ylsulfanyl)-piperidine-1-carboxylic acid 4-[2-(pyridin-2-ylsulfanyl)-ethyl]-phenyl ester,
4-(Imidazol-1-yloxy)-piperidine-1-carboxylic acid 4-[2-(pyridin-2-ylsulfanyl)-ethyl]-phenyl ester,
4-[1-(4-Carboxy-phenyl)-1H-tetrazol-5-ylsulfanyl]-piperidine-1-carboxylic acid 4-[2-(pyridin-2-ylsulfanyl)-ethyl]-phenyl ester,
4-(4-Carboxymethyl-phenoxy)-piperidine-1-carboxylic acid 4-[2-(pyridin-2-ylsulfanyl)-ethyl]-phenyl ester,
4-(Pyrazol-1-yloxy)-piperidine-1-carboxylic acid 4-[2-(pyridin-2-ylsulfanyl)-ethyl]-phenyl ester,
4-(4-Carboxy-phenoxy)-piperidine-1-carboxylic acid 4-[2-(pyridin-2-ylsulfanyl)-ethyl]-phenyl ester,
4-[4-(Pyridin-2-yloxy)-phenoxy]-piperidine-1-carboxylic acid 4-[2-(pyridin-2-ylsulfanyl)-ethyl]-phenyl ester,
4-[4-(5-Methyl-pyridin-2-yloxy)-phenoxy]-piperidine-1-carboxylic acid 4-[2-(pyridin-2-ylsulfanyl)-ethyl]-phenyl ester,
4-(4-Carboxy-phenoxymethyl)-piperidine-1-carboxylic acid 4-[2-(pyridin-2-ylsulfanyl)-ethyl]-phenyl ester,
4-(4-Carboxymethyl-phenoxymethyl)-piperidine-1-carboxylic acid 4-[2-(pyridin-2-ylsulfanyl)-ethyl]-phenyl ester,
4-(1H-Imidazol-2-ylsulfanylmethyl)-piperidine-1-carboxylic acid 4-[2-(pyridin-2-ylsulfanyl)-ethyl]-phenyl ester,
4-(1-Methyl-1H-imidazol-2-ylsulfanylmethyl)-piperidine-1-carboxylic acid 4-[2-(pyridin-2-ylsulfanyl)-ethyl]-phenyl ester,
4-(Imidazol-1-yloxymethyl)-piperidine-1-carboxylic acid 4-[2-(pyridin-2-ylsulfanyl)-ethyl]-phenyl ester,
4-[4-(Pyridin-2-yloxy)-phenoxymethyl]-piperidine-1-carboxylic acid 4-[2-(pyridin-2-ylsulfanyl)-ethyl]-phenyl ester,
4-[4-(5-Methyl-pyridin-2-yloxy)-phenoxymethyl]-piperidine-1-carboxylic acid 4-[2-(pyridin-2-ylsulfanyl)-ethyl]-phenyl ester,
4-(1-Methyl-1H-tetrazol-5-ylsulfanylmethyl)-piperidine-1-carboxylic acid 4-[2-(pyridin-2-ylsulfanyl)-ethyl]-phenyl ester,
4-[1-(2-Dimethylamino-ethyl)-1H-tetrazol-5-ylsulfanyl]-piperidine-1-carboxylic acid 4-[2-(pyridin-2-ylsulfanyl)-ethyl]-phenyl ester,
4-[1-(2-Carboxy-ethyl)-1H-tetrazol-5-ylsulfanyl]-piperidine-1-carboxylic acid 4-[2-(pyridin-2-ylsulfanyl)-ethyl]-phenyl ester,
4-(1-Carboxymethyl-1H-tetrazol-5-ylsulfanyl)-piperidine-1-carboxylic acid 4-[2-(pyridin-2-ylsulfanyl)-ethyl]-phenyl ester,
4-(Pyrazol-1-yloxymethyl)-piperidine-1-carboxylic acid 4-[2-(pyridin-2-ylsulfanyl)-ethyl]-phenyl ester,
4-(1H-[1,2,4]Triazol-3-ylsulfanylmethyl)-piperidine-1-carboxylic acid 4-[2-(pyridin-2-ylsulfanyl)-ethyl]-phenyl ester,
4-[2-(Benzyl-methyl-amino)-ethyl]-piperidine-1-carboxylic acid 4-[2-(pyridin-2-ylsulfanyl)-ethyl]-phenyl ester,
4-(1H-[1,2,4]Triazol-3-ylsulfanyl)-piperidine-1-carboxylic acid 4-[2-(pyridin-2-yloxy)-ethyl]-phenyl ester,
4-(1H-Imidazol-2-ylsulfanyl)-piperidine-1-carboxylic acid 4-[2-(pyridin-2-yloxy)-ethyl]-phenyl ester,
4-(1-Methyl-1H-imidazol-2-ylsulfanyl)-piperidine-1-carboxylic acid 4-[2-(pyridin-2-yloxy)-ethyl]-phenyl ester,
4-(1-Methyl-1H-tetrazol-5-ylsulfanyl)-piperidine-1-carboxylic acid 4-[2-(pyridin-2-yloxy)-ethyl]-phenyl ester,
4-(Imidazol-1-yloxy)-piperidine-1-carboxylic acid 4-[2-(pyridin-2-yloxy)-ethyl]-phenyl ester,
4-[1-(4-Carboxy-phenyl)-1H-tetrazol-5-ylsulfanyl]-piperidine-1-carboxylic acid 4-[2-(pyridin-2-yloxy)-ethyl]-phenyl ester,
4-(4-Carboxymethyl-phenoxy)-piperidine-1-carboxylic acid 4-[2-(pyridin-2-yloxy)-ethyl]-phenyl ester,
4-(Pyrazol-1-yloxy)-piperidine-1-carboxylic acid 4-[2-(pyridin-2-yloxy)-ethyl)-phenyl ester,
4-(4-Carboxy-phenoxy)-piperidine-1-carboxylic acid 4-[2-(pyridin-2-yloxy)-ethyl]-phenyl ester,
4-[4-(Pyridin-2-yloxy)-phenoxy]-piperidine-1-carboxylic acid 4-[2-(pyridin-2-yloxy)-ethyl]-phenyl-ester,
4-[4-(5-Methyl-pyridin-2-yloxy)-phenoxy]-piperidine-1-carboxylic acid 4-[2-(pyridin-2-yloxy)-ethyl]-phenyl ester,
4-(4-Carboxy-phenoxymethyl)-piperidine-1-carboxylic acid 4-[2-(pyridin-2-yloxy)-ethyl]-phenyl ester,
4-(4-Carboxymethyl-phenoxymethyl)-piperidine-1-carboxylic acid 4-[2-(pyridin-2-yloxy)-ethyl]-phenyl ester,
4-(1H-Imidazol-2-ylsulfanylmethyl)-piperidine-1-carboxylic acid 4-[2-(pyridin-2-yloxy)-ethyl]-phenyl ester,
4-(1-Methyl-1H-imidazol-2-ylsulfanylmethyl)-piperidine-1-carboxylic acid 4-[2-(pyridin-2-yloxy)-ethyl]-phenyl ester,
4-(Imidazol-1-yloxymethyl)-piperidine-1-carboxylic acid 4-[2-(pyridin-2-yloxy)-ethyl]-phenyl ester,
4-[4-(Pyridin-2-yloxy)-phenoxymethyl]-piperidine-1-carboxylic acid 4-[2-(pyridin-2-yloxy)-ethyl]-phenyl ester,
4-[4-(5-Methyl-pyridin-2-yloxy)-phenoxymethyl]-piperidine-1-carboxylic acid 4-[2-(pyridin-2-yloxy)-ethyl]-phenyl ester,
4-(1-Methyl-1H-tetrazol-5-ylsulfanylmethyl)-piperidine-1-carboxylic acid 4-[2-(pyridin-2-yloxy)-ethyl]-phenyl ester,
4-[1-(2-Dimethylamino-ethyl)-1H-tetrazol-5-ylsulfanyl]-piperidine-1-carboxylic acid 4-[2-(pyridin-2-yloxy)-ethyl]-phenyl ester,
4-[1-(2-Carboxy-ethyl)-1H-tetrazol-5-ylsulfanyl]-piperidine-1-carboxylic acid 4-[2-(pyridin-2-yloxy)-ethyl]-phenyl ester,
4-(1-Carboxymethyl-1H-tetrazol-5-ylsulfanyl)-piperidine-1-carboxylic acid 4-[2-(pyridin-2-yloxy)-ethyl]-phenyl ester,
4-(Pyrazol-1-yloxymethyl)-piperidine-1-carboxylic acid 4-[2-(pyridin-2-yloxy)-ethyl]-phenyl ester,
4-(1H-[1,2,4]Triazol-3-ylsulfanylmethyl)-piperidine-1-carboxylic acid 4-[2-(pyridin-2-ethyl]-phenyl ester,
4-[2-(Benzyl-methyl-amino)-ethyl]-piperidine-1-carboxylic acid 4-[2-(pyridin-2-yloxy)-ethyl]-phenyl ester,
4-(1H-[1,2,4]Triazol-3-ylsulfanyl)-piperidine-1-carboxylic acid 4-[2-(pyridin-3-yloxy)-ethyl]-phenyl ester,
4-(1H-Imidazol-2-ylsulfanyl)-piperidine-1-carboxylic acid 4-[2-(pyridin-3-yloxy)-ethyl]-phenyl ester,
4-(1-Methyl-1H-imidazol-2-ylsulfanyl)-piperidine-1-carboxylic acid 4-[2-(pyridin-3-yloxy)-ethyl]-phenyl ester,
4-(1-Methyl-1H-tetrazol-5-ylsulfanyl)-piperidine-1-carboxylic acid 4-[2-(pyridin-3-yloxy)-ethyl]-phenyl ester,
4-(Imidazol-1-yloxy)-piperidine-1-carboxylic acid 4-[2-(pyridin-3-yloxy)-ethyl]-phenyl ester,
4-[1-(4-Carboxy-phenyl)-1H-tetrazol-5-ylsulfanyl]-piperidine-1-carboxylic acid 4-[2-(pyridin-3-yloxy)-ethyl]-phenyl ester,
4-(4-Carboxymethyl-phenoxy)-piperidine-1-carboxylic acid 4-[2-(pyridin-3-yloxy)-ethyl]-phenyl ester,
4-(Pyrazol-1-yloxy)-piperidine-1-carboxylic acid 4-[2-(pyridin-3-yloxy)-ethyl]-phenyl ester,
4-(4-Carboxy-phenoxy)-piperidine-1-carboxylic acid 4-[2-(pyridin-3-yloxy)-ethyl]-phenyl ester,
4-[4-(Pyridin-2-yloxy)-phenoxy]-piperidine-1-carboxylic acid 4-[2-(pyridin-3-yloxy)-ethyl]-phenyl ester,
4-[4-(5-Methyl-pyridin-2-yloxy)-phenoxy]-piperidine-1-carboxylic acid 4-[2-(pyridin-3-yloxy)-ethyl]-phenyl ester,
4-(4-Carboxy-phenoxymethyl)-piperidine-1-carboxylic acid 4-[2-(pyridin-3-yloxy)-ethyl]-phenyl ester,
4-(4-Carboxymethyl-phenoxymethyl)-piperidine-1l-carboxylic acid 4-[2-(pyridin-3-yloxy)-ethyl]-phenyl ester,
4-(1H-Imidazol-2-ylsulfanylmethyl)-piperidine-1-carboxylic acid 4-[2-(pyridin-3-yloxy)-ethyl]-phenyl ester,
4-(1-Methyl-1H-imidazol-2-ylsulfanylmethyl)-piperidine-1-carboxylic acid 4-[2-(pyridin-3-yloxy)-ethyl]-phenyl ester,
4-(Imidazol-1-yloxymethyl)-piperidine-1-carboxylic acid 4-[2-(pyridin-3-yloxy)-ethyl]-phenyl ester, 4-[4-(Pyridin-2-yloxy)-phenoxymethyl]-piperidine-1-carboxylic acid 4-[2-(pyridin-3-yloxy)-ethyl]-phenyl ester,
4-[4-(5-Methyl-pyridin-2-yloxy)-phenoxymethyl]-piperidine-1-carboxylic acid 4-[2-(pyridin-3-yloxy)-ethyl]-phenyl ester,
4-(1-Methyl-1H-tetrazol-5-ylsulfanylmethyl)-piperidine-1-carboxylic acid 4-[2-(pyridin-3-yloxy)-ethyl]-phenyl ester,
4-[1-(2-Dimethylamino-ethyl)-1H-tetrazol-5-ylsulfanyl]-piperidine-1-carboxylic acid 4-[2-(pyridin-3-yloxy)-ethyl]-phenyl ester,
4-[1-(2-Carboxy-ethyl)-1H-tetrazol-5-ylsulfanyl]-piperidine-1-carboxylic acid 4-[2-(pyridin-3-yloxy)-ethyl]-phenyl ester,
4-(1-Carboxymethyl-1H-tetrazol-5-ylsulfanyl)-piperidine-1-carboxylic acid 4-[2-(pyridin-3-yloxy)-ethyl]-phenyl ester,
4-(Pyrazol-1-yloxymethyl)-piperidine-1-carboxylic acid 4-[2-(pyridin-3-yloxy)-ethyl]-phenyl ester,
4-(1H-[1,2,4]Triazol-3-ylsulfanylmethyl)-piperidine-1-carboxylic acid 4-[2-(pyridin-3-ethyl]-phenyl ester,
4-[2-(Benzyl-methyl-amino)-ethyl]-piperidine-1-carboxylic acid 4-[2-(pyridin-3-yloxy)-ethyl]-phenyl ester,
4-(1H-[1,2,4]Triazol-3-ylsulfanyl)-piperidine-1-carboxylic acid 4-[2-(pyridin-4-yloxy)-phenyl ester,
4-(1H-Imidazol-2-ylsulfanyl)-piperidine-1-carboxylic acid 4-[2-(pyridin-4-yloxy)-ethyl]-phenyl ester,
4-(1-Methyl-1H-imidazol-2-ylsulfanyl)-piperidine-1-carboxylic acid 4-[2-(pyridin-4-yloxy)-ethyl]-phenyl ester,
4-(1-Methyl-1H-tetrazol-5-ylsulfanyl)-piperidine-1-carboxylic acid 4-[2-(pyridin-4-yloxy)-ethyl]-phenyl ester,
4-(Imidazol-1-yloxy)-piperidine-1-carboxylic acid 4-[2-(pyridin-4-yloxy)-ethyl]-phenyl ester,
4-[1-(4-Carboxy-phenyl)-1H-tetrazol-5-ylsulfanyl]-piperidine-1-carboxylic acid 4-[2-(pyridin-4-yloxy)-ethyl]-phenyl ester,
4-(4-Carboxymethyl-phenoxy)-piperidine-1-carboxylic acid 4-[2-(pyridin-4-yloxy)-ethyl]-phenyl ester,
4-(Pyrazol-1-yloxy)-piperidine-1-carboxylic acid 4-[2-(pyridin-4-yloxy)-ethyl]-phenyl ester,
4-(4-Carboxy-phenoxy)-piperidine-1-carboxylic acid 4-[2-(pyridin-4-yloxy)-ethyl]-phenyl ester,
4-[4-(Pyridin-2-yloxy)-phenoxy]-piperidine-1-carboxylic acid 4-[2-(pyridin-4-yloxy)-ethyl]-phenyl ester,
4-[4-(5-Methyl-pyridin-2-yloxy)-phenoxy]-piperidine-1-carboxylic acid 4-[2-(pyridin-4-yloxy)-ethyl]-phenyl ester,
4-(4-Carboxy-phenoxymethyl)-piperidine-1-carboxylic acid 4-[2-(pyridin-4-yloxy)-ethyl]-phenyl ester,
4-(4-Carboxymethyl-phenoxymethyl)-piperidine-1l-carboxylic acid 4-[2-(pyridin-4-yloxy)-ethyl]-phenyl ester,
4-(1H-Imidazol-2-ylsulfanylmethyl)-piperidine-1-carboxylic acid 4-[2-(pyridin-4-yloxy)-ethyl]-phenyl ester,
4-(1-Methyl-1H-imidazol-2-ylsulfanylmethyl)-piperidine-1-carboxylic acid 4-[2-(pyridin-4-yloxy)-ethyl]-phenyl ester,
4-(Imidazol-1-yloxymethyl)-piperidine-1-carboxylic acid 4-[2-(pyridin-4-yloxy)-ethyl]-phenyl ester,
4-[4-(Pyridin-2-yloxy)-phenoxymethyl]-piperidine-1-carboxylic acid 4-[2-(pyridin-4-yloxy)-ethyl]-phenyl ester,
4-[4-(5-Methyl-pyridin-2-yloxy)-phenoxymethyl]-piperidine-1-carboxylic acid 4-[2-(pyridin-4-yloxy)-ethyl]-phenyl ester,
4-(1-Methyl-1H-tetrazol-5-ylsulfanylmethyl)-piperidine-1-carboxylic acid 4-[2-(pyridin-4-yloxy)-ethyl]-phenyl ester,
4-[1-(2-Dimethylamino-ethyl)-1H-tetrazol-5-ylsulfanyl]-piperidine-1-carboxylic acid 4-[2-(pyridin-4-yloxy)-ethyl]-phenyl ester,
4-[1-(2-Carboxy-ethyl)-1H-tetrazol-5-ylsulfanyl]-piperidine-1-carboxylic acid 4-[2-(pyridin-4-yloxy)-ethyl]-phenyl ester,
4-(1-Carboxymethyl-1H-tetrazol-5-ylsulfanyl)-piperidine-1-carboxylic acid 4-[2-(pyridin-4-yloxy)-ethyl]-phenyl ester,
4-(Pyrazol-1-yloxymethyl)-piperidine-1-carboxylic acid 4-[2-(pyridin-4-yloxy)-ethyl]-phenyl ester,
4-(1H-[1,2,4]Triazol-3-ylsulfanylmethyl)-piperidine-1-carboxylic acid 4-[2-(pyridin-4-ethyl]-phenyl ester,
4-[2-(Benzyl-methyl-amino)-ethyl]-piperidine-1-carboxylic acid 4-[2-(pyridin-4-yloxy)-ethyl]-phenyl ester,
4-(1H-[1,2,4]Triazol-3-ylsulfanyl)-piperidine-1-carboxylic acid 4-[2-(6-methyl-pyridin-2-yl)-ethyl]-phenyl ester,
4-(1H-Imidazol-2-ylsulfanyl)-piperidine-1-carboxylic acid 4-[2-(6-methyl-pyridin-2-yl)-ethyl]-phenyl ester,
4-(1-Methyl-1H-imidazol-2-ylsulfanyl)-piperidine-1-carboxylic acid 4-[2-(6-methyl-pyridin-2-yl)-ethyl]-phenyl ester,
4-(1-Methyl-1H-tetrazol-5-ylsulfanyl)-piperidine-1-carboxylic acid 4-[2-(6-methyl-pyridin-yl)-ethyl]-phenyl ester,
4-(Imidazol-1-yloxy)-piperidine-1-carboxylic acid 4-[2-(6-methyl-pyridin-2-yl)-ethyl]-phenyl ester,
4-[1-(4-Carboxy-phenyl)-1H-tetrazol-5-ylsulfanyl]-piperidine-1-carboxylic acid 4-[2-(6-methyl-pyridin-2-yl)-ethyl]-phenyl ester,
4-(4-Carboxymethyl-phenoxy)-piperidine-1-carboxylic acid 4-[2-(6-methyl-pyridin-2-yl)-ethyl]-phenyl ester,
4-(Pyrazol-1-yloxy)-piperidine-1-carboxylic acid 4-[2-(6-methyl-pyridin-2-yl)-ethyl]-phenyl ester,
4-(4-Carboxy-phenoxy)-piperidine-1-carboxylic acid 4-[2-(6-methyl-pyridin-2-yl)-ethyl]-phenyl ester,
4-[4-(Pyridin-2-yloxy)-phenoxy]-piperidine-1-carboxylic acid 4-[2-(6-methyl-pyridin-2-yl)-ethyl]-phenyl ester,
4-[4-(5-Methyl-pyridin-2-yloxy)-phenoxy]-piperidine-1-carboxylic acid 4-[2-(6-methyl-pyridin-2-yl)-ethyl]-phenyl ester,
4-(4-Carboxy-phenoxymethyl)-piperidine-1-carboxylic acid 4-[2-(6-methyl-pyridin-2-yl)-ethyl]-phenyl ester,
4-(4-Carboxymethyl-phenoxymethyl)-piperidine-1-carboxylic acid 4-[2-(6-methyl-pyridin-2-yl)-ethyl]-phenyl ester,
4-(1H-Imidazol-2-ylsulfanylmethyl)-piperidine-1-carboxylic acid 4-[2-(6-methyl-pyridin-2-yl)-ethyl]-phenyl ester,
4-(1-Methyl-1H-imidazol-2-ylsulfanylmethyl)-piperidine-1-carboxylic acid 4-[2-(6-methyl-pyridin-2-yl)-ethyl]-phenyl ester,
4-(Imidazol-1-yloxymethyl)-piperidine-1-carboxylic acid 4-[2-(6-methyl-pyridin-2-yl)-ethyl]-phenyl ester,
4-[4-(Pyridin-2-yloxy)-phenoxymethyl]-piperidine-1-carboxylic acid 4-[2-(6-methyl-pyridin-2-yl)-ethyl]-phenyl ester,
4-[4-(5-Methyl-pyridin-2-yloxy)-phenoxymethyl]-piperidine-1-carboxylic acid 4-[2-(6-methyl-pyridin-2-yl)-ethyl]-phenyl ester,
4-(1-Methyl-1H-tetrazol-5-ylsulfanylmethyl)-piperidine-1-carboxylic acid 4-[2-(6-methyl-pyridin-2-yl)-ethyl]-phenyl ester,
4-[1-(2-Dimethylamino-ethyl)-1H-tetrazol-5-ylsulfanyl]-piperidine-1-carboxylic acid 4-[2-(6-methyl-pyridin-2-yl)-ethyl]-phenyl ester,
4-[1-(2-Carboxy-ethyl)-1H-tetrazol-5-ylsulfanyl]-piperidine-1-carboxylic acid 4-[2-(6-methyl-pyridin-2-yl)-ethyl]-phenyl ester,
4-(1-Carboxymethyl-1H-tetrazol-5-ylsulfanyl)-piperidine-1-carboxylic acid 4-[2-(6-methyl-pyridin-2-yl)-ethyl]-phenyl ester,
4-(Pyrazol-1-yloxymethyl)-piperidine-1-carboxylic acid 4-[2-(6-methyl-pyridin-2-yl)-ethyl]-phenyl ester,
4-(1H-[1,2,4]Triazol-3-ylsulfanylmethyl)-piperidine-1-carboxylic acid 4-[2-(6-methyl-pyridin-2-yl)-ethyl]-phenyl ester,
4-[2-(Benzyl-methyl-amino)-ethyl]-piperidine-1-carboxylic acid 4-[2-(6-methyl-pyridin-2-yl)-ethyl]-phenyl ester,
4-(1H-[1,2,4]Triazol-3-ylsulfanyl)-piperidine-1-carboxylic acid 4-[2-(4-methyl-pyridin-2-yl)-ethyl]-phenyl ester,
4-(1H-Imidazol-2-ylsulfanyl)-piperidine-1-carboxylic acid 4-[2-(4-methyl-pyridin-2-yl)-ethyl]-phenyl ester,
4-(1-Methyl-1H-imidazol-2-ylsulfanyl)-piperidine-1-carboxylic acid 4-[2-(4-methyl-pyridin-2-yl)-ethyl]-phenyl ester,
4-(1-Methyl-1H-tetrazol-5-ylsulfanyl)-piperidine-1-carboxylic acid 4-[2-(4-methyl-pyridin-2-yl)-ethyl]-phenyl ester,
4-(Imidazol-1-yloxy)-piperidine-1-carboxylic acid 4-[2-(4-methyl-pyridin-2-yl)-ethyl]-phenyl ester,
4-[1-(4-Carboxy-phenyl)-1H-tetrazol-5-ylsulfanyl]-piperidine-1-carboxylic acid 4-[2-(4-methyl-pyridin-2-yl)-ethyl]-phenyl ester,
4-(4-Carboxymethyl-phenoxy)-piperidine-1-carboxylic acid 4-[2-(4-methyl-pyridin-2-yl)-ethyl]-phenyl ester,
4-(Pyrazol-1-yloxy)-piperidine-1-carboxylic acid 4-[2-(4-methyl-pyridin-2-yl)-ethyl]-phenyl ester,
4-(4-Carboxy-phenoxy)-piperidine-1-carboxylic acid 4-[2-(4-methyl-pyridin-2-yl)-ethyl]-phenyl ester,
4-[4-(Pyridin-2-yloxy)-phenoxy]-piperidine-1-carboxylic acid 4-[2-(4-methyl-pyridin-2-yl)-ethyl]-phenyl ester,
4-[4-(5-Methyl-pyridin-2-yloxy)-phenoxy]-piperidine-1-carboxylic acid 4-[2-(4-methyl-pyridin-2-yl)-ethyl]-phenyl ester,
4-(4-Carboxy-phenoxymethyl)-piperidine-1-carboxylic acid 4-[2-(4-methyl-pyridin-2-yl)-ethyl]-phenyl ester,
4-(4-Carboxymethyl-phenoxymethyl)-piperidine-1-carboxylic acid 4-[2-(4-methyl-pyridin-2-yl)-ethyl]-phenyl ester,
4-(1H-Imidazol-2-ylsulfanylmethyl)-piperidine-1-carboxylic acid 4-[2-(4-methyl-pyridin-2-yl)-ethyl]-phenyl ester,
4-(1-Methyl-1H-imidazol-2-ylsulfanylmethyl)-piperidine-1-carboxylic acid 4-[2-(4-methyl-pyridin-2-yl)-ethyl]-phenyl ester,
4-(Imidazol-1-yloxymethyl)-piperidine-1-carboxylic acid 4-[2-(4-methyl-pyridin-2-yl)-ethyl]-phenyl ester,
4-[4-(Pyridin-2-yloxy)-phenoxymethyl]-piperidine-1-carboxylic acid 4-[2-(4-methyl-pyridin-2-yl)-ethyl]-phenyl ester,
4-[4-(5-Methyl-pyridin-2-yloxy)-phenoxymethyl]-piperidine-1-carboxylic acid 4-[2-(4-methyl-pyridin-2-yl)-ethyl]-phenyl ester,
4-(1-Methyl-1H-tetrazol-5-ylsulfanylmethyl)-piperidine-1-carboxylic acid 4-[2-(4-methyl-pyridin-2-yl)-ethyl]-phenyl ester,
4-[1-(2-Dimethylamino-ethyl)-1H-tetrazol-5-ylsulfanyl]-piperidine-1-carboxylic acid 4-[2-(4-methyl-pyridin-2-yl)-ethyl]-phenyl ester,
4-[1-(2-Carboxy-ethyl)-1H-tetrazol-5-ylsulfanyl]-piperidine-1-carboxylic acid 4-[2-(4-methyl-pyridin-2-yl)-ethyl]-phenyl ester,
4-(1-Carboxymethyl-1H-tetrazol-5-ylsulfanyl)-piperidine-1-carboxylic acid 4-[2-(4-methyl-pyridin-2-yl)-ethyl]-phenyl ester,
4-(Pyrazol-1-yloxymethyl)-piperidine-1-carboxylic acid 4-[2-(4-methyl-pyridin-2-yl)-ethyl]-phenyl ester,
4-(1H-[1,2,4]Triazol-3-ylsulfanylmethyl)-piperidine-1-carboxylic acid 4-[2-(4-methyl-pyridin-2-yl)-ethyl]-phenyl ester,
4-[2-(Benzyl-methyl-amino)-ethyl]-piperidine-1-carboxylic acid 4-[2-(4-methyl-pyridin-2-yl)-ethyl]-phenyl ester,
4-(1H-[1,2,4]Triazol-3-ylsulfanyl)-piperidine-1-carboxylic acid 4-[2-(3-methyl-pyridin-2-yl)-ethyl]-phenyl ester,
4-(1H-Imidazol-2-ylsulfanyl)-piperidine-1-carboxylic acid 4-[2-(3-methyl-pyridin-2-yl)-ethyl]-phenyl ester,
4-(1-Methyl-1H-imidazol-2-ylsulfanyl)-piperidine-1-carboxylic acid 4-[2-(3-methyl-pyridin-2-yl)-ethyl]-phenyl ester,
4-(1-Methyl-1H-tetrazol-5-ylsulfanyl)-piperidine-1-carboxylic acid 4-[2-(3-methyl-pyridin-2-yl)-ethyl]-phenyl ester,
4-(Imidazol-1-yloxy)-piperidine-1-carboxylic acid 4-[2-(3-methyl-pyridin-2-yl)-ethyl]-phenyl ester,
4-[1-(4-Carboxy-phenyl)-1H-tetrazol-5-ylsulfanyl]-piperidine-1-carboxylic acid 4-[2-(3-methyl-pyridin-2-yl)-ethyl]-phenyl ester,
4-(4-Carboxymethyl-phenoxy)-piperidine-1-carboxylic acid 4-[2-(3-methyl-pyridin-2-yl)-ethyl]-phenyl ester,
4-(Pyrazol-1-yloxy)-piperidine-1-carboxylic acid 4-[2-(3-methyl-pyridin-2-yl)-ethyl]-phenyl ester,
4-(4-Carboxy-phenoxy)-piperidine-1-carboxylic acid 4-[2-(3-methyl-pyridin-2-yl)-ethyl]-phenyl ester,
4-[4-(Pyridin-2-yloxy)-phenoxy]-piperidine-1-carboxylic acid 4-[2-(3-methyl-pyridin-2-yl)-ethyl]-phenyl ester,
4-[4-(5-Methyl-pyridin-2-yloxy)-phenoxy]-piperidine-1-carboxylic acid 4-[2-(3-methyl-pyridin-2-yl)-ethyl]-phenyl ester,
4-(4-Carboxy-phenoxymethyl)-piperidine-1-carboxylic acid 4-[2-(3-methyl-pyridin-2-yl)-ethyl]-phenyl ester,
4-(4-Carboxymethyl-phenoxymethyl)-piperidine-1-carboxylic acid 4-[2-(3-methyl-pyridin-2-yl)-ethyl]-phenyl ester,
4-(1H-Imidazol-2-ylsulfanylmethyl)-piperidine-1-carboxylic acid 4-[2-(3-methyl-pyridin-2-yl)-ethyl]-phenyl ester,
4-(1-Methyl-1H-imidazol-2-ylsulfanylmethyl)-piperidine-1-carboxylic acid 4-[2-(3-methyl-pyridin-2-yl)-ethyl]-phenyl ester,
4-(Imidazol-1-yloxymethyl)-piperidine-1-carboxylic acid 4-[2-(3-methyl-pyridin-2-yl)-ethyl]-phenyl ester,
4-[4-(Pyridin-2-yloxy)-phenoxymethyl]-piperidine-1-carboxylic acid 4-[2-(3-methyl-pyridin-2-yl)-ethyl]-phenyl ester,
4-[4-(5-Methyl-pyridin-2-yloxy)-phenoxymethyl]-piperidine-1-carboxylic acid 4-[2-(3-methyl-pyridin-2-yl)-ethyl]-phenyl ester,
4-(1-Methyl-1H-tetrazol-5-ylsulfanylmethyl)-piperidine-1-carboxylic acid 4-[2-(3-methyl-pyridin-2-yl)-ethyl]-phenyl ester,
4-[1-(2-Dimethylamino-ethyl)-1H-tetrazol-5-ylsulfanyl]-piperidine-1-carboxylic acid 4-[2-(3-methyl-pyridin-2-yl)-ethyl]-phenyl ester,
4-[1-(2-Carboxy-ethyl)-1H-tetrazol-5-ylsulfanyl]-piperidine-1-carboxylic acid 4-[2-(3-methyl-pyridin-2-yl)-ethyl]-phenyl ester,
4-(1-Carboxymethyl-1H-tetrazol-5-ylsulfanyl)-piperidine-1-carboxylic acid 4-[2-(3-methyl-pyridin-2-yl)-ethyl]-phenyl ester,
4-(Pyrazol-1-yloxymethyl)-piperidine-1-carboxylic acid 4-[2-(3-methyl-pyridin-2-yl)-ethyl]-phenyl ester,
4-(1H-[1,2,4]Triazol-3-ylsulfanylmethyl)-piperidine-1-carboxylic acid 4-[2-(3-methyl-pyridin-2-yl)-ethyl]-phenyl ester,
4-[2-(Benzyl-methyl-amino)-ethyl]-piperidine-1-carboxylic acid 4-[2-(3-methyl-pyridin-2-yl)-ethyl]-phenyl ester,
4-(1H-[1,2,4]Triazol-3-ylsulfanyl)-piperidine-1-carboxylic acid 4-[2-(5-methyl-pyridin-2-yl)-ethyl]-phenyl ester,
4-(1H-Imidazol-2-ylsulfanyl)-piperidine-1-carboxylic acid 4-[2-(5-methyl-pyridin-2-yl)-ethyl]-phenyl ester,
4-(1-Methyl-1H-imidazol-2-ylsulfanyl)-piperidine-1-carboxylic acid 4-[2-(5-methyl-pyridin-2-yl)-ethyl]-phenyl ester,
4-(1-Methyl-1H-tetrazol-5-ylsulfanyl)-piperidine-1-carboxylic acid 4-[2-(5-methyl-pyridin-yl)-ethyl]-phenyl ester,
4-(Imidazol-1-yloxy)-piperidine-1-carboxylic acid 4-[2-(5-methyl-pyridin-2-yl)-ethyl]-phenyl ester,
4-[1-(4-Carboxy-phenyl)-1H-tetrazol-5-ylsulfanyl]-piperidine-1-carboxylic acid 4-[2-(5-methyl-pyridin-2-yl)-ethyl]-phenyl ester,
4-(4-Carboxymethyl-phenoxy)-piperidine-1-carboxylic acid 4-[2-(5-methyl-pyridin-2-yl)-ethyl]-phenyl ester,
4-(Pyrazol-1-yloxy)-piperidine-1-carboxylic acid 4-[2-(5-methyl-pyridin-2-yl)-ethyl]-phenyl ester,
4-(4-Carboxy-phenoxy)-piperidine-1-carboxylic acid 4-[2-(5-methyl-pyridin-2-yl)-ethyl]-phenyl ester,
4-[4-(Pyridin-2-yloxy)-phenoxy]-piperidine-1-carboxylic acid 4-[2-(5-methyl-pyridin-2-yl)-ethyl]-phenyl ester,
4-[4-(5-Methyl-pyridin-2-yloxy)-phenoxy]-piperidine-1-carboxylic acid 4-[2-(5-methyl-pyridin-2-yl)-ethyl]-phenyl ester,
4-(4-Carboxy-phenoxymethyl)-piperidine-1-carboxylic acid 4-[2-(5-methyl-pyridin-2-yl)-ethyl]-phenyl ester,
4-(4-Carboxymethyl-phenoxymethyl)-piperidine-1-carboxylic acid 4-[2-(5-methyl-pyridin-2-yl)-ethyl]-phenyl ester,
4-(1H-Imidazol-2-ylsulfanylmethyl)-piperidine-1-carboxylic acid 4-[2-(5-methyl-pyridin-2-yl)-ethyl]-phenyl ester,
4-(1-Methyl-1H-imidazol-2-ylsulfanylmethyl)-piperidine-1-carboxylic acid 4-[2-(5-methyl-pyridin-2-yl)-ethyl]-phenyl ester,
4-(Imidazol-1-yloxymethyl)-piperidine-1-carboxylic acid 4-[2-(5-methyl-pyridin-2-yl)-ethyl]-phenyl ester,
4-[4-(Pyridin-2-yloxy)-phenoxymethyl]-piperidine-1-carboxylic acid 4-[2-(5-methyl-pyridin-2-yl)-ethyl]-phenyl ester,
4-[4-(5-Methyl-pyridin-2-yloxy)-phenoxymethyl]-piperidine-1-carboxylic acid 4-[2-(5-methyl-pyridin-2-yl)-ethyl]-phenyl ester,
4-(1-Methyl-1H-tetrazol-5-ylsulfanylmethyl)-piperidine-1-carboxylic acid 4-[2-(5-methyl-pyridin-2-yl)-ethyl]-phenyl ester,
4-[1-(2-Dimethylamino-ethyl)-1H-tetrazol-5-ylsulfanyl]-piperidine-1-carboxylic acid 4-[2-(5-methyl-pyridin-2-yl)-ethyl]-phenyl ester,
4-[1-(2-Carboxy-ethyl)-1H-tetrazol-5-ylsulfanyl]-piperidine-1-carboxylic acid 4-[2-(5-methyl-pyridin-2-yl)-ethyl]-phenyl ester,
4-(1-Carboxymethyl-1H-tetrazol-5-ylsulfanyl)-piperidine-1-carboxylic acid 4-[2-(5-methyl-pyridin-2-yl)-ethyl]-phenyl ester,
4-(Pyrazol-1-yloxymethyl)-piperidine-1-carboxylic acid 4-[2-(5-methyl-pyridin-2-yl)-ethyl]-phenyl ester,
4-(1H-[1,2,4]Triazol-3-ylsulfanylmethyl)-piperidine-1-carboxylic acid 4-[2-(5-methyl-pyridin-2-yl)-ethyl]-phenyl ester, and
4-[2-(Benzyl-methyl-amino)-ethyl]-piperidine-1-carboxylic acid 4-[2-(5-methyl-pyridin-2-yl)-ethyl]-phenyl ester.

68. A pharmaceutical composition comprising a compound according to claim 1, or a pharmaceutically acceptable salt thereof together with a pharmaceutically acceptable carrier or diluent.

69. The composition according to claim 68, wherein said composition is in unit dosage form, comprising from about 0.05 to about 2000 mg, from about 0.1 to about 500 mg, or from about 1.0 to about 100 mg of said compound, or pharmaceutically acceptable salt thereof.

70. A pharmaceutical composition for use as a medicament for inhibiting the lipolytic activity of hormone-sensitive lipase against triacylglycerols, diacylglycerols, cholesterol acyl esters or steroid acyl esters, said composition comprising a compound according to claim 1, or a pharmaceutically acceptable salt thereof together with a pharmaceutically acceptable carrier or diluent.

71. A pharmaceutical composition according to claim 68, which is for oral administration.

72. A pharmaceutical composition according to claim 68, for nasal, transdermal, pulmonal, or parenteral administration.

73. A method of treating a disorder of a patient where modulation of the activity of hormone-sensitive lipase is desired, the method comprising administering to a subject in need thereof a therapeutically effective amount of a compound according to claim 1, or a pharmaceutically acceptable salt thereof.

74. A method of treating a disorder of a patient where lowering of the activity of hormone-sensitive lipase is desired, the method comprising administering to a subject in need thereof a therapeutically effective amount of a compound according to claim 1, or a pharmaceutically acceptable salt thereof.

75. The method according to claim 73, wherein said administration is carried out by the oral, nasal, transdermal, pulmonal, or parenteral route.

76. The method according to claim 73, wherein said disorder is selected from the group consisting of insulin resistance, diabetes type 1, diabetes type 2, metabolic syndrome X, impaired glucose tolerance, hyperglycemia, dyslipidemia, obesity, atheroschlerosis, hypertension, abnormalities of lipoprotein metabolism and any combination thereof.

77. The method according to claim 73, wherein the therapeutically effective amount of the compound is from about 0.05 to about 2000 mg, from about 0.1 to about 500 mg, or from about 1.0 to about 100 mg of said compound per day.

78. The method according to claim 73, wherein a further antidiabetic, antiobesity, antihypertensive or appetite regulating drug is administered to the patient.

79. The method according to claim 73, wherein mefformin is also administered to the patient.

80. A process for the preparation of a compound according to claim 1, or its pharmaceutically acceptable salt, which comprises reacting the appropriate alcohol with the appropriate carbamoylating reagent in a solvent according to the reaction scheme P₁ 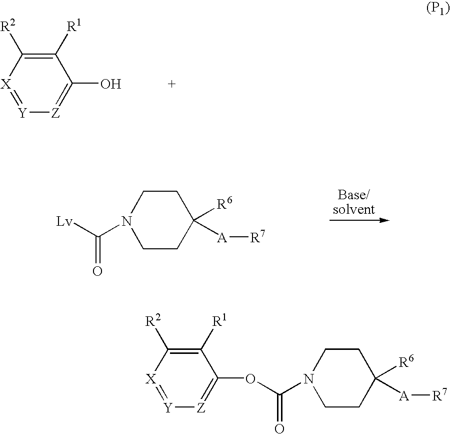
and isolating the disubstituted carbamate product.

81. The process according to claim 80, wherein said carbamoylating reagent 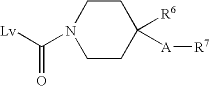
is selected from the group consisting of

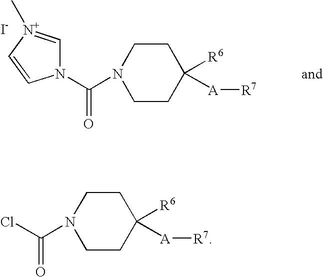

82. The process according to claim 80, wherein said solvent is selected from the group consisting of tetrahydrofurane, dimethylformamide and N-methylpyrolidone.

83. The process according to claim 80, wherein said base is selected from the group consisting of triethylamine, N,N-diisopropyl-N-ethylamine and DABCO.

84. A process for the preparation of a compound according to claim 1, said process comprising the treatment of the appropriate amine with the appropriate acylating reagent in a solvent and in the presence of a base according to the reaction scheme P₂ 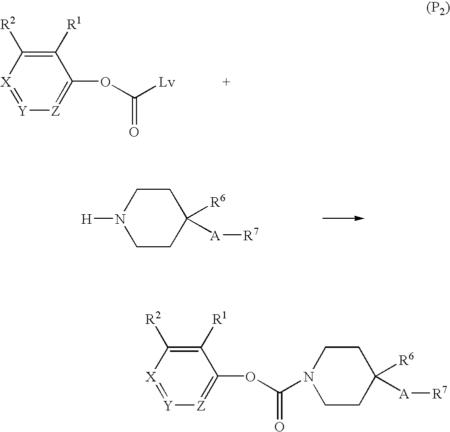
and isolating the disubstituted carbamate.

85. The process according to claim 84, wherein Lv is Cl.

86. The process according to claim 84, wherein said solvent is selected from the group consisting of diethyl ether, tetrahydrofuran and dichloromethane.

87. The process according to claim 84, wherein said base is selected from the group consisting of trimethylamine, triethylamine, ethyl-diisopropyl-amine and 1,4-diazabicyclo[2.2.2]octane.

88. The process according to claim 84, wherein said base is present as a functionality in one or both of the substituents R⁶ and R⁷, thus forming a salt with the acid H-Lv.

89. A process for the preparation of a compound according to claim 1, said process comprising the treatment of the appropriate alcohol (A′ is selected from OH or CH₂OH) with the appropriate hydroxy containing Mitzunobu reagent (HO—R⁷) or thiol containing Mitzunobu reagent (HS—R⁷) under standard mitzunobu conditions as described in reaction scheme P₃ or P₄: 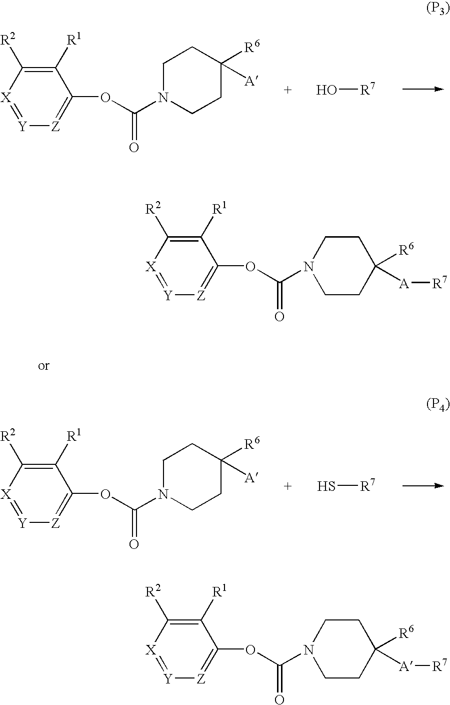
and isolating the disubstituted carbamate.

90. A compound according to claim 1, where the compound is selected from the group consisting of
4-(1-Methyl-1H-tetrazol-5-ylsulfanyl)-piperidine-1-carboxylic acid 4-(5-methyl-pyridin-2-ylmethyl)-phenyl ester,
4-Pyridin-2-ylmethyl-piperidine-1-carboxylic acid 4-(5-trifluoromethyl-pyridin-2-yloxy)-phenyl ester,
4-Pyridin-2-ylmethyl-piperidine-1-carboxylic acid 4-(4-trifluoromethyl-benzyl)-phenyl ester,
4-Pyrimidin-2-ylmethyl-piperidine-1-carboxylic acid 4-(5-trifluoromethyl-pyridin-2-yloxy)-phenyl ester,
4-Pyrimidin-2-ylmethyl-piperidine-1-carboxylic acid 4-(4-trifluoromethyl-benzyl)-phenyl ester,
4-Thiophen-3-ylmethyl-piperidine-1-carboxylic acid 4-(5-trifluoromethyl-pyridin-2-yloxy)-phenyl ester,
4-Thiophen-3-ylmethyl-piperidine-1-carboxylic acid 4-(4-trifluoromethyl-benzyl)-phenyl ester,
4-Thiophen-3-ylmethyl-piperidine-1-carboxylic acid 4-(5-methyl-pyridin-2-ylmethyl)-phenyl ester,
4-(5-Methyl-pyridin-2-ylmethyl)-piperidine-1-carboxylic acid 4-(4-trifluoromethyl-benzyl)-phenyl ester,
4-(5-Methyl-pyridin-2-ylmethyl)-piperidine-1-carboxylic acid 4-(5-trifluoromethyl-pyridin-2-yloxy)-phenyl ester,
4-(4-Carboxymethyl-benzyl)-piperidine-1-carboxylic acid 4-(5-trifluoromethyl-pyridin-2-yloxy)-phenyl ester,
4-(4-Carboxymethyl-benzyl)-piperidine-1-carboxylic acid 4-(4-trifluoromethyl-benzyl)-phenyl ester,
4-[4-(2H-Tetrazol-5-ylmethyl)-benzyl]-piperidine-1-carboxylic acid 4-(5-trifluoromethyl-pyridin-2-yloxy)-phenyl ester,
4-[4-(2H-Tetrazol-5-ylmethyl)-benzyl]-piperidine-1-carboxylic acid 4-(4-trifluoromethyl-benzyl)-phenyl ester,
4-(4-Methyl-pyridin-2-ylmethyl)-piperidine-1-carboxylic acid 4-(5-trifluoromethyl-pyridin-2-yloxy)-phenyl ester,
4-[4-(1H-Tetrazol-5-ylmethyl)-phenoxy]-piperidine-1-carboxylic acid 4-(5-trifluoromethyl-pyridin-2-yloxy)-phenyl ester,
4-(6-Hydroxymethyl-pyridin-2-ylmethyl)-piperidine-1-carboxylic acid 4-(5-trifluoromethyl-pyridin-2-yloxy)-phenyl ester,
4-[4-(1H-Tetrazol-5-ylmethyl)-phenoxy]-piperidine-1-carboxylic acid 4-(4-trifluoromethyl-benzyl)-phenyl ester,
4-(6-Hydroxymethyl-pyridin-2-ylmethyl)-piperidine-1-carboxylic acid 4-(4-trifluoromethyl-benzyl)-phenyl ester,
4-(4-Methyl-pyridin-2-ylmethyl)-piperidine-1-carboxylic acid 4-(4-trifluoromethyl-benzyl)-phenyl ester,
4-(4-Methyl-pyridin-2-ylmethyl)-piperidine-1-carboxylic acid 4-(5-methyl-pyridin-2-ylmethyl)-phenyl ester,
(Carboxymethyl-phenoxy)-piperidine-1-carboxylic acid 4-(3-methyl-butylcarbamoyl)-phenyl ester,
4-(4-Carboxymethyl-phenoxy)-piperidine-1-carboxylic acid 4-phenethylcarbamoyl-phenyl ester,
4-(4-Carboxymethyl-phenoxy)-piperidine-1-carboxylic acid 4-benzylcarbamoyl-phenyl ester,
4-(4-Carboxymethyl-phenoxy)-piperidine-1-carboxylic acid 4-[(bicyclo[2.2.1]hept-2-ylmethyl)-carbamoyl]-phenyl ester,
4-(4-Carboxymethyl-phenoxy)-piperidine-1-carboxylic acid 4-(cyclohexylmethyl-carbamoyl)-phenyl ester,
4-(4-Carboxymethyl-phenoxy)-piperidine-1-carboxylic acid 4-(3,3-dimethyl-butylcarbamoyl)-phenyl ester,
4-(4-Carboxymethyl-phenoxy)-piperidine-1-carboxylic acid 4-[(thiophen-2-ylmethyl)-carbamoyl]-phenyl ester,
4-(4-Carboxymethyl-phenoxy)-piperidine-1-carboxylic acid 4-(2-thiophen-2-yl-ethylcarbamoyl)-phenyl ester,
4-(4-Carboxymethyl-phenoxy)-piperidine-1-carboxylic acid 4-(2-phenoxy-ethyl)-phenyl ester,
4-(4-Carboxymethyl-phenoxy)-piperidine-1-carboxylic acid 4-[2-(4-chloro-phenoxy)-ethyl]-phenyl ester,
4-(4-Carboxymethyl-phenoxy)-piperidine-1-carboxylic acid 4-[2-(1-methyl-1H-tetrazol-5-yloxy)-ethyl]-phenyl ester,
4-(4-Carboxymethyl-phenoxy)-piperidine-1-carboxylic acid 4-[2-(3-chloro-phenoxy)-ethyl]-phenyl ester,
4-(4-Carboxymethyl-phenoxy)-piperidine-1-carboxylic acid 4-[2-(4-trifluoromethoxy-phenoxy)-ethyl]-phenyl ester,
4-(4-Carboxymethyl-phenoxy)-piperidine-1-carboxylic acid 4-[2-(4-[1,2,4]triazol-4-yl-phenoxy)-ethyl]-phenyl ester,
4-(4-Carboxymethyl-phenoxy)-piperidine-1-carboxylic acid 4-(2-phenylsulfanyl-ethyl)-phenyl ester,
4-(4-Carboxymethyl-phenoxy)-piperidine-1-carboxylic acid 4-[2-(2-chloro-phenoxy)-ethyl]-phenyl ester,
4-(4-Carboxymethyl-phenoxy)-piperidine-1-carboxylic acid 4-[2-(4-methyl4H-[1,2,4]triazol-3-ylsulfanyl)-ethyl]-phenyl ester,
4-(4-Carboxymethyl-phenoxy)-piperidine-1-carboxylic acid 4-[2-(4-pyrrol-1-yl-phenoxy)-ethyl]-phenyl ester,
4-(4-Carboxymethyl-phenoxy)-piperidine-1-carboxylic acid 4-(3-chloro-5-trifluoromethyl-pyridin-2-ylmethyl)-phenyl ester,
4-(4-Carboxymethyl-phenoxy)-piperidine-1-carboxylic acid 4-(4-trifluoromethyl-phenoxy)-phenyl ester,
4-(1H-Imidazol-2-ylsulfanyl)-piperidine-1-carboxylic acid 4-(3,3-dimethyl-butylcarbamoyl)-phenyl ester,
4-(4-Carboxy-phenoxy)-piperidine-1-carboxylic acid 4-(3,3-dimethyl-butylcarbamoyl)-phenyl ester,
4-(4-Carboxy-benzyl)-piperidine-1-carboxylic acid 4-(5-trifluoromethyl-pyridin-2-yloxy)-phenyl ester,
4-(1H-Benzoimidazol-2-ylsulfanyl)-piperidine-1-carboxylic acid 4-(5-trifluoromethyl-pyridin-2-yloxy)-phenyl ester,
4-(4-Carboxy-1H-imidazol-2-ylsulfanyl)-piperidine-1-carboxylic acid 4-(5-trifluoromethyl-pyridin-2-yloxy)-phenyl ester,
6-{1-[4-(5-Trifuoromethyl-pyridin-2-yloxy)-phenoxycarbonyl]-piperidin-4-yloxy}-nicotinic acid,
4-(3-Carboxymethyl-phenoxy)-piperidine-1-carboxylic acid 4-(5-trifluoromethyl-pyridin-2-yloxy)-phenyl ester,
4-(4-Carboxymethyl-2-fluoro-phenoxy)-piperidine-1-carboxylic acid 4-(5-trifluoromethyl-pyridin-2-yloxy)-phenyl ester,
4-[3-(2-Carboxy-ethyl)-phenoxy]-piperidine-1-carboxylic acid 4-(5-trifluoromethyl-pyridin-2-yloxy)-phenyl ester,
4-(5-Carboxymethylsulfanyl-[1,3,4]thiadiazol-2-ylsulfanyl)-piperidine-1-carboxylic acid 4-(5-trifluoromethyl-pyridin-2-yloxy)-phenyl ester,
4-(5-carboxymethyl-4-methyl-oxazol-2-ylsulfanyl)-piperidine-1-carboxylic acid 4-(5-trifluoromethyl-pyridin-2-yloxy)-phenyl ester,
4-(4-Carboxymethoxy-phenoxy)-piperidine-1-carboxylic acid 4-(5-trifluoromethyl-pyridin-2-yloxy)-phenyl ester,
4-(4-Carboxymethyl-phenoxy)-piperidine-1-carboxylic acid 4-(3-trifluoromethyl-phenoxy)-phenyl ester,
4-(4-Carboxymethyl-phenoxy)-piperidine-1-carboxylic acid 4-(5-chloro-pyridin-2-yloxy)-phenyl ester,
4-(4-Carboxymethyl-phenoxy)-piperidine-1-carboxylic acid 4-(3-chloro-5-trifluoromethyl-pyridin-2-yloxy)-phenyl ester, and
4-(4-Carboxymethyl-phenoxy)-piperidine-1-carboxylic acid 4-p-tolyloxy-phenyl ester.
